(19) United States
(12) Patent Application Publication
(10) Pub. No.: US 2025/0320496 A1
(43) Pub. Date: Oct. 16, 2025

(54) MODULATION OF ABCB11 GENE TRANSCRIPTION USING ANTISENSE OLIGONUCLEOTIDES TARGETING REGULATORY RNAS

(71) Applicant: CAMP4 Therapeutics Corporation, Cambridge, MA (US)

(72) Inventors: Brian E. Schwartz, Somerville, MA (US); Alfica Sehgal, Belmont, MA (US); David A. Bumcrot, Belmont, MA (US); Keerthana Gnanapradeepan, Stoneham, MA (US); Mateo John Rodriguez, Cambridge, MA (US); Rachana S. Kelkar, Malden, MA (US); Yuting Liu, Lexington, MA (US); Rutuja Sudhakar Pai, Charlestown, MA (US)

(21) Appl. No.: 19/196,169

(22) Filed: May 1, 2025

Related U.S. Application Data
(63) Continuation of application No. PCT/US2023/078736, filed on Nov. 3, 2023.
(60) Provisional application No. 63/382,281, filed on Nov. 3, 2022.

(51) Int. Cl. C12N 15/113; A61P 1/16

(52) U.S. Cl. CPC C12N 15/113; A61P 1/16; C12N 2310/113; C12N 2310/315; C12N 2310/321; C12N 2310/3231; C12N 2310/3341; C12N 2310/351

(57) ABSTRACT

Described herein are methods of modulating ABCB11 gene transcription using antisense oligonucleotides (ASOs) targeting regulatory RNAs, such as promoter-associated RNAs and enhancer RNAs. These methods are useful for increasing the expression of ABCB11 mRNA and protein to treat subjects having or at risk of developing cholestasis or a cholestatic liver disease, such as primary biliary cholangitis and progressive familial intrahepatic cholestasis.

### CROSS REFERENCE TO RELATED APPLICATIONS

This application is a continuation of International Application No. PCT/US2023/078736, filed on Nov. 3, 2023, which claims the benefit of U.S. Provisional Application No. 63/382,281, filed Nov. 3, 2022, each of which are hereby incorporated in their entirety by reference.

### SEQUENCE LISTING

The instant application contains a Sequence Listing which has been submitted via PatentCenter and is hereby incorporated by reference in its entirety. Said XML copy, created on Dec. 13, 2023, is named CTC-031WO_SL.xml, and is 8,339,905 bytes in size.

### BACKGROUND

Transcription factors bind specific sequences in promoter and enhancer DNA elements to regulate gene transcription. It was recently reported that active promoters and enhancer elements are themselves transcribed, generating noncoding regulatory RNAs (regRNAs) such as promoter-associated RNAs (paRNAs) and enhancer RNAs (eRNAs) (see Sartorelli and Lauberth, Nat. Struct. Mol. Biol. (2020) 27, 521-28). Unlike coding RNAs, regRNAs are transcribed bi-directionally. Various models have been proposed for the functions of regRNAs, including nucleosome remodeling (see Mousavi et al., Mol. Cell (2013) 51 (5): 606-17), modulation of enhancer-promoter looping (see Lai et al., Nature (2013) 494 (7438): 497-501), and direct interaction with transcription regulators (see Sigova et al., Science (2015) 350, 978-81).

Primary biliary cholangitis (PBC) is an autoimmune disease characterized by the progressive destruction of bile duct cells, leading to accumulation of bile acids and ultimately intrahepatic cholestasis. Cholestasis is the impairment of bile flow from the liver and is primarily responsible for liver injury. If left untreated, patients with PBC or cholestasis may ultimately require a liver transplant. There is no cure for PBC, and 30-40% of patients will not respond to the first line treatment, ursodeoxycholic acid (UDCA). Currently, the only approved second line therapy is obeticholic acid (OCA), which has demonstrated limited efficacy and a poor tolerability profile. Therefore, there is significant unmet need for new PBC and cholestasis therapeutics.

Gene expression has been generally known as an undruggable biological process. Despite on-going efforts into understanding the biology of gene transcription and regRNAs, clinically suitable methods of modulating gene expression are limited. There remains a need for new and useful methods for treating diseases associated with aberrant gene expression.

### SUMMARY

In one aspect, provided herein are antisense oligonucleotides (ASO) complementary to at least 8 contiguous nucleotides of a regulatory RNA of human ABCB11, wherein the regulatory RNA has a nucleotide sequence selected from the group consisting of SEQ ID NOs: 1-5 and 7-10.

In some embodiments, the ASO is complementary to a sequence in the regRNA that is no more than 200 nucleotides from the 3′ end of the regRNA.

In some embodiments, the ASO is complementary to a sequence in the regRNA that is no more than 200 nucleotides from the 5′ end of the regRNA.

In some embodiments, the regRNA is not a polyadenylated RNA.

In some embodiments, the regulatory RNA has a nucleotide sequence of SEQ ID NO: 1, and the ASO comprises a nucleotide sequence selected from the group consisting of SEQ ID NOs: 104-138, 217-272, 516-528, 645-1517, 5610-5644, 5723-5778, and 6018-6021.

In some embodiments, the regulatory RNA has a nucleotide sequence of SEQ ID NO: 2, and the ASO comprises a nucleotide sequence selected from the group consisting of SEQ ID NOs: 14-103, 273-381, 390-407, 463, 467-478, 515, 644, 1518-2057, 5520-5609, 5779-5887, 5896-5913, 5969, 5971-5980, and 6017.

In some embodiments, the regulatory RNA has a nucleotide sequence of SEQ ID NO: 3, and the ASO comprises a nucleotide sequence selected from the group consisting of SEQ ID NOs: 14-103, 139-148, 273-407, 463, 467-478, 515, 530-623, 625-644, 1518-2308, 5520-5609, 5645-5654, 5779-5913, 5969, 5971-5980, and 6017.

In some embodiments, the regulatory RNA has a nucleotide sequence of SEQ ID NO: 4, and the ASO comprises a nucleotide sequence selected from the group consisting of SEQ ID NOs: 149-168, 408-462, 464, 479-514, 2309-3956, 5655-5674, 5914-5968, 5970, and 5981-6016.

In some embodiments, the regulatory RNA has a nucleotide sequence of SEQ ID NO: 5, and the ASO comprises a nucleotide sequence selected from the group consisting of SEQ ID NOs: 169-216, 4062-5228, and 5675-5722.

In some embodiments, the regulatory RNA has a nucleotide sequence of SEQ ID NO: 7, and the ASO comprises a nucleotide sequence selected from the group consisting of SEQ ID NOs: 645-954.

In some embodiments, the regulatory RNA has a nucleotide sequence of SEQ ID NO: 8, and the ASO comprises a nucleotide sequence selected from the group consisting of SEQ ID NOs: 2309-2806.

In some embodiments, the regulatory RNA has a nucleotide sequence of SEQ ID NO: 9, and the ASO comprises a nucleotide sequence selected from the group consisting of SEQ ID NOs: 3957-4061.

In some embodiments, the regulatory RNA has a nucleotide sequence of SEQ ID NO: 10, and the ASO comprises a nucleotide sequence selected from the group consisting of SEQ ID NOs: 5229-5519.

In some embodiments, the ASO is no more than 50, 40, 30, or 25 nucleotides in length.

In some embodiments, the ASO comprises a RNA polynucleotide comprising one or more chemical modifications.

In some embodiments, at least 3, 4, or 5 nucleotides at the 5′ end and at least 3, 4, or 5 nucleotides at the 3′ end of the ASO comprise ribonucleotides with one or more chemical modifications.

In some embodiments, the one or more chemical modifications comprise a nucleotide sugar modification comprising one or more of 2′-O-C1-4alkyl such as 2′-O-methyl (2′-OMe), 2′-deoxy (2′-H), 2′-O—C1-3alkyl-O-C1-3alkyl such as 2′-methoxyethyl (“2′-MOE”), 2′-fluoro (“2′-F”), 2′-amino (“2′—NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (CET), 2′-fluoro-arabino (FANA), or thiomorpholino.

In some embodiments, the one or more chemical modifications comprise an internucleotide linkage modification comprising one or more of phosphorothioate (“PS” or (P(S))), phosphoramidate (P(NR₁R₂) such as dimethylaminophosphoramidate (P(N(CH₃)₂)), phosphonocarboxylate (P(CH₂)ₙCOOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂COO⁻)), alkylphosphonate (P(C1-3alkyl) such as methylphosphonate —P(CH₃), boranophosphonate (P(BH₃)), or phosphorodithioate (P(S)₂).

In some embodiments, the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

In some embodiments, the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

In some embodiments, the ASO further comprises a GalNAc moiety, optionally a GalNAc3 moiety.

In some embodiments, the ASO does not comprise 10 or more contiguous nucleotides of unmodified DNA.

In some embodiments, the ASO does not comprise a deoxyribonucleotide.

In some embodiments, the ASO does not comprise an unmodified ribonucleotide.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

In some embodiments, each ribonucleotide of the ASO is modified by 2′-O-methoxyethyl.

In some embodiments, each nucleotide of the ASO is a ribonucleotide modified by 2′-O-methoxyethyl.

In some embodiments, the ASO comprises 10 or more contiguous nucleotides of unmodified DNA flanked by at least 3 nucleotides of modified ribonucleotides at each of the 5′ end and the 3′ end.

In some embodiments, each cytidine in the ASO is modified by 5-methyl.

In some embodiments, the regRNA is an eRNA.

In some embodiments, the regRNA is an paRNA.

In another aspect, provided herein are pharmaceutical compositions comprising the ASO as described herein and a pharmaceutically acceptable carrier or excipient carrier.

In another aspect, provided herein are methods of increasing transcription of ABCB11 in a human cell, the method comprising contacting the cell with an ASO as described herein or a pharmaceutical composition as described herein.

In some embodiments, the cell is a hepatocyte.

In some embodiments, the ASO increases the amount of the regulatory RNA in the cell.

In some embodiments, the ASO increases the stability of the regulatory RNA in the cell.

In another aspect, provided herein are methods of treating or preventing cholestasis in a subject, the method comprising administering to a subject in need thereof an effective amount of the ASO disclosed herein or the pharmaceutical composition disclosed herein.

In some embodiments, the subject has or is at risk of developing a cholestatic liver disease.

In some embodiments, the cholestatic liver disease is progressive familial intrahepatic cholestasis (PFIC), Alagille Syndrome (ALGS), primary sclerosing cholangitis (PSC), neonatal sclerosing cholangitis, pediatric intrahepatic cholestasis (e.g., pediatric primary intrahepatic cholestasis or pediatric secondary intrahepatic cholestasis), benign recurrent intrahepatic cholestasis (BRIC), total parenteral nutrition associated cholestasis, paraneoplastic cholestasis, Stauffer syndrome, drug-associated cholestasis, infection-associated cholestasis, biliary atresia, intrahepatic cholestasis of pregnancy, primary biliary cholangitis (PBC), Dubin-Johnson syndrome, post-Kasai biliary atresia, post-liver transplantation biliary atresia, post-liver transplantation cholestasis, post-liver transplantation associated liver disease, intestinal failure associated liver disease, bile acid mediated liver injury, MRP3 deficiency syndrome, or gallstone disease.

In some embodiments, the subject has or is at risk of developing PFIC, and wherein the PFIC is PFIC type 3.

In some embodiments, the subject has or is at risk of developing PBC.

In some embodiments, the method results in a reduction in a symptom or a change in a disease-relevant laboratory measure of a cholestatic liver disease in the subject.

In some embodiments, the reduction in a symptom or change in the disease-relevant laboratory measure of cholestatic liver disease comprises a reduction in serum bile acid concentration, an increase in serum concentration of 7α-hydroxy-4-cholesten-3-one (7αC4), an increase in the ratio of serum 7αC4: serum bile acid, an increase in fecal bile acid excretion, a reduction in pruritis, a reduction in serum alanine aminotransferase (ALT) concentration, or a combination thereof.

In another aspect, provided herein are methods of treating primary biliary cholangitis (PBC) or cholestasis, the method comprising administering to a subject in need thereof an effective amount of the ASO as described herein or the pharmaceutical composition as described herein.

In some embodiments, administration of the ASO increases ABCB11 gene expression relative to a pre-administration baseline level.

In some embodiments, the ASO increases the amount of the regulatory RNA in a cell of the subject.

In some embodiments, the ASO increases the stability of the regulatory RNA in a cell of the subject.

In some embodiments, the cell is a hepatocyte.

### BRIEF DESCRIPTION OF THE DRAWINGS

FIG. 1A shows an illustrative schematic of eRNA, paRNA, mRNA, and natural antisense transcript (NAT) of a gene on the chromosome. The eRNA, paRNA, and NAT are all non-coding RNAs. The eRNA is transcribed bidirectionally from an enhancer of the gene. The paRNA is transcribed from the promoter of the gene, same as the mRNA, but in the antisense direction. The NAT is transcribed from a downstream promoter of its own in the antisense direction, such that the transcript overlaps at least partially with the mRNA. Generally, eRNAs and paRNAs upregulate gene expression whereas NATs downregulate gene expression. FIG. 1B shows an illustrative schematic of the interaction of regRNA with enhancer and promoter regions to recruit transcription factors and regulators that modulate gene expression. FIG. 1C provides a diagram of the identified regRNAs aligned with the human ABCB11 gene and active transcription sites.

FIG. 2A shows a dose-dependent increase in ABCB11 mRNA levels in primary human hepatocytes (PHHs) after treatment with ASOs CO-3986 (targeting paRNA RR63) or CO-4529 (targeting eRNA RR64). FIG. 2B shows a dose-dependent increase in ABCB11 mRNA levels (relative to beta-2-microglobulin (B2M)) in primary human hepatocytes after treatment with ASOs CO-3986, CO-4535 or CO-4556.

FIGS. 3A and 3B show that treatment of PHHs with ABCB11 regRNA targeting ASOs CO-3986, CO-4535, or CO-4556 induced increased expression of ABCB11 mRNA levels (relative to B2M) (FIG. 3A), and also induce increased expression of CYP7A1mRNA levels (relative to B2M) (FIG. 3B), both in a dose dependent manner.

FIGS. 4A, 4B, 4C, and 4D show that treatment of PHHs with ASOs including additional chemical modifications to parent ASOs CO-4535, CO-4529, CO-4517, and CO-3986 upregulated human ABCB11 mRNA expression greater than or equal to 1.5-fold as compared to untreated PHHs (“Untreated”). FIG. 4A shows ABCB11 mRNA levels in PHHs after treatment with derivative ASOs based on parent ASO CO-4535 (i.e., CO-7166, CO-7172, CO-7186, CO-7187, CO-7188, CO-7189, CO-7190, CO-7191, CO-7193, and CO-7194) as compared to untreated PHHs. FIG. 4B shows ABCB11 mRNA levels in PHHs after treatment with derivative ASOs based on parent ASO CO-4517 (i.e., CO-7141, CO-7148, CO-7149, CO-7150, and CO-7151) as compared to untreated PHHs. FIG. 4C shows ABCB11 mRNA levels of PHHs treated with derivative ASOs based on parent ASO CO-3986 (i.e., CO-7081 and CO-7082) as compared to untreated PHHs. FIG. 4D shows ABCB11 mRNA levels of PHHs treated with derivative ASOs based on parent ASO CO-4529 (i.e., CO-7175, CO-7178, CO-7179, CO-7180, CO-7181, CO-7182, and CO-7184) as compared to untreated PHHs. FC=fold change.

FIGS. 5A and 5B show dose-responsive increases in ABCB11 mRNA levels (FIG. 5A) and CYP7A1 mRNA levels (FIG. 5B), relative to B2M levels, on Day 4 (D4) in PHHs following treatment with the indicated concentrations of ASOs CO-4513, CO-4535 or CO-4556, as compared to untreated PHHs (“Untreated”), and PHHs treated with a control downregulating gapmer ASO targeting ABCB11 mRNA (CO-3760), or either 0.3 μM or 3 μM OBA.

FIGS. 6A and 6B show ABCB11 (FIG. 6A) or CYP7A1 (FIG. 6B) mRNA levels (normalized to respective untreated timepoint) in PHHs contacted with ASO CO-4535 as compared with control untreated PHHs at Days 2 (D2), 3 (D3), 4 (D4), 5 (D5), 6 (D6), 7 (D7). Strong ABCB11 and CYP7A1 gene upregulation was observed on Day 4 of treatment with ASO CO-4535 indicating effective bile acid efflux.

FIGS. 7A, 7B and 7C show that ABCB11 mRNA levels (FIG. 7A) and CYP7A1 mRNA levels (FIG. 7B) were upregulated in PHHs in a dose-responsive manner after treatment with ASO CO-3986 at the indicated concentrations, and that treatment of PHHs with 5 μM and 10 μM of ASO CO-3986 reduced cellular and extracellular bile acid levels as measured using a bile acid efflux assay (FIG. 7C).

FIG. 8A shows that treatment of mouse hepatocytes with ASOs CO-4755, CO-4765 and CO-4758, which target a mouse ABCB11 regRNA (RR54_v2) induced increased expression of Abcb11 mRNA. FIG. 8B shows increased ABCB11 mRNA levels (relative to geomean) in mouse hepatocytes following treatment with derivative ASOs based on parent ASO CO-4758 (i.e., CO-7212, CO-7214, CO-7215, CO-7216, CO-7237, CO-7238 and CO-7242) as compared to untreated PHHs (“Untreated”). FIG. 8C shows increased ABCB11 mRNA levels (relative to geomean) in mouse hepatocytes following treatment with derivative ASOs based on parent ASO CO-4765 (i.e., CO-7219, CO-7221 and CO-7227) as compared to untreated PHHs (“Untreated”). FC=fold-change.

FIGS. 9A and 9B shows that in vivo administration of ASO CO-3986 to humanized liver FRGR knockout mouse model mice induced increased human ABCB11 mRNA expression (FIG. 9A) and resulted in a decreased ratio of liver bile acid (BA) to serum BA (FIG. 9B), indicative of increased liver bile acid efflux.

FIGS. 10A and 10B show that ASOs targeting a mouse Abcb11 regRNA (RR54_v2), CO-4758 and CO-4765, increased both mouse Abcb11 and mouse Cyp7a1 gene expression in vivo in an alpha-naphthylisothiocyanate (ANIT)-induced cholestasis model (FIG. 10B), as compared to wild-type mice (FIG. 10B).

FIG. 11 provides ASO sequences and chemistries for exemplary ASOs targeting human ABCB11 regRNAs. Light gray shading indicates 2′-MOE; * indicates 5Me-C; dark gray shading indicates LNA; dark gray line indicates phosphodiester (PO) bond; white indicates DNA, black indicates GalNAc. FIG. 11 discloses SEQ ID NOs: 159, 418, 424,438-443, 445, 446, 153, 427, 430, 431-434, 436, 141, 393, 400-403, 26, 333 and 334, respectively, in order of appearance.

FIG. 12 provides ASO sequences and chemistries for exemplary ASOs targeting mouse ABCB11 regRNAs. Light gray shading indicates 2′-MOE; * indicates 5Me-C; dark gray shading indicates LNA; dark gray line indicates phosphodiester (PO) bond; white indicates DNA, black indicates GalNAc. FIG. 12 discloses SEQ ID NOs: 6022-6033, respectively, in order of appearance.

FIG. 13 provides ASO sequences and chemistries for exemplary ASOs targeting human ABCB11 regRNAs. Light gray shading indicates 2′-MOE; * indicates 5Me-C; dark gray shading indicates LNA; dark gray line indicates phosphodiester (PO) bond; white indicates DNA, black indicates GalNAc. FIG. 13 discloses SEQ ID NOs: 104-208, 18, 22, 19, 21, 20, 23, 24, 15, 17, 29, 34, 27, 33, 35, 31, 30, 32, 39, 46, 43, 38, 45, 37, 41, 42, 44, 53, 57, 48, 54, 56, 55, 51, 49, 52, 64, 63, 68, 61, 66, 59, 62, 67, 74, 78, 76, 73, 70, 77, 79, 75, 86, 87, 89, 81, 88, 83, 90, 84, 82, 97, 94, 98, 100, 92, 96, 95, 99, 14, 26, 36, 58, 60, 102, 91, 93, 80, 47, 71, 65, 85, 28, 40, 25, 72, 69, 101, 16, 50, 103, 209-216, 218-221, 223-231, 237-241, 233-236, 242-272, 274-280, 282-290, 292-300, 302-381, 383, 384, 390-394, 386-389, 395-407, 409-413, 415-423, 427-433, 425, 426, 434-462, 217, 222, 232, 273, 281, 291, 301, 382, 385, 408, 414, 424, 463 and 464, respectively, in order of appearance.

FIG. 14 shows ABCB11 mRNA levels in PHHs following treatment with ASO CO-11202 at the indicated concentration, or treatment with obeticholic acid (OCA; 5 μM), elafibranor (ELA; 10 μM), or fenofibrate (FF; 10 μM). FC=fold-change.

FIGS. 15A and 15B shows that in vivo administration of ASO CO-12367 to the humanized liver FRGR knockout mouse model mice induced increased human ABCB11 mRNA expression without affecting mouse Abcb11 mRNA expression (FIG. 15A) and resulted in a decreased serum bile acid (FIG. 15B). FC=fold-change.

### DETAILED DESCRIPTION

The present disclosure provides antisense oligonucleotides (ASOs) targeting regulatory RNAs, such as promoter-associated RNAs and enhancer RNAs, and methods of using these ASOs to regulate gene expression. These methods are useful for modulating the levels of gene products, for example, modulating expression levels of ABCB11, to thereby treat diseases associated with the accumulation of bile acids, such as primary biliary cholangitis (PBC) and cholestasis.

Various aspects of the compositions and methods described in the present application are set forth in the sections below.

### I. Definitions

To facilitate an understanding of the present application, a number of terms and phrases are defined below.

The terms “a” and “an” as used herein mean “one or more” and include the plural unless the context is inappropriate.

As used herein, the term “bile salt export pump,” “BSEP,” “ATP Binding Cassette Subfamily B Member 11,” or “ABCB11” refers to the gene of NCBI Gene ID: 8647 or Hugo Gene Nomenclature Committee (HGNC) ID: 42, or the protein of UniProt Accession No. O95342 (human) when used in reference to a human version of the protein, and to the protein of UniProt Accession No. Q9JL39 (mouse), when used in reference to a mouse version of the protein, and related isoforms and orthologs.

As used herein, the terms “regulatory RNA” and “regRNA” are used interchangeably to refer to a noncoding RNA transcribed from a regulatory element of a gene (e.g., a protein-coding gene), wherein the gene is not the noncoding RNA itself. Exemplary regulatory elements include but are not limited to promoters, enhancers, and super-enhancers. A noncoding RNA transcribed from a promoter, in the antisense direction, is also called “promoter RNA” or “paRNA.” A noncoding RNA transcribed from an enhancer or super-enhancer, in either the sense direction or the anti-sense direction, is also called “enhancer RNA” or “eRNA.”

As used herein, the term “nascent RNA” refers to an RNA that is still being transcribed or has just been transcribed by RNA polymerase and remains tethered to the DNA from which it is transcribed. An RNA that has dissociated from the DNA from which it is transcribed is also called an “untethered RNA.”

As used herein, the term “antisense oligonucleotide” or “ASO” refers to a single-stranded oligonucleotide having a nucleotide sequence that hybridizes with a target nucleic acid under suitable conditions or a conjugate comprising such single-stranded oligonucleotide. In some embodiments, the disclosure encompasses pharmaceutically acceptable salts of any of the ASOs described herein. Suitable pharmaceutically acceptable salts include, but are not limited to, sodium, potassium, calcium, and magnesium salts. In some embodiments, the ASOs provided herein are lyophilized and isolated as salts (e.g., sodium salts).

As used herein, in some embodiments, the stability of a regRNA is reversely correlated with the degradation rate of the regRNA. In some embodiments, where an ASO increases the stability of a regRNA, it reduces the degradation rate of the regRNA. In some embodiments, where an ASO decreases the stability of a regRNA, it increases the degradation rate of the regRNA. In some embodiments, the degradation rate of a regRNA can be measured by blocking synthesis of new regRNA and assessing the half-life of the existing regRNA.

As used herein, the terms “subject” and “patient” refer to an organism to be treated by the methods and compositions described herein. Such organisms preferably include, but are not limited to, mammals (e.g., rodents, primates, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### II. Antisense Oligonucleotides (ASOs)

ABCB11 (also known as the bile salt export pump or BSEP) is the only bile salt efflux transporter. Loss of both alleles leads to cholestasis (PFIC2). ABCB11 overexpression shifts the bile composition to more hydrophobic bile acids with higher affinity for FXR. This results in increased expression of bile acid responsive genes without increasing the bile acid pool and a decrease in new bile acid synthesis. Loss of one ABCB11 allele (resulting in 50% activity) is not disease-causing. However, loss of one allele in addition to polymorphisms on the remaining allele can result in sub-clinical forms of cholestasis (˜25% activity) (see, e.g., Yang and Park (2016) Korean J. Obes. 25 (4): 167-75, and Bull and Thompson (2018) Clin. Liver Dis. 22 (4): 657-669, both incorporated by reference in their entirety). Without wishing to be bound by theory, increasing the expression of the ABCB11 hepatic bile acid regulator will enhance bile flow from the liver and restore enterohepatic circulation of bile acids in subjects having or at risk of developing cholestasis or a cholestatic liver disease, including, e.g., PBC, Alagille Syndrome (ALGS), primary sclerosing cholangitis (PSC), biliary atresia, intrahepatic cholestasis of pregnancy (ICP), or progressive familial intrahepatic cholestasis (PFIC) (e.g., PFIC type 1 (PFIC1), PFIC type 2 (PFIC2) and PFIC type 3 (PFIC3).

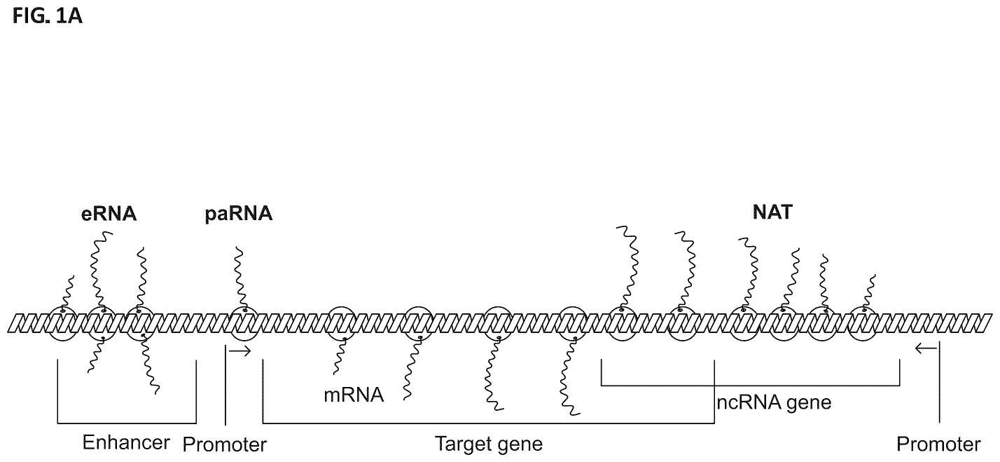

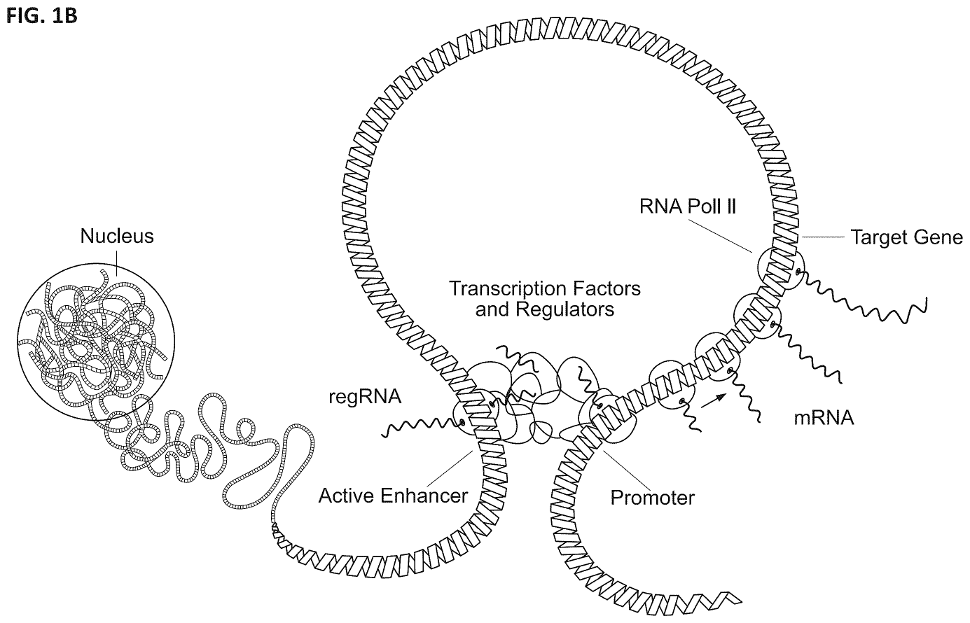

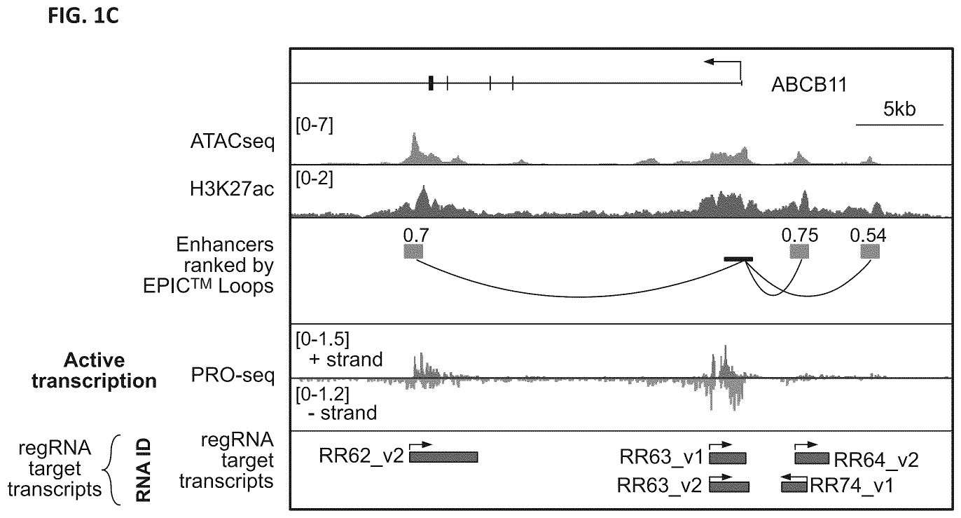

In some embodiments, the antisense oligonucleotide (ASO) disclosed herein hybridize with or targets a regRNA (e.g., an eRNA or a paRNA) transcribed from a regulatory element of the ABCB11 gene, also referred to herein as an “ABCB11 regRNA”. It is understood that both eRNAs and paRNAs are regRNAs modulating (e.g., facilitating or upregulating) gene expression (FIG. 1). In some embodiments, the ABCB11 regRNA is a human ABCB11 regRNA. In some embodiments, the ABCB11 regRNA is a mouse ABCB11 regRNA. In certain embodiments, the target ABCB11 regRNA is an eRNA. In certain embodiments, the target ABCB11 regRNA is a paRNA. In certain embodiments, the ABCB11 regRNA is not a polyadenylated RNA. eRNAs can be identified using methods known in the art, such as Assay for Transposase-Accessible Chromatin using sequencing (ATAC-seq), global run-on sequencing, precision run-on sequencing, cap analysis gene expression, and histone modification analysis (see, e.g., Sartorelli & Lauberth, Nat. Struct. Mol. Biol. (2020) 27:521-28; PCT Application Publication No. WO2013/177248). paRNAs are RNAs transcribed from promoters of target genes in the antisense direction (transcripts in the sense direction are mRNAs of the target genes). They can be identified by similar methods, taking into account of their specific location and orientation. For the human ABCB11 gene, eRNAs have been identified to transcribe from the same bidirectional enhancer region. The nucleotide sequences of exemplary human and mouse ABCB11 regRNAs (eRNA and paRNA) are provided in Table 1 below. Any of these regRNAs are contemplated as a target ABCB11 regRNA of an ASO disclosed herein.

TABLE 1
Exemplary regRNAs

| SEQ ID | | |
|---|---|---|
| NO | Name | Sequence |
| 1 | RR62 v2 | AAAATTTCAAAAATGAAGGTGAAAACTATAAATTCCTCTGAGGGAGCCATTCCTAGTGTCTTTC |
| | (+) strand | TAGCCAAGATGTGCCCCACCCTTGATTCTTGAACCTGAATATGTGGGTTCCTATGTGCAAATAA |
| | eRNA | GCTGTTCTGAACTTGAGCCAGCATGAGGCACACTGCAAAGGCCATGCTATGACAGTGAGTCAGC |
| | Human | CCTGAGTACACGACAGACTCACTGATTACAAGGTGGCCTGTCAGGCTTATCACAAAGAAAACAG |
| | | TAGCATAGTGACTGTGTCTGTGTGAGTTCTGGCTACTTCTGCGTGGTTGCCTCTGATCACAGCA |
| | | CTGCCCTAGTTTTCTCACGCCTGTAATCTCAGCACTTTGGGAGGCTGAGGCAGGCAGATGACCT |
| | | GAGGAGATTGAGACCAGCCTGTCCAAAATGGTGAAACCCCATCTCTACTAAAAATACAAAATTT |
| | | AGCTGGGCATAGTGGCGGGCGCCTGTAATCCCAGCTACTCAGGAGGCTGAGTCATGAGAATCAC |
| | | TTGAACCCAAGAGGCAGAGGCTGCAGTGAGCCAAGATCACACCACTGTACTCCAGCCTGGGCAA |
| | | AAAAGGAGACTCCATCTTAAAAAAAAAAAAAAAAGAAAAAAGAAAAAAACAAACTCAACTACTG |
| | | GTTGAGACTAGTGTACCTTACTCAAAGTAATGCAGCCTGTTCATGAAATTCCCATTACAGATGG |
| | | AAGGGTTAAGATCAAGTGGATCACCAGCCACAGCCACAAAGCTAAGGGGCAGAGGGGAGTTGGT |
| | | GGGGGGGAAAAATAGAACTCTTGGAGATGATACTCAAGTAAAATTCATGGGTCACCTCCCTAGT |
| | | GACGTTAAGTATTGGTAGTGTCATGCTCAAGCCTATACTCAATAAAGTGGTAACATTTTGGCAA |
| | | TGTATTTGGTGAGTGACTTTCAATCCTGTTTCTATTAAGATTTTAAAACATAGTAAGATAGAAG |
| | | GAAACTTGAGGCAGAGTAATTTCTAGAAATATGGGTAAACTCATGTTTCTATTCTCTTTGTGTT |
| | | AGATACCACTCCAGCTCAGCCAGTAAAATCCCCTCTATACATTTTGAATTACAATTTAAGATAT |
| | | GAGCAAAAAAGTAAAAAATTAAAAACAAAAACAACCTACCCACAACGTGTTCCATTTGTCATGT |
| | | TCTGGTTGAGGGAACTGTTAGTCCATACAATGGTGTTATTCACACATGCTTTTCCTGGAATCTG |
| | | GAGTTCTTGTAACTCAACGTCGTAGTCAATAAAAACATCTGTCATTGTGCCAAAAATGAGTAGC |
| | | ACGCCTGGCTGGGCTATTCCATGGAGAAATGCACACAAACTTCCCACAAACATCAGCCAAATGT |
| | | CAGTTGATGAAGAAAACCGAAACTTGAAAAACAAAGGGTTCAGAGATCATCTATGGGTGAAGAG |
| | | CAGGAGAGGTAGGAGGACTACTTAATTTAGTTACTAGATTCTCATGAATAGTACTCAACACCAA |
| | | ATTTCATGGGAATCACCTTAATTGAGTGGCAGAGTTCGTATACTAATTATATTGAAATAAACGG |
| | | CACGGTACATCAATTATCTAAAACATTAGACTCTTACATTGTTAGATCATGTTATAAATTTCCT |
| | | GCTCTCTGAATTCATTCTGTTTATTTCAGGAGCTGACTTTCGTTTTTAGCTACTCATCTACAAC |
| | | TTAGGGGAAAATAATCACTGTCATTGATGCCATTGCCAAGTAGATAAACTGTGTGGGGCATTGA |
| | | GAAATACTATAAATTGTTTTGTTGAACTGATAGTGACTATGAAGAATGCCAATTTCTGCCTAGA |
| | | AATGCCTCCATCCTGAGAAAATTTGGAGTAAACACCTATACTTTAAAATTTCTAAAGATTTGTC |
| | | ATAAGAATAAATATATATATGTGATTGTTGTGATGACTTTCCTTACAAATATGATAACCATGGG |
| | | CTTAGTGATAATTTTCCAAGACATTTTTCTGATCTATTGGATAGGATTTGTATGCTTTATGAGC |
| | | AATATATTTCTAATAGTTATTCAGCATGTTATCACATAGGCAAAGCCTATGACTGAAAAAGTGA |
| | | TCACTGAAGTTAAAAACCTGCCAATATGACTAAAGATTTAACACTCCCCTCATGATCTAAACAA |
| | | TTTATAGCTGCACACCCACTGCCATAAATCAACACAGTTTTATTACCAATTGAAAGAAGCCAAC |
| | | TCTAACGCCATCACCTTTCTTCTCATCTTGTAACCTGATGAGAAAAACATAAGGATTTAAAGAC |
| | | CACCCTTTCCAATACAATGGGAAATTCTCCCTAGGTGGTGATTTTATGAGTTATTCTTTAGTAA |
| | | GAAAGAAAATCCCCACTGGCTGGAAAATTCTGGCCAAACCTAGTATCTTAAACACAAGCTTTAT |
| | | TCTTCTAGATTTCCCTCTTCTAGAGATTTCAGTATAGCTCAATCTCAGGTTCACTAGCGATTCA |
| | | CAGAGATGAACACCTGAGAGGTATGGAGAGAAAGTAAAAAGTCCTCTATACTCCCAGTTATACC |
| | | AACAAATAACTCTTTGCCCTCAGTCATGACCCTCAATCTATTTGTGACTGCATTTCCTCATGGG |
| | | TAAGTTGAAGTGGTTGGATTAGCTAATTTTAAAGCTTCCTCATGCTTAAAAATATTACAACTAC |
| | | ATTAAATAATGTTGATAATGATGAAAAATTCAAGAACATGCCCACACTTTTCATAGAATCATAT |
| | | TTCCTTAATATTTCCCATTCATTTCATTTCCCATAAGGCCATGTTTAACCTGTATTTGACCTCT |
| | | TCAACTTCGGGGATCTTGACTTAACCTGAGTCACGCAGTCCATGAATACACCCTGGACCTTGCT |
| | | GTCACCAGGAACTACTGTCTGACATGTTTAACTCCTTGGCACTCTGTGACTGCCCATGTACCTC |
| | | ATTTTCTCGCTTCCTTCCTCCTTCCACACCTATTTGTCAACCTCACTTGAGCCATGCAGTCCTT |
| | | TAAATCCTTCATTTCCTTCCATTGCTAAACCCCATTAAGATTTTGCTTCCTTCCCCATGCAGCC |
| | | AAGGTCCCTCTGCCAGGCATTTGAACCACACTCTCACCATCATCCTCAATTATTTTCTCTACAT |
| | | CATCCTTGTAAACTTCATCCTGCATCCATCCAGCTGTTTGCCCTCTTCTCCTTAAGCCAGGCTG |
| | | CTGGGCCTGCTGGACACAACCTCACCACCTTGTGAATGGATACCAACACAGGCAGAGTCTAGTA |
| | | CTCAGCTGCCCTAAGCTTCCCTTCTGGCCCAGAAAAGAATCCTAAATTTTTTCTGAGACTCCCC |
| | | ACGGTCTGGCCCTTGGCTCTCTGACCTCACCTCCTCCAACTATATTCTAGTTCTCAACACCCCA |
| | | CTTGCCTGGCCTCTTGCTGACCTTTGTCCATGCCCAACTGTTCCCACCTCTGGGTGCGGGCACC |
| | | TGCTACTCCTTCTCCCCAGGTGCATTTTCTTTAGGTAACCACAGGGACAGCTCCATCACCTCCG |
| | | CTAGACCTTCCTTTGAAATCACCTACTCAACAGAGTCTTGCCTGGCCAAAGTCTTTAAAATTTC |
| | | AACCCCACCCCCCACGCACCATTTCATAGCTCCATTTCCAGTTTATTTTTCCTTCTCAGAAAGA |
| | | AAGTGATAGATTATCACTAGCTACCATGCTATACATTTGATTTATTTACAATGTTTATTATATC |
| | | TCCCTCACTAAAATGTAAACTCCTCAAGGGCCAACATTTTTGCCTATTTTGTTCTTTTCTATAC |
| | | CCGCAGCACCTACAATAGTGTCTGATACCAAATAGACACTCAATATTTGCAAAACGAATGAATT |
| | | AATGGAGTGAATGTGTGCTCTCCAGCTCAACATGCAATGCACACTTCATTTCTCTGCGGATTGC |
| | | TCCCATTCTTCTCAGCAGCTCTGGCAAAATTTGCTGTGATCTTCTCATTCCATGCTCACCTCCT |
| | | CTAAAAACAAAGCAACTCATTCAACTTCCTACTCATCCTCTCCTTGCCATTCATCTACATCCAC |
| | | ACCCATTTTTTAAACTTCCCACGCAATAAGAAACGTGTGGCTCTCAGAGGCCCAGGCTGACCTG |
| | | ACCACTCCCAGCCCAAAGCTAATCTTACCACTCCCACTTCCTCCCTCTGCTGTGAATTACTTAA |
| | | AATTTCTAAAATACCTTTAACCCTTCCGTTTCCACCACCTCCACTTCAGGAACTTACCTGATGC |
| | | CTCACTTCTGTCTTCTAAATTAGACCTCATCTAATCCCGC |
| 2 | RR63 v1 | AAACATTTAATATATGCTAAGAAACAAAGAAAATAGAAAGGCATTATTTTCCAGATAAGCTCAC |
| | (+) strand | TGTCGGTTGATGCTGTGTGTCAACACTGTGGGTGACCCAGTCTGGGAATAGACTAAAATGAGGC |
| | paRNA | CCTTCCAGCCCTGAGGGCTATGGCTGATAGGGAAGGGAAATAAATACACGGTGATCCCATTGGC |
| | human | CACGTAAGTCCTTTCTTCAGAGGCACCGTTGAGGTAGAGGACTAAAGAATTGCTTAGGAAGAAA |
| | | ACAGAATGTAACACCAAAGTTAACTGCCACAGCGATTCTTTGTCTCTAAATGTACAGTTCTTTC |
| | | CTCTTTTTTTTTTTTTTTTTTTTTTTTTTTTGAGAGGAGTCTCGCTCTGTCGCCCAGGCCGGAC |
| | | TGCGGACTGCAGTGGCGCAATCTCGGCTCACTGCAAGCTCCGCTTCCCGGGTTCACGCCATTCT |
| | | CCTGCCTCAGCCTCCCGAGTAGCTGGGACTACAGGCGCCCGCCACCGCGCCCGGCTAATTTTTT |
| | | GTATTTTTAGTAGAGACGGGGTTTCACCGTGTTAGCCAGGATGGTCTCGATCTCTTGACCTCAT |
| | | GATCCACCCGCCTCGGCCTCCCAAAGTGCTGAGATTACAGGCGTGAGCCACCGCGCCCGGCCAG |
| | | TTCTTTCCTCTTGACCAAGCAAAACACAACTCCAGTATTTGATTTCTGCAATGAAACTTGGGCA |
| | | TTTTATGGCAATTGTAGCATCTTTTAAAACCACAAAATGGGCATTTTCCCCCTAAATAGCTTCT |
| | | TAATCCATTTTTAGGAGTCATCCCCTAATATTTGTCACCATTATTCTTGGTACTAGGAAACAAA |
| | | AATTATTCAAGGGGCTTGCAATCTAGAGTGGGGTGGAGTGTAAACAGAAGCTCCTAGACAGGGA |
| | | AATGGAGGCTTCAACCAGAGACAATCACAGTGTTACAGAGCACAGCAGGGGGAACCAAGGTGAG |
| | | GGGGGTGAGCAGTGAGGGGACGAGGTTGCCCACAGAAAAGAACTTCTATTAAACAACAGCTCAT |
| | | AAATAAGTTATCACTTCAAATGTTTAATAAAAACCACTACTAACTCCTAATATTAAAAAGTTTT |
| | | AGGTATAACCACTATAATTTGTCTCTAACATCTCTAAAAAGCTATTCCCCCTCAAAATGTTGAC |
| | | AAATACTGTCTTTTATTCCCAGTCTGAAGTGTGAATAATATTTGATCTCTGTGCTCAAGCCATT |
| | | AGCAATATGTGACTCCCACTGGCTTGGGTGACCTAAAAACTGAAATTAGATCCTAGAAACAAAC |
| | | TTTCTCCCCTCTAAACATGCATCCACAGCTCATTTTTTTTTGCATCTATATTTTTGAAAACCGG |
| | | GACAATGAAGAAATGAGTACGGTGTTGGAGAAAAGATAAAGTCGGTGTAGTTCTGCATTAAACC |
| | | AGCTTTCAAGGGTAGAATTTGTGATCATTGAATCTGTGATTATCAGAGCAAACAGAATTGTGCT |
| | | TTCACAAAGCCAAGTTAAAAAAGAAGCCCACTGGTATTTTTACTTTGAGCAGATTATTCAATCT |
| | | ATTCGGCCATTTGTACCACACACCCATGTACACCACTCTCTCTCTCTCTAAGAGATTGATACAT |
| | | GAAATAGCATTGCTATAAAGAAGACAGCAACCAAGAGTCATCTGTATTGTATACAATGTTGTGA |
| | | AATCATTAATGCATCCTGTAAGTTTCTATCCAAGTATTGTAGCGTTGTTTACACTGGACAAGAG |
| | | ACCAATGTTTAATAAACCAGAAACACTCCACTCCACAAAGTAAGAGTTAAAGGGAAGAGGGAAT |
| | | GGATTTGCAGAGACAAGTATAAGTTACTTACCTGTGAATATGAATTTGAGGAAGCCAGAGGAAA |
| | | TAATGGACTCCACTGTGGAGAGTTAAAAGGTTTCACAACCTTTTCCAACCTCGGTTTTCATCAT |
| | | TCTAAAGCACTGAACAGAATTCAAACTTTTTCAAGAACATTATCTATTTGCCTAAGGATCAATG |
| | | TCCCTAAGGGCAGCCCAAGCACAGTTCAGTGACATACGAGAAACATACTCAGAGCAATAACCCC |
| | | AAACACAGAGTGTGCTTGGGAAACCCTCTGAGGGTGTCAACAGGCCTTGGAAATTGCTGATTCA |
| | | CCTTGGAACGAGAGGCAAATTGAGCAGAGTGGGCCCAGCAAGAGCCCTGGAATTCAGGCTATCC |
| | | CAACCCTTATTTTTTACAGGCCTGTAGTTGTGAAAAGTTACTTAAAAACATAACAAGACAAAAA |
| | | AAAAAAATACCTTTAAACTTTCCCCCTCAATATAAAAGATTGCTCTTGGAAAATCCCTGGTTGC |
| | | AGGATCCAGATGCTCCTGGCCCAGCTATAGAGTTTTGTGGAGACAAATACCCTAAAAGAACATT |
| | | TGAAACAGCCTTGCTCAAAAGTCTGAGTGCACAGACAACTCTTGCACCCTGGCATGGACACACC |
| | | AGATGAAGCTCTGTATAAGCAAAGATGAACGATTTTCTAAAGGTTCAAATGAACAGGTCTTGAG |
| | | TCAAATAAGCATGTGGAGCTCCTCCAATATATTTGTGCTGCCTCTTCCTCTCCCTTCTTGTGAA |
| | | TCTTTCCATTTTTCCTACAATGTTCTTATTTTTTCTCCTTTGCCTGAGCTTACTTTTCCCTAAA |
| | | TGCCTCTCACTCCAGCCTTCAGCAATGAATAATAATAGGCAACATTGGTCAAGACTTGGTTGGG |
| | | TGCCTGGCCCAC |
| 3 | RR63 v2 | AAACATTTAATATATGCTAAGAAACAAAGAAAATAGAAAGGCATTATTTTCCAGATAAGCTCAC |
| | (+) strand | TGTCGGTTGATGCTGTGTGTCAACACTGTGGGTGACCCAGTCTGGGAATAGACTAAAATGAGGC |
| | paRNA | CCTTCCAGCCCTGAGGGCTATGGCTGATAGGGAAGGGAAATAAATACACGGTGATCCCATTGGC |
| | human | CACGTAAGTCCTTTCTTCAGAGGCACCGTTGAGGTAGAGGACTAAAGAATTGCTTAGGAAGAAA |
| | | ACAGAATGTAACACCAAAGTTAACTGCCACAGCGATTCTTTGTCTCTAAATGTACAGTTCTTTC |
| | | CTCTTTTTTTTTTTTTTTTTTTTTTTTTTTTGAGAGGAGTCTCGCTCTGTCGCCCAGGCCGGAC |
| | | TGCGGACTGCAGTGGCGCAATCTCGGCTCACTGCAAGCTCCGCTTCCCGGGTTCACGCCATTCT |
| | | CCTGCCTCAGCCTCCCGAGTAGCTGGGACTACAGGCGCCCGCCACCGCGCCCGGCTAATTTTTT |
| | | GTATTTTTAGTAGAGACGGGGTTTCACCGTGTTAGCCAGGATGGTCTCGATCTCTTGACCTCAT |
| | | GATCCACCCGCCTCGGCCTCCCAAAGTGCTGAGATTACAGGCGTGAGCCACCGCGCCCGGCCAG |
| | | TTCTTTCCTCTTGACCAAGCAAAACACAACTCCAGTATTTGATTTCTGCAATGAAACTTGGGCA |
| | | TTTTATGGCAATTGTAGCATCTTTTAAAACCACAAAATGGGCATTTTCCCCCTAAATAGCTTCT |
| | | TAATCCATTTTTAGGAGTCATCCCCTAATATTTGTCACCATTATTCTTGGTACTAGGAAACAAA |
| | | AATTATTCAAGGGGCTTGCAATCTAGAGTGGGGTGGAGTGTAAACAGAAGCTCCTAGACAGGGA |
| | | AATGGAGGCTTCAACCAGAGACAATCACAGTGTTACAGAGCACAGCAGGGGGAACCAAGGTGAG |
| | | GGGGGTGAGCAGTGAGGGGACGAGGTTGCCCACAGAAAAGAACTTCTATTAAACAACAGCTCAT |
| | | AAATAAGTTATCACTTCAAATGTTTAATAAAAACCACTACTAACTCCTAATATTAAAAAGTTTT |
| | | AGGTATAACCACTATAATTTGTCTCTAACATCTCTAAAAAGCTATTCCCCCTCAAAATGTTGAC |
| | | AAATACTGTCTTTTATTCCCAGTCTGAAGTGTGAATAATATTTGATCTCTGTGCTCAAGCCATT |
| | | AGCAATATGTGACTCCCACTGGCTTGGGTGACCTAAAAACTGAAATTAGATCCTAGAAACAAAC |
| | | TTTCTCCCCTCTAAACATGCATCCACAGCTCATTTTTTTTTGCATCTATATTTTTGAAAACCGG |
| | | GACAATGAAGAAATGAGTACGGTGTTGGAGAAAAGATAAAGTCGGTGTAGTTCTGCATTAAACC |
| | | AGCTTTCAAGGGTAGAATTTGTGATCATTGAATCTGTGATTATCAGAGCAAACAGAATTGTGCT |
| | | TTCACAAAGCCAAGTTAAAAAAGAAGCCCACTGGTATTTTTACTTTGAGCAGATTATTCAATCT |
| | | ATTCGGCCATTTGTACCACACACCCATGTACACCACTCTCTCTCTCTCTAAGAGATTGATACAT |
| | | GAAATAGCATTGCTATAAAGAAGACAGCAACCAAGAGTCATCTGTATTGTATACAATGTTGTGA |
| | | AATCATTAATGCATCCTGTAAGTTTCTATCCAAGTATTGTAGCGTTGTTTACACTGGACAAGAG |
| | | ACCAATGTTTAATAAACCAGAAACACTCCACTCCACAAAGTAAGAGTTAAAGGGAAGAGGGAAT |
| | | GGATTTGCAGAGACAAGTATAAGTTACTTACCTGTGAATATGAATTTGAGGAAGCCAGAGGAAA |
| | | TAATGGACTCCACTGTGGAGAGTTAAAAGGTTTCACAACCTTTTCCAACCTCGGTTTTCATCAT |
| | | TCTAAAGCACTGAACAGAATTCAAACTTTTTCAAGAACATTATCTATTTGCCTAAGGATCAATG |
| | | TCCCTAAGGGCAGCCCAAGCACAGTTCAGTGACATACGAGAAACATACTCAGAGCAATAACCCC |
| | | AAACACAGAGTGTGCTTGGGAAACCCTCTGAGGGTGTCAACAGGCCTTGGAAATTGCTGATTCA |
| | | CCTTGGAACGAGAGGCAAATTGAGCAGAGTGGGCCCAGCAAGAGCCCTGGAATTCAGGCTATCC |
| | | CAACCCTTATTTTTTACAGGCCTGTAGTTGTGAAAAGTTACTTAAAAACATAACAAGACAAAAA |
| | | AAAAAAATACCTTTAAACTTTCCCCCTCAATATAAAAGATTGCTCTTGGAAAATCCCTGGTTGC |
| | | AGGATCCAGATGCTCCTGGCCCAGCTATAGAGTTTTGTGGAGACAAATACCCTAAAAGAACATT |
| | | TGAAACAGCCTTGCTCAAAAGTCTGAGTGCACAGACAACTCTTGCACCCTGGCATGGACACACC |
| | | AGATGAAGCTCTGTATAAGCAAAGATGAACGATTTTCTAAAGGTTCAAATGAACAGGTCTTGAG |
| | | TCAAATAAGCATGTGGAGCTCCTCCAATATATTTGTGCTGCCTCTTCCTCTCCCTTCTTGTGAA |
| | | TCTTTCCATTTTTCCTACAATGTTCTTATTTTTTCTCCTTTGCCTGAGCTTACTTTTCCCTAAA |
| | | TGCCTCTCACTCCAGCCTTCAGCAATGAATAATAATAGGCAACATTGGTCAAGACTTGGTTGGG |
| | | TGCCTGGCCCACTTCAAAAATGTTATTTGTATCACCTCATTTGGGCCTCTCAACAGCCCTAGGA |
| | | GTTGGGTGCTATCATGTCATTTCTATTTTATAGTTGGGGAAACTGAAGCACAGAGAGGTTAAAT |
| | | AATCCTCCTAAGGTGTAACAACTAGTAGATGTGAAGCCACATGCCAGTCTGTGCTAACCTTCTT |
| | | CCACTCTGTGCTCTGAAATGCAATTGATGGGCCAGTGCTTCTTAATTTGCAGAAATTAAACTGT |
| | | AAGATCAAATTTTCTCAATTTTAGGTTTCTCTGTCATAAATTTTTTATTAAAATTC |
| 4 | RR64 v2 | AGTGCACACAGGCAACATTGAAAGGACCAAGATGGCTGCTGAGAGAAGAGGCAAAAAGTGGCTT |
| | (+) strand | GTAAATTTGCTTAAGTAGGTGAAGAAAGACAAAGAAGGGCTTTCAGTGCCAGTAGGATGAGTTG |
| | eRNA | CATGCAGTCTAAGATAAGAACTTCTGTTCACGGCCACCCAGGGCCAGTGTTTACAAAGCTGAGC |
| | human | CACAAGAAGGATGGGTTAGGCAGGAAGTCTTGCACATGCACACCAATCACAGAAGCTGGTTATT |
| | | ACCACCCTGAAAATCAATATAAATTGTGTAAAGCCAGCATGAGTCAGTTAACACAATATAATTG |
| | | GCTATGTTTTTTTGTGGCATAGAGACTAGCAGAGAGAAAAATGTCTAAATTAGAGCTTTCTTAG |
| | | GTAGCTTATGTATTTTTAAAGCAAGCTGTGTGGAAGGACACAATTTCTGTAGTTTAGTTGACTG |
| | | CAGAGGGAGGCAGAATGGTATAGTGAAAAGAGCACAGGCTTGTGAAGTCACAAAGAACTGGCTT |
| | | TAGATCCTGGTTTTGCCACTTACTTGTTGGATGATTTGGTTAAGGTAGTGATACTTGCTAATTA |
| | | TTTCCCCTCATGTGAAGTCCAGTAGGCCTTGAATAAGGGGAAATCGGTGTTGGGCAAAGGTGCT |
| | | CTGCTCCATGCAGTCATTCAGAGACCCAGGCTGACAGAAGCTCTACCATCCTCAACATGTGGCT |
| | | TTTAAGGTCTCCTTGGACATGGACATGTATCCATCACACAGGGAAGAGAAAATATGGAGCATTT |
| | | TGTATGGAAAGTTCCTGTGGGCCAATCCTGGAAGTGGCATCCATTACTTCTACTCAGATTCCAC |
| | | TGGAGAGGACTGTGCCACATAACCACACCTAACTGCAAGAAAGATTGGAAGATAAGGTCAGGCT |
| | | GTGTGTCTGGGAGGTTTAGTTAAAAGTTAACCAATCTCAAGAGATGGTCTTTGAAATCACAAAG |
| | | GTTTGTGTTCACTTACTAGCTATCTAACTTTGGACAAGTTACTTAATCTCCATTAGCCTCAGTT |
| | | TCTTCATCTGTAAAATGGGAGTGAGAATACCACCTGCACAGGATGATGAGGCCTGCCTGAACTT |
| | | CCCTGTGCCATAGATTCTGGATTGGGAGTTCCTCCCCCAAGTAGCCATTTAATTGAATTGTGTA |
| | | TATTTTTTTACCTACCAAAATTAGTGTATTCCTAATTTTGTCATGTACACATGTAGAAAAGACT |
| | | GGAAGAAGTACAATATAAAAAGTGCTTATCTGTAGGTTATGCAAATGTGAATGAGGGTGGTTTT |
| | | CATTTTCTTCCTCATGCTTTTCTGCATCTTCTAAATTATATACAATAAGGGTAAACTATTTTTT |
| | | AAACATATAAGTTATTAAATGAAAATAATGAATATGACAAAGGAAATATCAATAATAATGCATA |
| | | ATAAAATTTTATTAATGATTTTTAGAAATACATAGAGAGTACAACTATGTGGGTGTTCTCCAAA |
| | | TATTAGCTGTAGTTCAGTCATTGCTTTTTTTCTGGGAAAACTCTGACTTCTGCAGAAGGGATGG |
| | | GGCCGTCATCTGGACATCACAAGCAAAGTTTCTGTGGATTCAAAGCCCATTAAGAACTTGCTTT |
| | | ATCCTAACACAGACCAGCCCTGTGTCATGGTACCTGATGCTCCTGCTCAGTGGTGTGCCCTGAA |
| | | TGTCCTGAACCTGGAGGTAGATCCACCCTATCCCAGCTCTCTGCCTCATCTGGCTCTCCCAAAA |
| | | GCTGGCTATCACTGATCCCCTTTGGCTCAGTGCCTAGTTTGCTCCACCTCCACATCATGTCTGG |
| | | GTAGGGCTCCTGGAATTCCTTCAGGCCTCACTTGTGGGCCATTTAGAGTAAAATAGTGGGCACA |
| | | TACAACACTTCATTATGGTCTTCAAACCATGTTTCCTTTAAGATGATCTTAAGGACGAAAGTAA |
| | | GAAATAGTGAGTTGAGTAAGTCCAATGTATTATTAAATGTTTTATGCTCCCTCTGTAGAATTAA |
| | | TAACGCCAAATAAGACTAATTACAATCCTAGCAAGTTGTCATTTATAGTTATCTAAATGAAATA |
| | | CATTGATCTCCACAGCACTCAGTACTTGGTTAGAATTTATATTTTTGGTTCTCCCTCTAATGAG |
| | | GAAAACCAGCACAAAAAATATGGACTTCATTCATAAGCAAATCTTTTCTGATGTTTTTGAGTGC |
| | | GGTAGTTCTTGTCTTTCCCTGAAGGCTCATTATTGTGCATAAAGTTATAAAAATTATAAGGGTA |
| | | CCTTTATAATTGCCAAATGAAAAGAAATAGGTGAAATAAATAAAGAAGAGGAATCAGAGAGAAT |
| | | TCTGCTACAGAACAAAAGTCAATTATAAGTAATTGTGCCTGCAGTGTTGAATACTCATTTGAAC |
| | | TGTAGAATTTATCACTCGCAAGGGGCAAGTCTGCCTGGGGGTATGGGTAGATTGGATTTTCATG |
| | | TCTTGGCTTTATCCAGGACACCTCCCTTCTTAAAGTCATGTTTCAGTCGTGGAAACTATTCCTT |
| | | TGTT |
| 5 | RR74 v1 | AACTTGTCCAAAGTTAGATAGCTAGTAAGTGAACACAAACCTTTGTGATTTCAAAGACCATCTC |
| | (−) strand | TTGAGATTGGTTAACTTTTAACTAAACCTCCCAGACACACAGCCTGACCTTATCTTCCAATCTT |
| | eRNA | TCTTGCAGTTAGGTGTGGTTATGTGGCACAGTCCTCTCCAGTGGAATCTGAGTAGAAGTAATGG |
| | human | ATGCCACTTCCAGGATTGGCCCACAGGAACTTTCCATACAAAATGCTCCATATTTTCTCTTCCC |
| | | TGTGTGATGGATACATGTCCATGTCCAAGGAGACCTTAAAAGCCACATGTTGAGGATGGTAGAG |
| | | CTTCTGTCAGCCTGGGTCTCTGAATGACTGCATGGAGCAGAGCACCTTTGCCCAACACCGATTT |
| | | CCCCTTATTCAAGGCCTACTGGACTTCACATGAGGGGAAATAATTAGCAAGTATCACTACCTTA |
| | | ACCAAATCATCCAACAAGTAAGTGGCAAAACCAGGATCTAAAGCCAGTTCTTTGTGACTTCACA |
| | | AGCCTGTGCTCTTTTCACTATACCATTCTGCCTCCCTCTGCAGTCAACTAAACTACAGAAATTG |
| | | TGTCCTTCCACACAGCTTGCTTTAAAAATACATAAGCTACCTAAGAAAGCTCTAATTTAGACAT |
| | | TTTTCTCTCTGCTAGTCTCTATGCCACAAAAAAACATAGCCAATTATATTGTGTTAACTGACTC |
| | | ATGCTGGCTTTACACAATTTATATTGATTTTCAGGGTGGTAATAACCAGCTTCTGTGATTGGTG |
| | | TGCATGTGCAAGACTTCCTGCCTAACCCATCCTTCTTGTGGCTCAGCTTTGTAAACACTGGCCC |
| | | TGGGTGGCCGTGAACAGAAGTTCTTATCTTAGACTGCATGCAACTCATCCTACTGGCACTGAAA |
| | | GCCCTTCTTTGTCTTTCTTCACCTACTTAAGCAAATTTACAAGCCACTTTTTGCCTCTTCTCTC |
| | | AGCAGCCATCTTGGTCCTTTCAATGTTGCCTGTGTGCACTCATGCTGTATTTATGACCTGGTCC |
| | | CTCTCATACTTTGAGATATAACTCAAGTCCTTCCTTCTTTGAGAATCCATACTGATGGGTCACT |
| | | TTTTAAATACTTAAGGTCTGCATCAAACATTATAACATTTGCTGTTCTTCTTTTTTATTCTTAC |
| | | TTGGTTTATATACTAAGCTTTATTATCCTTTTAAGGTTAAAACTTTCTCAAGGACACTAACTGT |
| | | AATGTGTACAACTTGGTAGCCAGCGTAACATTTGACATAGTGTCTTGTAAAAAGAAGATACTCA |
| | | ATCAGTTTTTGAATGATTCAAAGAGTAAAATAATGGCAACTTCTTTTATCTCTTCCATAGTGCA |
| | | ATCTTCAATATTTTTTAAACAACTATTGGCTGAATTCTACCATTGTGGTTTAGAGCTTTCTCTG |
| | | AGACATTTTTGCTAAGGTTTTGAAACAGGTTCCAAATTGGTCCACAGTGACCTCCAAGGCTCTA |
| | | CAGGAGTGGTCCTCAAGCTTCAGCTCATTGGGCCCCACTCCCAGAGTTTCTGATTCAGTAGGTC |
| | | TCAGGAAAGGACCAAGAACATACATTCCTACTGGGTAATGAGGAGGCTGCTGCTGGTCCAGTAG |
| | | TCACACTTTCAGAGCCACTGCCCTGTGAGCTCATGGCACTCCATGATTCCCGCCACACTGACAT |
| | | AATCCCCAGCTGACTTCAGTCTTGCTTACTCCACTCCAGGTTCAGCTTCCTTACATACACCAGG |
| | | CATGCTCCTGCCCAGAGCTTTGCCCATGTGGTTTCCCCAGATACCCATGTACTTGTTCCTTTAC |
| | | CTCCCTCAGGTCATTCCTCATATGTCACCATCTCAGTGGGCCCTTCCATAAAACCCAGTTAAAA |
| | | ATGGCAACATCCTTCTCCCCTCACACGCTAACTGACACACACACATACACAAACACACTCTCTC |
| | | TCTCACACAGCATACGTACACACACTCCCTATCATATCCCTTTCCTGCAATGCTTTTCTCCATA |
| | | CCATTTACACCATCTGA |
| 6 | RR54 v2 | TTTTAATGACTTCAAGTCACAAAGACACTGAAGAATTTTTAGCATTAAATGTGGTGAAAAGCCG |
| | (+) strand | GGCAGCACTCAGGAGGCAGAGGCAGGCAGATTTCTGAGTTCGAGGCCAGCCTGGTCTACAGAGT |
| | paRNA | GAATTCCAGGACAGCCAGGGCTACACAGAGAAACCCTGTCTCGAAAAACCAAAAAGAAAAAAAA |
| | Mouse | TGTGGTGAAAGTACACTGAAAACAGAAAATTAGTAGAAAATAAATAAGGTCTTGTTTGTGAATT |
| | | TCCCCAGAAAAAAAAAATTCTAAAATTCTAAAAACAAATACAAAAAGAAGGAAGGAAGGAAGGA |
| | | AGGAAGGAAGGAGGGAGGGAGGGAGGGAGGGAGGGAGGGAGGGAGGGAGGGAGGAGAGGGGAGG |
| | | GGAGGGGAGGGGAGGGAAGGGAAGGGAAGGGAAGGGAAGGGAAGGGAAGGGAAGGGAAGGGAAG |
| | | GGAAGGGAAGGGAAGGAGAAAGAAAAAGAAAGAAAGAGAGAGAGAGAAAGAAAGAGAGAGAGAG |
| | | AATAAAAGAAAATATGTCTTAGGACTGGATGTTCGATGCCTTGCCTCATATAATTACAGTGTAG |
| | | AATGCAACCAATCTGTATTAGACCACATCTTCACCCATTGGCTGACTCAGGAACAGTCCTGGAA |
| | | ACGGCTTGTAGTAGTCCTGTTAGTCTCTGAAACCCTAGCAATGAGGTAGGAATCCGATTCTCAA |
| | | CGACAAACAGGTTTGGCAGGTGCTTGAGACTATGAGAAGGTTTTTTTTGTTGTTTGGTTTGTTA |
| | | GTTGTTTTGTTTTGTTTTGTTATCAACTGAAGCAGTCACAATGTTGCTGAGTCCTACTTTTTCA |
| | | GCTCATATCTTATAATGTCCAATTAGCACCTAAACTATTTTAATCCATTGCCAAAAACTATAAT |
| | | CAGAATTTCTTAATAATTTACAGTAATAAAAATTTTAAATTAATAACACATAATTCAGAGCTGA |
| | | AAACAAAATTTAATGTTATTCGCTTTTCCTCTGAAATTCCATGTAATGAATTATTTTTATCTCT |
| | | CCCAAATCTCTGAGGAAAGGACTGAGAATTTTCAAGCCTTTCAGAAAAATCAATTTTCTGTAAC |
| | | ATCGGAGAATCTAGCAGTTTAAAGCAGGAAATTAAGATTTCTACATAATGGTTATCTGACTTGG |
| | | CTGTGTAAGTAGGAACACCTCAAGAACTCTTATTTGACCACAAAACCCCCAGTGTCCCTCTCTG |
| | | TTCCATGACATGTGATATTTAACCTGCCTACTATATATATGTGCTTCTGTTCAACTAGGGTGAA |
| | | GTTTCCTTTTGATAAAAGATGGAATCTTATCAATTCAGAGCCGAACACTGGAGGTCAGTCCCAT |
| | | GACCATAACTGGTAGCCTCAGCAGTGACTTTTCTATCTTAGAACATTTGCATCAGCTCCTGGAA |
| | | GAGCACAGTCGAGCAGAAGGGTGGAGCCCAGCGTCTGCATGGATTGTCCTTGAGAACCTAAGGG |
| | | ATGAGAGCATCCAGCGATGGGGACGAATGAGGGGCCTCCGAGATACTCCAGATGAAAGGGGACA |
| | | AACACAGGCTCCTGTGAGCCACGCAGGTACAGGACAAAAAGTCAAGGTCCTCCAGTTTCTTGTG |
| | | TGCATCTCATGGTGGCCAAGGTGCTTTGTGACTTGGTGGAAATACCAGGCTAGAGCAGCACATG |
| | | AATGGGCTGAACTTCTCTAAGGGCCTGGTCCACACTAACAGCTGAGTCTGGTATTGCAAATAAG |
| | | AAGGAAGGGAGGGAGGGAGGAAGGGAGGGAGAGAGGGAGGAAGGAAGGGGGAGAGGGAGGAAGG |
| | | AAGAAGAAATGGATTCTCAGAAGCTGGTCTCAGAATGAAGAATCCTACAATCCTACTGCAGTAA |
| | | GAGCAAGGACCCCGCCGTTCCCCCTCCCTCTCTGAATCATGATTTTATTGAAATATAGTTATTA |
| | | CACTCATCATTTTGTTGTCTTTGGTTGCGTCATCGAGGATCTCTGACACAAAGTGAATGTTCCT |
| | | GCTGCATTCACTCTCGGGCTCTTTACGGAAGAGCCTTCTGCACTGGGTGTCCTCTTCCCATCCC |
| | | AGCCACACAAAGTCAGTTCAGAACAGTGGGGGAGTTCTCAAGGAGGTTACTTCCCGACTAAAAT |
| | | ATTTAGTGTGCCAAACCTTGTAACCTGATTTACCCTAGTGGAGCTTCCGGAGGGGACAGAAGAC |
| | | AGGTGACAGAATTCATGGAAAACTACAGACCTTGGCTGAGGGAGAAGCATGGGCACTAGCCTGG |
| | | GAAGGCTCTGGAATGTGCATATTCACCTTGGATTTGGGAGGAAAACCCAAAGTAGACAAGGGAA |
| | | GGTGGAGAGGAGGGAAGAACACATAGAAGGCCCAGACCCCAGAGACAGAATAGGAGCAAGGGAC |
| | | GAAGGATGGACTCCCCACTAGGTCTGGGGAGTACAGGGCCATCTCTGGAGCAGCTGTCGATCCC |
| | | TTGGTTCCTTGTGATCTTTAACCCTGGGGATTGGGGCTTTATCCTGAGAACAACGGAAGCTAGT |
| | | GAGGGGTTAACACTTTAACATCTCCGATAAGTCTTGGGATTTTGTGATTATTAGTTCAAATCAT |
| | | GCTTCTATAATGCCAAGTTTAAAGGACATCTCTCTGGGGTTTTTATTTTGGGTGATCTCTTTTT |
| | | TCAATCCATTCACCTAATTTTGCCACACACACACACACATTTTTGAGACAACTGAAGTACAAAA |
| | | TAGCACTGCTGTAAGACCGTTACCAGGCCTCCCTCACTGTATATAATACTAAGAAGTCAGCCAT |
| | | TTGTCTTCACACGTCTACCCAAGCACTGTAGCTTTACTGGATGACAAGGTTGGGCTTTTGGTAA |
| | | ATCTGAACCACAACCCTCCCCCATAAACTGAGACTACCAAATAAAACAGACTCGGAAAGCAAAG |
| | | ACTTAAAAAACGCCATAGGCAAATGAGAGTCACTTACCTACAGATGTGAATTTGAGGGAGCCAG |
| | | GAGAAGCGAGAGACGCCACTGTGGAAAGTCAGGGTTGTACACCCCTTTCCAAGTTCAGTTTTCA |
| | | TGGTAATAAAGCATTGAACAGAAATCAGGCTTTTTTAAGAACACTATCTATTTGCTTATAGGTC |
| | | AATGGCCTAAAGTCCAGATCTAGCACAGTTCAGTGACATGAGAGGAAAGGTCGAACATACTCAA |
| | | AGCAGGAACCCAAACCCAGAGTGCGTGGGGACCTTCTGAGGGTATCAACAAGCTTTAGAAAATG |
| | | CCCAGTTGCCCGAGAGTGGGGCCTCATGGTAGAGAGCCTGGTGAGAGGCCCGACTCTTCTTTTT |
| | | TAAGCCCTAGGTTTTAAAAAGTAACTTTAAAAAAATCTAACAGGACCAAACTACTTCTTATCGT |
| | | CTCTGCTCTATTTGAAGTGACCCTTGTGACATCCTGGGTTGCAGGCCCTCAGTGATCTGGAGCC |
| | | AGCCCCAAAGTTTCATGGAGAATTGATAACATGCAATTGCCTTCTGCTCAGAACTCTGTGCACA |
| | | CAGATGACCCTTGAACTCTGGTCTGAGCAGTCAATAGCCTCAAGCTCTGCCCACTAGCTGCAGC |
| | | GCTGTATAAGTAGAAATGCATTACTTTAAAAAACACATTCCCATTAACAAAACCTCAAAGCAAA |
| | | TAAGCTTAGAACTGGCTGTGACTTTTCGTATGCCTTCTTACTATTAATCTCCACATATTTCTTT |
| | | AAAAAAACAAGTTTTACCATTTTTCTTCTTCATTAATTTCAATTGCATTGTATTATAAAAAGTA |
| | | ATATGGCCATATGATTCAAAATGCTATTTAGTTTTTATATTTCTTCCTGGAAAGGGGATGTAAG |
| | | ATTGTTACTGGACATGGGGTCCCAACTTGTGCTCCAAGTCAGAACCGATCAGCTGCCTTCTATA |
| | | GGCCAATAAAATAGCCAAGTTAGTAATAGAAAGCAGAGAAAGGAGATTTATTCAGTGTTCCACA |
| | | TTGGGAAAAAAATACAGAGATCCAGTGTATATCCAGTTTTGGGCTCCTGAAATGAGACCAGAAT |
| | | TTAATTAGAACACCAAATGCACATCTTAGCAGTGCCAGTTAAAATATGATCCTGCCCACCACTG |
| | | ACTGGGCTTCAGGCTGGTTCCTGAAGTGGTCTACCAAGTTGTGAGACTTGTGTGAAGTCTCAAG |
| | | AGACTAGTTCTTGCTCTGGCTGGTATCTCTTTCTTCTCCCAGAGTGAGATTCCTGAGGAAATTT |
| | | CAATTTGGAGGTGGGAGGACCAGATTCACTACAGTAGTCTGATGGTCAGATGGAGAGACACATA |
| | | GCATCTCTTTACACTATCTTCATTTCTTCCAGGCCAGAGAGAGGACAAGAAAAAAGGCTGAGCT |
| | | TGCAAGACTGTGGTTCTTAAAGCATACCCACCACACCAGAGATGGCTACTGAGCATAAAGGTTA |
| | | CAATACAGTGGTATGCATGCTTACCTAAGAAGGTGAAGGTAGGCCAAGAACAGTTTTCAGAATC |
| | | AAGGAGATGAATTGCAGACAGACTGGAATGTTAGCCCTGTGGCTCAGAATAATAGGAATGGTTT |
| | | TGTGGAGAAGTCTTCCACCAACTATCAACTCTTGTTTGGTTATTATAGCCCTGCAGATCAAAAT |
| | | GAATTGGAAAAACTCAACACCAATTATCTAACACAACATAATTGGCTGTACTTTGGTAGAGTAG |
| | | GGAGGTTTAACTCATTACAGAGAGAAGCAAAAGGAACTGGCTTCATAATTAGGTTCTAGCACTT |
| | | GCTTCTCGATTTAGCTATGGTAATGATGCTTGCTCTACTGGCTGGTTTTGTGTGTCAACTTGAC |
| | | ACAAGCTGGAGTTATCACAGAGAAAGGAACCTCCCTTGAGGAAAGACCTCCATAAGATCCAGCT |
| | | GCAAGGCATTTTCTCAATTAGTGATCAAGGGGGGAGGGCCCATTGTGGGTGGTGCCTTCCCTGG |
| | | GCTGGTAGTCTTGGATTCTAGAAGAAAGCAAGCTGAGCAAGCCAGGGGAAGCAAACCAGTAAGG |
| | | AACATCCCTCCATGGCCTCTGCATCCGCTTCTGCTTCCTGACCTGTGTGAGTTCCAGTCCTGAC |
| | | TTCTTTTGGTGATGAACAGCAATGTGGAAATGTAAGCTGAATAAACCCTTTCCTCCCC |
| 7 | RR62 v1 | AAAATTTCAAAAATGAAGGTGAAAACTATAAATTCCTCTGAGGGAGCCATTCCTAGTGTCTTTC |
| | (+) strand | TAGCCAAGATGTGCCCCACCCTTGATTCTTGAACCTGAATATGTGGGTTCCTATGTGCAAATAA |
| | eRNA | GCTGTTCTGAACTTGAGCCAGCATGAGGCACACTGCAAAGGCCATGCTATGACAGTGAGTCAGC |
| | Human | CCTGAGTACACGACAGACTCACTGATTACAAGGTGGCCTGTCAGGCTTATCACAAAGAAAACAG |
| | | TAGCATAGTGACTGTGTCTGTGTGAGTTCTGGCTACTTCTGCGTGGTTGCCTCTGATCACAGCA |
| | | CTGCCCTAGTTTTCTCACGCCTGTAATCTCAGCACTTTGGGAGGCTGAGGCAGGCAGATGACCT |
| | | GAGGAGATTGAGACCAGCCTGTCCAAAATGGTGAAACCCCATCTCTACTAAAAATACAAAATTT |
| | | AGCTGGGCATAGTGGCGGGCGCCTGTAATCCCAGCTACTCAGGAGGCTGAGTCATGAGAATCAC |
| | | TTGAACCCAAGAGGCAGAGGCTGCAGTGAGCCAAGATCACACCACTGTACTCCAGCCTGGGCAA |
| | | AAAAGGAGACTCCATCTTAAAAAAAAAAAAAAAAGAAAAAAGAAAAAAACAAACTCAACTACTG |
| | | GTTGAGACTAGTGTACCTTACTCAAAGTAATGCAGCCTGTTCATGAAATTCCCATTACAGATGG |
| | | AAGGGTTAAGATCAAGTGGATCACCAGCCACAGCCACAAAGCTAAGGGGCAGAGGGGAGTTGGT |
| | | GGGGGGGAAAAATAGAACTCTTGGAGATGATACTCAAGTAAAATTCATGGGTCACCTCCCTAGT |
| | | GACGTTAAGTATTGGTAGTGTCATGCTCAAGCCTATACTCAATAAAGTGGTAACATTTTGGCAA |
| | | TGTATTTGGTGAGTGACTTTCAATCCTGTTTCTATTAAGATTTTAAAACATAGTAAGATAGAAG |
| | | GAAACTTGAGGCAGAGTAATTTCTAGAAATATGGGTAAACTCATGTTTCTATTCTCTTTGTGTT |
| | | AGATACCACTCCAGCTCAGCCAGTAAAATCCCCTCTATACATTTTGAATTACAATTTAAGATAT |
| | | GAGCAAAAAAGTAAAAAATTAAAAACAAAAACAACCTACCCACAACGTGTTCCATTTGTCATGT |
| | | TCTGGTTGAGGGAACTGTTAGTCCATACAATGGTGTTATTCACACATGCTTTTCCTGGAATCTG |
| | | GAGTTCTTGTAACTCAACGTCGTAGTCAATAAAAACATCTGTCATTGTGCCAAAAATGAGTAGC |
| | | ACGCCTGGCTGGGCTATTCCATGGAGAAATGCACACAAACTTCCCACAAACATCAGCCAAATGT |
| | | CAGTTGATGAAGAAAACCGAAACTTGAAAAACAAAGGGTTCAGAGATCATCTATGGGTGAAGAG |
| | | CAGGAGAGGTAGGAGGACTACTTAATTTAGTTACTAGATTCTCATGAATAGTACTCAACACCAA |
| | | ATTTCATGGGAATCACCTTAATTGAGTGGCAGAGTTCGTATACTAATTATATTGAAATAAACGG |
| | | CACGGTACATCAATTATCTAAAACATTAGACTCTTACATTGTTAGATCATGTTATAAATTTCCT |
| | | GCTCTCTGAATTCATTCTGTTTATTTCAGGAGCTGACTTTCGTTTTTAGCTACTCATCTACAAC |
| | | TTAGGGGAAAATAATCACTGTCATTGATGCCATTGCCAAGTAGATAAACTGTGTGGGGCATTGA |
| | | GAAATACTATAAATTGTTTTGTTGAACTGATAGTGACTATGAAGAATGCCAATTTCTGCCTAGA |
| | | AATGCCTCCATCCTGAGAAAATTTGGAGTAAACACCTATACTTTAAAATTTCTAAAGATTTGTC |
| | | ATAAGAATAAATATATATATGTGATTGTTGTGATGACTTTCCTTACAAATATGATAACCATGGG |
| | | CTTAGTGATAATTTTCCAAGACATTTTTCTGATCTATTGGATAGGATTTGTATGCTTTATGAGC |
| | | AATATATTTCTAATAGTTATTCAGCATGTTATCACATAGGCAAAGCCTATGACTGAAAAAGTGA |
| | | TCACTGAAGTTAAAAACCTGCCAATATGACTAAAGATTTAACACTCCCCTCATGATCTAAACAA |
| | | TTTATAGCTGCACACCCACTGCCATAAATCAACACAGTTTTATTACCAATTGAAAGAAGCCAAC |
| | | TCTAACGCCATCACCTTTCTTCTCATCTTGTAACCTGATGAGAAAAACATAAGGATTTAAAGAC |
| | | CACCCTTTCCAATACAATGGGAAATTCTCCCTAGGTGGTGATTTTATGAGTTATTCTTTAGTAA |
| | | GAAAGAAAATCCCCACTGGCTGGAAAATTCTGGCCAAACCTAGTATCTTAAACACAAGCTTTAT |
| | | TCTTCTAGATTTCCCTCTTCTAGAGATTTCAG |
| 8 | RR64 v1 | AGTGCACACAGGCAACATTGAAAGGACCAAGATGGCTGCTGAGAGAAGAGGCAAAAAGTGGCTT |
| | (+) strand | GTAAATTTGCTTAAGTAGGTGAAGAAAGACAAAGAAGGGCTTTCAGTGCCAGTAGGATGAGTTG |
| | eRNA | CATGCAGTCTAAGATAAGAACTTCTGTTCACGGCCACCCAGGGCCAGTGTTTACAAAGCTGAGC |
| | human | CACAAGAAGGATGGGTTAGGCAGGAAGTCTTGCACATGCACACCAATCACAGAAGCTGGTTATT |
| | | ACCACCCTGAAAATCAATATAAATTGTGTAAAGCCAGCATGAGTCAGTTAACACAATATAATTG |
| | | GCTATGTTTTTTTGTGGCATAGAGACTAGCAGAGAGAAAAATGTCTAAATTAGAGCTTTCTTAG |
| | | GTAGCTTATGTATTTTTAAAGCAAGCTGTGTGGAAGGACACAATTTCTGTAGTTTAGTTGACTG |
| | | CAGAGGGAGGCAGAATGGTATAGTGAAAAGAGCACAGGCTTGTGAAGTCACAAAGAACTGGCTT |
| | | TAGATCCTGGTTTTGCCACTTACTTGTTGGATGATTTGGTTAAGGTAGTGATACTTGCTAATTA |
| | | TTTCCCCTCATGTGAAGTCCAGTAGGCCTTGAATAAGGGGAAATCGGTGTTGGGCAAAGGTGCT |
| | | CTGCTCCATGCAGTCATTCAGAGACCCAGGCTGACAGAAGCTCTACCATCCTCAACATGTGGCT |
| | | TTTAAGGTCTCCTTGGACATGGACATGTATCCATCACACAGGGAAGAGAAAATATGGAGCATTT |
| | | TGTATGGAAAGTTCCTGTGGGCCAATCCTGGAAGTGGCATCCATTACTTCTACTCAGATTCCAC |
| | | TGGAGAGGACTGTGCCACATAACCACACCTAACTGCAAGAAAGATTGGAAGATAAGGTCAGGCT |
| | | GTGTGTCTGGGAGGTTTAGTTAAAAGTTAACCAATCTCAAGAGATGGTCTTTGAAATCACAAAG |
| | | GTTTGTGTTCACTTACTAGCTATCTAACTTTGGACAAGTTACTTAATCTCCATTAGCCTCAGTT |
| | | TCTTCATCTGTAAAATGGGAGTGAGAATACCACCTGCACAGGATGATGAGGCCTGCCTGAACTT |
| | | CCCTGTGCCATAGATTCTGGATTGGGAGTTCCTCCCCCAAGTAGCCATTTAATTGAATTGTGTA |
| | | TATTTTTTTACCTACCAAAATTAGTGTATTCCTAATTTTGTCATGTACACATGTAGAAAAGACT |
| | | GGAAGAAGTACAATATAAAAAGTGCTTATCTGTAGGTTATGCAAATGTGAATGAGGGTGGTTTT |
| | | CATTTTCTTCCTCATGCTTTTCTGCATCTTCTAAATTATATACAATAAGGGTAAACTATTTTTT |
| | | AAACATATAAGTTATTAAATGAAAATAATGAATATGACAAAGGAAATATCAATAATAATGCATA |
| | | ATAAAATTTTATTAATGATTTTTAGAAATACATAGAGAGTACAACTATGTGGGTGTTCTCCAAA |
| | | TATTAGCTGTAGTTCAGTCATTGCTTTT |
| 9 | RR65 v1 | AAAGAACAGTACAACTAAGTGATTCTTCAATGACTCATCTCAAATCTTCATTCCGATGACTTCT |
| | (+) strand | CAAGGCTTCGCTGAGTGATCCCTCACCCAAATGATTATTGACTTCCCTCTCCTCCCTCACCCCA |
| | eRNA | GATTTCTCATGTGTCACTGCCAATTCAAATTCATGGCTAAAGCACTAATCTCTTTCCACACCTC |
| | | TACTTCCTGGAATCCTTTCTTGTTATCAGTCTTATATCTATGCCCAATTCAGCCAGCCATTGGG |
| | | GTAATGAACTTTCCATTCTAGAACCACAGTCCAATTTGCCTGTTACCTCACTGAGAAGGCTCTG |
| | | GGGCTCTGACAAACACTGCTGTGAGTTTCCTGAAACCTACTTTTTTTTTTTTTTTTTTTTTTTT |
| | | TTTTAACTTTTATTTCAGGCTCAGGAGTACCTGTGCAGGCTTGTTATATAGGCAAACTCCTGTC |
| | | ATGAGGATTTGATGTACAGATTATTTCATGACCCAGGTACTAAGCCTAGTAACTCAATAGTTAA |
| | | TTTTTCTGCTCCTCTCCCTCCCCCCATCCTCCACCCTCCAATGGGCCCCCAGTGTCTGTTGTTC |
| | | CCCTCTTTGTGTCCATGAGTTCTCATCATTTAGTTCACACTTACTGGAAACCTCCTTTGGCCAC |
| | | ATTTTGACACTATGTCAGGAGAGGTTGGATCCTCCAACCCCTGCCGAGATCACTGCAGATACTT |
| | | AGAGCCTGGATAAGATGATCAACAGTGCTTTAAATAGCTTATATTTTAAAAAGCTGTGCTGAAT |
| | | GTTTTGCAGGCCACTGAGGTGCGGACATGGTTTTATATCAGTGAGATCACAGGTGAGCAAGGTG |
| | | TTATTGGCTATAATGTTTGAAAATCAGCCTTTAACATCTGATTATATTTGACTTCTTATTTGAG |
| | | TGTCCTTGGACCATGTGTGATCAGACTGCTATGTGAATAAAATAAGATAATGTATAAATTTTCT |
| | | ATAACTTCTTCATGCATAAAATCCTATTAACATTGCTGAT |
| 10 | human | ATGCAGTTTTTCAGTTAAATTCTATTCTCATTTATAGCACAGTTTTAGTATGCCTGATTCCATA |
| | RR84 v1 | ACGTGCTATCTAATGTGGCTGCTCTAATAATAATTTCTCAGTCCTTGGGAGTGTTTTTAGCTGA |
| | (+) strand | TGTGACCCGAAATTCCTATTCCTATATGTTGTCCAGGCCTCAATTCCTGGGGAACCGAGACAGT |
| | eRNA | GACTGGGATAACTCTTGGAATGGGTCAGATCACCTCTCTCTGAAAGAGGTTATCTGAAAGATAA |
| | human | GATGAAATGGAGAACACAGTTGTGATTGCAGAAATCCTTCTTGTGTGCTTGTTAATGCTCTTTG |
| | | TCTGTTGTGCCTTTTCAGGGAAGTGCCCAAGCTTGGGAGGACATGGAGAACAATAGGCATATTG |
| | | GAGAGTGAGTGGGGTTTGGAGTCAGAGCCCCTTAGACTTGAATCCTACTCTGCCAGCTGTGCAG |
| | | TCCCAGACAAATCCCCTTACCTTTCTGAGCCTAGTTTCTTATATTTAAAATGATGTTAGTGCTC |
| | | ACTTTATAAGATTGTTGTAAAGATTAGAGATAATGACTATTAAGCTCCTAGTAGTGCCTAGAGA |
| | | AACAGTGAATGCTCAATAAATGTAATTGCTGTTAATAAAACAATATTTACCTATTAAAACATCA |
| | | TTAATCCCTATTCGATGAAATGTCATTTGACACTGGGGAGTAATCTAACAAACTACATGAAGAT |
| | | AGTGATAATTATGTTGCTAACTGTACTCAGGAAAAGGGACTCAAGCTTCACATTTTTGGCTGTT |
| | | TATGAAGGCAAATGTCTTCCCATGGAGAGATGCAAAAAGACATTCTTACCAGACCAATGGTGGC |
| | | TGCTCCAATCCCAATGAGAGGGCTGACAGAAATAATAACCAAGGTCAGTTTCCAACCCCTGAAA |
| | | AATCCCAACAGGAAACCACAGATGGTCGAGGTCATGCGCTGAATGAAAAGGGCCATTTGGTCAG |
| | | CTATGGCATCATTGATTTTATTAATATCACTAGAAACCAGAAAGATTTTGGTCACTAAAACTGA |
| | | ATTTGATCATTCAAATATCTTGCTGAAAGTCTCAGTCTTTCCCTTTTAAACATTCCCATCTCTC |
| | | TATCCCTATCACCCTTGGATAGCATTAACAGCCTCTCAGATCTTGGAAAATTTGCTGCACCTTG |
| | | GGATGTTATCAAGCCTCTTTCCTGATAGCCAATCAATTTTGTTCTTAATCTAGGTTTCCCAGCT |
| | | GGTACTCAGTGTTGATAAAGGCTTTTGGAAACCATCTTATTTGTCTTTTTCTTTTACATAGCTA |
| | | ATATCCTTGCTCTGTCTTGCAAAGCTTTTCTTTCTTCATGCCTGCTGAATCCTTTCAGCCTCAA |
| | | GGTTCTAGTCACATACCTTCTCGTGCATCAATCTTGTATAATCCCCCAAGAAGAAGTAACACCT |
| | | TTCTCCCTGTGTTTCCGTAACACTGTAATTAATCCTTGTGCATTTGTATGTGTGTGGAATTATT |
| | | GCATTCTGCATATAGGCAGTATAGAGTAGTGGTGAAGAGTGCAGATTCTGGAGCCAGACTAGGA |
| | | ATTTGAATACAGACTCTTGTTGGGCAAGTTACTTAACCTTTTTGTGGCTCAATTTTCTTGTCTG |
| | | TAAAATAAGAACAATAATAGAACCTACCCCAATGGGTAATAATGAGGATTAAAGTAGCGAATAT |
| | | ATGTAAAGCCTATAGGACAGCGCCTATCATATAATAAACAATATGTGTTAGTTCTGATTATTTG |
| | | CTTGCATTTCTTATTTCCCCTACGAGGCTGTAAGTTCCTCAAGAGCACAAGTGCAAGGCAATTG |
| | | CTCCAGAATATTTATTGAATATAATTATATTAAATTCATATTCCTGAGAACATTTCCTTAATCT |
| | | CTTGTCCAGTTGGAGTGAAGAGAGCTTGGAACTAAACAGCAAGAAAGAACCTAAGACATCACCC |
| | | AGGTATTAGGCCATTGTGTTCCAGCAGGGTTAGATAACTGCTCAAGGTCACAGTAAGGAGCAGA |
| | | GTAAGTGCCGGAAGTCAGGGGTCCTGAATTTCAGTGTGCTTTATCTACACTGTAGAGTGACTGT |
| | | CTTTCAGGGGACCTCAAAATTCA |
| 11 | RR54 v1 | AGAGAGAGAGAGAATAAAAGAAAATATGTCTTAGGACTGGATGTTCGATGCCTTGCCTCATATA |
| | (−) strand | ATTACAGTGTAGAATGCAACCAATCTGTATTAGACCACATCTTCACCCATTGGCTGACTCAGGA |
| | paRNA | ACAGTCCTGGAAACGGCTTGTAGTAGTCCTGTTAGTCTCTGAAACCCTAGCAATGAGGTAGGAA |
| | Mouse | TCCGATTCTCAACGACAAACAGGTTTGGCAGGTGCTTGAGACTATGAGAAGGTTTTTTTTGTTG |
| | | TTTGGTTTGTTAGTTGTTTTGTTTTGTTTTGTTATCAACTGAAGCAGTCACAATGTTGCTGAGT |
| | | CCTACTTTTTCAGCTCATATCTTATAATGTCCAATTAGCACCTAAACTATTTTAATCCATTGCC |
| | | AAAAACTATAATCAGAATTTCTTAATAATTTACAGTAATAAAAATTTTAAATTAATAACACATA |
| | | ATTCAGAGCTGAAAACAAAATTTAATGTTATTCGCTTTTCCTCTGAAATTCCATGTAATGAATT |
| | | ATTTTTATCTCTCCCAAATCTCTGAGGAAAGGACTGAGAATTTTCAAGCCTTTCAGAAAAATCA |
| | | ATTTTCTGTAACATCGGAGAATCTAGCAGTTTAAAGCAGGAAATTAAGATTTCTACATAATGGT |
| | | TATCTGACTTGGCTGTGTAAGTAGGAACACCTCAAGAACTCTTATTTGACCACAAAACCCCCAG |
| | | TGTCCCTCTCTGTTCCATGACATGTGATATTTAACCTGCCTACTATATATATGTGCTTCTGTTC |
| | | AACTAGGGTGAAGTTTCCTTTTGATAAAAGATGGAATCTTATCAATTCAGAGCCGAACACTGGA |
| | | GGTCAGTCCCATGACCATAACTGGTAGCCTCAGCAGTGACTTTTCTATCTTAGAACATTTGCAT |
| | | CAGCTCCTGGAAGAGCACAGTCGAGCAGAAGGGTGGAGCCCAGCGTCTGCATGGATTGTCCTTG |
| | | AGAACCTAAGGGATGAGAGCATCCAGCGATGGGGACGAATGAGGGGCCTCCGAGATACTCCAGA |
| | | TGAAAGGGGACAAACACAGGCTCCTGTGAGCCACGCAGGTACAGGACAAAAAGTCAAGGTCCTC |
| | | CAGTTTCTTGTGTGCATCTCATGGTGGCCAAGGTGCTTTGTGACTTGGTGGAAATACCAGGCTA |
| | | GAGCAGCACATGAATGGGCTGAACTTCTCTAAGGGCCTGGTCCACACTAACAGCTGAGTCTGGT |
| | | ATTGCAAATAAGAAGGAAGGGAGGGAGGGAGGAAGGGAGGGAGAGAGGGAGGAAGGAAGGGGGA |
| | | GAGGGAGGAAGGAAGAAGAAATGGATTCTCAGAAGCTGGTCTCAGAATGAAGAATCCTACAATC |
| | | CTACTGCAGTAAGAGCAAGGACCCCGCCGTTCCCCCTCCCTCTCTGAATCATGATTTTATTGAA |
| | | ATATAGTTATTACACTCATCATTTTGTTGTCTTTGGTTGCGTCATCGAGGATCTCTGACACAAA |
| | | GTGAATGTTCCTGCTGCATTCACTCTCGGGCTCTTTACGGAAGAGCCTTCTGCACTGGGTGTCC |
| | | TCTTCCCATCCCAGCCACACAAAGTCAGTTCAGAACAGTGGGGGAGTTCTCAAGGAGGTTACTT |
| | | CCCGACTAAAATATTTAGTGTGCCAAACCTTGTAACCTGATTTACCCTAGTGGAGCTTCCGGAG |
| | | GGGACAGAAGACAGGTGACAGAATTCATGGAAAACTACAGACCTTGGCTGAGGGAGAAGCATGG |
| | | GCACTAGCCTGGGAAGGCTCTGGAATGTGCATATTCACCTTGGATTTGGGAGGAAAACCCAAAG |
| | | TAGACAAGGGAAGGTGGAGAGGAGGGAAGAACACATAGAAGGCCCAGACCCCAGAGACAGAATA |
| | | GGAGCAAGGGACGAAGGATGGACTCCCCACTAGGTCTGGGGAGTACAGGGCCATCTCTGGAGCA |
| | | GCTGTCGATCCCTTGGTTCCTTGTGATCTTTAACCCTGGGGATTGGGGCTTTATCCTGAGAACA |
| | | ACGGAAGCTAGTGAGGGGTTAACACTTTAACATCTCCGATAAGTCTTGGGATTTTGTGATTATT |
| | | AGTTCAAATCATGCTTCTATAATGCCAAGTTTAAAGGACATCTCTCTGGGGTTTTTATTTTGGG |
| | | TGATCTCTTTTTTCAATCCATTCACCTAATTTTGCCACACACACACACACATTTTTGAGACAAC |
| | | TGAAGTACAAAATAGCACTGCTGTAAGACCGTTACCAGGCCTCCCTCACTGTATATAATACTAA |
| | | GAAGTCAGCCATTTGTCTTCACACGTCTACCCAAGCACTGTAGCTTTACTGGATGACAAGGTTG |
| | | GGCTTTTGGTAAATCTGAACCACAACCCTCCCCCATAAACTGAGACTACCAAATAAAACAGACT |
| | | CGGAAAGCAAAGACTTAAAAAACGCCATAGGCAAATGAGAGTCACTTACCTACAGATGTGAATT |
| | | TGAGGGAGCCAGGAGAAGCGAGAGACGCCACTGTGGAAAGTCAGGGTTGTACACCCCTTTCCAA |
| | | GTTCAGTTTTCATGGTAATAAAGCATTGAACAGAAATCAGGCTTTTTTAAGAACACTATCTATT |
| | | TGCTTATAGGTCAATGGCCTAAAGTCCAGATCTAGCACAGTTCAGTGACATGAGAGGAAAGGTC |
| | | GAACATACTCAAAGCAGGAACCCAAACCCAGAGTGCGTGGGGACCTTCTGAGGGTATCAACAAG |
| | | CTTTAGAAAATGCCCAGTTGCCCGAGAGTGGGGCCTCATGGTAGAGAGCCTGGTGAGAGGCCCG |
| | | ACTCTTCTTTTTTAAGCCCTAGGTTTTAAAAAGTAACTTTAAAAAAATCTAACAGGACCAAACT |
| | | ACTTCTTATCGTCTCTGCTCTATTTGAAGTGACCCTTGTGACATCCTGGGTTGCAGGCCCTCAG |
| | | TGATCTGGAGCCAGCCCCAAAGTTTCATGGAGAATTGATAACATGCAATTGCCTTCTGCTCAGA |
| | | ACTCTGTGCACACAGATGACCCTTGAACTCTGGTCTGAGCAGTCAATAGCCTCAAGCTCTGCCC |
| | | ACTAGCTGCAGCGCTGTATAAGTAGAAATGCATTACTTTAAAAAACACATTCCCATTAACAAAA |
| | | CCTCAAAGCAAATAAGCTTAGAACTGGCTGTGACTTTTCGTATGCCTTCTTACTATTAATCTCC |
| | | ACATATTTCTTTAAAAAAACAAGTTTTACCATTTTTCTTCTTCATTAATTTCAATTGCATTGTA |
| | | TTATAAAAAGTAATATGGCCATATGATTCAAAATGCTATTTAGTTTTTATATTTCTTCCTGGAA |
| | | AGGGGATGTAAGATTGTTACTGGACATGGGGTCCCAACTTGTGCTCCAAGTCAGAACCGATCAG |
| | | CTGCCTTCTATAGGCCAATAAAATAGCCAAGTTAGTAATAGAAAGCAGAGAAAGGAGATTTATT |
| | | CAGTGTTCCACATTGGGAAAAAAATACAGAGATCCAGTGTATATCCAGTTTTGGGCTCCTGAAA |
| | | TGAGACCAGAATTTAATTAGAACACCAAATGCACATCTTAGCAGT |
| 12 | RR75 v1 | AATCCTGTCCTTACAACCACTGGAGGAAACAAAAAACTCAAAGCTAAGCAGCTCTCTCTCTCTC |
| | (−) strand | TCTCTCTCTCTCTCTCTCTCCCTCCCTCCCTCCCCCCCTCTCTCAGTTCTCTCTCTCTCTGCCT |
| | eRNA | CATCTTTTCCACTTCCCATGTAACCTCCTCTCTAAACAAACAGTTTTCCCTGGTGATAAGGAAC |
| | Mouse | AGAGAGGGACTTTTTAGTCATTCAGGATGCACCCTCTATACTGTCATTGAGCACCCCTCAGGGC |
| | | TGACGGTCATCTCCAAGACTGGTGTCCCAAAGGACATGTAACCCTGACCTTGAGTATCATTGAT |
| | | TTGTGAGGCCCACCTTTACTATTGTCTGTAAATGTTAGTCTTTCTTTTAGTCTCTGCATTCTGT |
| | | ATTCTTCTAGGTCACTGGACTAGGCCAGCTGCAAACCAACATGGATCTTTGACCCCTAGTAGAA |
| | | CAACAATCCCAGGTCAGGGCTTTCAGGGCCATGACCTGCATGACCTGCATAGAGAAAGGGACAG |
| | | CCAGTCAGCTCACCTGAGAAGTAAATGAATAACCACAGCTAGCACAACTGCCCCAACCACTCTT |
| | | CCATGAACTGTGGTCACATCTCTGCAGTTCCCGGGTGCCAGGTGACTTATCAGTAGGCCTTTGA |
| | | CCTCGGTTGCTTTAGTTGTTTATCAGCTGTGTGTTTCAATTTATCTCACACCAACCTGTTTGCT |
| | | AAAGAGTGCCCATTCCTTGGCTCCAAGCACTAAAGCTGTTTTGGGGAGTACATCCCTCAGAAAA |
| | | CTCAGAGTACTTTATGCATCCTCTCTGAGCCTTGCCCAGTGGCAGCTCAGGGCTAGGATGTTTT |
| | | CATTAGAGAGCACCTATCGAAACAACATGGCTTAATATGTTGCACCTTCCCTGCTGAAAGGGCT |
| | | GCTCTGTCTATTTCTTGGATGTAAGCAAAGGCTTTCCAATTACAGCCCTTTTGATATTTTGAAT |
| | | TCCATAATTAATAAGGTGAAGTCTGGCTAGTGTATGGCATGCTCAGCAGGATCCTTGACTTCTA |
| | | CACATGACATGTCAGTAACACTTCTGATAACGATGTCACCAGACAGTGCTAAGTGTCCCTTAGG |
| | | GGACAAAATTGCTCCTCCTTGAGAACTGCTGTCATAGAGATTCCTTTCTTGAGGGTCCAGAATG |
| | | TCACTGTACCCAGGACCACTTGTTTGGAAACTCAGTCATTACTGTTGTAGTGATGTTTCCTTGA |
| | | TGGGCGGTTATATTCGTTAATGACACTAACAGAGAGGTTTTGCTGCTTAAAAAACTGCTCATAT |
| | | CCTGTTTTGGCAAGCACTAACTTTTGTAATGTAAAAAATATCTACTTTTCTTATTATGAGGATA |
| | | GAAGGTATCCAACAAACCGAGTTATATATAATGACATTTATGGATTTAGAAATGACCACATTGG |
| | | CAAGCACAGCATTGAGTGCTACTAAGGCCTGGATCCTGGCTGAGCTGGGGCTATGACAGCTCGC |
| | | ACTTGGTAGAAAATCGAGCCTCTAGGATTCTGCAAATGCTCCCAGGAACCCCATAGTCCTCAGA |
| | | TTTCCGGTTCATAAAGTCAGGTGCCAATGTGAACAGCAGCTGTGGTTGACTGGATGCCACAGCC |
| | | TTTTGAACTGTGGCTATAGGGTCAGGTAGCTGAATGTGGGATTAAAAAAAAAGCAACAGTGAGA |
| | | GGTTATTGAATGAACAATAATTAAAAATTATTTCAAAACCAAAGATAATGGATCCAAGGTTTGG |
| | | GTAGGACTTGTCCTTCTTGTGTTGAATGGTTTCCCCGTTAGCCAAGGTTCTGAGCACACAGCAC |
| | | ACACACTTTGCCTAGACCACCTTGGCTCATAGTGGTGACTCTTGTATGTGAAGATCTTGTATGT |
| | | GAAATATTTTCAGCATATGTAGGATAGGAATTTTTTTTTCTTTTTAGAATGTAATAAATTACGG |
| | | GAAGGAAGGGCTTAATATTTTCTTAGTTATTCTTCCTCATGGAAGTACCACACTGGGATGTCTT |
| | | TGGATTGGATTGTGATG |
| 13 | RR76 v1 | GTTAGTGCTTGCCAAAACAGGATATGAGCAGTTTTTTAAGCAGCAAAACCTCTCTGTTAGTGTC |
| | (+) strand | ATTAACGAATATAACCGCCCATCAAGGAAACATCACTACAACAGTAATGACTGAGTTTCCAAAC |
| | eRNA | AAGTGGTCCTGGGTACAGTGACATTCTGGACCCTCAAGAAAGGAATCTCTATGACAGCAGTTCT |
| | Mouse | CAAGGAGGAGCAATTTTGTCCCCTAAGGGACACTTAGCACTGTCTGGTGACATCGTTATCAGAA |
| | | GTGTTACTGACATGTCATGTGTAGAAGTCAAGGATCCTGCTGAGCATGCCATACACTAGCCAGA |
| | | CTTCACCTTATTAATTATGGAATTCAAAATATCAAAAGGGCTGTAATTGGAAAGCCTTTGCTTA |
| | | CATCCAAGAAATAGACAGAGCAGCCCTTTCAGCAGGGAAGGTGCAACATATTAAGCCATGTTGT |
| | | TTCGATAGGTGCTCTCTAATGAAAACATCCTAGCCCTGAGCTGCCACTGGGCAAGGCTCAGAGA |
| | | GGATGCATAAAGTACTCTGAGTTTTCTGAGGGATGTACTCCCCAAAACAGCTTTAGTGCTTGGA |
| | | GCCAAGGAATGGGCACTCTTTAGCAAACAGGTTGGTGTGAGATAAATTGAAACACACAGCTGAT |
| | | AAACAACTAAAGCAACCGAGGTCAAAGGCCTACTGATAAGTCACCTGGCACCCGGGAACTGCAG |
| | | AGATGTGACCACAGTTCATGGAAGAGTGGTTGGGGCAGTTGTGCTAGCTGTGGTTATTCATTTA |
| | | CTTCTCAGGTGAGCTGACTGGCTGTCCCTTTCTCTATGCAGGTCATGCAGGTCATGGCCCTGAA |
| | | AGCCCTGACCTGGGATTGTTGTTCTACTAGGGGTCAAAGATCCATGTTGGTTTGCAGCTGGCCT |
| | | AGTCCAGTGACCTAGAAGAATACAGAATGCAGAGACTAAAAGAAAGACTAACATTTACAGACAA |
| | | TAGTAAAGGTGGGCCTCACAAATCAATGATACTCAAGGTCAGGGTTACATGTCCTTTGGGACAC |
| | | CAGTCTTGGAGATGACCGTCAGCCCTGAGGGGTGCTCAATGACAGTATAGAGGGTGCATCCTGA |
| | | ATGACTAAAAAGTCCCTCTCTGTTCCTTATCACCAGGGAAAACTGTTTGTTTAGAGAGGAGGTT |
| | | ACATGGGAAGTGGAAAAGATGAGGCAGAGAGAGAGAGAACTGAGAGAGGGGGGGAGGGAGGGAG |
| | | GGAGAGAGAGAGAGAGAGAGAGAGAGAGAGAGAGCTGCTTAGCTTTGAGTTTTTTGTTTCCTCC |
| | | AGTGGTTGTAAGGACAGGATTTTCTAGAGGGCAGAAGACAGGCAGCCCAGGTCACTACAAAACT |
| | | CTGTCATTATGTTTTGTCTGTAGAGCCCAAAGCCGTTCCAATAAAGTCCTTGGGGTGGGAGGTT |
| | | TCATTTTTCTTTCCCCAATTTTTACTTTGTATTTTTTTGAGATACAGTCATTCACATAGGGGGT |
| | | CCTGACTCCAGGACCCTCTCACACGTGGTGACTCTACAGCCCTCTTACGCACGGGCTTCCGAAC |
| | | CTGCGGATTGCTAATGTATAAAATGGCACAGTTCAAGCATATGACCTGTGTGGCACCTTTGGGC |
| | | GTACATTAAATTGCTTCTAGATCATTTGTAATGCCTAGTATGAATGCTGTATACATAGTAACAC |
| | | TGTTGTTCAGGAGAGAAGAACAATGAAAAGGTCTGTGTGTGGTCAGTGGAGATTCAGTTTACAA |
| | | AACTACTCAAACCTGCAGTCGGTTGAATCTGTGGGCATGATTTCACACTTAAAAAAAGTCAAAA |
| | | GAATGGTATGATCTAGAGGCTCTAAACATTAGCATCTTACCACATTTTACTCTCATTCTTTTTT |
| | | ATATATACATATGAGTTTTTGTTTATATATACATATATTATTTTCTAAACTTGCATTTTGTTGT |
| | | GTATTGAATTTTTGATCATTTGTTACCGTTTGGTTTGTTTTTGAGACAGAATCTTACTATGTAG |
| | | CCTTGGCTGGCCTGGAA |

The present disclosure describes ASOs that may be used to increase the expression of the target gene ABCB11 (e.g., human ABCB11 or murine Abcb11). Without wishing to be bound by theory, this increased gene expression may be due to increasing the amount or stability of the targeted ABCB11 regRNA, or interference with regRNA-associated repressors that inhibit the expression of the gene to thereby increase ABCB11 gene expression. These ASOs are different from the ASOs previously described that were designed to inhibit eRNAs (see, e.g., PCT Application Publication No. WO2013/177248 and PCT Application Publication No. WO2017/075406). Without wishing to be bound by theory, it is hypothesized that the ASOs' ability to upregulate ABCB11 regRNAs is attributable to the selection of a target sequence in the regRNA and/or the chemical modifications of the ASOs.

Increased ABCB11 gene expression can be at least about 0.1%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 350%, 400%, 450%, 500%, 550%, 600%, 650%, 700%, 750%, 800%, 850%, 900%, 950%, or 1000% or more increase in expression as compared to baseline gene expression, gene expression prior to treatment, or gene expression after treatment with a control ASO. Increased ABCB11 gene expression can be at least about 0.1-fold, 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold or more increase in expression as compared to baseline gene expression, gene expression prior to treatment, or gene expression after treatment with a control ASO.

ASOs that hybridize to (e.g., is complementary to) a portion of any of the regulatory RNAs provided herein (e.g., as described in Table 1 above) are contemplated by the present disclosure. In some embodiments, the regulatory RNA has a nucleotide sequence of any one of SEQ ID NOs: 1, 2, 3, 4, 5, 6, 7, 8, 9, 10, 11, 12, or 13.

### Sequences of ASOs

In certain embodiments, the ASO disclosed herein is complementary to a sequence in the ABCB11 regRNA that is no more than 300, 250, 200, 150, 100, 50, 40, 30, 20, or 10 nucleotides from the 5′ or 3′ end of the ABCB11 regRNA. In certain embodiments, the ASO disclosed herein is complementary to a sequence in the ABCB11 regRNA that is no more than 300, 250, 200, 150, 100, 50, 40, 30, 20, or 10 nucleotides from the 5′ end of the ABCB11 regRNA (i.e., the 5′ most nucleotide of the ABCB11 regRNA sequence forming a duplex with the ASO is no more than 300, 250, 200, 150, 100, 50, 40, 30, 20, or 10 nucleotides from the 5′ end of the ABCB11 regRNA). In certain embodiments, the ASO disclosed herein is complementary to a sequence in the ABCB11 regRNA that is no more than 300, 250, 200, 150, 100, 50, 40, 30, 20, or 10 nucleotides from the 3′ end of the target regRNA (i.e., the 3′ most nucleotide of the regRNA sequence forming a duplex with the ASO is no more than 300, 250, 200, 150, 100, 50, 40, 30, 20, or 10 nucleotides from the 3′ end of the ABCB11 regRNA).

In certain embodiments, the ASO is no more than 8, 10, 15, 20, 25, 30, 40, 50, 60, 70, 80, 90, or 100 nucleotides in length. In certain embodiments, the ASO is at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100 nucleotides in length. In certain embodiments, the ASO is at least 8, 9, 10, 11, 12, 13, 14, 15, 16, 17, 18, 19, or 20 nucleotides in length. In certain embodiments, the ASO is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In certain embodiments, the ASO is designed to lack a stable secondary structure formed within itself or between each other, thereby increasing the amount of the ASO in a single-stranded form ready to hybridize with the ABCB11 regRNA. Methods to predict secondary structures are known in the art (see, e.g., Seetin and Mathews, Methods Mol. Biol. (2012) 905:99-122; Zhao et al., PLOS Comput. Biol. (2021) 17 (8): e1009291) and web-based programs (e.g., RNAfold) are available to public users. For example, ASOs have been designed to target a human ABCB11 regulatory RNA (e.g., an eRNA or a paRNA). The nucleotide sequences of some of these ASOs are provided in Tables 2-11 below. In some embodiments, an ASO of the disclosure comprises or consists of a nucleotide sequence provided in any one of Tables 2-11. In some embodiments, an ASO of the disclosure comprises or consists of a nucleotide sequence and/or a chemistry modification as provided in Table 2. In some embodiments, an ASO of the disclosure comprises or consists of a nucleotide sequence and/or a chemistry modification of any one of SEQ ID NOs: 14-6021.

TABLE 2
Exemplary ABCB11 ASO sequences and/or chemical modifications

| SEQ | | Sequence | SEQ | |
|---|---|---|---|---|
| ID | | MOE (M); DNA (d); LNA (L); 2′ OMethyl (m); | ID | |
| NO | Name | PS (=); PO(-); 5-MethylCytosine (5C); Teg-GalNAc [TEG] | NO | Sequence |
| 14 | CO- | MT=M5C=MT=MG=MG=MA=MA=MA=MA=MT=MA=MA=MT=MG=M5C=M5C=MT= | 5520 | TCTGGAAAAT |
| | 3974 | MT=MT=M5C | | AATGCCTTTC |
| 15 | CO- | MT=M5C=MA=MA=M5C=M5C=MG=MA=M5C=MA=MG=MT=MG=MA=MG=M5C=MT | 5521 | TCAACCGACA |
| | 3975 | =MT=MA=MT | | GTGAGCTTAT |
| 16 | CO- | MA=MT=MT=MT=MT=MA=MG=MT=M5C=MT=MA=MT=MT=M5C=M5C=M5C=MA= | 5522 | ATTTTAGTCT |
| | 3976 | MG=MA=M5C | | ATTCCCAGAC |
| 17 | CO- | M5C=M5C=MT=MT=M5C=M5C=M5C=MT=MA=MT=M5C=MA=MG=M5C=M5C=MA= | 5523 | CCTTCCCTAT |
| | 3977 | MT=MA=MG=M5C | | CAGCCATAGC |
| 18 | CO- | MT=M5C=MA=M5C=M5C=MG=MT=MG=MT=MA=MT=MT=MT=MA=MT=MT=MT=M | 5524 | TCACCGTGTA |
| | 3978 | 5C=M5C=M5C | | TTTATTTCCC |
| 19 | CO- | M5C=MT=MT=MA=M5C=MG=MT=MG=MG=M5C=M5C=MA-MA=MT=MG=MG=MG= | 5525 | CTTACGTGGC |
| | 3979 | MA=MT=M5C | | CAATGGGATC |
| 20 | CO- | MG=MG=MT=MG=M5C=M5C=MT=M5C=MT=MG=MA-MA=MG-MA=MA=MA=MG= | 5526 | GGTGCCTCTG |
| | 3980 | MG=MA=M5C | | AAGAAAGGAC |
| 21 | CO- | MT=MT=MA=MG=MT=M5C=M5C=MT=M5C=MT=MA=M5C=M5C=MT=M5C=MA=MA | 5527 | TTAGTCCTCT |
| | 3981 | =M5C=MG=MG | | ACCTCAACGG |
| 22 | CO- | M5C=MA=MT=MT=M5C=MT=MG=MT=MT=MT=MT=M5C=MT=MT=M5C=M5C=MT= | 5528 | CATTCTGTTT |
| | 3982 | MA=MA=MG | | TCTTCCTAAG |
| 23 | CO- | MG=MT=MG=MG=M5C=MA=MG=MT=MT=MA=MA=M5C=MT=MT=MT=MG=MG=M | 5529 | GTGGCAGTTA |
| | 3983 | T=MG=MT | | ACTTTGGTGT |
| 24 | CO- | M5C=MA=MT=MT=MT=MA=MG=MA=MG=MA=M5C=MA=MA=MA=MG=MA=MA=M | 5530 | CATTTAGAGA |
| | 3984 | T=M5C=MG | | CAAAGAATCG |
| 25 | CO- | MA=MA=MG=MA=MG=MG=MA=MA=MA=MG=MA=MA=M5C=MT=MG=MT=MA=M | 5531 | AAGAGGAAAG |
| | 3985 | 5C=MA=MT | | AACTGTACAT |
| 26 | CO- | MT=MG=MT=MT=MT=MT=MG=M5C=MT=MT=MG=MG=MT=M5C=MA=MA=MG=M | 5532 | TGTTTTGCTT |
| | 3986 | A=MG=MG | | GGTCAAGAGG |
| 27 | CO- | MA=MT=M5C=MA=MA=MA=MT=MA=M5C=MT=MG=MG=MA=MG=MT=MT=MG=M | 5533 | ATCAAATACT |
| | 3987 | T=MG=MT | | GGAGTTGTGT |
| 28 | CO- | MA=MA=MA=MT=MG=M5C=M5C=M5C=MA=MA=MG=MT=MT=MT=M5C=MA=MT= | 5534 | AAATGCCCAA |
| | 3988 | MT=MG=M5C | | GTTTCATTGC |
| 29 | CO- | MA=MA=MG=MA=MT=MG=M5C=MT=MA=M5C=MA=MA=MT=MT=MG=M5C=M5C= | 5535 | AAGATGCTAC |
| | 3989 | MA=MT=MA | | AATTGCCATA |
| 30 | CO- | MG=MG=MA=MT=MT=MA=MA=MG=MA=MA=MG=M5C=MT=MA=MT=MT=MT=MA | 5536 | GGATTAAGAA |
| | 3990 | =MG=MG | | GCTATTTAGG |
| 31 | CO- | MG=MG=MG=MA=MT=MG=MA=M5C-MT=M5C=M5C-MT=MA=MA=MA=MA=MA= | 5537 | GGGATGACTC |
| | 3991 | MT=MG=MG | | CTAAAAATGG |
| 32 | CO- | MT=MG=MT=MT=MT=M5C=M5C=MT=MA=MG=MT=MA=M5C=M5C=MA=MA=MG= | 5538 | TGTTTCCTAG |
| | 3992 | MA=MA=MT | | TACCAAGAAT |
| 33 | CO- | MG=M5C=M5C=M5C=M5C=MT=MT=MG=MA=MA=MT=MA=MA=MT=MT=MT=MT= | 5539 | GCCCCTTGAA |
| | 3993 | MT=MG=MT | | TAATTTTTGT |
| 34 | CO- | M5C=M5C=M5C=MA=M5C=MT=M5C=MT=MA=MG=MA=MT=MT=MG=M5C=MA=M | 5540 | CCCACTCTAG |
| | 3994 | A=MG=M5C=M5C | | ATTGCAAGCC |
| 35 | CO- | MG=MT=M5C=MT=MA=MG=MG=MA=MG=M5C=MT=MT=M5C=MT=MG=MT=MT=M | 5541 | GTCTAGGAGC |
| | 3995 | T=MA=M5C | | TTCTGTTTAC |
| 36 | CO- | MT=MT=MG=MT=M5C=MT=M5C=MT=MG=MG=MT=MT=MG=MA-MA=MG=M5C=M | 5542 | TTGTCTCTGG |
| | 3996 | 5C=MT=M5C | | TTGAAGCCTC |
| 37 | CO- | MG=M5C=MT=MG=MT=MG=M5C=MT=M5C=MT=MG=MT=MA=MA=M5C=MA=M5C | 5543 | GCTGTGCTCT |
| | 3997 | =MT=MG=MT | | GTAACACTGT |
| 38 | CO- | MA=M5C=M5C=M5C=M5C=M5C=M5C=MT=M5C=MA=M5C=M5C=MT=MT=MG=MG- | 5544 | ACCCCCCTCA |
| | 3998 | MT=MT=M5C=M5C | | CCTTGGTTCC |
| 39 | CO- | MG=MG=M5C=MA=MA=M5C=M5C=MT=M5C=MG=MT=M5C=M5C=M5C=M5C=MT= | 5545 | GGCAACCTCG |
| | 3999 | M5C=MA=M5C=MT | | TCCCCTCACT |
| 40 | CO- | MA=MA=M5C=MT=MT=MA=MT=MT=MT=MA=MT=MG=MA=MG=M5C=MT=MG=M | 5546 | GGATTAAGAA |
| | 4000 | T=MT=MG | | GCTATTTAGG |
| 41 | CO- | MT=MA=MT=MT=MA=MG=MG=MA=MG=MT=MT=MA=MG=MT=MA=MG=MT=MG= | 5547 | AACTTATTTA |
| | 4001 | MG=MT | | TGAGCTGTTG |
| 42 | CO- | MG=MA=MG=MA=M5C=MA=MA=MA=MT=MT=MA=MT=MA=MG=MT=MG=MG=MT | 5548 | TATTAGGAGT |
| | 4002 | =MT=MA | | TAGTAGTGGT |
| 43 | CO- | MG=MG=MG=MA=MA=MT=MA=MG=M5C=MT=MT=MT=MT=MT=MA=MG=MA=MG | 5549 | GAGACAAATT |
| | 4003 | =MA=MT | | ATAGTGGTTA |
| 44 | CO- | MA=MT=MT=MT=MG=MT=M5C=MA=MA=M5C=MA=MT=MT=MT=MT=MG=MA=M | 5550 | GGGAATAGCT |
| | 4004 | G=MG=MG | | TTTTAGAGAT |
| 45 | CO- | MT=M5C=MA=M5C=MA=M5C=MT=MT=M5C=MA=MG=MA=M5C=MT=MG=MG=MG | 5551 | ATTTGTCAAC |
| | 4005 | =MA=MA=MT | | ATTTTGAGGG |
| 46 | CO- | MA=MT=MA=MT=MT=MG=M5C=MT=MA=MA=MT=MG=MG=M5C=MT=MT=MG=M | 5552 | TCACACTTCA |
| | 4006 | A=MG=M5C | | GACTGGGAAT |
| 47 | CO- | MG=MT=MT=MT=MT=MT=MA=MG=MG=MT=M5C=MA=M5C=M5C=M5C=MA=MA= | 5553 | ATATTGCTAA |
| | 4007 | MG=M5C=M5C | | TGGCTTGAGC |
| 48 | CO- | MT=MT=M5C=MT=MA=MG=MG=MA=MT=M5C=MT=MA=MA=MT=MT=MT=M5C=M | 5554 | GTTTTTAGGT |
| | 4008 | A=MG=MT | | CACCCAAGCC |
| 49 | CO- | MG=MT=MG=MG=MA=MT=MG=M5C=MA=MT=MG=MT=MT=MT=MA=MG=MA=MG | 5555 | TTCTAGGATC |
| | 4009 | =MG=MG | | TAATTTCAGT |
| 50 | CO- | M5C=MA=MT=MT=MG=MT=M5C=M5C=M5C=MG=MG-MT=MT=MT=MT=M5C=MA= | 5556 | GTGGATGCAT |
| | 4010 | MA=MA=MA | | GTTTAGAGGG |
| 51 | CO- | MT=M5C=M5C=MA=MA=M5C=MA=M5C=M5C=MG=MT=MA=M5C=MT=M5C=MA=M | 5557 | TCCAACACCG |
| | 4011 | T=MT=MT=M5C | | TACTCATTTC |
| 52 | CO- | MG=MA=MA=M5C=MT=MA=M5C=MA=M5C=M5C=MG=MA=M5C=MT=MT=MT=MA | 5558 | GAACTACACC |
| | 4012 | =MT=M5C=MT | | GACTTTATCT |
| 53 | CO- | MT=MT=MG=MA=MA=MA=MG=M5C=MT=MG=MG=MT=MT=MT=MA=MA=MT=MG | 5559 | TTGAAAGCTG |
| | 4013 | =M5C=MA | | GTTTAATGCA |
| 54 | CO- | MG=MA=MT=M5C=MA=M5C=MA=MA=MA=MT=MT=M5C=MT=MA=M5C=M5C=M5 | 5560 | GATCACAAAT |
| | 4014 | C=MT=MT=MG | | TCTACCCTTG |
| 55 | CO- | MG=MT=MT=MT=MG=M5C=MT=M5C=MT=MG=MA=MT=MA=MA=MT=M5C=MA=M | 5561 | GTTTGCTCTG |
| | 4015 | 5C=MA=MG | | ATAATCACAG |
| 56 | CO- | MT=MT=MT=MT=MA=MA=M5C=MT=MT=MG=MG=M5C=MT=MT=MT=MG=MT=MG | 5562 | TTTTAACTTG |
| | 4016 | =MA=MA | | GCTTTGTGAA |
| 57 | CO- | M5C=MT=MG=M5C=MT=M5C=MA=MA=MA=MG=MT=MA=MA=MA=MA=MA=MT= | 5563 | CTGCTCAAAG |
| | 4017 | MA=M5C=M5C | | TAAAAATACC |
| 58 | CO- | MA=MT=MG=MG=M5C=M5C=MG=MA=MA=MT=MA=MG=MA=MT=MT=MG=MA=M | 5564 | ATGGCCGAAT |
| | 4018 | A=MT=MA | | AGATTGAATA |
| 59 | CO- | MA=MG=MA=MG=MT=MG=MG=MT=MG=MT=MA=M5C=MA=MT=MG=MG=MG=MT | 5565 | AGAGTGGTGT |
| | 4019 | =MG=MT | | ACATGGGTGT |
| 60 | CO- | M5C=MA=MT=MG=MT=MA=MT=M5C=MA=MA=MT=M5C=MT=M5C=MT=MT=MA= | 5566 | CATGTATCAA |
| | 4020 | MG=MA=MG | | TCTCTTAGAG |
| 61 | CO- | MG=MT=M5C=MT=MT=M5C=MT=MT=MT=MA=MT=MA=MG=M5C=MA=MA=MT=M | 5567 | GTCTTCTTTA |
| | 4021 | G=M5C=MT | | TAGCAATGCT |
| 62 | CO- | MG=MA=MT=MG=MA=M5C=MT=M5C=MT=MT=MG=MG=MT=MT=MG=M5C=MT=M | 5568 | GATGACTCTT |
| | 4022 | G=MT=M5C | | GGTTGCTGTC |
| 63 | CO- | M5C=MA=M5C=MA=MA=M5C=MA=MT=MT=MG=MT=MA=MT=MA=M5C=MA=MA= | 5569 | CACAACATTG |
| | 4023 | MT=MA=M5C | | TATACAATAC |
| 64 | CO- | MA=M5C=MT=MT=MA=M5C=MA=MG=MG=MA=MT=MG=M5C=MA=MT=MT=MA= | 5570 | ACTTACAGGA |
| | 4024 | MA=MT=MG | | TGCATTAATG |
| 65 | CO- | MA=M5C=MG=M5C=MT=MA=M5C=MA=MA=MT=MA=M5C=MT=MT=MG=MG=MA= | 5571 | ACGCTACAAT |
| | 4025 | MT=MA=MG | | ACTTGGATAG |
| 66 | CO- | M5C=MT=MT=MG=MT=M5C=M5C=MA=MG=MT=MG=MT=MA=MA=MA=M5C=MA= | 5572 | CTTGTCCAGT |
| | 4026 | MA=M5C=MG | | GTAAACAACG |
| 67 | CO- | MG=MT=MG=MG=MA=MG=MT=MG-MT=MT=MT=M5C=MT=MG=MG=MT=MT=MT | 5573 | GTGGAGTGTT |
| | 4027 | =MA=MT | | TCTGGTTTAT |
| 68 | CO- | MT=MT=MA=MA=M5C=MT=M5C=MT=MT=MA=M5C=MT=MT=MT=MG=MT=MG=M | 5574 | TTAACTCTTA |
| | 4028 | G=MA=MG | | CTTTGTGGAG |
| 69 | CO- | MA=MT=MA=M5C=MT=MT=MG=MT=M5C=MT=M5C=MT=MG=M5C-MA=MA=MA= | 5575 | ATACTTGTCT |
| | 4029 | MT=M5C=M5C | | CTGCAAATCC |
| 70 | CO- | MT=M5C=MA=M5C=MA=MG=MG-MT=MA=MA=MG=MT=MA=MA=M5C=MT=MT= | 5576 | TCACAGGTAA |
| | 4030 | MA=MT=MA | | GTAACTTATA |
| 71 | CO- | MG=MG=M5C=MT=MT=M5C=M5C=MT=M5C=MA=MA=MA=MT=MT=M5C=MA=MT | 5577 | GGCTTCCTCA |
| | 4031 | =MA=MT=MT | | AATTCATATT |
| 72 | CO- | MA=MG=MT=M5C=M5C=MA=MT=MT=MA=MT=MT=MT=M5C=M5C=MT=M5C=MT= | 5578 | AGTCCATTAT |
| | 4032 | MG=MG=M5C | | TTCCTCTGGC |
| 73 | CO- | MG=MA=MA=MA=M5C=M5C=MT=MT=MT=MT=MA=MA=M5C=MT=M5C=MT=M5C | 5579 | GAAACCTTTT |
| | 4033 | =M5C=MA=M5C | | AACTCTCCAC |
| 74 | CO- | M5C=MG=MA=MG=MG=MT=MT=MG=MG=MA=MA=MA=MA=MG=MG=MT=MT=M | 5580 | CGAGGTTGGA |
| | 4034 | G=MT=MG | | AAAGGTTGTG |
| 75 | CO- | MG=MT=MT=M5C=MA=MG=MT=MG=M5C=MT=MT=MT=MA=MG=MA=MA=MT=M | 5581 | GTTCAGTGCT |
| | 4035 | G=MA=MT | | TTAGAATGAT |
| 76 | CO- | MG=M5C=MA=MA=MA=MT=MA=MG=MA=MT=MA=MA=MT=MG=MT=MT=M5C=M | 5582 | GCAAATAGAT |
| | 4036 | T=MT=MG | | AATGTTCTTG |
| 77 | CO- | MG=MG=MG=MA=M5C=MA=MT=MT=MG=MA=MT=M5C=M5C=MT=MT=MA=MG= | 5583 | GGGACATTGA |
| | 4037 | MG=M5C=MA | | TCCTTAGGCA |
| 78 | CO- | M5C=MT=M5C=MG=MT=MA=MT=MG=MT=M5C=MA=M5C=MT=MG=MA=MA=M5C | 5584 | CTCGTATGTC |
| | 4038 | =MT=MG=MT | | ACTGAACTGT |
| 79 | CO- | MG=MG=MT=MT=MA=MT=MT=MG=M5C=MT=M5C=MT=MG=MA=MG=MT=MA=M | 5585 | GGTTATTGCT |
| | 4039 | T=MG=MT | | CTGAGTATGT |
| 80 | CO- | MG=MT=MT=MT=M5C=M5C=M5C=MA=MA=MG=M5C=MA=M5C=MA=M5C=MT=M5 | 5586 | GTTTCCCAAG |
| | 4040 | C=MT=MG=MT | | CACACTCTGT |
| 81 | CO- | MT=M5C=M5C=MA=MA=MG=MG=M5C=M5C=MT=MG=MT=MT=MG=MA=M5C=MA | 5587 | TCCAAGGCCT |
| | 4041 | =M5C=M5C=M5C | | GTTGACACCC |
| 82 | CO- | M5C=MT=M5C=MT=M5C=MG=MT=MT=M5C=M5C=MA=MA=MG=MG=MT=MG=MA | 5588 | CTCTCGTTCC |
| | 4042 | =MA=MT=M5C | | AAGGTGAATC |
| 83 | CO- | MG=M5C=M5C=M5C=MA=M5C=MT=M5C=MT=MG=M5C=MT=M5C=MA=MA=MT=M | 5589 | GCCCACTCTG |
| | 4043 | T=MT=MG=M5C | | CTCAATTTGC |
| 84 | CO- | MG=MT=MT=MG=MG=MG=MA=MT=MA=MG=M5C=M5C=MT=MG=MA=MA=MT=M | 5590 | GTTGGGATAG |
| | 4044 | T=M5C=M5C | | CCTGAATTCC |
| 85 | CO- | MA=M5C=MT=MT=MT=MT=M5C=MA=M5C=MA=MA=M5C=MT=MA=M5C=MA=MG | 5591 | ACTTTTCACA |
| | 4045 | =MG=M5C=M5C | | ACTACAGGCC |
| 86 | CO- | MG=MG=MG=MA-MA=MA=MG=MT=MT=MT=MA=MA=MA=MG-MG-MT=MA=MT | 5592 | GGGAAAGTTT |
| | 4046 | =MT=MT | | AAAGGTATTT |
| 87 | CO- | MG=M5C=MA=MA=MT=M5C=MT=MT=MT=MT=MA=MT=MA=MT=MT=MG=MA=M | 5593 | GCAATCTTTT |
| | 4047 | G=MG=MG | | ATATTGAGGG |
| 88 | CO- | MA=MG=M5C=MA=MT=M5C=MT=MG=MG=MA=MT=M5C=M5C=MT=MG=M5C=MA | 5594 | AGCATCTGGA |
| | 4048 | =MA=M5C=M5C | | TCCTGCAACC |
| 89 | CO- | MG=MT=M5C=MT=M5C=M5C=MA=M5C=MA=MA=MA=MA=M5C=MT=M5C=MT=M | 5595 | GTCTCCACAA |
| | 4049 | A=MT=MA=MG | | AACTCTATAG |
| 90 | CO- | M5C=MA=MA=MA=MT=MG=MT=MT=M5C=MT=MT=MT=MT=MA=MG=MG=MG=M | 5596 | CAAATGTTCT |
| | 4050 | T=MA=MT | | TTTAGGGTAT |
| 91 | CO- | MG=MA=M5C=MT=MT=MT=MT=MG=MA=MG=M5C=MA=MA=MG=MG=M5C=MT= | 5597 | GACTTTTGAG |
| | 4051 | MG=MT=MT | | CAAGGCTGTT |
| 92 | CO- | M5C=M5C=MA=MG=MG=MG=MT=MG=M5C=MA=MA=MG=MA=MG=MT=MT=MG= | 5598 | CCAGGGTGCA |
| | 4052 | MT=M5C=MT | | AGAGTTGTCT |
| 93 | CO- | MG=MA=MG=M5C=MT=MT=M5C=MA=MT=M5C=MT=MG=MG=MT=MG=MT=MG= | 5599 | GAGCTTCATC |
| | 4053 | MT=M5C=M5C | | TGGTGTGTCC |
| 94 | CO- | M5C=MG=MT=MT=M5C=MA=MT=M5C=MT=MT=MT=MG=M5C=MT=MT=MA=MT= | 5600 | CGTTCATCTT |
| | 4054 | MA=M5C=MA | | TGCTTATACA |
| 95 | CO- | MT=MT=MG=MA=MA=M5C=M5C=MT=MT=MT=MA=MG=MA=MA=MA=MA=MT=M | 5601 | TTGAACCTTT |
| | 4055 | 5C=MG=MT | | AGAAAATCGT |
| 96 | CO- | MG=M5C=MT=MT=MA=MT=MT=MT=MG=MA=M5C=MT=M5C=MA=MA=MG=MA= | 5602 | GCTTATTTGA |
| | 4056 | M5C=M5C=MT | | CTCAAGACCT |
| 97 | CO- | M5C=MA=MG=M5C=MA=M5C=MA=MA=MA=MT=MA=MT=MA=MT=MT=MG=MG= | 5603 | CAGCACAAAT |
| | 4057 | MA=MG=MG | | ATATTGGAGG |
| 98 | CO- | MG=MA=MT=MT=M5C=MA=M5C=MA=MA=MG=MA=MA=MG=MG=MG=MA=MG= | 5604 | GATTCACAAG |
| | 4058 | MA=MG=MG | | AAGGGAGAGG |
| 99 | CO- | MG=MT=MA=MA=MG=M5C=MT=M5C=MA=MG=MG=M5C=MA-MA=MA=MG=MG= | 5605 | GTAAGCTCAG |
| | 4059 | MA=MG=MA | | GCAAAGGAGA |
| 100 | CO- | MG=MG=MA=MG-MT=MG-MA=MG-MA=MG=MG=M5C=MA=MT=MT=MT=MA=M | 5606 | GGAGTGAGAG |
| | 4060 | G=MG=MG | | GCATTTAGGG |
| 101 | CO- | MA=MT=MT=M5C=MA=MT=MT=MG=M5C=MT=MG=MA=MA=MG=MG=M5C=MT= | 5607 | ATTCATTGCT |
| | 4061 | MG=MG=MA | | GAAGGCTGGA |
| 102 | CO- | MG=MA=M5C=M5C=MA=MA=MT=MG=MT=MT=MG=M5C=M5C=MT=MA=MT=MT= | 5608 | GACCAATGTT |
| | 4062 | MA=MT=MT | | GCCTATTATT |
| 103 | CO- | MG=M5C=MA=M5C=M5C=M5C=MA=MA=M5C=M5C=MA=MA=MG=MT=M5C=MT= | 5609 | GCACCCAACC |
| | 4063 | MT=MG=MA=M5C | | AAGTCTTGAC |
| 104 | CO- | M5C=M5C=MT=MG=MA=MG=MA=MT=MT=MG=MA=MG=M5C=MT=MA=MT=MA= | 5610 | CCTGAGATTG |
| | 4480 | M5C=MT=MG | | AGCTATACTG |
| 105 | CO- | M5C=MA=MT=M5C=MT=M5C=MT=MG=MT=MG=MA=MA=MT=M5C=MG=M5C=MT | 5611 | CATCTCTGTG |
| | 4481 | =MA=MG=MT | | AATCGCTAGT |
| 106 | CO- | MT=MA=MA=M5C=MT=MG=MG=MG=MA=MG=MT=MA=MT=MA=MG=MA=MG=M | 5612 | TAACTGGGAG |
| | 4482 | G=MA=M5C | | TATAGAGGAC |
| 107 | CO- | MT=M5C=MA=M5C=MA=MA=MA=MT=MA=MG=MA=MT=MT=MG=MA=MG=MG=M | 5613 | TCACAAATAG |
| | 4483 | G=MT=M5C | | ATTGAGGGTC |
| 108 | CO- | MG=M5C=MT=MA=MA=MT=M5C=M5C=MA=MA=M5C=M5C=MA=M5C=MT=MT=M5 | 5614 | GCTAATCCAA |
| | 4484 | C=MA=MA=M5C | | CCACTTCAAC |
| 109 | CO- | MG=MT=M5C=MA=MA=MG=MA=MT=M5C=M5C=M5C=M5C=MG=MA=MA=MG=MT | 5615 | GTCAAGATCC |
| | 4485 | =MT=MG=MA | | CCGAAGTTGA |
| 110 | CO- | MG=MG=MA=M5C=MT=MG=M5C=MG=MT=MG=MA=M5C=MT=M5C=MA=MG=MG= | 5616 | GGACTGCGTG |
| | 4486 | MT=MT=MA | | ACTCAGGTTA |
| 111 | CO- | MG=M5C=MA=MA=MG=MG=MT=M5C=M5C=MA=MG=MG=MG=MT=MG=MT=MA= | 5617 | GCAAGGTCCA |
| | 4487 | MT=MT=M5C | | GGGTGTATTC |
| 112 | CO- | MA=MA=M5C=MA=MT=MG=MT=M5C=MA=MG=MA=M5C=MA=MG=MT=MA=MG= | 5618 | AACATGTCAG |
| | 4488 | MT=MT=M5C | | ACAGTAGTTC |
| 113 | CO- | M5C=MA=M5C=MA=MG=MA=MG=MT=MG=M5C=M5C=MA=MA=MG=MG=MA=MG | 5619 | CACAGAGTGC |
| | 4489 | =MT=MT=MA | | CAAGGAGTTA |
| 114 | CO- | M5C=MG=MA=MG=MA=MA=MA=MA=MT=MG=MA=MG=MG=MT=MA=M5C=MA= | 5620 | CGAGAAAATG |
| | 4490 | MT=MG=MG | | AGGTACATGG |
| 115 | CO- | MG=MA=MG=MG=MT=MT=MG=MA=M5C=MA=MA=MA=MT=MA=MG=MG=MT=M | 5621 | GAGGTTGACA |
| | 4491 | G=MT=MG | | AATAGGTGTG |
| 116 | CO- | MG=MA=MT=MT=MT=MA=MA=MA=MG=MG=MA=M5C=MT=MG=M5C=MA=MT=M | 5622 | GATTTAAAGG |
| | 4492 | G=MG=M5C | | ACTGCATGGC |
| 117 | CO- | MA=MT=MG=MG=MT=MG=MA=MG=MA=MG=MT=MG=MT=MG=MG=MT=MT=M5C | 5623 | ATGGTGAGAG |
| | 4493 | =MA=MA | | TGTGGTTCAA |
| 118 | CO- | MG=M5C=MA=MG=MG=MA=MT=MG=MA=MA=MG=MT=MT=MT=MA=M5C=MA=M | 5624 | GCAGGATGAA |
| | 4494 | A=MG=MG | | GTTTACAAGG |
| 119 | CO- | MT=MG=MG=MT=MA=MT=M5C=M5C=MA=MT=MT=M5C=MA=M5C=MA=MA=MG= | 5625 | TGGTATCCAT |
| | 4495 | MG=MT=MG | | TCACAAGGTG |
| 120 | CO- | MG=MT=MA=M5C=MT=MA=MG=MA=M5C=MT=M5C=MT=MG=M5C=M5C=MT=MG | 5626 | GTACTAGACT |
| | 4496 | =MT=MG=MT | | CTGCCTGTGT |
| 121 | CO- | MG=MG=MT=MG=MT=MT=MG=MA=MG=MA=MA=M5C=MT=MA=MG=MA=MA=MT | 5627 | GGTGTTGAGA |
| | 4497 | =MA=MT | | ACTAGAATAT |
| 122 | CO- | MG=MT=M5C=M5C=M5C=MT=MG=MT=MG=MG=MT=MT=MA=M5C=M5C=MT=MA | 5628 | GTCCCTGTGG |
| | 4498 | =MA=MA=MG | | TTACCTAAAG |
| 123 | CO- | MA=MA=MG=MG=MA=MA=MG=MG=MT=M5C=MT=MA=MG=M5C=MG=MG=MA= | 5629 | AAGGAAGGTC |
| | 4499 | MG=MG=MT | | TAGCGGAGGT |
| 124 | CO- | MA=MG=MA=M5C=MT=MT=MT=MG=MG=M5C=M5C=MA=MG=MG=M5C=MA=MA= | 5630 | AGACTTTGGC |
| | 4500 | MG=MA=M5C | | CAGGCAAGAC |
| 125 | CO- | MG=M5C=MA=MT=MG=MG=MT=MA=MG=M5C=MT=MA=MG=MT=MG=MA=MT=M | 5631 | GCATGGTAGC |
| | 4501 | A=MA=MT | | TAGTGATAAT |
| 126 | CO- | MA=MA=MA=MT=MG-MT=MT=MG=MG=M5C=M5C-M5C=MT=MT=MG=MA=MG= | 5632 | AAATGTTGGC |
| | 4502 | MG=MA=MG | | CCTTGAGGAG |
| 127 | CO- | MA=MT=MT=MG=MT=MA=MG=MG=MT=MG=M5C-MT=MG=M5C=MG=MG=MG=M | 5633 | ATTGTAGGTG |
| | 4503 | T=MA=MT | | CTGCGGGTAT |
| 128 | CO- | MG=MT=MG=MT=M5C=MT=MA=MT=MT=MT=MG=MG=MT=MA=MT=M5C=MA=M | 5634 | GTGTCTATTT |
| | 4504 | G=MA=M5C | | GGTATCAGAC |
| 129 | CO- | MG=MA=MG=M5C=MA=M5C=MA=M5C=MA=MT=MT=M5C=MA=M5C=MT=M5C=M | 5635 | GAGCACACAT |
| | 4505 | 5C=MA=MT=MT | | TCACTCCATT |
| 130 | CO- | MT=MG=M5C=MA=MT=MT=MG=M5C=MA=MT=MG=MT=MT=MG=MA=MG=M5C= | 5636 | TGCATTGCAT |
| | 4506 | MT=MG=MG | | GTTGAGCTGG |
| 131 | CO- | MG=MA=MA=MG=MA=MA=MT=MG=MG=MG=MA=MG=M5C=MA=MA=MT=M5C= | 5637 | GAAGAATGGG |
| | 4507 | M5C=MG=M5C | | AGCAATCCGC |
| 132 | CO- | MG=MT=MA=MG=MG=MA=MA=MG=MT=MT=MG=MA=MA=MT=MG=MA=MG=MT | 5638 | GTAGGAAGTT |
| | 4508 | =MT=MG | | GAATGAGTTG |
| 133 | CO- | MT=MG=MG=MA=MT=MG=MT=MA=MG=MA=MT=MG=MA=MA=MT=MG=MG=M5C | 5639 | TGGATGTAGA |
| | 4509 | =MA=MA | | TGAATGGCAA |
| 134 | CO- | MG=M5C=M5C=MA=M5C=MA=M5C=MG=MT=MT=MT=M5C=MT=MT=MA=MT=MT= | 5640 | GCCACACGTT |
| | 4510 | MG=M5C=MG | | TCTTATTGCG |
| 135 | CO- | MG=MT=MG=MG=MG=MA=MG=MT=MG=MG=MT=MA=MA=MG=MA=MT=MT=MA | 5641 | GTGGGAGTGG |
| | 4511 | =MG=M5C | | TAAGATTAGC |
| 136 | CO- | MA=MA=M5C=MG=MG-MA-MA-MG=MG=MG-MT-MT=MA-MA=MA=MG=MG=M | 5642 | AACGGAAGGG |
| | 4512 | T=MA=MT | | TTAAAGGTAT |
| 137 | CO- | MG=MT=MG=MA=MG=MG=M5C=MA=MT=M5C=MA=MG=MG=MT=MA=MA=MG=M | 5643 | GTGAGGCATC |
| | 4513 | T=MT=M5C | | AGGTAAGTTC |
| 138 | CO- | MG=M5C=MG=MG=MG=MA=MT=MT=MA=MG=MA=MT=MG=MA=MG=MG=MT=M5 | 5644 | GCGGGATTAG |
| | 4514 | C=MT=MA | | ATGAGGTCTA |
| 139 | CO- | MG=M5C=M5C=M5C=MA=MA=MA=MT=MG=MA=MG=MG=MT=MG=MA=MT=MA= | 5645 | GCCCAAATGA |
| | 4515 | M5C=MA=MA | | GGTGATACAA |
| 140 | CO- | MA=MT=MG=MA=M5C=MA=MT=MG=MA=MT=MA=MG=M5C=MA=M5C=M5C=M5C | 5646 | ATGACATGAT |
| | 4516 | =MA=MA=M5C | | AGCACCCAAC |
| 141 | CO- | M5C=MT=MT=MA=MG=MG=MA=MG=MG=MA=MT=MT=MA=MT=MT=MT=MA=MA | 5647 | CTTAGGAGGA |
| | 4517 | =M5C=M5C | | TTATTTAACC |
| 142 | CO- | M5C=MT=MA=M5C=MT=MA=MG=MT=MT=MG=MT=MT=MA=M5C=MA=M5C=M5C | 5648 | CTACTAGTTG |
| | 4518 | =MT=MT=MA | | TTACACCTTA |
| 143 | CO- | MA=MG=MA=M5C=MT=MG=MG=M5C=MA=MT=MG-MT=MG=MG=M5C=MT=MT= | 5649 | AGACTGGCAT |
| | 4519 | M5C=MA=M5C | | GTGGCTTCAC |
| 144 | CO- | MG=MG=MA=MA=MG=MA=MA=MG=MG=MT=MT=MA=MG=M5C=MA=M5C=MA= | 5650 | GGAAGAAGGT |
| | 4520 | MG=MA=M5C | | TAGCACAGAC |
| 145 | CO- | MG=M5C=MA=MT=MT=MT=M5C=MA=MG=MA=MG=M5C=MA=M5C=MA=MG=MA= | 5651 | GCATTTCAGA |
| | 4521 | MG=MT=MG | | GCACAGAGTG |
| 146 | CO- | M5C=MA=M5C=MT=MG=MG=M5C=M5C=M5C=MA=MT=M5C=MA=MA=MT=MT=M | 5652 | CACTGGCCCA |
| | 4522 | G=M5C=MA=MT | | TCAATTGCAT |
| 147 | CO- | MT=M5C=MT=MG=M5C=MA=MA=MA=MT=MT=MA=MA=MG=MA=MA=MG=M5C= | 5653 | TCTGCAAATT |
| | 4523 | MA=M5C-MT | | AAGAAGCACT |
| 148 | CO- | M5C=MA=MG=MA=MG=MA=MA=MA=M5C=M5C=MT=MA=MA=MA=MA=MT=MT= | 5654 | CAGAGAAACC |
| | 4524 | MG=MA=MG | | TAAAATTGAG |
| 149 | CO- | MA=M5C=M5C=M5C=MA=M5C=MA=MT=MA=MG=MT=MT=MG=MT=MA=M5C=MT | 5655 | ACCCACATAG |
| | 4525 | =M5C=MT=M5C | | TTGTACTCTC |
| 150 | CO- | MG=M5C=MT=MT=MG=MT=MG=MA=MT=MG=MT=M5C=M5C=MA=MG=MA=MT= | 5656 | GCTTGTGATG |
| | 4526 | MG=MA=M5C | | TCCAGATGAC |
| 151 | CO- | MG=MA=MG=M5C=MA=MT=M5C=MA=MG=MG=MT=MA=M5C=M5C=MA=MT=MG= | 5657 | GAGCATCAGG |
| | 4527 | MA=M5C=MA | | TACCATGACA |
| 152 | CO- | M5C=MT=MG=MG=MG=MA=MT=MA=MG=MG=MG=MT=MG=MG=MA=MT=M5C=M | 5658 | CTGGGATAGG |
| | 4528 | T=MA=M5C | | GTGGATCTAC |
| 153 | CO- | MG=MT=MG=MG=MA=MG=M5C=MA=MA=MA=M5C=MT=MA=MG=MG=M5C-MA= | 5659 | GTGGAGCAAA |
| | 4529 | M5C=MT=MG | | CTAGGCACTG |
| 154 | CO- | MG=MA=MG=M5C=M5C=M5C=MT=MA=M5C=M5C=M5C=MA=MG=MA=M5C=MA= | 5660 | GAGCCCTACC |
| | 4530 | MT=MG=MA=MT | | CAGACATGAT |
| 155 | CO- | MT=MA=MA=MA=MT=MG=MG=M5C=M5C=M5C=MA=M5C=MA=MA=MG=MT=MG= | 5661 | TAAATGGCCC |
| | 4531 | MA=MG=MG | | ACAAGTGAGG |
| 156 | CO- | MA=MT=MG=MA=MA=MG=MT=MG=MT=MT=MG=MT=MA=MT=MG=MT=MG=M5C | 5662 | ATGAAGTGTT |
| | 4532 | =M5C=M5C | | GTATGTGCCC |
| 157 | CO- | MA=M5C=MT=MT=MT=M5C=MG=MT=M5C=M5C=MT=MT=MA=MA=MG=MA=MT= | 5663 | ACTTTCGTCC |
| | 4533 | M5C=MA=MT | | TTAAGATCAT |
| 158 | CO- | MA=M5C=MA=MT=MT=MG=MG=MA=M5C=MT=MT=MA=M5C=MT=M5C=MA=MA= | 5664 | ACATTGGACT |
| | 4534 | M5C=MT=M5C | | TACTCAACTC |
| 159 | CO- | MT=MG=MG=M5C=MG=MT=MT=MA=MT=MT=MA=MA=MT=MT=M5C=MT=MA=M5 | 5665 | TGGCGTTATT |
| | 4535 | C=MA=MG | | AATTCTACAG |
| 160 | CO- | MT=MG=M5C=MT=MA=MG=MG=MA=MT=MT=MG=MT=MA=MA=MT=MT=MA=MG | 5666 | TGCTAGGATT |
| | 4536 | =MT=M5C | | GTAATTAGTC |
| 161 | CO- | MG=MA=MG=MT=MG=M5C=MT=MG=MT=MG=MG=MA=MG=MA=MT=M5C=MA=M | 5667 | GAGTGCTGTG |
| | 4537 | A=MT=MG | | GAGATCAATG |
| 162 | CO- | MT=MG=M5C=MT=MT=MA=MT=MG=MA=MA=MT=MG=MA=MA=MG=MT=M5C=M | 5668 | TGCTTATGAA |
| | 4538 | 5C=MA=MT | | TGAAGTCCAT |
| 163 | CO- | MA=M5C=MA=MA=MG=MA=MA=M5C=MT=MA=M5C=M5C=MG=M5C=MA=M5C=M | 5669 | ACAAGAACTA |
| | 4539 | T=M5C=MA=MA | | CCGCACTCAA |
| 164 | CO- | MT=MT=MT=MA=MT=MG=M5C=MA=M5C=MA=MA=MT=MA=MA=MT=MG=MA=M | 5670 | TTTATGCACA |
| | 4540 | G=M5C=M5C | | ATAATGAGCC |
| 165 | CO- | MG=MG=M5C=MA=MA=MT=MT=MA=MT=MA=MA=MA=MG=MG=MT=MA=M5C=M | 5671 | GGCAATTATA |
| | 4541 | 5C=M5C=MT | | AAGGTACCCT |
| 166 | CO- | MG=M5C=M5C=M5C=M5C=MT=MT=MG=M5C=MG=MA=MG=MT=MG=MA=MT=MA | 5672 | GCCCCTTGCG |
| | 4542 | =MA=MA=MT | | AGTGATAAAT |
| 167 | CO- | MA=MA=MT=M5C=M5C=MA=MA=MT=M5C=MT=MA=M5C=M5C=M5C=MA=MT=M | 5673 | AATCCAATCT |
| | 4543 | A=M5C=M5C=M5C | | ACCCATACCC |
| 168 | CO- | MT=M5C=M5C=MA=M5C=MG=MA=M5C=MT=MG=MA=MA=MA=M5C=MA=MT=MG | 5674 | TCCACGACTG |
| | 4544 | =MA=M5C=MT | | AAACATGACT |
| 169 | CO- | MG=MT=MG=MT=MT=M5C=MA=M5C=MT=MT=MA=M5C=MT=MA=MG=M5C=MT= | 5675 | GTGTTCACTT |
| | 4545 | MA=MT=M5C | | ACTAGCTATC |
| 170 | CO- | MG=MT=MG=MT=M5C=MT=MG=MG=MG=MA=MG=MG=MT=MT=MT=MA=MG=MT | 5676 | GTGTCTGGGA |
| | 4546 | =MT=MA | | GGTTTAGTTA |
| 171 | CO- | MG=MA=MG=MG=MA=M5C=MT=MG=MT=MG=M5C=M5C=MA=M5C=MA=MT=MA= | 5677 | GAGGACTGTG |
| | 4547 | MA=M5C=M5C | | CCACATAACC |
| 172 | CO- | M5C=MA=MA=MT=M5C=M5C=MT=MG=MG=MA=MA=MG=MT=MG=MG=M5C=MA= | 5678 | CAATCCTGGA |
| | 4548 | MT=M5C=M5C | | AGTGGCATCC |
| 173 | CO- | MA=MT=MA=MT=MG=MG=MA=MG=M5C=MA=MT=MT=MT=MT=MG=MT=MA=MT | 5679 | ATATGGAGCA |
| | 4549 | =MG=MG | | TTTTGTATGG |
| 174 | CO- | MT=MT=MA=MA=MG=MG=MT=M5C=MT=M5C=M5C=MT=MT=MG=MG=MA=M5C= | 5680 | TTAAGGTCTC |
| | 4550 | MA=MT=MG | | CTTGGACATG |
| 175 | CO- | MT=MG=MA=M5C=MA=MG=MA=MA=MG=M5C=MT=M5C=MT=MA=M5C=M5C=MA | 5681 | TGACAGAAGC |
| | 4551 | =MT=M5C=M5C | | TCTACCATCC |
| 176 | CO- | M5C=M5C=MT=M5C=MA=MT=MG=MT=MG=MA=MA=MG=MT=M5C=M5C=MA=MG | 5682 | CCTCATGTGA |
| | 4552 | =MT=MA=MG | | AGTCCAGTAG |
| 177 | CO- | MG=MG=MT=MA=MG=MT=MG=MA=MT=MA=M5C=MT=MT=MG=M5C=MT=MA=M | 5683 | GGTAGTGATA |
| | 4553 | A=MT=MT | | CTTGCTAATT |
| 178 | CO- | M5C=MT=MT=MG=MT=MT=MG=MG=MA=MT=MG=MA=MT=MT=MT=MG=MG=MT | 5684 | CTTGTTGGAT |
| | 4554 | =MT=MA | | GATTTGGTTA |
| 179 | CO- | MT=MT=MA=MG=MT=MT=MG=MA=M5C=MT=MG=M5C=MA=MG=MA=MG=MG=M | 5685 | TTAGTTGACT |
| | 4555 | G=MA=MG | | GCAGAGGGAG |
| 180 | CO- | MG=MA=MA=MG=MG=MA=M5C=MA=M5C=MA=MA=MT=MT=MT=M5C=MT=MG= | 5686 | GAAGGACACA |
| | 4556 | MT=MA=MG | | ATTTCTGTAG |
| 181 | CO- | MG=M5C=MT=MT=MT=M5C=MT=MT=MA=MG=MG=MT=MA=MG=M5C=MT=MT=M | 5687 | GCTTTCTTAG |
| | 4557 | A=MT=MG | | GTAGCTTATG |
| 182 | CO- | MT=MT=MT=MG-MT=MG=MG=M5C=MA=MT=MA=MG=MA=MG=MA=M5C=MT=M | 5688 | TTTGTGGCAT |
| | 4558 | A=MG=M5C | | AGAGACTAGC |
| 183 | CO- | MG=MT=MG=MT=MA=MA=MA=MG=M5C=M5C=MA=MG=M5C=MA=MT=MG-MA= | 5689 | GTGTAAAGCC |
| | 4559 | MG=MT=M5C | | AGCATGAGTC |
| 184 | CO- | MT=MT=MA=MT=MT=MA=M5C=M5C=MA=M5C=M5C=M5C=MT=MG=MA=MA=MA | 5690 | TTATTACCAC |
| | 4560 | =MA=MT=M5C | | CCTGAAAATC |
| 185 | CO- | MG=M5C=MA=M5C=MA=M5C=M5C=MA=MA=MT=M5C=MA=M5C=MA=MG=MA=M | 5691 | GCACACCAAT |
| | 4561 | A=MG=M5C=MT | | CACAGAAGCT |
| 186 | CO- | MG=MA=MT=MG=MG=MG=MT=MT=MA=MG=MG=M5C=MA=MG=MG=MA=MA=M | 5692 | GATGGGTTAG |
| | 4562 | G=MT=M5C | | GCAGGAAGTC |
| 187 | CO- | MA=MA=M5C=MT=MT=M5C-MT=MG=MT=MT=M5C=MA=M5C=MG=MG=M5C=M5 | 5693 | AACTTCTGTT |
| | 4563 | C=MA=M5C=M5C | | CACGGCCACC |
| 188 | CO- | MA=MG=MT=MG=M5C=M5C=MA=MG=MT=MA=MG=MG=MA=MT=MG=MA=MG=M | 5694 | AGTGCCAGTA |
| | 4564 | T=MT=MG | | GGATGAGTTG |
| 189 | CO- | MG=MA=M5C=M5C=MA=MG=MG=MT=M5C=MA=MT=MA=MA=MA=MT=MA=M5C= | 5695 | GACCAGGTCA |
| | 4565 | MA=MG=M5C | | TAAATACAGC |
| 190 | CO- | MG=MG=MA=MA=MG=MG=MA=M5C=MT=MT=MG=MA=MG=MT=MT=MA=MT=MA | 5696 | GGAAGGACTT |
| | 4566 | =MT=M5C | | GAGTTATATC |
| 191 | CO- | MA=MA=MA=MA=MG=MT=MG=MA=M5C-M5C=M5C=MA=MT=M5C=MA=MG=MT= | 5697 | AAAAGTGACC |
| | 4567 | MA=MT=MG | | CATCAGTATG |
| 192 | CO- | MT=MG=MT=MT=MT=MG=MA=MT=MG=M5C=MA=MG=MA=M5C=M5C=MT=MT=M | 5698 | TGTTTGATGC |
| | 4568 | A=MA=MG | | AGACCTTAAG |
| 193 | CO- | MT=MT=MA=M5C=MA=MG=MT=MT=MA=MG=MT=MG=MT=M5C=M5C=MT=MT=M | 5699 | TTACAGTTAG |
| | 4569 | G=MA=MG | | TGTCCTTGAG |
| 194 | CO- | M5C=MA=MA=MA=MT=MG=MT=MT=MA=M5C=MG=M5C=MT=MG=MG=M5C=MT= | 5700 | CAAATGTTAC |
| | 4570 | MA=M5C=M5C | | GCTGGCTACC |
| 195 | CO- | MA=MT=MT=MG=MA=MA=MG=MA=MT=MT=MG=M5C=MA=M5C=MT=MA=MT=M | 5701 | ATTGAAGATT |
| | 4571 | G=MG=MA | | GCACTATGGA |
| 196 | CO- | MG=MT=MA=MG=MA=MA=MT=MT=M5C=MA=MG=M5C=M5C=MA=MA=MT=MA= | 5702 | GTAGAATTCA |
| | 4572 | MG=MT=MT | | GCCAATAGTT |
| 197 | CO- | M5C=MT=MG=MT=MG=MG=MA=M5C=M5C=MA=MA=MT=MT=MT=MG=MG=MA= | 5703 | CTGTGGACCA |
| | 4573 | MA=M5C=M5C | | ATTTGGAACC |
| 198 | CO- | MA=MG=M5C=M5C=MT=M5C=M5C=MT=M5C=MA=MT=MT=MA=M5C=M5C=M5C= | 5704 | AGCCTCCTCA |
| | 4574 | MA=MG=MT=MA | | TTACCCAGTA |
| 199 | CO- | MG=M5C=MT=M5C=MT=MG=MA=MA=MA=MG=MT=MG=MT=MG=MA=M5C=MT= | 5705 | GCTCTGAAAG |
| | 4575 | MA=M5C=MT | | TGTGACTACT |
| 200 | CO- | MT=MG=MG=M5C=MG=MG=MG=MA=MA=MT=M5C=MA=MT=MG=MG=MA=MG=M | 5706 | TGGCGGGAAT |
| | 4576 | T=MG=M5C | | CATGGAGTGC |
| 201 | CO- | MG=MG=MA=MG=MT=MG=MG=MA=MG=MT=MA=MA=MG=M5C=MA=MA=MG=M | 5707 | GGAGTGGAGT |
| | 4577 | A=M5C=MT | | AAGCAAGACT |
| 202 | CO- | M5C=MT=MG=MG=MT=MG=MT=MA=MT=MG=MT=MA=MA=MG=MG=MA=MA=MG | 5708 | CTGGTGTATG |
| | 4578 | =M5C=MT | | TAAGGAAGCT |
| 203 | CO- | MG=MG=MA=MA=M5C=MA=MA=MG=MT=MA=M5C=MA=MT=MG=MG=MG=MT=M | 5709 | GGAACAAGTA |
| | 4579 | A=MT=M5C | | CATGGGTATC |
| 204 | CO- | MT=MA=MA=M5C=MT=MG=MG=MG=MT=MT=MT=MT=MA=MT=MG=MG=MA=MA | 5710 | TAACTGGGTT |
| | 4580 | =MG=MG | | TTATGGAAGG |
| 205 | CO- | MT=MG=MT=M5C=MA=MG=MT=MT=MA=MG=M5C=MG=MT=MG=MT=MG=MA=M | 5711 | TGTCAGTTAG |
| | 4581 | G=MG=MG | | CGTGTGAGGG |
| 206 | CO- | MA=M5C=MG=MT=MA=MT=MG=M5C=MT=MG=MT=MG=MT=MG=MA=MG=MA=M | 5712 | ACGTATGCTG |
| | 4582 | G=MA=MG | | TGTGAGAGAG |
| 207 | CO- | MA=MG=MG=MG=MA=MT=MA=MT=MG-MA=MT=MA=MG=MG=MG=MA=MG=MT | 5713 | AGGGATATGA |
| | 4583 | =MG=MT | | TAGGGAGTGT |
| 208 | CO- | MA=MT=MG=MG=MT=MG=MT=MA=MA=MA=MT=MG=MG=MT=MA=MT=MG=MG= | 5714 | ATGGTGTAAA |
| | 4584 | MA=MG | | TGGTATGGAG |
| 209 | CO- | MG=MT=MT=MT=MA=MG=MT=MT=MG=MA=M5C=MT=MG=M5C=MA=MG=MA=M | 5715 | GTTTAGTTGA |
| | 6669 | G=MG=MG | | CTGCAGAGGG |
| 210 | CO- | MT=MA=MG=MT=MT=MT=MA=MG=MT=MT=MG=MA=M5C=MT=MG=M5C=MA=M | 5716 | TAGTTTAGTT |
| | 6670 | G=MA=MG | | GACTGCAGAG |
| 211 | CO- | MT=MT=M5C=MT=MG=MT=MA=MG=MT=MT=MT=MA=MG=MT=MT=MG=MA=M5C | 5717 | TTCTGTAGTT |
| | 6671 | =MT=MG | | TAGTTGACTG |
| 212 | CO- | MA=MG=MG=MA=M5C=MA=M5C=MA=MA=MT=MT=MT=M5C=MT=MG=MT=MA= | 5718 | AGGACACAAT |
| | 6672 | MG=MT=MT | | TTCTGTAGTT |
| 213 | CO- | MG=MA=MA=MG=MG=dA=d5C=dA=d5C=dA=dA=dT=dT=dT=d5C=MT=MG= | 5719 | GAAGGACACA |
| | 6673 | MT=MA=MG | | ATTTCTGTAG |
| 214 | CO- | MT=MG=MG=MA=MA=MG=MG=MA=M5C=MA=M5C=MA=MA=MT=MT=MT=M5C= | 5720 | TGGAAGGACA |
| | 6674 | MT=MG=MT | | CAATTTCTGT |
| 215 | CO- | MA=MG=M5C=MA=MA=MG=M5C=MT=MG=MT=MG=MT=MG=MG=MA=MA=MG=M | 5721 | AGCAAGCTGT |
| | 6675 | G=MA=M5C | | GTGGAAGGAC |
| 216 | CO- | MT=MA=MA=MA=MG=M5C=MA=MA=MG=M5C=MT=MG=MT=MG=MT=MG=MG=M | 5722 | TAAAGCAAGC |
| | 6676 | A=MA=MG | | TGTGTGGAAG |
| 217 | CO- | MT=MG=MG=M5C=MA=MA=MG=MG=MA=MG=MA=MG=MG=MA=MT=MG=MA=M | 5723 | TGGCAAGGAG |
| | 6965 | G=MT=MA | | AGGATGAGTA |
| 218 | CO- | MA=MT=MG=MA=MA=MT=MG=MG=M5C=MA=MA=MG=MG=MA=MG=MA=MG=M | 5724 | ATGAATGGCA |
| | 6966 | G=MA=MT | | AGGAGAGGAT |
| 219 | CO- | MG=MA=MT=MG=MT=MA=MG=MA=MT=MG=MA=MA=MT=MG=MG=M5C=MA=M | 5725 | GATGTAGATG |
| | 6967 | A=MG=MG | | AATGGCAAGG |
| 220 | CO- | MT=MG=MG=MA=MT=dG=dT=dA=dG=dA=dT=dG=dA=dA=dT=MG=MG=M5C | 5726 | TGGATGTAGA |
| | 6968 | =MA=MA | | TGAATGGCAA |
| 221 | CO- | MT=MG=MT=MG=MG=MA=MT=MG=MT=MA=MG=MA=MT=MG=MA=MA=MT=MG= | 5727 | TGTGGATGTA |
| | 6969 | MG=M5C | | GATGAATGGC |
| 222 | CO- | MG=MG=MG=MT=MG=MT=MG=MG=MA=MT=MG=MT=MA=MG=MA=MT=MG=MA | 5728 | GGGTGTGGAT |
| | 6970 | =MA=MT | | GTAGATGAAT |
| 223 | CO- | MA=MT=MG=MG=MG=MT=MG=MT=MG=MG=MA=MT=MG=MT=MA=MG=MA=MT= | 5729 | ATGGGTGTGG |
| | 6971 | MG=MA | | ATGTAGATGA |
| 224 | CO- | MA=MA=MA=MT=MG=MG=MG=MT=MG=MT=MG=MG=MA=MT=MG=MT=MA=MG | 5730 | AAATGGGTGT |
| | 6972 | =MA=MT | | GGATGTAGAT |
| 225 | CO- | MA=MA=MA=MA=MA=MT=MG=MG-MG-MT=MG-MT=MG=MG=MA=MT=MG=MT | 5731 | AAAAATGGGT |
| | 6973 | =MA=MG | | GTGGATGTAG |
| 226 | CO- | MT=MG=MG=MG=MA=MA=MG=MT=MT=MT=MA=MA=MA=MA=MA=MA=MT=MG | 5732 | TGGGAAGTTT |
| | 6974 | =MG=MG | | AAAAAATGGG |
| 227 | CO- | M5C=MT=MG=MA=MA=MG=MT=MG=MG=MA=MG=MG=MT=MG=MG=MT=MG=M | 5733 | CTGAAGTGGA |
| | 6975 | G=MA=MA | | GGTGGTGGAA |
| 228 | CO- | MT=M5C=M5C=MT=MG=MA=MA=MG=MT=MG=MG=MA=MG=MG=MT=MG=MG=M | 5734 | TCCTGAAGTG |
| | 6976 | T=MG=MG | | GAGGTGGTGG |
| 229 | CO- | MG=MG=MT=MA=MA=MG=MT=MT=M5C=M5C=MT=MG=MA=MA=MG=MT=MG=M | 5735 | GGTAAGTTCC |
| | 6977 | G=MA=MG | | TGAAGTGGAG |
| 230 | CO- | MG=MA=MG=MG=M5C=MA=MT=M5C=MA=MG=MG=MT=MA=MA=MG=MT=MT=M | 5736 | GAGGCATCAG |
| | 6978 | 5C=M5C=MT | | GTAAGTTCCT |
| 231 | CO- | MG=MT=MG=MA=MG=dG=d5C=dA=dT=d5C=dA=dG=dG=dT=dA=MA=MG= | 5737 | GTGAGGCATC |
| | 6979 | MT=MT=M5C | | AGGTAAGTTC |
| 232 | CO- | MA=MA=MG=MT=MG=MA=MG=MG=M5C=MA=MT=M5C=MA=MG=MG=MT=MA=M | 5738 | AAGTGAGGCA |
| | 6980 | A=MG=MT | | TCAGGTAAGT |
| 233 | CO- | MA=M5C=MA=MG=MA=MA=MG=MT=MG=MA=MG=MG=M5C=MA=MT=M5C=MA= | 5739 | ACAGAAGTGA |
| | 6981 | MG=MG=MT | | GGCATCAGGT |
| 234 | CO- | MA=MA=MG=MA=M5C=MA=MG=MA=MA=MG=MT=MG=MA=MG=MG=M5C=MA= | 5740 | AAGACAGAAG |
| | 6982 | MT=M5C=MA | | TGAGGCATCA |
| 235 | CO- | MA=MG=MA=MA=MG=MA=M5C=MA=MG=MA=MA=MG=MT=MG=MA=MG=MG=M | 5741 | AGAAGACAGA |
| | 6983 | 5C=MA=MT | | AGTGAGGCAT |
| 236 | CO- | MT=MG=MA=MG=MG=MT=M5C=MT=MA=MA=MT=MT=MT=MA=MG=MA=MA=MG | 5742 | TGAGGTCTAA |
| | 6984 | =MA=M5C | | TTTAGAAGAC |
| 237 | CO- | MT=MG=MG=MA=MT=.LG=dT=dA=.LG=dA=dT=.LG=dA=dA=.LT=MG= | 5743 | TGGATGTAGA |
| | 6985 | MG=M5C=MA=MA | | TGAATGGCAA |
| 238 | CO- | MT=MG=MG=.LA=MT=dG=dT=.LA=dG=dA=dT=.LG=dA=dA=dT=.LG= | 5744 | TGGATGTAGA |
| | 6986 | MG=M5C=MA=MA | | TGAATGGCAA |
| 239 | CO- | MT=MG=MG=MA=.LT=dG=dT=dA=.LG=dA=dT=dG=.LA=dA=dT=MG= | 5745 | TGGATGTAGA |
| | 6987 | .LG=M5C=MA=MA | | TGAATGGCAA |
| 240 | CO- | MT=MG=MG=.LA=MT=.LG=MT=.LA=MG=.LA=MT=.LG=MA=.LA=MT= | 5746 | TGGATGTAGA |
| | 6988 | .LG=MG=.L5C=MA=MA | | TGAATGGCAA |
| 241 | CO- | MT=MG=.LG=MA=.LT=MG=.LT=MA=.LG=MA=.LT=MG=.LA=MA=.LT=MG= | 5747 | TGGATGTAGA |
| | 6989 | .LG=M5C=MA=MA | | TGAATGGCAA |
| 242 | CO- | MT=MG=.LG=MA=MT=.LG=MT=MA=.LG=MA=MT=.LG=MA=MA=.LT=MG=MG= | 5748 | TGGATGTAGA |
| | 6990 | .L5C=MA=MA | | TGAATGGCAA |
| 243 | CO- | MT=MG=MG=MA=.LT=MG=MT=MA=.LG=MA=MT=MG=.LA=MA=MT=MG=.LG=M5C | 5749 | TGGATGTAGA |
| | 6991 | =MA=MA | | TGAATGGCAA |
| 244 | CO- | MT=MG=MG=.LA=MT=MG-MT=.LA=MG=MA=MT=.LG=MA=MA=MT=.LG=MG=M5C | 5750 | TGGATGTAGA |
| | 6992 | =MA=MA | | TGAATGGCAA |
| 245 | CO- | MT=MG=MG=MA=MT=.LG=MT=MA=MG=MA=.LT=MG=MA=MA=MT=.LG=MG=M5C | 5751 | TGGATGTAGA |
| | 6993 | =MA=MA | | TGAATGGCAA |
| 246 | CO- | MT=MG=MG=MA=.LT=MG=MT=MA=MG=.LA=MT=MG=MA=MA=.LT=MG=MG=M5C | 5752 | TGGATGTAGA |
| | 6994 | =MA=MA | | TGAATGGCAA |
| 247 | CO- | MG=MT=MG=MA=MG=.LG=d5C=dA=.LT=d5C=dA=.LG=dG=dT=.LA=MA= | 5753 | GTGAGGCATC |
| | 6995 | MG=MT=MT=M5C | | AGGTAAGTTC |
| 248 | CO- | MG=MT=MG=.LA=MG=dG=d5C=.LA=dT=d5C=dA=.LG=dG=dT=dA=.LA= | 5754 | GTGAGGCATC |
| | 6996 | MG=MT=MT=M5C | | AGGTAAGTTC |
| 249 | CO- | MG=MT=MG=MA=.LG=dG=d5C=dA=.LT=d5C=dA=dG=.LG=dT=dA=MA= | 5755 | GTGAGGCATC |
| | 6997 | .LG=MT=MT=M5C | | AGGTAAGTTC |
| 250 | CO- | MG=MT=MG=.LA=MG=.LG=M5C=.LA=MT=.L5C=MA=.LG-MG=.LT=MA= | 5756 | GTGAGGCATC |
| | 6998 | .LA=MG=.LT=MT=M5C | | AGGTAAGTTC |
| 251 | CO- | MG=MT=.LG=MA=.LG=MG=.L5C=MA=.LT=M5C=.LA=MG=.LG-MT=.LA= | 5757 | GTGAGGCATC |
| | 6999 | MA=.LG=MT=MT=M5C | | AGGTAAGTTC |
| 252 | CO- | MG=MT=.LG=MA=MG=.LG=M5C=MA=.LT=M5C=MA=.LG=MG=MT=.LA=MA= | 5758 | GTGAGGCATC |
| | 7000 | MG=.LT=MT=M5C | | AGGTAAGTTC |
| 253 | CO- | MG=MT=MG=MA=.LG=MG=M5C=MA=.LT=M5C=MA=MG=.LG-MT=MA=MA=.LG=M | 5759 | GTGAGGCATC |
| | 7001 | T=MT=M5C | | AGGTAAGTTC |
| 254 | CO- | MG=MT=MG=.LA=MG=MG=M5C=.LA=MT=M5C=MA=.LG=MG=MT=MA=.LA=MG=M | 5760 | GTGAGGCATC |
| | 7002 | T=MT=M5C | | AGGTAAGTTC |
| 255 | CO- | MG=MT=MG=MA=MG=.LG=M5C=MA=MT=M5C=.LA=MG=MG=MT=MA=.LA=MG=M | 5761 | GTGAGGCATC |
| | 7003 | T=MT=M5C | | AGGTAAGTTC |
| 256 | CO- | MG=MT=MG=MA=.LG=MG=M5C=MA=MT=.L5C=MA=MG=MG=MT=.LA=MA=MG=M | 5762 | GTGAGGCATC |
| | 7004 | T=MT=M5C | | AGGTAAGTTC |
| 257 | CO- | MG=MT=MG=MG=MA=MT=dG=dT=dA=dG=dA=dT=dG=dA=dA=dT=MG=MG=M5C=M | 5763 | GTGGATGTAG |
| | 7005 | A=MA=MG | | ATGAATGGCA |
| | | | | AG |
| 258 | CO- | MG=MT=MG=MG=MA=MT=MG=MT=MA=MG=MA=MT=MG=MA=MA=MT=MG=MG | 5764 | GTGGATGTAG |
| | 7006 | =M5C=MA=MA=MG | | ATGAATGGCA |
| | | | | AG |
| 259 | CO- | MG=MT=MG=MG=MA=dT=dG=dT=MA=dG=dA=dT=dG=MA=dA=dT=dG=MG=M5C=M | 5765 | GTGGATGTAG |
| | 7007 | A=MA=MG | | ATGAATGGCA |
| | | | | AG |
| 260 | CO- | MG=MT=MG=MG=MA=.LT=dG=dT=.LA=dG=dA=dT=dG=.LA=dA=dT= | 5766 | GTGGATGTAG |
| | 7008 | .LG=MG=M5C=MA=MA=MG | | ATGAATGGCA |
| | | | | AG |
| 261 | CO- | MT=MG=MT=MG=MG=MA=MT=dG=dT=dA=dG-dA=dT=dG=dA=dA=dT=MG=MG=M5 | 5767 | TGTGGATGTA |
| | 7009 | C=MA=MA=MG=MG | | GATGAATGGC |
| | | | | AAGG |
| 262 | CO- | MT=MG=MT=MG=MG=MA=MT=MG=MT=MA=MG=MA=MT=MG=MA=MA=MT=MG= | 5768 | TGTGGATGTA |
| | 7010 | MG=M5C=MA=MA=MG=MG | | GATGAATGGC |
| | | | | AAGG |
| 263 | CO- | MT=MG=MT=MG=MG=dA=dT=MG=dT=dA=MG=dA=dT=MG=dA=dA=MT=dG=dG=M5 | 5769 | TGTGGATGTA |
| | 7011 | C=MA=MA=MG=MG | | GATGAATGGC |
| | | | | AAGG |
| 264 | CO- | MT=MG=MT=MG=MG=dA=dT=.LG=dT=dA=.LG=dA=dT=.LG=dA=dA=.LT | 5770 | TGTGGATGTA |
| | 7012 | =dG=dG=M5C=MA=MA=MG=MG | | GATGAATGGC |
| | | | | AAGG |
| 265 | CO- | MA=MG=MT=MG=MA=MG=dG=d5C=dA=dT=d5C=dA=dG=dG=dT=dA=MA=MG=MT= | 5771 | AGTGAGGCAT |
| | 7013 | MT=M5C=M5C | | CAGGTAAGTT |
| | | | | CC |
| 266 | CO- | MA=MG=MT=MG=MA=MG=MG=M5C=MA=MT=M5C=MA=MG=MG=MT=MA=MA=M | 5772 | AGTGAGGCAT |
| | 7014 | G=MT=MT=M5C=M5C | | CAGGTAAGTT |
| | | | | CC |
| 267 | CO- | MA=MG=MT=MG=MA=dG=dG=d5C=MA=dT=d5C=dA=dG=MG=dT=dA=dA=MG=MT= | 5773 | AGTGAGGCAT |
| | 7015 | MT=M5C=M5C | | CAGGTAAGTT |
| | | | | CC |
| 268 | CO- | MA=MG=MT=MG=MA=.LG=dG=d5C=.LA=dT=d5C=dA=dG=.LG=dT=dA= | 5774 | AGTGAGGCAT |
| | 7016 | .LA=MG=MT=MT=M5C=M5C | | CAGGTAAGTT |
| | | | | CC |
| 269 | CO- | MA=MA=MG=MT=MG=MA=MG=dG=d5C=dA=dT=d5C=dA=dG=dG=dT=dA=MA=MG= | 5775 | AAGTGAGGCA |
| | 7017 | MT=MT=M5C=M5C=MT | | TCAGGTAAGT |
| | | | | TCCT |
| 270 | CO- | MA=MA=MG=MT=MG=MA=MG=MG=M5C=MA=MT=M5C=MA=MG=MG=MT=MA=M | 5776 | AAGTGAGGCA |
| | 7018 | A=MG=MT=MT=M5C=M5C=MT | | TCAGGTAAGT |
| | | | | TCCT |
| 271 | CO- | MA=MA=MG=MT=MG=dA=dG=MG=d5C=dA=MT=d5C=dA=MG=dG=dT=MA=dA=dG= | 5777 | AAGTGAGGCA |
| | 7019 | MT=MT=M5C=M5C=MT | | TCAGGTAAGT |
| | | | | TCCT |
| 272 | CO- | MA=MA=MG=MT=MG=dA=dG=.LG=d5C=dA=.LT=d5C=dA=.LG=dG=dT= | 5778 | AAGTGAGGCA |
| | 7020 | .LA=dA=dG=MT=MT=M5C=M5C=MT | | TCAGGTAAGT |
| | | | | TCCT |
| 273 | CO- | MT=MG=MG=MA=MA=MA=MA=MT=MA=MA=MT=MG=M5C=M5C=MT=MT=MT=M | 5779 | TGGAAAATAA |
| | 7021 | 5C=MT=MA | | TGCCTTTCTA |
| 274 | CO- | MT=M5C=MT=MG=MG=dA=dA=dA=dA=dT=dA=dA=dT=dG=d5C=M5C=MT= | 5780 | TCTGGAAAAT |
| | 7022 | MT=MT=M5C | | AATGCCTTTC |
| 275 | CO- | M5C=MT=MT=MA=MT=M5C=MT=MG=MG=MA=MA=MA=MA=MT=MA=MA=MT=M | 5781 | CTTATCTGGA |
| | 7023 | G=M5C=M5C | | AAATAATGCC |
| 276 | CO- | MG=M5C=MT=MT=MA=MT=M5C=MT=MG=MG=MA=MA=MA=MA=MT=MA=MA=M | 5782 | GCTTATCTGG |
| | 7024 | T=MG=M5C | | AAAATAATGC |
| 277 | CO- | MA=MG=MT=MG=MA=MG=M5C=MT=MT=MA=MT=M5C=MT=MG=MG=MA=MA=M | 5783 | AGTGAGCTTA |
| | 7025 | A=MA=MT | | TCTGGAAAAT |
| 278 | CO- | MA=M5C=MA=MG=MT=MG=MA=MG=M5C=MT=MT=MA=MT=M5C=MT=MG=MG= | 5784 | ACAGTGAGCT |
| | 7026 | MA=MA=MA | | TATCTGGAAA |
| 279 | CO- | M5C=MG=MA=M5C=MA=MG=MT=MG=MA=MG=M5C=MT=MT=MA=MT=M5C=MT= | 5785 | CGACAGTGAG |
| | 7027 | MG=MG=MA | | CTTATCTGGA |
| 280 | CO- | MA=M5C=M5C=MG=MA=M5C=MA=MG=MT=MG=MA=MG=M5C=MT=MT=MA=MT= | 5786 | ACCGACAGTG |
| | 7028 | M5C=MT=MG | | AGCTTATCTG |
| 281 | CO- | MA=MT=M5C=MG=M5C=MT=MG=MT=MG=MG=M5C=MA=MG=MT=MT=MA=MA= | 5787 | ATCGCTGTGG |
| | 7029 | M5C=MT=MT | | CAGTTAACTT |
| 282 | CO- | MA=MG=MA=MA=MT=M5C=MG=M5C=MT=MG-MT=MG-MG=M5C=MA=MG=MT= | 5788 | AGAATCGCTG |
| | 7030 | MT=MA=MA | | TGGCAGTTAA |
| 283 | CO- | MA=MA=MA=MG=MA=MA=MT=M5C=MG=M5C=MT=MG=MT=MG=MG=M5C=MA= | 5789 | AAAGAATCGC |
| | 7031 | MG=MT=MT | | TGTGGCAGTT |
| 284 | CO- | MA=M5C=MA=MA=MA=MG=MA=MA=MT=M5C=MG=M5C=MT=MG=MT=MG=MG= | 5790 | ACAAAGAATC |
| | 7032 | M5C=MA=MG | | GCTGTGGCAG |
| 285 | CO- | MG=MA=MG=MA=M5C=MA=MA=MA=MG=MA=MA=MT=M5C=MG=M5C=MT=MG= | 5791 | GAGACAAAGA |
| | 7033 | MT=MG=MG | | ATCGCTGTGG |
| 286 | CO- | MT=MT=MA=MG=MA=MG=MA=M5C=MA=MA=MA=MG=MA=MA=MT=M5C=MG=M | 5792 | TTAGAGACAA |
| | 7034 | 5C=MT=MG | | AGAATCGCTG |
| 287 | CO- | M5C=MA=MT=MT=MT=dA=dG=dA=dG=dA=d5C=dA=dA=dA=dG=MA=MA=MT= | 5793 | CATTTAGAGA |
| | 7035 | M5C=MG | | CAAAGAATCG |
| 288 | CO- | MG=MT=MA=M5C=MA=MT=MT=MT=MA=MG=MA=MG=MA=M5C=MA=MA=MA=M | 5794 | GTACATTTAG |
| | 7036 | G=MA=MA | | AGACAAAGAA |
| 289 | CO- | M5C=MT=MG=MT=MA=M5C=MA=MT=MT=MT=MA=MG=MA=MG=MA=M5C=MA= | 5795 | CTGTACATTT |
| | 7037 | MA=MA=MG | | AGAGACAAAG |
| 290 | CO- | MA=MA=M5C=MT=MG=MT=MA=M5C=MA=MT=MT=MT=MA=MG=MA=MG=MA=M | 5796 | AACTGTACAT |
| | 7038 | 5C=MA=MA | | TTAGAGACAA |
| 291 | CO- | MG=MG=MA=MA=MA=MG=MA=MA=M5C=MT=MG=MT=MA=M5C=MA=MT=MT=M | 5797 | GGAAAGAACT |
| | 7039 | T=MA=MG | | GTACATTTAG |
| 292 | CO- | MA=MA=MG=MA=MG=MG=MA=MA=MA=MG=MA=MA=M5C=MT=MG=MG=M5C= | 5798 | AAGAGGAAAG |
| | 7040 | M5C=MG=MG | | AACTGGCCGG |
| 293 | CO- | MG=MT=M5C=MA=MA=MG=MA=MG-MG=MA=MA=MA=MG=MA=MA=M5C=MT= | 5799 | GTCAAGAGGA |
| | 7041 | MG=MG=M5C | | AAGAACTGGC |
| 294 | CO- | M5C=MT=MT=MG=MG=MT=M5C=MA=MA=MG=MA=MG=MG=MA=MA=MA=MG=M | 5800 | CTTGGTCAAG |
| | 7042 | A=MA=M5C | | AGGAAAGAAC |
| 295 | CO- | MT=MT=MG=M5C=MT=MT=MG=MG=MT=M5C=MA=MA=MG=MA=MG=MG=MA=M | 5801 | TTGCTTGGTC |
| | 7043 | A=MA=MG | | AAGAGGAAAG |
| 296 | CO- | MT=MT=MT=MT=MG=M5C=MT=MT=MG=MG=MT=M5C=MA=MA=MG=MA=MG=M | 5802 | TTTTGCTTGG |
| | 7044 | G=MA=MA | | TCAAGAGGAA |
| 297 | CO- | MT=MG=MT=MT=MT=dT=dG=d5C=dT=dT=dG=dG=dT=d5C=dA=MA=MG= | 5803 | TGTTTTGCTT |
| | 7045 | MA=MG=MG | | GGTCAAGAGG |
| 298 | CO- | MT=MG=MT=MG=MT=MT=MT=MT=MG=M5C=MT=MT=MG=MG=MT=M5C=MA=MA | 5804 | TGTGTTTTGC |
| | 7046 | =MG=MA | | TTGGTCAAGA |
| 299 | CO- | MG=MA=MG=MT=MT=MG=MT=MG=MT=MT=MT=MT=MG=M5C=MT=MT=MG=MG | 5805 | GAGTTGTGTT |
| | 7047 | =MT=M5C | | TTGCTTGGTC |
| 300 | CO- | MT=MG=MG=MA=MG=MT=MT=MG=MT=MG=MT=MT=MT=MT=MG=M5C=MT=MT= | 5806 | TGGAGTTGTG |
| | 7048 | MG=MG | | TTTTGCTTGG |
| 301 | CO- | MT=MA=M5C=MT=MG=MG=MA=MG=MT=MT=MG=MT=MG=MT=MT=MT=MT=MG | 5807 | TACTGGAGTT |
| | 7049 | =M5C=MT | | GTGTTTTGCT |
| 302 | CO- | MT=MT=MG=MG=MT=MT=M5C=M5C=M5C=M5C=M5C=MT=MG=M5C=MT=MG=MT | 5808 | TTGGTTCCCC |
| | 7050 | =MG=M5C=MT | | CTGCTGTGCT |
| 303 | CO- | MA=M5C=M5C=MT=MT=MG=MG=MT=MT=M5C=M5C=M5C=M5C=M5C=MT=MG=M | 5809 | ACCTTGGTTC |
| | 7051 | 5C=MT=MG=MT | | CCCCTGCTGT |
| 304 | CO- | MT=M5C=MA=M5C=M5C=MT=MT=MG=MG=MT=MT=M5C=M5C=M5C=M5C=M5C= | 5810 | TCACCTTGGT |
| | 7052 | MT=MG=M5C=MT | | TCCCCCTGCT |
| 305 | CO- | M5C=M5C=MT=M5C=MA=M5C=M5C=MT=MT=MG=MG=MT=MT=M5C=M5C=M5C= | 5811 | CCTCACCTTG |
| | 7053 | M5C=M5C=MT=MG | | GTTCCCCCTG |
| 306 | CO- | MA=M5C=M5C=M5C=M5C=d5C=d5C=dT=d5C=dA=d5C=d5C=dT=dT=dG=MG= | 5812 | ACCCCCCTCA |
| | 7054 | MT=MT=M5C=M5C | | CCTTGGTTCC |
| 307 | CO- | MT=M5C=MA=M5C=M5C=M5C=M5C=M5C=M5C-MT=M5C=MA=M5C=M5C=MT=MT | 5813 | TCACCCCCCT |
| | 7055 | =MG=MG=MT=MT | | CACCTTGGTT |
| 308 | CO- | MT=MG=M5C=MT=M5C=MA=M5C=M5C=M5C=M5C=M5C=M5C=MT=M5C=MA=M5C | 5814 | TGCTCACCCC |
| | 7056 | =M5C=MT=MT=MG | | CCTCACCTTG |
| 309 | CO- | MA=M5C=MT=MG=M5C=MT=M5C=MA=M5C=M5C=M5C=M5C=M5C=M5C=MT=M5C | 5815 | ACTGCTCACC |
| | 7057 | =MA=M5C=M5C=MT | | CCCCTCACCT |
| 310 | CO- | MT=M5C=MT=MG=MG=.LA=dA=dA=.LA=dT=dA=.LA=dT=dG=.L5C=M5C= | 5816 | TCTGGAAAAT |
| | 7058 | MT=MT=MT=M5C | | AATGCCTTTC |
| 311 | CO- | MT=M5C=MT=.LG=MG=dA=dA=.LA=dA=dT=dA=.LA=dT=dG=d5C=.L5C= | 5817 | TCTGGAAAAT |
| | 7059 | MT=MT=MT=M5C | | AATGCCTTTC |
| 312 | CO- | MT=M5C=MT=MG=.LG=dA=dA=dA=.LA=dT=dA=dA=.LT=dG=d5C=M5C= | 5818 | TCTGGAAAAT |
| | 7060 | .LT=MT=MT=M5C | | AATGCCTTTC |
| 313 | CO- | MT=M5C=MT=.LG=MG=.LA=MA=.LA=MA=.LT=MA=.LA=MT=.LG=M5C= | 5819 | TCTGGAAAAT |
| | 7061 | .L5C=MT=.LT=MT=M5C | | AATGCCTTTC |
| 314 | CO- | MT=M5C=.LT=MG=.LG=MA=.LA=MA=.LA=MT=.LA=MA=.LT=MG=.L5C=M5C= | 5820 | TCTGGAAAAT |
| | 7062 | .LT=MT=MT=M5C | | AATGCCTTTC |
| 315 | CO- | MT=M5C=.LT=MG=MG=.LA=MA=MA=.LA=MT=MA=.LA=MT=MG=.L5C=M5C= | 5821 | TCTGGAAAAT |
| | 7063 | MT=.LT=MT=M5C | | AATGCCTTTC |
| 316 | CO- | MT=M5C=MT=MG=.LG=MA=MA=MA=.LA=MT=MA=MA=.LT=MG=M5C=M5C=.LT= | 5822 | TCTGGAAAAT |
| | 7064 | MT=MT=M5C | | AATGCCTTTC |
| 317 | CO- | MT=M5C=MT=.LG=MG=MA=MA=.LA=MA=MT=MA=.LA=MT=MG=M5C=.L5C=MT= | 5823 | TCTGGAAAAT |
| | 7065 | MT=MT=M5C | | AATGCCTTTC |
| 318 | CO- | MT=M5C=MT=MG=MG=.LA=MA=MA=MA=MT=.LA=MA=MT=MG=M5C=.L5C=MT= | 5824 | TCTGGAAAAT |
| | 7066 | MT=MT=M5C | | AATGCCTTTC |
| 319 | CO- | MT=M5C=MT=MG=.LG=MA=MA=MA=MA=.LT=MA=MA=MT=MG=.L5C=M5C=MT= | 5825 | TCTGGAAAAT |
| | 7067 | MT=MT=M5C | | AATGCCTTTC |
| 320 | CO- | M5C=MA=MT=MT=MT=.LA=dG=dA=.LG=dA=d5C=.LA=dA=dA=.LG=MA= | 5826 | CATTTAGAGA |
| | 7068 | MA=MT=M5C=MG | | CAAAGAATCG |
| 321 | CO- | M5C=MA=MT=.LT=MT=dA=dG=.LA=dG=dA=d5C=.LA=dA=dA=dG=.LA=MA | 5827 | CATTTAGAGA |
| | 7069 | =MT=M5C=MG | | CAAAGAATCG |
| 322 | CO- | M5C=MA=MT=MT=.LT=dA=dG=dA=.LG=dA=d5C=dA=.LA=dA=dG=MA=.LA= | 5828 | CATTTAGAGA |
| | 7070 | MT=M5C=MG | | CAAAGAATCG |
| 323 | CO- | M5C=MA=MT=.LT=MT=.LA=MG=.LA=MG=.LA=M5C=.LA=MA=.LA=MG= | 5829 | CATTTAGAGA |
| | 7071 | .LA=MA=.LT=M5C=MG | | CAAAGAATCG |
| 324 | CO- | M5C=MA=.LT=MT=.LT=MA=.LG=MA=.LG=MA=.L5C=MA=.LA=MA=.LG= | 5830 | CATTTAGAGA |
| | 7072 | MA=.LA=MT=M5C=MG | | CAAAGAATCG |
| 325 | CO- | M5C=MA=.LT=MT=MT=.LA=MG=MA=.LG=MA=M5C=.LA=MA=MA=.LG=MA=MA=.L | 5831 | CATTTAGAGA |
| | 7073 | T=M5C=MG | | CAAAGAATCG |
| 326 | CO- | M5C=MA=MT=MT=.LT=MA=MG=MA=.LG=MA=M5C=MA=.LA=MA=MG=MA=.LA=M | 5832 | CATTTAGAGA |
| | 7074 | T=M5C=MG | | CAAAGAATCG |
| 327 | CO- | M5C=MA=MT=.LT=MT=MA=MG=.LA=MG=MA=M5C=.LA=MA=MA=MG=.LA=MA=M | 5833 | CATTTAGAGA |
| | 7075 | T=M5C=MG | | CAAAGAATCG |
| 328 | CO- | M5C=MA=MT=MT=MT=.LA=MG=MA=MG=MA=.L5C=MA=MA=MA=MG=.LA=MA=M | 5834 | CATTTAGAGA |
| | 7076 | T=M5C=MG | | CAAAGAATCG |
| 329 | CO- | M5C=MA=MT=MT=.LT=MA=MG=MA=MG=.LA=M5C=MA=MA=MA=.LG=MA=MA=M | 5835 | CATTTAGAGA |
| | 7077 | T=M5C=MG | | CAAAGAATCG |
| 330 | CO- | MT=MG=MT=MT=MT=.LT=dG=d5C=.LT=dT=dG=.LG=dT=d5C=.LA=MA= | 5836 | TGTTTTGCTT |
| | 7078 | MG=MA=MG=MG | | GGTCAAGAGG |
| 331 | CO- | MT=MG=MT=.LT=MT=dT=dG=.L5C=dT=dT=dG=.LG=dT=d5C=dA= | 5837 | TGTTTTGCTT |
| | 7079 | .LA=MG=MA=MG=MG | | GGTCAAGAGG |
| 332 | CO- | MT=MG=MT=MT=.LT=dT=dG=d5C=.LT=dT=dG=dG=.LT=d5C=dA=MA= | 5838 | TGTTTTGCTT |
| | 7080 | .LG=MA=MG=MG | | GGTCAAGAGG |
| 333 | CO- | MT=MG=MT=.LT=MT=.LT=MG=.L5C=MT=.LT=MG=.LG=MT=.L5C=MA=.LA= | 5839 | TGTTTTGCTT |
| | 7081 | MG=.LA=MG=MG | | GGTCAAGAGG |
| 334 | CO- | MT=MG=.LT=MT=.LT=MT=.LG=M5C=.LT=MT=.LG=MG=.LT=M5C=.LA=MA= | 5840 | TGTTTTGCTT |
| | 7082 | .LG=MA=MG=MG | | GGTCAAGAGG |
| 335 | CO- | MT=MG=.LT=MT=MT=.LT=MG=M5C=.LT=MT=MG=.LG=MT=M5C=.LA=MA=MG=.LA | 5841 | TGTTTTGCTT |
| | 7083 | =MG=MG | | GGTCAAGAGG |
| 336 | CO- | MT=MG=MT=MT=.LT=MT=MG=M5C=.LT=MT=MG=MG=.LT=M5C=MA=MA=.LG=MA | 5842 | TGTTTTGCTT |
| | 7084 | =MG=MG | | GGTCAAGAGG |
| 337 | CO- | MT=MG=MT=.LT=MT=MT=MG=.L5C=MT=MT=MG=.LG-MT=M5C=MA=.LA=MG=MA | 5843 | TGTTTTGCTT |
| | 7085 | =MG=MG | | GGTCAAGAGG |
| 338 | CO- | MT=MG=MT=MT=MT=.LT=MG=M5C=MT=MT=.LG-MG-MT=M5C=MA=.LA=MG=MA | 5844 | TGTTTTGCTT |
| | 7086 | =MG=MG | | GGTCAAGAGG |
| 339 | CO- | MT=MG=MT=MT=.LT=MT=MG=M5C=MT=.LT=MG=MG=MT=M5C=.LA=MA=MG=MA | 5845 | TGTTTTGCTT |
| | 7087 | =MG=MG | | GGTCAAGAGG |
| 340 | CO- | MA=M5C=M5C=M5C=M5C=.L5C=d5C=dT=.L5C=dA=d5C=.L5C=dT=dT= | 5846 | ACCCCCCTCA |
| | 7088 | .LG=MG=MT=MT=M5C=M5C | | CCTTGGTTCC |
| 341 | CO- | MA=M5C=M5C=.L5C=M5C=d5C=d5C=.LT=d5C=dA=d5C=.L5C=dT=dT= | 5847 | ACCCCCCTCA |
| | 7089 | dG=.LG=MT=MT=M5C=M5C | | CCTTGGTTCC |
| 342 | CO- | MA=M5C=M5C=M5C=.L5C=d5C=d5C=dT=.L5C=dA=d5C=d5C=.LT=dT= | 5848 | ACCCCCCTCA |
| | 7090 | dG=MG=.LT=MT=M5C=M5C | | CCTTGGTTCC |
| 343 | CO- | MA=M5C=M5C=.L5C=M5C=.L5C=M5C=.LT=M5C=.LA=M5C=.L5C=MT= | 5849 | ACCCCCCTCA |
| | 7091 | .LT=MG=.LG=MT=.LT=M5C=M5C | | CCTTGGTTCC |
| 344 | CO- | MA=M5C=.L5C=M5C=.L5C=M5C=.L5C=MT=.L5C=MA=.L5C=M5C=.LT=MT= | 5850 | ACCCCCCTCA |
| | 7092 | .LG=MG=.LT=MT=M5C=M5C | | CCTTGGTTCC |
| 345 | CO- | MA=M5C=.L5C=M5C=M5C=.L5C=M5C=MT=.L5C=MA=M5C=.L5C=MT=MT= | 5851 | ACCCCCCTCA |
| | 7093 | .LG=MG=MT=.LT=M5C=M5C | | CCTTGGTTCC |
| 346 | CO- | MA=M5C=M5C=M5C=.L5C=M5C=M5C=MT=.L5C=MA=M5C-M5C=.LT=MT=MG=MG= | 5852 | ACCCCCCTCA |
| | 7094 | LT=MT=M5C=M5C | | CCTTGGTTCC |
| 347 | CO- | MA=M5C=M5C=.L5C=M5C=M5C=M5C=.LT=M5C=MA=M5C=.L5C=MT=MT=MG=.LG= | 5853 | ACCCCCCTCA |
| | 7095 | MT=MT=M5C=M5C | | CCTTGGTTCC |
| 348 | CO- | MA=M5C=M5C=M5C=M5C=.L5C=M5C=MT=M5C=MA=.L5C=M5C=MT=MT=MG=.LG= | 5854 | ACCCCCCTCA |
| | 7096 | MT=MT=M5C=M5C | | CCTTGGTTCC |
| 349 | CO- | MA=M5C=M5C=M5C=.L5C=M5C=M5C=MT=M5C=.LA=M5C=M5C=MT=MT=.LG=MG= | 5855 | ACCCCCCTCA |
| | 7097 | MT=MT=M5C=M5C | | CCTTGGTTCC |
| 350 | CO- | MA=MT=M5C-MT=MG=MG=dA=dA=dA=dA=dT=dA=dA=dT=dG=d5C=M5C=MT=MT= | 5856 | ATCTGGAAAA |
| | 7098 | MT=M5C=MT | | TAATGCCTTT |
| | | | | CT |
| 351 | CO- | MA=MT=M5C=MT=MG=MG=MA=MA=MA=MA=MT=MA=MA=MT=MG=M5C=M5C= | 5857 | ATCTGGAAAA |
| | 7099 | MT=MT=MT=M5C=MT | | TAATGCCTTT |
| | | | | CT |
| 352 | CO- | MA=MT=M5C=MT=MG=dG=dA=dA=MA=dA=dT=dA=dA=MT=dG=d5C=d5C=MT=MT= | 5858 | ATCTGGAAAA |
| | 7100 | MT=M5C=MT | | TAATGCCTTT |
| | | | | CT |
| 353 | CO- | MA=MT=M5C=MT=MG=.LG=dA=dA=.LA=dA=dT=dA=dA=.LT=dG=d5C= | 5859 | ATCTGGAAAA |
| | 7101 | .L5C=MT=MT=MT=M5C=MT | | TAATGCCTTT |
| | | | | CT |
| 354 | CO- | MT=MA=MT=M5C=MT=MG=MG=dA=dA=dA=dA=dT=dA=dA=dT=dG=d5C=M5C=MT= | 5860 | TATCTGGAAA |
| | 7102 | MT=MT=M5C=MT=MA | | ATAATGCCTT |
| | | | | TCTA |
| 355 | CO- | MT=MA=MT=M5C=MT=MG=MG=MA=MA=MA=MA=MT=MA=MA=MT=MG=M5C=M | 5861 | TATCTGGAAA |
| | 7103 | 5C=MT=MT=MT=M5C=MT=MA | | ATAATGCCTT |
| | | | | TCTA |
| 356 | CO- | MT=MA=MT=M5C=MT=dG=dG=MA=dA=dA=MA=dT=dA=MA=dT=dG=M5C=d5C=dT= | 5862 | TATCTGGAAA |
| | 7104 | MT=MT=M5C=MT=MA | | ATAATGCCTT |
| | | | | TCTA |
| 357 | CO- | MT=MA=MT=M5C=MT=dG=dG=.LA=dA=dA=.LA=dT=dA=.LA=dT=dG= | 5863 | TATCTGGAAA |
| | 7105 | .L5C=d5C=dT=MT=MT=M5C=MT=MA | | ATAATGCCTT |
| | | | | TCTA |
| 358 | CO- | MA=M5C=MA=MT=MT=MT=dA=dG=dA=dG=dA=d5C=dA=dA=dA=dG=MA=MA=MT= | 5864 | ACATTTAGAG |
| | 7106 | M5C=MG=M5C | | ACAAAGAATC |
| | | | | GC |
| 359 | CO- | MA=M5C=MA=MT=MT=MT=MA=MG=MA=MG=MA=M5C=MA=MA=MA=MG=MA=M | 5865 | ACATTTAGAG |
| | 7107 | A=MT=M5C=MG=M5C | | ACAAAGAATC |
| | | | | GC |
| 360 | CO- | MA=M5C=MA=MT=MT=dT=dA=dG=MA=dG=dA=d5C=dA=MA=dA=dG=dA=MA=MT= | 5866 | ACATTTAGAG |
| | 7108 | M5C=MG=M5C | | ACAAAGAATC |
| | | | | GC |
| 361 | CO- | MA=M5C=MA=MT=MT=.LT=dA=dG=.LA=dG=dA=d5C=dA=.LA=dA=dG=.LA= | 5867 | ACATTTAGAG |
| | 7109 | MA=MT=M5C=MG=M5C | | ACAAAGAATC |
| | | | | GC |
| 362 | CO- | MT=MA=M5C=MA=MT=MT=MT=dA=dG=dA=dG=dA=d5C=dA=dA=dA=dG=MA=MA= | 5868 | TACATTTAGA |
| | 7110 | MT=M5C=MG=M5C=MT | | GACAAAGAAT |
| | | | | CGCT |
| 363 | CO- | MT=MA=M5C=MA=MT=MT=MT=MA=MG=MA=MG=MA=M5C=MA-MA=MA=MG=M | 5869 | TACATTTAGA |
| | 7111 | A=MA=MT=M5C=MG=M5C=MT | | GACAAAGAAT |
| | | | | CGCT |
| 364 | CO- | MT=MA=M5C=MA=MT=dT=dT=MA=dG=dA=MG=dA=d5C=MA=dA=dA=MG=dA=dA= | 5870 | TACATTTAGA |
| | 7112 | MT=M5C=MG=M5C=MT | | GACAAAGAAT |
| | | | | CGCT |
| 365 | CO- | MT=MA=M5C=MA=MT=dT=dT=.LA=dG=dA=.LG=dA=d5C=.LA=dA=dA= | 5871 | TACATTTAGA |
| | 7113 | .LG-dA=dA=MT=M5C=MG=M5C=MT | | GACAAAGAAT |
| | | | | CGCT |
| 366 | CO- | MG=MT=MG=MT=MT=MT=dT=dG=d5C=dT=dT=dG=dG=dT=d5C=dA=MA=MG= | 5872 | GTGTTTTGCT |
| | 7114 | MA=MG=MG=MA | | TGGTCAAGAG |
| | | | | GA |
| 367 | CO- | MG=MT=MG=MT=MT=MT=MT=MG=M5C=MT=MT=MG=MG=MT=M5C=MA=MA=M | 5873 | GTGTTTTGCT |
| | 7115 | G=MA=MG=MG=MA | | TGGTCAAGAG |
| | | | | GA |
| 368 | CO- | MG=MT=MG=MT=MT=dT=dT=dG=M5C=dT=dT=dG=dG=MT=d5C=dA=dA=MG= | 5874 | GTGTTTTGCT |
| | 7116 | MA=MG=MG=MA | | TGGTCAAGAG |
| | | | | GA |
| 369 | CO- | MG=MT=MG=MT=MT=.LT=dT=dG=.L5C=dT=dT=dG=dG=.LT=d5C=dA | 5875 | GTGTTTTGCT |
| | 7117 | =.LA=MG=MA=MG=MG=MA | | TGGTCAAGAG |
| | | | | GA |
| 370 | CO- | MT=MG=MT=MG=MT=MT=MT=dT=dG=d5C=dT=dT=dG=dG=dT=d5C=dA=MA= | 5876 | TGTGTTTTGC |
| | 7118 | MG=MA=MG=MG=MA=MA | | TTGGTCAAGA |
| | | | | GGAA |
| 371 | CO- | MT=MG=MT=MG=MT=MT=MT=MT=MG=M5C=MT=MT=MG=MG=MT=M5C=MA=MA | 5877 | TGTGTTTTGC |
| | 7119 | =MG=MA=MG=MG=MA=MA | | TTGGTCAAGA |
| | | | | GGAA |
| 372 | CO- | MT=MG=MT=MG=MT=dT=dT=MT=dG=d5C=MT=dT=dG=MG=dT=d5C=MA=dA= | 5878 | TGTGTTTTGC |
| | 7120 | dG=MA=MG=MG=MA=MA | | TTGGTCAAGA |
| | | | | GGAA |
| 373 | CO- | MT=MG=MT=MG=MT=dT=dT=.LT=dG=d5C=.LT=dT=dG=.LG=dT=d5C= | 5879 | TGTGTTTTGC |
| | 7121 | .LA=dA=dG=MA=MG=MG=MA=MA | | TTGGTCAAGA |
| | | | | GGAA |
| 374 | CO- | M5C=MA=M5C=M5C=M5C=M5C=d5C=d5C=dT=d5C=dA=d5C=d5C=dT=dT= | 5880 | CACCCCCCTC |
| | 7122 | dG=MG=MT=MT=M5C=M5C=M5C | | ACCTTGGTTC |
| | | | | CC |
| 375 | CO- | M5C=MA=M5C=M5C=M5C=M5C=M5C=M5C=MT=M5C=MA=M5C=M5C=MT=MT= | 5881 | CACCCCCCTC |
| | 7123 | MG=MG=MT=MT=M5C=M5C=M5C | | ACCTTGGTTC |
| | | | | CC |
| 376 | CO- | M5C=MA=M5C=M5C=M5C=d5C=d5C=d5C=MT=d5C=dA=d5C=d5C=MT= | 5882 | CACCCCCCTC |
| | 7124 | dT=dG=dG=MT=MT=M5C=M5C=M5C | | ACCTTGGTTC |
| | | | | CC |
| 377 | CO- | M5C=MA=M5C=M5C=M5C=.L5C=d5C=d5C=.LT=d5C=dA=d5C=d5C= | 5883 | CACCCCCCTC |
| | 7125 | .LT=dT=dG=.LG=MT=MT=M5C=M5C=M5C | | ACCTTGGTTC |
| | | | | CC |
| 378 | CO- | MT=M5C=MA=M5C=M5C=M5C=M5C=d5C=d5C=dT=d5C=dA=d5C=d5C=dT= | 5884 | TCACCCCCCT |
| | 7126 | dT=dG=MG=MT=MT=M5C=M5C=M5C=M5C | | CACCTTGGTT |
| | | | | CCCC |
| 379 | CO- | MT=M5C=MA=M5C=M5C=M5C=M5C=M5C=M5C=MT=M5C=MA=M5C=M5C=MT= | 5885 | TCACCCCCCT |
| | 7127 | MT=MG=MG=MT=MT=M5C=M5C=M5C=M5C | | CACCTTGGTT |
| | | | | CCCC |
| 380 | CO- | MT=M5C=MA=M5C=M5C=d5C=d5C=M5C=d5C=dT=M5C=dA=d5C=M5C=dT= | 5886 | TCACCCCCCT |
| | 7128 | dT=MG=dG=dT=MT=M5C=M5C=M5C=M5C | | CACCTTGGTT |
| | | | | CCCC |
| 381 | CO- | MT=M5C=MA=M5C=M5C=d5C=d5C=.L5C=d5C=dT=.L5C=dA=d5C=.L5C= | 5887 | TCACCCCCCT |
| | 7129 | dT=dT=.LG=dG=dT=MT=M5C=M5C=M5C=M5C | | CACCTTGGTT |
| | | | | CCCC |
| 382 | CO- | MT=MT=MA=MG=MG=MA=MG=MG=MA=MT=MT=MA=MT=MT=MT=MA=MA=M5C | 5888 | TTAGGAGGAT |
| | 7130 | =M5C=MT | | TATTTAACCT |
| 383 | CO- | M5C=MT=MT=MA=MG=dG=dA=dG=dG=dA=dT=dT=dA=dT=dT=MT=MA=MA= | 5889 | CTTAGGAGGA |
| | 7131 | M5C=M5C | | TTATTTAACC |
| 384 | CO- | M5C=M5C=MT=MT=MA=MG=MG=MA=MG=MG=MA=MT=MT=MA=MT=MT=MT=M | 5890 | CCTTAGGAGG |
| | 7132 | A=MA=M5C | | ATTATTTAAC |
| 385 | CO- | MA=M5C=MA=M5C=M5C=MT=MT=MA=MG=MG=MA=MG=MG=MA=MT=MT=MA= | 5891 | ACACCTTAGG |
| | 7133 | MT=MT=MT | | AGGATTATTT |
| 386 | CO- | MT=MT=MA=M5C=MA=M5C=M5C=MT=MT=MA=MG=MG=MA=MG=MG=MA=MT= | 5892 | TTACACCTTA |
| | 7134 | MT=MA=MT | | GGAGGATTAT |
| 387 | CO- | MT=MG=MT=MT=MA=M5C=MA=M5C=M5C=MT=MT=MA=MG=MG=MA=MG=MG= | 5893 | TGTTACACCT |
| | 7135 | MA=MT=MT | | TAGGAGGATT |
| 388 | CO- | MT=MA=MG=MT=MT=MG=MT=MT=MA=M5C=MA=M5C=M5C=MT=MT=MA=MG=M | 5894 | TAGTTGTTAC |
| | 7136 | G=MA=MG | | ACCTTAGGAG |
| 389 | CO- | MA=M5C=MT=MA=MG=MT=MT=MG=MT=MT=MA=M5C=MA=M5C=M5C=MT=MT= | 5895 | ACTAGTTGTT |
| | 7137 | MA=MG=MG | | ACACCTTAGG |
| 390 | CO- | M5C=MT=MT=MA=MG=.LG=dA=dG=.LG=dA=dT=.LT=dA=dT=.LT=MT=MA= | 5896 | CTTAGGAGGA |
| | 7138 | MA=M5C=M5C | | TTATTTAACC |
| 391 | CO- | M5C=MT=MT=.LA=MG=dG=dA=.LG=dG=dA=dT=.LT=dA=dT=dT=.LT=MA= | 5897 | CTTAGGAGGA |
| | 7139 | MA=M5C=M5C | | TTATTTAACC |
| 392 | CO- | M5C=MT=MT=MA=.LG=dG=dA=dG=.LG=dA=dT=dT=.LA=dT=dT=MT=.LA= | 5898 | CTTAGGAGGA |
| | 7140 | MA=M5C=M5C | | TTATTTAACC |
| 393 | CO- | M5C=MT=MT=.LA=MG=.LG=MA=.LG=MG=.LA=MT=.LT=MA=.LT=MT=.LT= | 5899 | CTTAGGAGGA |
| | 7141 | MA=.LA=M5C=M5C | | TTATTTAACC |
| 394 | CO- | M5C=MT=.LT=MA=.LG=MG=.LA=MG=.LG=MA=.LT=MT=.LA=MT=.LT=MT= | 5900 | CTTAGGAGGA |
| | 7142 | .LA=MA=M5C=M5C | | TTATTTAACC |
| 395 | CO- | M5C=MT=.LT=MA=MG=.LG-MA=MG=.LG=MA=MT=.LT=MA=MT=.LT=MT=MA= | 5901 | CTTAGGAGGA |
| | 7143 | .LA=M5C=M5C | | TTATTTAACC |
| 396 | CO- | M5C=MT=MT=MA=.LG=MG=MA=MG=.LG=MA=MT=MT=.LA=MT=MT=MT=.LA=MA | 5902 | CTTAGGAGGA |
| | 7144 | =M5C=M5C | | TTATTTAACC |
| 397 | CO- | M5C=MT=MT=.LA=MG=MG=MA=.LG=MG=MA=MT=.LT=MA=MT=MT=.LT=MA=MA | 5903 | CTTAGGAGGA |
| | 7145 | =M5C=M5C | | TTATTTAACC |
| 398 | CO- | M5C=MT=MT=MA=MG=.LG=MA=MG=MG=MA=.LT=MT=MA=MT=MT=.LT=MA=MA | 5904 | CTTAGGAGGA |
| | 7146 | =M5C=M5C | | TTATTTAACC |
| 399 | CO- | M5C=MT=MT=MA=.LG=MG=MA=MG=MG=.LA=MT=MT=MA=MT=.LT=MT=MA=MA | 5905 | CTTAGGAGGA |
| | 7147 | =M5C=M5C | | TTATTTAACC |
| 400 | CO- | M5C=M5C=MT=MT=MA=MG=dG=dA=dG=dG=dA=dT=dT=dA=dT=dT=MT=MA= | 5906 | CCTTAGGAGG |
| | 7148 | MA=M5C=M5C=MT | | ATTATTTAAC |
| | | | | CT |
| 401 | CO- | M5C=M5C=MT=MT=MA=MG=MG=MA=MG=MG=MA=MT=MT=MA=MT=MT=MT=M | 5907 | CCTTAGGAGG |
| | 7149 | A=MA=M5C=M5C=MT | | ATTATTTAAC |
| | | | | CT |
| 402 | CO- | M5C=M5C=MT=MT=MA=dG=dG=dA=MG=dG=dA=dT=dT=MA=dT=dT=dT=MA= | 5908 | CCTTAGGAGG |
| | 7150 | MA=M5C=M5C=MT | | ATTATTTAAC |
| | | | | CT |
| 403 | CO- | M5C=M5C=MT=MT=MA=.LG=dG=dA=.LG=dG=dA=dT=dT=.LA=dT=dT= | 5909 | CCTTAGGAGG |
| | 7151 | .LT=MA=MA=M5C=M5C=MT | | ATTATTTAAC |
| | | | | CT |
| 404 | CO- | MA=M5C=M5C=MT=MT=MA=MG=dG=dA=dG=dG=dA=dT=dT=dA=dT=dT= | 5910 | ACCTTAGGAG |
| | 7152 | MT=MA=MA=M5C=M5C=MT=M5C | | GATTATTTAA |
| | | | | CCTC |
| 405 | CO- | MA=M5C=M5C=MT=MT=MA=MG=MG=MA=MG=MG=MA=MT=MT=MA=MT=MT=M | 5911 | ACCTTAGGAG |
| | 7153 | T=MA=MA=M5C=M5C=MT=M5C | | GATTATTTAA |
| | | | | CCTC |
| 406 | CO- | MA=M5C=M5C=MT=MT=dA=dG=MG=dA=dG=MG=dA=dT=MT=dA=dT=MT= | 5912 | ACCTTAGGAG |
| | 7154 | dT=dA=MA=M5C=M5C=MT=M5C | | GATTATTTAA |
| | | | | CCTC |
| 407 | CO- | MA=M5C=M5C=MT=MT=dA=dG=.LG=dA=dG=.LG=dA=dT=.LT=dA=dT= | 5913 | ACCTTAGGAG |
| | 7155 | .LT=dT=dA=MA=M5C=M5C=MT=M5C | | GATTATTTAA |
| | | | | CCTC |
| 408 | CO- | MG=MG=MA=MG=M5C=MA=MA=MA=M5C=MT=MA=MG=MG=M5C=MA=M5C=MT | 5914 | GGAGCAAACT |
| | 7156 | =MG=MA=MG | | AGGCACTGAG |
| 409 | CO- | MG=MT=MG=MG=MA=dG=d5C=dA=dA=dA=d5C=dT=dA=dG=dG=M5C=MA= | 5915 | GTGGAGCAAA |
| | 7157 | M5C=MT=MG | | CTAGGCACTG |
| 410 | CO- | MA=MG=MG=MT=MG=MG=MA=MG=M5C=MA=MA=MA=M5C=MT=MA=MG=MG= | 5916 | AGGTGGAGCA |
| | 7158 | M5C=MA=M5C | | AACTAGGCAC |
| 411 | CO- | MG=MG=MA=MG=MG=MT=MG=MG=MA=MG=M5C=MA=MA=MA=M5C=MT=MA= | 5917 | GGAGGTGGAG |
| | 7159 | MG=MG=M5C | | CAAACTAGGC |
| 412 | CO- | MT=MG=MT=MG=MG=MA=MG=MG=MT=MG=MG=MA=MG=M5C=MA=MA=MA=M5 | 5918 | TGTGGAGGTG |
| | 7160 | C=MT=MA | | GAGCAAACTA |
| 413 | CO- | MG=MA=MT=MG=MT=MG=MG=MA=MG=MG=MT=MG=MG=MA=MG=M5C=MA=M | 5919 | GATGTGGAGG |
| | 7161 | A=MA=M5C | | TGGAGCAAAC |
| 414 | CO- | MA=MT=MG=MA=MT=MG=MT=MG=MG=MA=MG=MG=MT=MG=MG=MA=MG=M5 | 5920 | ATGATGTGGA |
| | 7162 | C=MA=MA | | GGTGGAGCAA |
| 415 | CO- | M5C=MA=MG=MA=M5C=MA=MT=MG=MA=MT=MG=MT=MG=MG=MA=MG=MG=M | 5921 | CAGACATGAT |
| | 7163 | T=MG=MG | | GTGGAGGTGG |
| 416 | CO- | MA=MT=MT=M5C=MT=MA=M5C=MA=MG=MA=MG=MG=MG=MA=MG=M5C=MA= | 5922 | ATTCTACAGA |
| | 7164 | MT=MA=MA | | GGGAGCATAA |
| 417 | CO- | MT=MT=MA=MA=MT=MT=M5C=MT=MA=M5C=MA=MG=MA=MG=MG=MG=MA=M | 5923 | TTAATTCTAC |
| | 7165 | G=M5C=MA | | AGAGGGAGCA |
| 418 | CO- | MG=MT=MT=MA=MT=MT=MA=MA=MT=MT=M5C=MT=MA=M5C=MA=MG=MA=M | 5924 | GTTATTAATT |
| | 7166 | G=MG=MG | | CTACAGAGGG |
| 419 | CO- | MG=MG=M5C=MG=MT=MT=MA=MT=MT=MA=MA=MT=MT=M5C=MT=MA=M5C=M | 5925 | GGCGTTATTA |
| | 7167 | A=MG=MA | | ATTCTACAGA |
| 420 | CO- | MT=MG=MG=M5C=MG=dT=dT=dA=dT=dT=dA=dA=dT=dT=d5C=MT=MA=M5C | 5926 | TGGCGTTATT |
| | 7168 | =MA=MG | | AATTCTACAG |
| 421 | CO- | MT=MT=MA=MT=MT=MT=MG=MG=M5C=MG=MT=MT=MA=MT=MT=MA=MA=MT= | 5927 | TTATTTGGCG |
| | 7169 | MT=M5C | | TTATTAATTC |
| 422 | CO- | MT=M5C=MT=MT=MA=MT=MT=MT=MG=MG=M5C=MG=MT=MT=MA=MT=MT=MA | 5928 | TCTTATTTGG |
| | 7170 | =MA=MT | | CGTTATTAAT |
| 423 | CO- | MA=MG=MT=M5C=MT=MT=MA=MT=MT=MT=MG=MG=M5C=MG=MT=MT=MA=MT | 5929 | AGTCTTATTT |
| | 7171 | =MT=MA | | GGCGTTATTA |
| 424 | CO- | MT=MT=MA=MG=MT=M5C=MT=MT=MA=MT=MT=MT=MG=MG=M5C=MG=MT=MT | 5930 | TTAGTCTTAT |
| | 7172 | =MA=MT | | TTGGCGTTAT |
| 425 | CO- | MA=MA=MT=MT=MA=MG=MT=M5C=MT=MT=MA=MT=MT=MT=MG=MG=M5C=M | 5931 | AATTAGTCTT |
| | 7173 | G=MT=MT | | ATTTGGCGTT |
| 426 | CO- | MT=MG=MT=MA=MA-MT=MT=MA=MG=MT=M5C=MT=MT=MA=MT=MT=MT=MG= | 5932 | TGTAATTAGT |
| | 7174 | MG=M5C | | CTTATTTGGC |
| 427 | CO- | MG=MT=MG=MG=MA=.LG=d5C=dA=.LA=dA=d5C=.LT=dA=dG=.LG=M5C= | 5933 | GTGGAGCAAA |
| | 7175 | MA=M5C=MT=MG | | CTAGGCACTG |
| 428 | CO- | MG=MT=MG=.LG=MA=dG=d5C=.LA=dA=dA=d5C=.LT=dA=dG=dG=.L5C= | 5934 | GTGGAGCAAA |
| | 7176 | MA=M5C=MT=MG | | CTAGGCACTG |
| 429 | CO- | MG=MT=MG=MG=.LA=dG=d5C=dA=.LA=dA=d5C=dT=.LA=dG=dG=M5C= | 5935 | GTGGAGCAAA |
| | 7177 | .LA=M5C=MT=MG | | CTAGGCACTG |
| 430 | CO- | MG=MT=MG=.LG=MA=.LG=M5C=.LA=MA=.LA=M5C=.LT=MA=.LG=MG= | 5936 | GTGGAGCAAA |
| | 7178 | .L5C=MA=.L5C=MT=MG | | CTAGGCACTG |
| 431 | CO- | MG=MT=.LG=MG=.LA=MG=.L5C=MA=.LA=MA=.L5C=MT=.LA=MG=.LG= | 5937 | GTGGAGCAAA |
| | 7179 | M5C=.LA=M5C=MT=MG | | CTAGGCACTG |
| 432 | CO- | MG=MT=.LG=MG=MA=.LG=M5C=MA=.LA=MA=M5C=.LT=MA=MG=.LG=M5C=MA=. | 5938 | GTGGAGCAAA |
| | 7180 | L5C=MT=MG | | CTAGGCACTG |
| 433 | CO- | MG=MT=MG=MG=.LA=MG=M5C=MA=.LA=MA=M5C=MT=.LA=MG=MG=M5C=.LA= | 5939 | GTGGAGCAAA |
| | 7181 | M5C=MT=MG | | CTAGGCACTG |
| 434 | CO- | MG=MT=MG=.LG=MA=MG=M5C=.LA=MA=MA=M5C=.LT=MA=MG=MG=.L5C=MA= | 5940 | GTGGAGCAAA |
| | 7182 | M5C=MT=MG | | CTAGGCACTG |
| 435 | CO- | MG=MT=MG=MG=MA=.LG=M5C=MA=MA=MA=.L5C=MT=MA=MG=MG=.L5C=MA= | 5941 | GTGGAGCAAA |
| | 7183 | M5C=MT=MG | | CTAGGCACTG |
| 436 | CO- | MG=MT=MG=MG=.LA=MG=M5C=MA=MA=.LA=M5C=MT=MA=MG=.LG=M5C=MA= | 5942 | GTGGAGCAAA |
| | 7184 | M5C=MT=MG | | CTAGGCACTG |
| 437 | CO- | MT=MG=MG=M5C=MG=.LT=dT=dA=.LT=dT=dA=.LA=dT=dT=.L5C=MT=MA= | 5943 | TGGCGTTATT |
| | 7185 | M5C=MA=MG | | AATTCTACAG |
| 438 | CO- | MT=MG=MG=.L5C=MG=dT=dT=.LA=dT=dT=dA=.LA=dT=dT=d5C=.LT=MA= | 5944 | TGGCGTTATT |
| | 7186 | M5C=MA=MG | | AATTCTACAG |
| 439 | CO- | MT=MG=MG=M5C=.LG=dT=dT=dA=.LT=dT=dA=dA=.LT=dT=d5C=MT= | 5945 | TGGCGTTATT |
| | 7187 | .LA=M5C=MA=MG | | AATTCTACAG |
| 440 | CO- | MT=MG=MG=.L5C=MG=.LT=MT=.LA=MT=.LT=MA=.LA=MT=.LT=M5C=.LT= | 5946 | TGGCGTTATT |
| | 7188 | MA=.L5C=MA=MG | | AATTCTACAG |
| 441 | CO- | MT=MG=.LG=M5C=.LG=MT=.LT=MA=.LT=MT=.LA=MA=.LT=MT=.L5C=MT= | 5947 | TGGCGTTATT |
| | 7189 | .LA=M5C=MA=MG | | AATTCTACAG |
| 442 | CO- | MT=MG=.LG=M5C=MG=.LT=MT=MA=.LT=MT=MA=.LA=MT=MT=.L5C=MT=MA= | 5948 | TGGCGTTATT |
| | 7190 | .L5C=MA=MG | | AATTCTACAG |
| 443 | CO- | MT=MG=MG=M5C=.LG=MT=MT=MA=.LT=MT=MA=MA=.LT=MT=M5C=MT=.LA=M5 | 5949 | TGGCGTTATT |
| | 7191 | C=MA=MG | | AATTCTACAG |
| 444 | CO- | MT=MG=MG=.L5C=MG=MT=MT=.LA=MT=MT=MA=.LA=MT=MT=M5C=.LT=MA=M5 | 5950 | TGGCGTTATT |
| | 7192 | C=MA=MG | | AATTCTACAG |
| 445 | CO- | MT=MG=MG=M5C=MG=.LT=MT=MA=MT=MT=.LA=MA=MT=MT=M5C=.LT=MA=M5 | 5951 | TGGCGTTATT |
| | 7193 | C=MA=MG | | AATTCTACAG |
| 446 | CO- | MT=MG=MG=M5C=.LG=MT=MT=MA=MT=.LT=MA=MA=MT=MT=.L5C=MT=MA=M5 | 5952 | TGGCGTTATT |
| | 7194 | C=MA=MG | | AATTCTACAG |
| 447 | CO- | MG=MG=MT=MG=MG=MA=dG=d5C=dA=dA=dA=d5C=dT=dA=dG=dG=M5C=MA= | 5953 | GGTGGAGCAA |
| | 7195 | M5C=MT=MG=MA | | ACTAGGCACT |
| | | | | GA |
| 448 | CO- | MG=MG=MT=MG=MG=MA=MG=M5C=MA=MA=MA=M5C=MT=MA=MG=MG=M5C= | 5954 | GGTGGAGCAA |
| | 7196 | MA=M5C=MT=MG=MA | | ACTAGGCACT |
| | | | | GA |
| 449 | CO- | MG=MG=MT=MG=MG=dA=dG=d5C=MA=dA=dA=d5C=dT=MA=dG=dG=d5C=MA= | 5955 | GGTGGAGCAA |
| | 7197 | M5C=MT=MG=MA | | ACTAGGCACT |
| | | | | GA |
| 450 | CO- | MG=MG=MT=MG=MG=.LA=dG=d5C=.LA=dA=dA=d5C=dT=.LA=dG=dG=.L5C= | 5956 | GGTGGAGCAA |
| | 7198 | MA=M5C=MT=MG=MA | | ACTAGGCACT |
| | | | | GA |
| 451 | CO- | MA=MG=MG=MT=MG=MG=MA=dG=d5C=dA=dA=dA=d5C=dT=dA=dG=dG=M5C=MA | 5957 | AGGTGGAGCA |
| | 7199 | =M5C=MT=MG=MA=MG | | AACTAGGCAC |
| | | | | TGAG |
| 452 | CO- | MA=MG=MG=MT=MG=MG=MA=MG=M5C=MA=MA=MA=M5C=MT=MA=MG=MG= | 5958 | AGGTGGAGCA |
| | 7200 | M5C=MA=M5C=MT=MG=MA=MG | | AACTAGGCAC |
| | | | | TGAG |
| 453 | CO- | MA=MG=MG=MT=MG=dG=dA=MG=d5C=dA=MA=dA=d5C=MT=dA=dG=MG=d5C= | 5959 | AGGTGGAGCA |
| | 7201 | dA=M5C=MT=MG=MA=MG | | AACTAGGCAC |
| | | | | TGAG |
| 454 | CO- | MA=MG=MG=MT=MG=dG=dA=.LG=d5C=dA=.LA=dA=d5C=.LT=dA=dG=. | 5960 | AGGTGGAGCA |
| | 7202 | LG=d5C=dA=M5C=MT=MG=MA=MG | | AACTAGGCAC |
| | | | | TGAG |
| 455 | CO- | MT=MT=MG=MG=M5C=MG=dT=dT=dA=dT=dT=dA=dA=dT=dT=d5C=MT=MA=M5C= | 5961 | TTGGCGTTAT |
| | 7203 | MA=MG=MA | | TAATTCTACA |
| | | | | GA |
| 456 | CO- | MT=MT=MG=MG=M5C=MG=MT=MT=MA=MT=MT=MA=MA=MT=MT=M5C=MT=MA | 5962 | TTGGCGTTAT |
| | 7204 | =M5C=MA=MG=MA | | TAATTCTACA |
| | | | | GA |
| 457 | CO- | MT=MT=MG=MG=M5C=dG=dT=dT=MA=dT=dT=dA=dA=MT=dT=d5C=dT=MA=M5C= | 5963 | TTGGCGTTAT |
| | 7205 | MA=MG=MA | | TAATTCTACA |
| | | | | GA |
| 458 | CO- | MT=MT=MG=MG=M5C=.LG=dT=dT=.LA=dT=dT=dA=dA=.LT=dT=d5C=.LT= | 5964 | TTGGCGTTAT |
| | 7206 | MA=M5C=MA=MG=MA | | TAATTCTACA |
| | | | | GA |
| 459 | CO- | MT=MT=MT=MG=MG=M5C=MG=dT=dT=dA=dT=dT=dA=dA=dT=dT=d5C=MT= | 5965 | TTTGGCGTTA |
| | 7207 | MA=M5C=MA=MG=MA=MG | | TTAATTCTAC |
| | | | | AGAG |
| 460 | CO- | MT=MT=MT=MG=MG=M5C=MG=MT=MT=MA=MT=MT=MA=MA=MT=MT=M5C=MT | 5966 | TTTGGCGTTA |
| | 7208 | =MA=M5C=MA=MG=MA=MG | | TTAATTCTAC |
| | | | | AGAG |
| 461 | CO- | MT=MT=MT=MG=MG=d5C=dG=MT=dT=dA=MT=dT=dA=MA=dT=dT=M5C=dT= | 5967 | TTTGGCGTTA |
| | 7209 | dA=M5C=MA=MG=MA=MG | | TTAATTCTAC |
| | | | | AGAG |
| 462 | CO- | MT=MT=MT=MG=MG=d5C=dG=.LT=dT=dA=.LT=dT=dA=.LA=dT=dT= | 5968 | TTTGGCGTTA |
| | 7210 | .L5C=dT=dA=M5C=MA=MG=MA=MG | | TTAATTCTAC |
| | | | | AGAG |
| 463 | CO- | MT=MG=MT=MT=MT=MT=MG=M5C=MT=MT=MG=MG=MT=M5C=MA=MA=MG=M | 5969 | TGTTTTGCTT |
| | 7378 | A=MG=MG-.[TEG] | | GGTCAAGAGG |
| 464 | CO- | MT=MG=MG=M5C=MG-MT=MT=MA=MT=MT=MA=MA=MT=MT=M5C=MT=MA=M5 | 5970 | TGGCGTTATT |
| | 7379 | C=MA=MG-.[TEG] | | AATTCTACAG |
| 465 | CO- | MA=M5C=M5C=MT=MT=MT=MT=M5C=M5C=MA=MA=M5C=M5C=MT=M5C=MG=M | | |
| | 10800 | G=MT=MT=MT | | |
| 466 | CO- | MT=MG=MG=MT=MG=MT=MT=MA=MT=MT=MA=MA=MT=MT=M5C=MT=MA=M5C | | |
| | 10831 | =MA=MG | | |
| 467 | CO- | MT=MG=MT-MT=MT=MT-MG=M5C=MT-MT=MG=MG-MT=M5C=MA-MA=MG=MA- | | |
| | 11207 | MG=MG | | |
| 468 | CO- | MT=MG=MT-MT=MT-MT=MG-M5C=MT-MT=MG-MG=MT-M5C=MA-MA=MG- | | |
| | 11208 | MA=MG=MG | | |
| 469 | CO- | MT=MG=MT=MT=MT-MT-MG=M5C=MT=MT=MG=MG=MT=M5C-MA- | 5971 | TGTTTTGCTT |
| | 11198 | MA=MG=MA=MG=MG | | GGTCAAGAGG |
| 470 | CO- | MT=MG=MT=MT-MT-MT-MG=M5C=MT=MT=MG=MG=MT=M5C-MA-MA- | 5972 | TGTTTTGCTT |
| | 11199 | MG=MA=MG=MG | | GGTCAAGAGG |
| 471 | CO- | MT=MG=MT-MT-MT-MT-MG=M5C=MT=MT=MG=MG=MT=M5C-MA-MA-MG- | 5973 | TGTTTTGCTT |
| | 11200 | MA=MG=MG | | GGTCAAGAGG |
| 472 | CO- | MT=MG-MT-MT-MT-MT-MG=M5C=MT=MT=MG=MG=MT=M5C-MA-MA-MG-MA- | 5974 | TGTTTTGCTT |
| | 11201 | MG=MG | | GGTCAAGAGG |
| 473 | CO- | MT=MG=MT-MT=MT=MT=MG=M5C-MT=MT=MG=MG=MT-M5C=MA=MA=MG=MA- | 5975 | TGTTTTGCTT |
| | 11202 | MG=MG | | GGTCAAGAGG |
| 474 | CO- | MT=MG=MT=MT-MT=MT=MG=M5C-MT=MT=MG=MG-MT=M5C=MA=MA- | 5976 | TGTTTTGCTT |
| | 11203 | MG=MA=MG=MG | | GGTCAAGAGG |
| 475 | CO- | MT=MG=MT=MT=MT-MT=MG=M5C=MT-MT=MG=MG=MT-M5C=MA=MA=MG- | 5977 | TGTTTTGCTT |
| | 11204 | MA=MG=MG | | GGTCAAGAGG |
| 476 | CO- | MT=MG-MT=MT=MT=MT-MG=M5C=MT=MT-MG=MG=MT=M5C-MA=MA=MG=MA- | 5978 | TGTTTTGCTT |
| | 11205 | MG=MG | | GGTCAAGAGG |
| 477 | CO- | MT=MG-MT=MT=MT-MT=MG=M5C-MT=MT=MG-MG=MT=M5C-MA=MA=MG- | 5979 | TGTTTTGCTT |
| | 11206 | MA=MG=MG | | GGTCAAGAGG |
| 478 | CO- | MT=MG-MT=MT-MT=MT-MG=M5C-MT=MT-MG=MG-MT=M5C-MA=MA-MG=MA- | 5980 | TGTTTTGCTT |
| | 11209 | MG=MG | | GGTCAAGAGG |
| 479 | CO- | MG=MT=.LG=MG=.LA-MG-.L5C=MA=.LA=MA=.L5C=MT=.LA=MG-.LG- | 5981 | GTGGAGCAAA |
| | 11210 | M5C=.LA=M5C=MT=MG | | CTAGGCACTG |
| 480 | CO- | MG=MT=.LG=MG-.LA-MG-.L5C=MA=.LA=MA=.L5C=MT=.LA=MG-.LG-M5C- | 5982 | GTGGAGCAAA |
| | 11211 | .LA=M5C=MT=MG | | CTAGGCACTG |
| 481 | CO- | MG=MT=.LG-MG-.LA-MG-.L5C=MA=.LA=MA=.L5C=MT=.LA=MG-.LG- | 5983 | GTGGAGCAAA |
| | 11212 | M5C-.LA-M5C=MT=MG | | CTAGGCACTG |
| 482 | CO- | MG=MT-.LG-MG-.LA-MG-.L5C=MA=.LA=MA=.L5C=MT=.LA=MG-.LG-M5C- | 5984 | GTGGAGCAAA |
| | 11213 | .LA-M5C-MT=MG | | CTAGGCACTG |
| 483 | CO- | MG=MT=.LG-MG=.LA=MG=.L5C=MA-.LA=MA=.L5C=MT=.LA- | 5985 | GTGGAGCAAA |
| | 11214 | MG=.LG=M5C=.LA=M5C-MT=MG | | CTAGGCACTG |
| 484 | CO- | MG=MT=.LG=MG-.LA=MG=.L5C=MA-.LA=MA=.L5C=MT-.LA=MG=.LG=M5C- | 5986 | GTGGAGCAAA |
| | 11215 | .LA=M5C=MT=MG | | CTAGGCACTG |
| 485 | CO- | MG=MT=.LG=MG=.LA-MG=.L5C=MA=.LA-MA=.L5C=MT=.LA-MG=.LG=M5C= | 5987 | GTGGAGCAAA |
| | 11216 | .LA-M5C=MT=MG | | CTAGGCACTG |
| 486 | CO- | MG=MT-.LG=MG=.LA=MG-.L5C=MA=.LA=MA-.L5C-MT=.LA=MG- | 5988 | GTGGAGCAAA |
| | 11217 | LG=M5C=.LA=M5C-MT=MG | | CTAGGCACTG |
| 487 | CO- | MG=MT-.LG=MG=.LA-MG=.L5C=MA-.LA=MA=.L5C-MT=.LA=MG-.LG=M5C= | 5989 | GTGGAGCAAA |
| | 11218 | .LA-M5C=MT=MG | | CTAGGCACTG |
| 488 | CO- | MG=MT=.LG-MG=.LA=MG-.L5C=MA=.LA-MA=.L5C=MT-.LA=MG=.LG- | 5990 | GTGGAGCAAA |
| | 11219 | M5C=.LA=M5C-MT=MG | | CTAGGCACTG |
| 489 | CO- | MG=MT=.LG-MG=.LA-MG=.L5C-MA=.LA-MA=.L5C-MT=.LA-MG=.LG-M5C= | 5991 | GTGGAGCAAA |
| | 11220 | .LA-M5C=MT=MG | | CTAGGCACTG |
| 490 | CO- | MG=MT-.LG=MG-.LA=MG-.L5C=MA-.LA=MA-.L5C=MT-.LA=MG-.LG= | 5992 | GTGGAGCAAA |
| | 11221 | M5C-.LA=M5C-MT=MG | | CTAGGCACTG |
| 491 | CO- | MG=MT=.LG=MG=MA-.LG-M5C=MA=.LA=MA=M5C=.LT=MA=MG-.LG- | 5993 | GTGGAGCAAA |
| | 11222 | M5C=MA=.L5C=MT=MG | | CTAGGCACTG |
| 492 | CO- | MG=MT=.LG=MG-MA-.LG-M5C=MA=.LA=MA=M5C=.LT=MA=MG-.LG-M5C- | 5994 | GTGGAGCAAA |
| | 11223 | MA=.L5C=MT=MG | | CTAGGCACTG |
| 493 | CO- | MG=MT=.LG-MG-MA-.LG-M5C=MA=.LA=MA=M5C=.LT=MA=MG-.LG-M5C-MA- | 5995 | GTGGAGCAAA |
| | 11224 | L5C=MT=MG | | CTAGGCACTG |
| 494 | CO- | MG=MT-.LG-MG-MA-.LG-M5C=MA=.LA=MA=M5C=.LT=MA=MG-.LG-M5C- | 5996 | GTGGAGCAAA |
| | 11225 | MA-.L5C-MT=MG | | CTAGGCACTG |
| 495 | CO- | MG=MT=.LG-MG=MA=.LG=M5C=MA-.LA=MA=M5C=.LT=MA- | 5997 | GTGGAGCAAA |
| | 11226 | MG=.LG=M5C=MA=.L5C-MT=MG | | CTAGGCACTG |
| 496 | CO- | MG=MT=.LG=MG-MA=.LG=M5C=MA-.LA=MA=M5C=.LT-MA=MG=.LG=M5C- | 5998 | GTGGAGCAAA |
| | 11227 | MA=.L5C=MT=MG | | CTAGGCACTG |
| 497 | CO- | MG=MT=.LG=MG=MA-.LG=M5C=MA=.LA-MA=M5C=.LT=MA-MG=.LG=M5C= | 5999 | GTGGAGCAAA |
| | 11228 | MA-L5C=MT=MG | | CTAGGCACTG |
| 498 | CO- | MG=MT-.LG=MG=MA=.LG-M5C=MA=.LA=MA-M5C=.LT=MA=MG- | 6000 | GTGGAGCAAA |
| | 11229 | LG=M5C=MA=.L5C-MT=MG | | CTAGGCACTG |
| 499 | CO- | MG=MT-.LG=MG=MA-.LG=M5C=MA-.LA=MA=M5C-.LT=MA=MG-.LG=M5C=MA- | 6001 | GTGGAGCAAA |
| | 11230 | .L5C=MT=MG | | CTAGGCACTG |
| 500 | CO- | MG=MT=.LG-MG=MA=.LG-M5C=MA=.LA-MA=M5C=.LT-MA=MG=.LG- | 6002 | GTGGAGCAAA |
| | 11231 | M5C=MA=.L5C-MT=MG | | CTAGGCACTG |
| 501 | CO- | MG=MT=.LG-MG=MA-.LG=M5C-MA=.LA-MA=M5C-.LT=MA-MG=.LG-M5C= | 6003 | GTGGAGCAAA |
| | 11232 | MA-L5C=MT=MG | | CTAGGCACTG |
| 502 | CO- | MG=MT-.LG=MG-MA=.LG-M5C=MA-.LA=MA-M5C=.LT-MA=MG-.LG=M5C | 6004 | GTGGAGCAAA |
| | 11233 | -MA=.L5C-MT=MG | | CTAGGCACTG |
| 503 | CO- | MG=MT=MG=MG=.LA-MG-M5C=MA=MA=.LA=M5C=MT=MA=MG-.LG- | 6005 | GTGGAGCAAA |
| | 11234 | M5C=MA=M5C=MT=MG | | CTAGGCACTG |
| 504 | CO- | MG=MT=MG=MG-.LA-MG-M5C=MA=MA=.LA=M5C=MT=MA=MG-.LG-M5C- | 6006 | GTGGAGCAAA |
| | 11235 | MA=M5C=MT=MG | | CTAGGCACTG |
| 505 | CO- | MG=MT=MG-MG-.LA-MG-M5C=MA=MA=.LA=M5C=MT=MA=MG-.LG-M5C-MA- | 6007 | GTGGAGCAAA |
| | 11236 | M5C=MT=MG | | CTAGGCACTG |
| 506 | CO- | MG=MT-MG-MG-.LA-MG-M5C=MA=MA=.LA=M5C=MT=MA=MG-.LG-M5C-MA- | 6008 | GTGGAGCAAA |
| | 11237 | M5C-MT=MG | | CTAGGCACTG |
| 507 | CO- | MG=MT=MG-MG=.LA=MG=M5C=MA-MA=.LA=M5C=MT=MA- | 6009 | GTGGAGCAAA |
| | 11238 | MG=.LG=M5C=MA=M5C-MT=MG | | CTAGGCACTG |
| 508 | CO- | MG=MT=MG=MG-.LA=MG=M5C=MA-MA=.LA=M5C=MT-MA=MG=.LG=M5C- | 6010 | GTGGAGCAAA |
| | 11239 | MA=M5C=MT=MG | | CTAGGCACTG |
| 509 | CO- | MG=MT=MG=MG=.LA-MG=M5C=MA=MA-.LA=M5C=MT=MA-MG=.LG=M5C=MA- | 6011 | GTGGAGCAAA |
| | 11240 | M5C=MT=MG | | CTAGGCACTG |
| 510 | CO- | MG=MT-MG=MG=.LA=MG-M5C=MA=MA=.LA-M5C=MT=MA=MG- | 6012 | GTGGAGCAAA |
| | 11241 | LG=M5C=MA=M5C-MT=MG | | CTAGGCACTG |
| 511 | CO- | MG=MT-MG=MG=.LA-MG=M5C=MA-MA=.LA=M5C-MT=MA=MG-.LG=M5C=MA- | 6013 | GTGGAGCAAA |
| | 11242 | M5C=MT=MG | | CTAGGCACTG |
| 512 | CO- | MG=MT=MG-MG=.LA=MG-M5C=MA=MA-.LA=M5C=MT-MA=MG=.LG- | 6014 | GTGGAGCAAA |
| | 11243 | M5C=MA=M5C-MT=MG | | CTAGGCACTG |
| 513 | CO- | MG=MT=MG-MG=.LA-MG=M5C-MA=MA-.LA=M5C-MT=MA-MG=.LG-M5C=MA- | 6015 | GTGGAGCAAA |
| | 11244 | M5C=MT=MG | | CTAGGCACTG |
| 514 | CO- | MG=MT-MG=MG-.LA=MG-M5C=MA-MA=.LA-M5C=MT-MA=MG-.LG=M5C- | 6016 | GTGGAGCAAA |
| | 11245 | MA=M5C-MT=MG | | CTAGGCACTG |
| 515 | CO- | MT=MG=MT-MT=MT=MT=MG=M5C-MT=MT=MG=MG=MT-M5C=MA=MA=MG=MA- | 6017 | TGTTTTGCTT |
| | 12367 | MG=MG-.[TEG] | | GGTCAAGAGG |
| 516 | CO- | MG=MT=MG=MA=MG=MG=M5C=MA=MT=M5C=MA=MG=MG=MT=MA=MA=MG=M | 6018 | GTGAGGCATC |
| | 12368 | T=MT=M5C-.[TEG] | | AGGTAAGTTC |
| 517 | CO- | MG=MT=MG-MA=MG=MG-M5C=MA=MT-M5C=MA=MG-MG=MT=MA-MA=MG=MT- | 6019 | GTGAGGCATC |
| | 12754 | MT=M5C | | AGGTAAGTTC |
| 518 | CO- | MG=MT=MG-MA=MG-MG=M5C-MA=MT-M5C=MA-MG=MG-MT=MA-MA=MG- | 6020 | GTGAGGCATC |
| | 12755 | MT=MT=M5C | | AGGTAAGTTC |
| 519 | CO- | MG=MT=MG=MA=MG-MG-M5C=MA=MT=M5C=MA=MG=MG=MT-MA- | 6021 | GTGAGGCATC |
| | 12745 | MA=MG=MT=MT=M5C | | AGGTAAGTTC |
| 520 | CO- | MG=MT=MG=MA-MG-MG-M5C=MA=MT=M5C=MA=MG=MG=MT-MA-MA- | | |
| | 12746 | MG=MT=MT=M5C | | |
| 521 | CO- | MG=MT=MG-MA-MG-MG-M5C=MA=MT=M5C=MA=MG=MG=MT-MA-MA-MG- | | |
| | 12747 | MT=MT=M5C | | |
| 522 | CO- | MG=MT-MG-MA-MG-MG-M5C=MA=MT=M5C=MA=MG=MG=MT-MA-MA-MG-MT- | | |
| | 12748 | MT=M5C | | |
| 523 | CO- | MG=MT=MG-MA=MG=MG=M5C=MA-MT=M5C=MA=MG=MG- | | |
| | 12749 | MT=MA=MA=MG=MT-MT=M5C | | |
| 524 | CO- | MG=MT=MG=MA-MG=MG=M5C=MA-MT=M5C=MA=MG-MG=MT=MA=MA- | | |
| | 12750 | MG=MT=MT=M5C | | |
| 525 | CO- | MG=MT=MG=MA=MG-MG=M5C=MA=MT-M5C=MA=MG=MG-MT=MA=MA=MG- | | |
| | 12751 | MT=MT=M5C | | |
| 526 | CO- | MG=MT-MG=MA=MG=MG-M5C=MA=MT=M5C-MA=MG=MG=MT-MA=MA=MG=MT- | | |
| | 12752 | MT=M5C | | |
| 527 | CO- | MG=MT-MG=MA=MG-MG=M5C=MA-MT=M5C=MA-MG=MG=MT-MA=MA=MG- | | |
| | 12753 | MT=MT=M5C | | |
| 528 | CO- | MG=MT-MG=MA-MG=MG-M5C=MA-MT=M5C-MA=MG-MG=MT-MA=MA-MG=MT- | | |
| | 12756 | MT=M5C | | |
| 529 | CO- | MT=MA=MG=M5C=MT=dA=d5C=d5C=d5C=dT=dT=dT=dG=dT=dT=dT=M5C= | | |
| | 13566 | MT=MG=MG=M5C | | |
| 530 | CO- | MG=MA=M5C=MA=M5C=dA=d5C-dA=dG=d5C=dA=dT=d5C=dA=dA=M5C=M5C= | | |
| | 13575 | MG=MA=M5C | | |
| 531 | CO- | MT=MA=MT=M5C=MA=dG=d5C=d5C=dA=dT=dA=dG=d5C=d5C=d5C=MT=M5C=MA= | | |
| | 13576 | MG=MG | | |
| 532 | CO- | M5C=M5C=MT=M5C=MA=dA=d5C=dG=dG=dT=dG=d5C=d5C=dT=d5C=MT=MG=MA= | | |
| | 13577 | MA=MG | | |
| 533 | CO- | M5C=MT=MT=MT=MG=dG=dT=dG=dT=dT=dA=d5C=dA=dT=dT=M5C=MT=MG=MT= | | |
| | 13578 | MT | | |
| 534 | CO- | MT=MG=M5C=M5C=MA=dT=dA=dA=dA=dA=dT=dG=d5C=d5C=d5C=MA=MA=MG=M | | |
| | 13579 | T=MT | | |
| 535 | CO- | MG=MA=MT=MT=MG=d5C=dA=dA=dG=d5C=d5C=d5C=d5C=dT=dT=MG=MA=MA=M | | |
| | 13580 | T=MA | | |
| 536 | CO- | MG=MT=MT=MT=MA=d5C=dA=d5C=dT=d5C=d5C=dA=d5C=d5C=d5C=M5C=MA= | | |
| | 13581 | M5C=MT=M5C | | |
| 537 | CO- | M5C=M5C=MT=M5C=M5C=dA=dT=dT=dT=d5C=d5C=d5C=dT=dG=dT=M5C=MT= | | |
| | 13582 | MA=MG=MG | | |
| 538 | CO- | M5C=MA=M5C=MT=MG=dT=dG=dA=dT=dT=dG=dT=d5C=dT=d5C=MT=MG=MG=MT= | | |
| | 13583 | MT | | |
| 539 | CO- | MG=M5C=MT=MT=MT=dT=dT=dA=dG=dA=dG=dA=dT=dG=dT=MT=MA=MG=MA=M | | |
| | 13584 | G | | |
| 540 | CO- | MG=MG=MG=MA=MG=dT=d5C=dA=d5C=dA=dT=dA=dT=dT=dG=M5C=MT=MA=MA= | | |
| | 13585 | MT | | |
| 541 | CO- | MG=MG=MG=MA=MG=dA=dA=dA=dG=dT=dT=dT=dG=dT=dT=MT=M5C=MT=MA=M | | |
| | 13586 | G | | |
| 542 | CO- | M5C=MA=MT=MT=MT=d5C=dT=dT=d5C=dA=dT=dT=dG=dT=d5C=M5C=M5C= | | |
| | 13587 | MG=MG=MT | | |
| 543 | CO- | MT=MA=MT=M5C=MT=dT=dT=dT=d5C=dT=d5C=d5C=dA=dA=d5C=MA=M5C= | | |
| | 13588 | M5C=MG=MT | | |
| 544 | CO- | MT=MA=MA=MT=MG=d5C=dA=dG=dA=dA=d5C=dT=dA=d5C=dA=M5C=M5C=MG=M | | |
| | 13589 | A=M5C | | |
| 545 | CO- | MT=MT=M5C=MT=MA=d5C=d5C=d5C=dT=dT=dG=dA=dA=dA=dG=M5C=MT=MG=MG | | |
| | 13590 | =MT | | |
| 546 | CO- | MG=M5C=MA=M5C=MA=dA=dT=dT=d5C=dT=dG=dT=dT=dT=dG=M5C=MT=M5C=MT | | |
| | 13591 | =MG | | |
| 547 | CO- | MG=MT=MG=MT=MG=dG=dT=dA=d5C=dA=dA=dA=dT=dG=dG=M5C=M5C=MG=MA= | | |
| | 13592 | MA | | |
| 548 | CO- | MG=MA=MG=MA=MG-dA=dG=dA=dG=dA=dG=dT=dG=dG=dT=MG=MT=MA=M5C=M | | |
| | 13593 | A | | |
| 549 | CO- | M5C=MA=MA=MT=MA=d5C=dA=dG=dA=dT=dG=dA=d5C=dT=d5C=MT=MT=MG=MG | | |
| | 13594 | =MT | | |
| 550 | CO- | MT=MG=MG=MA=MT=dA=dG=dA=dA=dA=d5C=dT=dT=dA=d5C=MA=MG=MG=MA= | | |
| | 13595 | MT | | |
| 551 | CO- | MG=MT=MA=MA=MA=d5C=dA=dA=d5C=dG=d5C=dT=dA=d5C=dA=MA=MT=MA=M5 | | |
| | 13596 | C=MT | | |
| 552 | CO- | MT=MA=MA=MA=M5C=dA=dT=dT=dG=dG=dT=d5C=dT=d5C=dT=MT=MG=MT=M5C= | | |
| | 13597 | M5C | | |
| 553 | CO- | MT=MT=MG=MT=MG=dG=dA=dG=dT=dG=dG=dA=dG=dT=dG=MT=MT=MT=M5C=MT | | |
| | 13598 | | | |
| 554 | CO- | M5C=M5C=M5C=MT=M5C=dT=dT=d5C=d5C=d5C=dT=dT=dT=dA=dA=M5C= | | |
| | 13599 | MT=M5C=MT=MT | | |
| 555 | CO- | MA=MT=MT=M5C=MA=dT=dA=dT=dT=d5C=dA=d5C=dA=dG=dG=MT=MA=MA=MG= | | |
| | 13600 | MT | | |
| 556 | CO- | MA=MG=MA=MA=MT=dG=dA=dT=dG=dA=dA=dA=dA=d5C=d5C=MG=MA=MG=MG= | | |
| | 13601 | MT | | |
| 557 | CO- | MG=MT=MT=MT=MG=dA=dA=dT=dT=d5C=dT=dG-dT=dT=d5C-MA=MG=MT=MG=M | | |
| | 13602 | 5C | | |
| 558 | CO- | MG=MA=MT=M5C=M5C=dT=dT=dA=dG=dG=d5C=dA=dA=dA=dT=MA=MG=MA=MT= | | |
| | 13603 | MA | | |
| 559 | CO- | MG=MT=M5C=MA=M5C=dT=dG=dA=dA=d5C=dT=dG=dT=dG=d5C=MT=MT=MG= | | |
| | 13604 | MG=MG | | |
| 560 | CO- | MG=M5C=MT=M5C=MT=dG=dA=dG=dT=dA=dT=dG=dT=dT=dT=M5C=MT=M5C= | | |
| | 13605 | MG=MT | | |
| 561 | CO- | MT=M5C=MA=MG=M5C=dA=dA=dT=dT=dT=d5C=d5C=dA=dA=dG=MG=M5C= | | |
| | 13606 | M5C=MT=MG | | |
| 562 | CO- | MG=M5C=MT=M5C=MA=dA=dT=dT=dT=dG=d5C=d5C=dT=d5C=dT=M5C=MG= | | |
| | 13607 | MT=MT=M5C | | |
| 563 | CO- | MG=MT=MA=MA=MA=dA=dA=dA=dT=dA=dA=dG=dG=dG=dT=MT=MG=MG=MG=M | | |
| | 13608 | A | | |
| 564 | CO- | M5C=MT=MT=MT=MT=dA=dG=dG=dG=dT=dA=dT=dT=dT=dG=MT=M5C=MT=M5C= | | |
| | 13609 | M5C | | |
| 565 | CO- | MG=M5C=MA=MA=MG=dG=d5C=dT=dG=dT=dT=dT=d5C=dA=dA=MA=MT=MG=MT= | | |
| | 13610 | MT | | |
| 566 | CO- | MG=MT=MG=MT=MG=dT=d5C=d5C=dA=dT=dG=d5C=d5C=dA=dG=MG=MG= | | |
| | 13611 | MT=MG=M5C | | |
| 567 | CO- | MG=M5C=MT=MT=MA=dT=dA=d5C=dA=dG=dA=dG=d5C=dT=dT=M5C=MA= | | |
| | 13612 | MT=M5C=MT | | |
| 568 | CO- | M5C=MT=M5C=MA=MA=dG=dA=d5C=d5C=dT=dG=dT=dT=d5C=dA=MT=MT= | | |
| | 13613 | MT=MG=MA | | |
| 569 | CO- | MG=MA=MG=MG=MA=dA=dG=dA=dG=dG=d5C=dA=dG=d5C=dA=M5C=MA= | | |
| | 13614 | MA=MA=MT | | |
| 570 | CO- | MG=MG=MG=MA=MA=dA=dA=dG=dT=dA=dA=dG=d5C=dT=d5C=MA=MG=MG= | | |
| | 13615 | M5C=MA | | |
| 571 | CO- | MT=MG=MA=MA=MG=dG=d5C=dT=dG=dG=dA=dG=dT=dG=dA=MG=MA=MG- | | |
| | 13616 | MG=M5C | | |
| 572 | CO- | MG=MT=MT=MG=MA=dG=dA=dG=dG=d5C=d5C=d5C=dA=dA=dA=MT=MG= | | |
| | 13617 | MA=MG=MG | | |
| 573 | CO- | MG=MA=MT=MA=MG=d5C=dA=d5C=d5C=d5C=dA=dA=d5C=dT=d5C=M5C= | | |
| | 13618 | MT=MA=MG=MG | | |
| 574 | CO- | MG=MG=M5C=MT=MT=d5C=dA=d5C=dA=dT=d5C=dT=dA=d5C=dT=MA=MG= | | |
| | 13619 | MT=MT=MG | | |
| 575 | CO- | MG=M5C=MA=M5C=MA=dG=dA=dG=dT=dG=dG=dA=dA=dG=dA=MA=MG=MG= | | |
| | 13620 | MT=MT | | |
| 576 | CO- | MA=MT-MT=MA=MA=dG=dA=dA=dG=d5C=dA=d5C=dT=dG=dG=M5C=M5C= | | |
| | 13621 | M5C=MA=MT | | |
| 577 | CO- | MG=MA=M5C=MA=M5C=MA=M5C=MA=MG=M5C=MA=MT=M5C=MA=MA=M5C=M | | |
| | 13622 | 5C=MG=MA=M5C | | |
| 578 | CO- | MT=MA=MT=M5C=MA=MG=M5C=M5C=MA=MT=MA=MG=M5C=M5C=M5C=MT=M5 | | |
| | 13623 | C=MA=MG=MG | | |
| 579 | CO- | M5C=M5C=MT=M5C=MA=MA=M5C=MG=MG=MT=MG=M5C=M5C=MT=M5C=MT=M | | |
| | 13624 | G=MA=MA=MG | | |
| 580 | CO- | M5C=MT=MT=MT=MG=MG=MT=MG=MT=MT=MA=M5C=MA=MT=MT=M5C=MT=M | | |
| | 13625 | G=MT=MT | | |
| 581 | CO- | MT=MG=M5C=M5C=MA=MT=MA=MA=MA=MA=MT=MG=M5C=M5C=M5C=MA=MA | | |
| | 13626 | =MG=MT=MT | | |
| 582 | CO- | MG=MA=MT=MT=MG=M5C=MA=MA=MG=M5C=M5C=M5C=M5C=MT=MT=MG=MA | | |
| | 13627 | =MA=MT=MA | | |
| 583 | CO- | MG=MT=MT=MT=MA=M5C=MA=M5C=MT=M5C=M5C=MA=M5C=M5C=M5C=M5C= | | |
| | 13628 | MA=M5C=MT=M5C | | |
| 584 | CO- | M5C=M5C=MT=M5C=M5C=MA=MT=MT=MT=M5C=M5C=M5C=MT=MG=MT=M5C= | | |
| | 13629 | MT=MA=MG=MG | | |
| 585 | CO- | M5C=MA=M5C=MT=MG=MT=MG=MA=MT=MT=MG=MT=M5C=MT=M5C=MT=MG= | | |
| | 13630 | MG=MT=MT | | |
| 586 | CO- | MG=M5C=MT=MT=MT=MT=MT=MA=MG=MA=MG=MA=MT=MG=MT=MT=MA=MG | | |
| | 13631 | =MA=MG | | |
| 587 | CO- | MG=MG=MG=MA=MG=MT=M5C=MA=M5C=MA=MT=MA=MT=MT=MG=M5C=MT= | | |
| | 13632 | MA=MA=MT | | |
| 588 | CO- | MG=MG=MG=MA=MG=MA=MA=MA=MG=MT=MT=MT=MG=MT=MT=MT=M5C=MT | | |
| | 13633 | =MA=MG | | |
| 589 | CO- | M5C=MA=MT=MT=MT=M5C=MT=MT=M5C=MA=MT=MT=MG=MT=M5C=M5C=M5C | | |
| | 13634 | =MG=MG=MT | | |
| 590 | CO- | MT=MA=MT=M5C=MT=MT=MT=MT=M5C-MT=M5C=M5C=MA=MA=M5C=MA=M5C | | |
| | 13635 | =M5C=MG=MT | | |
| 591 | CO- | MT=MA=MA=MT=MG=M5C=MA=MG=MA=MA=M5C=MT=MA=M5C=MA=M5C=M5C | | |
| | 13636 | =MG=MA=M5C | | |
| 592 | CO- | MT=MT=M5C=MT=MA=M5C=M5C=M5C=MT=MT=MG=MA=MA=MA=MG=M5C=MT | | |
| | 13637 | =MG=MG=MT | | |
| 593 | CO- | MG=M5C=MA=M5C=MA=MA=MT=MT=M5C=MT=MG=MT=MT=MT=MG=M5C=MT= | | |
| | 13638 | M5C=MT=MG | | |
| 594 | CO- | MG=MT=MG=MT=MG=MG=MT=MA=M5C=MA=MA=MA=MT=MG=MG=M5C=M5C= | | |
| | 13639 | MG=MA=MA | | |
| 595 | CO- | MG=MA=MG=MA=MG=MA=MG=MA=MG=MA=MG=MT=MG=MG=MT=MG=MT=MA | | |
| | 13640 | =M5C=MA | | |
| 596 | CO- | M5C=MA=MA=MT=MA=M5C=MA=MG=MA=MT=MG=MA=M5C=MT=M5C=MT=MT= | | |
| | 13641 | MG=MG=MT | | |
| 597 | CO- | MT=MG=MG=MA=MT=MA=MG=MA=MA=MA=M5C=MT=MT=MA=M5C=MA=MG=M | | |
| | 13642 | G=MA=MT | | |
| 598 | CO- | MG=MT=MA=MA=MA=M5C=MA=MA=M5C=MG=M5C=MT=MA=M5C=MA=MA=MT= | | |
| | 13643 | MA=M5C=MT | | |
| 599 | CO- | MT=MA=MA=MA=M5C=MA=MT=MT=MG=MG=MT=M5C=MT=M5C=MT=MT=MG=M | | |
| | 13644 | T=M5C=M5C | | |
| 600 | CO- | MT=MT=MG=MT=MG=MG=MA=MG=MT=MG=MG=MA=MG=MT=MG=MT=MT=MT= | | |
| | 13645 | M5C=MT | | |
| 601 | CO- | M5C=M5C=M5C=MT=M5C=MT=MT=M5C=M5C=M5C=MT=MT=MT=MA=MA=M5C= | | |
| | 13646 | MT=M5C=MT=MT | | |
| 602 | CO- | MA=MT=MT=M5C=MA=MT=MA=MT=MT=M5C=MA=M5C=MA=MG=MG=MT=MA= | | |
| | 13647 | MA=MG=MT | | |
| 603 | CO- | MA=MG=MA=MA=MT=MG=MA=MT=MG=MA=MA=MA=MA=M5C=M5C=MG=MA= | | |
| | 13648 | MG=MG=MT | | |
| 604 | CO- | MG=MT=MT=MT=MG=MA=MA=MT=MT=M5C=MT=MG=MT=MT=M5C=MA=MG=MT | | |
| | 13649 | =MG=M5C | | |
| 605 | CO- | MG=MA=MT=M5C=M5C=MT=MT=MA=MG=MG=M5C=MA=MA-MA=MT=MA=MG= | | |
| | 13650 | MA=MT=MA | | |
| 606 | CO- | MG=MT=M5C=MA=M5C=MT=MG=MA=MA=M5C=MT=MG=MT=MG=M5C=MT=MT= | | |
| | 13651 | MG=MG=MG | | |
| 607 | CO- | MG=M5C=MT=M5C=MT=MG=MA=MG=MT=MA=MT=MG=MT=MT=MT=M5C=MT=M | | |
| | 13652 | 5C=MG=MT | | |
| 608 | CO- | MT=M5C=MA=MG=M5C=MA=MA=MT=MT=MT=M5C=M5C=MA=MA=MG=MG=M5C | | |
| | 13653 | =M5C=MT=MG | | |
| 609 | CO- | MG=M5C=MT=M5C=MA=MA=MT=MT=MT=MG=M5C=M5C=MT=M5C=MT=M5C=MG | | |
| | 13654 | MT=MT=M5C | | |
| 610 | CO- | MG=MT=MA=MA=MA=MA=MA=MA=MT=MA=MA=MG=MG=MG=MT=MT=MG=MG | | |
| | 13655 | =MG=MA | | |
| 611 | CO- | M5C=MT=MT=MT=MT=MA=MG=MG=MG=MT=MA=MT=MT=MT=MG=MT=M5C=MT | | |
| | 13656 | =M5C=M5C | | |
| 612 | CO- | MG=M5C=MA=MA=MG=MG=M5C=MT=MG=MT=MT=MT=M5C=MA=MA=MA=MT= | | |
| | 13657 | MG=MT=MT | | |
| 613 | CO- | MG=MT=MG=MT=MG=MT=M5C=M5C=MA=MT=MG=M5C=M5C=MA=MG=MG=MG= | | |
| | 13658 | MT=MG=M5C | | |
| 614 | CO- | MG=M5C=MT=MT=MA=MT=MA=M5C=MA=MG=MA=MG=M5C=MT=MT=M5C=MA= | | |
| | 13659 | MT=M5C=MT | | |
| 615 | CO- | M5C=MT=M5C=MA=MA=MG=MA=M5C=M5C=MT=MG=MT=MT=M5C=MA=MT=MT | | |
| | 13660 | =MT=MG=MA | | |
| 616 | CO- | MG=MA=MG=MG=MA=MA=MG=MA=MG=MG=M5C=MA=MG=M5C=MA=M5C=MA= | | |
| | 13661 | MA=MA=MT | | |
| 617 | CO- | MG=MG=MG=MA=MA=MA=MA=MG=MT=MA=MA=MG=M5C=MT=M5C=MA=MG= | | |
| | 13662 | MG=M5C=MA | | |
| 618 | CO- | MT=MG=MA=MA=MG=MG=M5C=MT=MG=MG=MA=MG=MT=MG-MA=MG=MA=M | | |
| | 13663 | G=MG=M5C | | |
| 619 | CO- | MG=MT=MT=MG=MA=MG=MA=MG=MG=M5C=M5C=M5C=MA=MA=MA=MT=MG= | | |
| | 13664 | MA=MG=MG | | |
| 620 | CO- | MG=MA=MT=MA=MG=M5C=MA=M5C=M5C=M5C=MA=MA=M5C=MT=M5C=M5C= | | |
| | 13665 | MT=MA=MG=MG | | |
| 621 | CO- | MG=MG=M5C=MT=MT=M5C=MA=M5C=MA=MT=M5C=MT=MA=M5C=MT=MA=MG | | |
| | 13666 | =MT=MT=MG | | |
| 622 | CO- | MG=M5C=MA=M5C=MA=MG=MA=MG=MT=MG=MG=MA=MA=MG=MA=MA=MG= | | |
| | 13667 | MG=MT=MT | | |
| 623 | CO- | MA=MT=MT=MA=MA=MG=MA=MA=MG=M5C=MA=M5C=MT=MG=MG=M5C=M5C= | | |
| | 13668 | M5C=MA=MT | | |
| 624 | CO- | MT=MG=MG=M5C=MT=dA=d5C=d5C=d5C=dT=dT=dT=dG=d5C=dT=dT=M5C= | | |
| | 13914 | MT=MG=M5C=M5C-.[TEG] | | |
| 625 | CO- | MT=MG=MG=MA=MG=dT=dG=dA=dG=dA=dG=dG=d5C=dA=dT=MT=MT=MA= | | |
| | 15363 | MG=MG | | |
| 626 | CO- | MA=MA=MG=MG=M5C=dT=dG=dG=dA=dG=dT=dG=dA=dG=dA=MG=MG=M5C= | | |
| | 15364 | MA=MT | | |
| 627 | CO- | MT=MG=MT=MT=MT=M5C=MT=M5C=MG=MT=MA=MT=MG=MT=M5C=MA=M5C= | | |
| | 15353 | MT=MG=MA | | |
| 628 | CO- | MT=MA=MT=MG=MT=MT=MT=M5C=MT=M5C=MG=MT=MA=MT=MG=MT=M5C=M | | |
| | 15354 | A=M5C=MT | | |
| 629 | CO- | MA=MG=MT=MA=MT=MG=MT=MT=MT=M5C=MT=M5C=MG=MT=MA=MT=MG=MT | | |
| | 15355 | =M5C=MA | | |
| 630 | CO- | MT=MG=MA=MG=MT=MA=MT=MG=MT=MT=MT=M5C=MT=M5C=MG=MT=MA=MT | | |
| | 15356 | =MG=MT | | |
| 631 | CO- | MT=M5C=MT=MG=MA=MG=MT=MA=MT=MG=MT=MT=MT=M5C=MT=M5C=MG=M | | |
| | 15357 | T=MA=MT | | |
| 632 | CO- | MT=MT=MG=M5C=MT=M5C=MT=MG=MA=MG=MT=MA=MT=MG=MT=MT=MT=M5 | | |
| | 15358 | C=MT=M5C | | |
| 633 | CO- | MA=MT=MT=MT=MG=MT=M5C=MT=M5C=M5C=MA=M5C=MA=MA=MA=MA=M5C | | |
| | 15359 | =MT=M5C=MT | | |
| 634 | CO- | MG=MG=MT=MA=MT=MT=MT=MG=MT=M5C=MT=M5C=M5C=MA=M5C=MA=MA= | | |
| | 15360 | MA=MA=M5C | | |
| 635 | CO- | MT=MT=MT=MA=MG=MG=MG=MT=MA=MT=MT=MT=MG=MT=M5C=MT=M5C=M5 | | |
| | 15361 | C=MA=M5C | | |
| 636 | CO- | MT=MG=MT=MT=M5C=MT=MT=MT=MT=MA=MG=MG=MG=MT=MA=MT=MT=MT= | | |
| | 15362 | MG=MT | | |
| 637 | CO- | MG=M5C=MT=MG=MA=dA=dG=dG=d5C=dT=dG=dG=dA=dG=dT=MG=MA=MG= | | |
| | 15365 | MA=MG | | |
| 638 | CO- | MT=MG=M5C=MT=MG=dA=dA=dG=dG=d5C=dT=dG=dG=dA=dG=MT=MG=MA=MG= | | |
| | 15366 | MA | | |
| 639 | CO- | M5C=MA=MT=MT=MG=d5C=dT=dG=dA=dA=dG=dG=d5C=dT=dG=MG=MA=MG= | | |
| | 15367 | MT=MG | | |
| 640 | CO- | MG=MA=MA=MG=M5C=dA=d5C=dT=dG=dG=d5C=d5C=d5C=dA=dT=M5C=MA= | | |
| | 15368 | MA=MT=MT | | |
| 641 | CO- | MA=MA=MG=MA=MA=dG=d5C=dA=d5C=dT=dG=dG=d5C=d5C=d5C=MA=MT= | | |
| | 15369 | M5C=MA=MA | | |
| 642 | CO- | MA=MA=MA=MT=MT=dA=dA=dG=dA=dA=dG=d5C=dA=d5C=dT=MG=MG=M5C= | | |
| | 15370 | M5C=M5C | | |
| 643 | CO- | MT=MG=M5C=MA=MA=dA=dT=dT=dA=dA=dG=dA=dA=dG=d5C=MA=M5C=MT= | | |
| | 15371 | MG=MG | | |
| 644 | CO- | MT=MG=MT-MT=MT=dT=dG=d5C-dT=dT=dG=dG=dT-d5C=dA=MA=MG=MA- | | |
| | 15373 | MG=MG | | |

Additional ASO sequences that target ABCB11 regRNAs RR62_v1, RR62_v2, RR63_v1, RR63_v2, RR64_v1, RR64_v2, RR65_v1, RR74_v1, and RR84_v1 are provided in Tables 3-11. In some embodiments, an ASO of the disclosure comprises or consists of a nucleotide sequence selected from any one of the ASOs provided in Tables 2-11. In some embodiments, the ASO comprises or consists of a nucleotide sequence as set for in any one of SEQ ID NOs: 14-644, 645-954, 955-1517, 1518-2057, 2058-2308, 2309-2806, 2807-3956, 3957-4061, 4062-5228, 5229-5519, or 5520-6021.

TABLE 3
Exemplary ASOs that target regRNA RR62_v1 and
RR62_v2

| SEQ ID | |
|---|---|
| NO | Sequence |
| 645 | GGAATTTATAGTTTTCACCT |
| 646 | TCAGAGGAATTTATAGTTTT |
| 647 | GGCTCCCTCAGAGGAATTTA |
| 648 | GGAATGGCTCCCTCAGAGGA |
| 649 | ACACTAGGAATGGCTCCCTC |
| 650 | GAAAGACACTAGGAATGGCT |
| 651 | CTTGGCTAGAAAGACACTAG |
| 652 | GCACATCTTGGCTAGAAAGA |
| 653 | GTGGGGCACATCTTGGCTAG |
| 654 | GAATCAAGGGTGGGGCACAT |
| 655 | GTTCAAGAATCAAGGGTGGG |
| 656 | ATTCAGGTTCAAGAATCAAG |
| 657 | CCCACATATTCAGGTTCAAG |
| 658 | TAGGAACCCACATATTCAGG |
| 659 | TTATTTGCACATAGGAACCC |
| 660 | ACAGCTTATTTGCACATAGG |
| 661 | TCAGAACAGCTTATTTGCAC |
| 662 | TGGCTCAAGTTCAGAACAGC |
| 663 | CTCATGCTGGCTCAAGTTCA |
| 664 | GTGTGCCTCATGCTGGCTCA |
| 665 | CTTTGCAGTGTGCCTCATGC |
| 666 | GCATGGCCTTTGCAGTGTGC |
| 667 | GTCATAGCATGGCCTTTGCA |
| 668 | ACTCACTGTCATAGCATGGC |
| 669 | GGGCTGACTCACTGTCATAG |
| 670 | ACTCAGGGCTGACTCACTGT |
| 671 | CGTGTACTCAGGGCTGACTC |
| 672 | GTCTGTCGTGTACTCAGGGC |
| 673 | AGTGAGTCTGTCGTGTACTC |
| 674 | TGTAATCAGTGAGTCTGTCG |
| 675 | GCCACCTTGTAATCAGTGAG |
| 676 | TGACAGGCCACCTTGTAATC |
| 677 | TGATAAGCCTGACAGGCCAC |
| 678 | TTCTTTGTGATAAGCCTGAC |
| 679 | CTGTTTTCTTTGTGATAAGC |
| 680 | TATGCTACTGTTTTCTTTGT |
| 681 | GTCACTATGCTACTGTTTTC |
| 682 | ACACAGTCACTATGCTACTG |
| 683 | CACAGACACAGTCACTATGC |
| 684 | GAACTCACACAGACACAGTC |
| 685 | GTAGCCAGAACTCACACAGA |
| 686 | CGCAGAAGTAGCCAGAACTC |
| 687 | GCAACCACGCAGAAGTAGCC |
| 688 | CAGAGGCAACCACGCAGAAG |
| 689 | GTGATCAGAGGCAACCACGC |
| 690 | GTGCTGTGATCAGAGGCAAC |
| 691 | ACTAGGGCAGTGCTGTGATC |
| 692 | AGAAAACTAGGGCAGTGCTG |
| 693 | AGGCGTGAGAAAACTAGGGC |
| 694 | AATCTCCTCAGGTCATCTGC |
| 695 | GTAGTTGAGTTTGTTTTTTT |
| 696 | CAACCAGTAGTTGAGTTTGT |
| 697 | CTAGTCTCAACCAGTAGTTG |
| 698 | GTACACTAGTCTCAACCAGT |
| 699 | GTAAGGTACACTAGTCTCAA |
| 700 | CTTTGAGTAAGGTACACTAG |
| 701 | TGCATTACTTTGAGTAAGGT |
| 702 | CAGGCTGCATTACTTTGAGT |
| 703 | TCATGAACAGGCTGCATTAC |
| 704 | GAATTTCATGAACAGGCTGC |
| 705 | GTAATGGGAATTTCATGAAC |
| 706 | CCATCTGTAATGGGAATTTC |
| 707 | ACCCTTCCATCTGTAATGGG |
| 708 | GATCTTAACCCTTCCATCTG |
| 709 | CCACTTGATCTTAACCCTTC |
| 710 | GTGATCCACTTGATCTTAAC |
| 711 | TGGCTGGTGATCCACTTGAT |
| 712 | GTGGCTGTGGCTGGTGATCC |
| 713 | TTAGCTTTGTGGCTGTGGCT |
| 714 | TCTGCCCCTTAGCTTTGTGG |
| 715 | TCCCCTCTGCCCCTTAGCTT |
| 716 | CCAACTCCCCTCTGCCCCTT |
| 717 | CCCCCACCAACTCCCCTCTG |
| 718 | ATTTTTCCCCCCCACCAACT |
| 719 | GAGTTCTATTTTTCCCCCCC |
| 720 | TCATCTCCAAGAGTTCTATT |
| 721 | GAGTATCATCTCCAAGAGTT |
| 722 | TTTACTTGAGTATCATCTCC |
| 723 | CCCATGAATTTTACTTGAGT |
| 724 | GGAGGTGACCCATGAATTTT |
| 725 | ACTAGGGAGGTGACCCATGA |
| 726 | ACGTCACTAGGGAGGTGACC |
| 727 | ACTTAACGTCACTAGGGAGG |
| 728 | CCAATACTTAACGTCACTAG |
| 729 | CACTACCAATACTTAACGTC |
| 730 | GCATGACACTACCAATACTT |
| 731 | GGCTTGAGCATGACACTACC |
| 732 | AGTATAGGCTTGAGCATGAC |
| 733 | TATTGAGTATAGGCTTGAGC |
| 734 | ACCACTTTATTGAGTATAGG |
| 735 | ATGTTACCACTTTATTGAGT |
| 736 | GCCAAAATGTTACCACTTTA |
| 737 | TACATTGCCAAAATGTTACC |
| 738 | ACCAAATACATTGCCAAAAT |
| 739 | CACTCACCAAATACATTGCC |
| 740 | GAAAGTCACTCACCAAATAC |
| 741 | CAGGATTGAAAGTCACTCAC |
| 742 | TAGAAACAGGATTGAAAGTC |
| 743 | TCTTAATAGAAACAGGATTG |
| 744 | TTACTATGTTTTAAAATCTT |
| 745 | CTATCTTACTATGTTTTAAA |
| 746 | CAAGTTTCCTTCTATCTTAC |
| 747 | TGCCTCAAGTTTCCTTCTAT |
| 748 | TACTCTGCCTCAAGTTTCCT |
| 749 | TTTCTAGAAATTACTCTGCC |
| 750 | GAGTTTACCCATATTTCTAG |
| 751 | AACATGAGTTTACCCATATT |
| 752 | ATAGAAACATGAGTTTACCC |
| 753 | AAAGAGAATAGAAACATGAG |
| 754 | ATCTAACACAAAGAGAATAG |
| 755 | GTGGTATCTAACACAAAGAG |
| 756 | GCTGGAGTGGTATCTAACAC |
| 757 | GGCTGAGCTGGAGTGGTATC |
| 758 | TTTACTGGCTGAGCTGGAGT |
| 759 | GGGGATTTTACTGGCTGAGC |
| 760 | TATAGAGGGGATTTTACTGG |
| 761 | TCAAAATGTATAGAGGGGAT |
| 762 | GTAATTCAAAATGTATAGAG |
| 763 | ATATCTTAAATTGTAATTCA |
| 764 | TTGCTCATATCTTAAATTGT |
| 765 | ACTTTTTTGCTCATATCTTA |
| 766 | AATTTTTTACTTTTTTGCTC |
| 767 | ACGTTGTGGGTAGGTTGTTT |
| 768 | ATGGAACACGTTGTGGGTAG |
| 769 | CATGACAAATGGAACACGTT |
| 770 | ACCAGAACATGACAAATGGA |
| 771 | TCCCTCAACCAGAACATGAC |
| 772 | CTAACAGTTCCCTCAACCAG |
| 773 | TATGGACTAACAGTTCCCTC |
| 774 | ACACCATTGTATGGACTAAC |
| 775 | GTGTGAATAACACCATTGTA |
| 776 | TCCAGGAAAAGCATGTGTGA |
| 777 | CCAGATTCCAGGAAAAGCAT |
| 778 | AAGAACTCCAGATTCCAGGA |
| 779 | AGTTACAAGAACTCCAGATT |
| 780 | CGACGTTGAGTTACAAGAAC |
| 781 | ATTGACTACGACGTTGAGTT |
| 782 | TGTTTTTATTGACTACGACG |
| 783 | TGACAGATGTTTTTATTGAC |
| 784 | CACAATGACAGATGTTTTTA |
| 785 | TTTGGCACAATGACAGATGT |
| 786 | TACTCATTTTTGGCACAATG |
| 787 | GCGTGCTACTCATTTTTGGC |
| 788 | GCCAGGCGTGCTACTCATTT |
| 789 | GCCCAGCCAGGCGTGCTACT |
| 790 | GGAATAGCCCAGCCAGGCGT |
| 791 | ATTTCTCCATGGAATAGCCC |
| 792 | GTGTGCATTTCTCCATGGAA |
| 793 | AGTTTGTGTGCATTTCTCCA |
| 794 | TTGTGGGAAGTTTGTGTGCA |
| 795 | TGATGTTTGTGGGAAGTTTG |
| 796 | TTGGCTGATGTTTGTGGGAA |
| 797 | GACATTTGGCTGATGTTTGT |
| 798 | CAACTGACATTTGGCTGATG |
| 799 | CTTCATCAACTGACATTTGG |
| 800 | TCGGTTTTCTTCATCAACTG |
| 801 | AAGTTTCGGTTTTCTTCATC |
| 802 | CTTTGTTTTTCAAGTTTCGG |
| 803 | GAACCCTTTGTTTTTCAAGT |
| 804 | ATGATCTCTGAACCCTTTGT |
| 805 | CCATAGATGATCTCTGAACC |
| 806 | GCTCTTCACCCATAGATGAT |
| 807 | CTCCTGCTCTTCACCCATAG |
| 808 | CTACCTCTCCTGCTCTTCAC |
| 809 | TAGTCCTCCTACCTCTCCTG |
| 810 | ACTAAATTAAGTAGTCCTCC |
| 811 | TAGTAACTAAATTAAGTAGT |
| 812 | TGAGAATCTAGTAACTAAAT |
| 813 | ATTCATGAGAATCTAGTAAC |
| 814 | GAGTACTATTCATGAGAATC |
| 815 | GGTGTTGAGTACTATTCATG |
| 816 | GAAATTTGGTGTTGAGTACT |
| 817 | CCCATGAAATTTGGTGTTGA |
| 818 | GTGATTCCCATGAAATTTGG |
| 819 | CAATTAAGGTGATTCCCATG |
| 820 | TCTGCCACTCAATTAAGGTG |
| 821 | CGAACTCTGCCACTCAATTA |
| 822 | GTATACGAACTCTGCCACTC |
| 823 | AATTAGTATACGAACTCTGC |
| 824 | GCCGTTTATTTCAATATAAT |
| 825 | TACCGTGCCGTTTATTTCAA |
| 826 | TGATGTACCGTGCCGTTTAT |
| 827 | ATAATTGATGTACCGTGCCG |
| 828 | TTTAGATAATTGATGTACCG |
| 829 | AGTCTAATGTTTTAGATAAT |
| 830 | GTAAGAGTCTAATGTTTTAG |
| 831 | ACAATGTAAGAGTCTAATGT |
| 832 | GATCTAACAATGTAAGAGTC |
| 833 | TATAACATGATCTAACAATG |
| 834 | GGAAATTTATAACATGATCT |
| 835 | GAGCAGGAAATTTATAACAT |
| 836 | GAATGAATTCAGAGAGCAGG |
| 837 | TAAACAGAATGAATTCAGAG |
| 838 | TCCTGAAATAAACAGAATGA |
| 839 | TCAGCTCCTGAAATAAACAG |
| 840 | CGAAAGTCAGCTCCTGAAAT |
| 841 | TAAAAACGAAAGTCAGCTCC |
| 842 | GAGTAGCTAAAAACGAAAGT |
| 843 | TAGATGAGTAGCTAAAAACG |
| 844 | CTAAGTTGTAGATGAGTAGC |
| 845 | TTCCCCTAAGTTGTAGATGA |
| 846 | GATTATTTTCCCCTAAGTTG |
| 847 | AATGACAGTGATTATTTTCC |
| 848 | GCATCAATGACAGTGATTAT |
| 849 | CAATGGCATCAATGACAGTG |
| 850 | TTATCTACTTGGCAATGGCA |
| 851 | ACACAGTTTATCTACTTGGC |
| 852 | GCCCCACACAGTTTATCTAC |
| 853 | TCTCAATGCCCCACACAGTT |
| 854 | GTATTTCTCAATGCCCCACA |
| 855 | TTTATAGTATTTCTCAATGC |
| 856 | TCAACAAAACAATTTATAGT |
| 857 | TCAGTTCAACAAAACAATTT |
| 858 | CACTATCAGTTCAACAAAAC |
| 859 | CATAGTCACTATCAGTTCAA |
| 860 | CATTCTTCATAGTCACTATC |
| 861 | AATTGGCATTCTTCATAGTC |
| 862 | GGCAGAAATTGGCATTCTTC |
| 863 | TTCTAGGCAGAAATTGGCAT |
| 864 | GGAGGCATTTCTAGGCAGAA |
| 865 | AGGATGGAGGCATTTCTAGG |
| 866 | TTTTCTCAGGATGGAGGCAT |
| 867 | CCAAATTTTCTCAGGATGGA |
| 868 | TTACTCCAAATTTTCTCAGG |
| 869 | GGTGTTTACTCCAAATTTTC |
| 870 | AAAGTATAGGTGTTTACTCC |
| 871 | GAAATTTTAAAGTATAGGTG |
| 872 | CTTATGACAAATCTTTAGAA |
| 873 | ATTTATTCTTATGACAAATC |
| 874 | TATATATTTATTCTTATGAC |
| 875 | CACAACAATCACATATATAT |
| 876 | GTCATCACAACAATCACATA |
| 877 | GGAAAGTCATCACAACAATC |
| 878 | TTTGTAAGGAAAGTCATCAC |
| 879 | TCATATTTGTAAGGAAAGTC |
| 880 | GGTTATCATATTTGTAAGGA |
| 881 | CCCATGGTTATCATATTTGT |
| 882 | CTAAGCCCATGGTTATCATA |
| 883 | TATCACTAAGCCCATGGTTA |
| 884 | GGAAAATTATCACTAAGCCC |
| 885 | AATGTCTTGGAAAATTATCA |
| 886 | GATCAGAAAAATGTCTTGGA |
| 887 | CAATAGATCAGAAAAATGTC |
| 888 | AATCCTATCCAATAGATCAG |
| 889 | ATACAAATCCTATCCAATAG |
| 890 | AAAGCATACAAATCCTATCC |
| 891 | CTCATAAAGCATACAAATCC |
| 892 | ATATTGCTCATAAAGCATAC |
| 893 | GAAATATATTGCTCATAAAG |
| 894 | CTATTAGAAATATATTGCTC |
| 895 | TGAATAACTATTAGAAATAT |
| 896 | CATGCTGAATAACTATTAGA |
| 897 | GATAACATGCTGAATAACTA |
| 898 | GCCTATGTGATAACATGCTG |
| 899 | GGCTTTGCCTATGTGATAAC |
| 900 | TCATAGGCTTTGCCTATGTG |
| 901 | TTTTCAGTCATAGGCTTTGC |
| 902 | GTGATCACTTTTTCAGTCAT |
| 903 | ACTTCAGTGATCACTTTTTC |
| 904 | GTTTTTAACTTCAGTGATCA |
| 905 | GGCAGGTTTTTAACTTCAGT |
| 906 | GTCATATTGGCAGGTTTTTA |
| 907 | CTTTAGTCATATTGGCAGGT |
| 908 | TAAATCTTTAGTCATATTGG |
| 909 | GGGGAGTGTTAAATCTTTAG |
| 910 | GATCATGAGGGGAGTGTTAA |
| 911 | ATTGTTTAGATCATGAGGGG |
| 912 | TATAAATTGTTTAGATCATG |
| 913 | GCAGCTATAAATTGTTTAGA |
| 914 | GGGTGTGCAGCTATAAATTG |
| 915 | GCAGTGGGTGTGCAGCTATA |
| 916 | TTATGGCAGTGGGTGTGCAG |
| 917 | GTTGATTTATGGCAGTGGGT |
| 918 | ACTGTGTTGATTTATGGCAG |
| 919 | TGGTAATAAAACTGTGTTGA |
| 920 | TCAATTGGTAATAAAACTGT |
| 921 | CTTCTTTCAATTGGTAATAA |
| 922 | GAGTTGGCTTCTTTCAATTG |
| 923 | GGCGTTAGAGTTGGCTTCTT |
| 924 | GTGATGGCGTTAGAGTTGGC |
| 925 | GAAAGGTGATGGCGTTAGAG |
| 926 | GAGAAGAAAGGTGATGGCGT |
| 927 | ACAAGATGAGAAGAAAGGTG |
| 928 | CATCAGGTTACAAGATGAGA |
| 929 | GTTTTTCTCATCAGGTTACA |
| 930 | CTTATGTTTTTCTCATCAGG |
| 931 | AAATCCTTATGTTTTTCTCA |
| 932 | GTGGTCTTTAAATCCTTATG |
| 933 | GAAAGGGTGGTCTTTAAATC |
| 934 | TTGTATTGGAAAGGGTGGTC |
| 935 | TCCCATTGTATTGGAAAGGG |
| 936 | GAATTTCCCATTGTATTGGA |
| 937 | CTAGGGAGAATTTCCCATTG |
| 938 | ATCACCACCTAGGGAGAATT |
| 939 | TCATAAAATCACCACCTAGG |
| 940 | GAATAACTCATAAAATCACC |
| 941 | GGGGATTTTCTTTCTTACTA |
| 942 | GCCAGTGGGGATTTTCTTTC |
| 943 | TTTTCCAGCCAGTGGGGATT |
| 944 | CAGAATTTTCCAGCCAGTGG |
| 945 | TTGGCCAGAATTTTCCAGCC |
| 946 | ATACTAGGTTTGGCCAGAAT |
| 947 | TTAAGATACTAGGTTTGGCC |
| 948 | GCTTGTGTTTAAGATACTAG |
| 949 | GAATAAAGCTTGTGTTTAAG |
| 950 | CTAGAAGAATAAAGCTTGTG |
| 951 | GAAATCTAGAAGAATAAAGC |
| 952 | GAAGAGGGAAATCTAGAAGA |
| 953 | TCTCTAGAAGAGGGAAATCT |
| 954 | CTGAAATCTCTAGAAGAGGG |

TABLE 4
Exemplary ASOs that target regRNA RR62_v2

| SEQ ID | |
|---|---|
| NO | Sequence |
| 955 | ATCATCTCCAAGAGTTCTAT |
| 956 | TGAGTATCATCTCCAAGAGT |
| 957 | TACTTAACGTCACTAGGGAG |
| 958 | GCTATACTGAAATCTCTAGA |
| 959 | GATTGAGCTATACTGAAATC |
| 960 | TAGTGAACCTGAGATTGAGC |
| 961 | ATCGCTAGTGAACCTGAGAT |
| 962 | CTGTGAATCGCTAGTGAACC |
| 963 | GTGTTCATCTCTGTGAATCG |
| 964 | CTCAGGTGTTCATCTCTGTG |
| 965 | ATACCTCTCAGGTGTTCATC |
| 966 | TCTCCATACCTCTCAGGTGT |
| 967 | TTACTTTCTCTCCATACCTC |
| 968 | GGACTTTTTACTTTCTCTCC |
| 969 | GAGTATAGAGGACTTTTTAC |
| 970 | GTTGGTATAACTGGGAGTAT |
| 971 | TATTTGTTGGTATAACTGGG |
| 972 | GGCAAAGAGTTATTTGTTGG |
| 973 | CTGAGGGCAAAGAGTTATTT |
| 974 | CATGACTGAGGGCAAAGAGT |
| 975 | AGGGTCATGACTGAGGGCAA |
| 976 | GATTGAGGGTCATGACTGAG |
| 977 | AAATAGATTGAGGGTCATGA |
| 978 | GCAGTCACAAATAGATTGAG |
| 979 | GGAAATGCAGTCACAAATAG |
| 980 | CCATGAGGAAATGCAGTCAC |
| 981 | ACTTACCCATGAGGAAATGC |
| 982 | ACCACTTCAACTTACCCATG |
| 983 | ATCCAACCACTTCAACTTAC |
| 984 | AATTAGCTAATCCAACCACT |
| 985 | GCTTTAAAATTAGCTAATCC |
| 986 | GAGGAAGCTTTAAAATTAGC |
| 987 | TTAAGCATGAGGAAGCTTTA |
| 988 | TAATATTTTTAAGCATGAGG |
| 989 | TAGTTGTAATATTTTTAAGC |
| 990 | TTAATGTAGTTGTAATATTT |
| 991 | CATTATTTAATGTAGTTGTA |
| 992 | ATCAACATTATTTAATGTAG |
| 993 | ATCATTATCAACATTATTTA |
| 994 | GAATTTTTCATCATTATCAA |
| 995 | GTTCTTGAATTTTTCATCAT |
| 996 | GTGGGCATGTTCTTGAATTT |
| 997 | AAAGTGTGGGCATGTTCTTG |
| 998 | TCTATGAAAAGTGTGGGCAT |
| 999 | ATGATTCTATGAAAAGTGTG |
| 1000 | TAAGGAAATATGATTCTATG |
| 1001 | TGAATGGGAAATATTAAGGA |
| 1002 | TGGGAAATGAAATGAATGGG |
| 1003 | CCTTATGGGAAATGAAATGA |
| 1004 | CATGGCCTTATGGGAAATGA |
| 1005 | GGTTAAACATGGCCTTATGG |
| 1006 | ATACAGGTTAAACATGGCCT |
| 1007 | GGTCAAATACAGGTTAAACA |
| 1008 | TGAAGAGGTCAAATACAGGT |
| 1009 | CCCGAAGTTGAAGAGGTCAA |
| 1010 | AGATCCCCGAAGTTGAAGAG |
| 1011 | GGTTAAGTCAAGATCCCCGA |
| 1012 | TGCGTGACTCAGGTTAAGTC |
| 1013 | GTATTCATGGACTGCGTGAC |
| 1014 | AGGGTGTATTCATGGACTGC |
| 1015 | GGTCCAGGGTGTATTCATGG |
| 1016 | TTCCTGGTGACAGCAAGGTC |
| 1017 | CAGTAGTTCCTGGTGACAGC |
| 1018 | TCAGACAGTAGTTCCTGGTG |
| 1019 | GTTAAACATGTCAGACAGTA |
| 1020 | AAGGAGTTAAACATGTCAGA |
| 1021 | GTGCCAAGGAGTTAAACATG |
| 1022 | CAGTCACAGAGTGCCAAGGA |
| 1023 | ATGGGCAGTCACAGAGTGCC |
| 1024 | GGTACATGGGCAGTCACAGA |
| 1025 | AAATGAGGTACATGGGCAGT |
| 1026 | GAAGCGAGAAAATGAGGTAC |
| 1027 | GTGGAAGGAGGAAGGAAGCG |
| 1028 | CAAATAGGTGTGGAAGGAGG |
| 1029 | CAAGTGAGGTTGACAAATAG |
| 1030 | ATGGCTCAAGTGAGGTTGAC |
| 1031 | AGGACTGCATGGCTCAAGTG |
| 1032 | TGAAGGATTTAAAGGACTGC |
| 1033 | GGAAGGAAATGAAGGATTTA |
| 1034 | AGCAATGGAAGGAAATGAAG |
| 1035 | GGGTTTAGCAATGGAAGGAA |
| 1036 | TTAATGGGGTTTAGCAATGG |
| 1037 | AAATCTTAATGGGGTTTAGC |
| 1038 | GAAGCAAAATCTTAATGGGG |
| 1039 | GCATGGGGAAGGAAGCAAAA |
| 1040 | TTGGCTGCATGGGGAAGGAA |
| 1041 | GGACCTTGGCTGCATGGGGA |
| 1042 | CAGAGGGACCTTGGCTGCAT |
| 1043 | ATGCCTGGCAGAGGGACCTT |
| 1044 | GTTCAAATGCCTGGCAGAGG |
| 1045 | GTGTGGTTCAAATGCCTGGC |
| 1046 | AGGATGATGGTGAGAGTGTG |
| 1047 | ATAATTGAGGATGATGGTGA |
| 1048 | TAGAGAAAATAATTGAGGAT |
| 1049 | TGATGTAGAGAAAATAATTG |
| 1050 | TTACAAGGATGATGTAGAGA |
| 1051 | GAAGTTTACAAGGATGATGT |
| 1052 | GGATGCAGGATGAAGTTTAC |
| 1053 | CAGCTGGATGGATGCAGGAT |
| 1054 | GGCAAACAGCTGGATGGATG |
| 1055 | AAGAGGGCAAACAGCTGGAT |
| 1056 | TAAGGAGAAGAGGGCAAACA |
| 1057 | TGGCTTAAGGAGAAGAGGGC |
| 1058 | AGCAGCCTGGCTTAAGGAGA |
| 1059 | TTGTGTCCAGCAGGCCCAGC |
| 1060 | GTGAGGTTGTGTCCAGCAGG |
| 1061 | CAAGGTGGTGAGGTTGTGTC |
| 1062 | ATTCACAAGGTGGTGAGGTT |
| 1063 | CCTGTGTTGGTATCCATTCA |
| 1064 | TAGACTCTGCCTGTGTTGGT |
| 1065 | GCTGAGTACTAGACTCTGCC |
| 1066 | GGGCAGCTGAGTACTAGACT |
| 1067 | AGCTTAGGGCAGCTGAGTAC |
| 1068 | GAAGGGAAGCTTAGGGCAGC |
| 1069 | GGGCCAGAAGGGAAGCTTAG |
| 1070 | TTCTTTTCTGGGCCAGAAGG |
| 1071 | TAGGATTCTTTTCTGGGCCA |
| 1072 | AAATTTAGGATTCTTTTCTG |
| 1073 | TCAGAAAAAATTTAGGATTC |
| 1074 | GGGGAGTCTCAGAAAAAATT |
| 1075 | GACCGTGGGGAGTCTCAGAA |
| 1076 | GGCCAGACCGTGGGGAGTCT |
| 1077 | GCCAAGGGCCAGACCGTGGG |
| 1078 | AGGTCAGAGAGCCAAGGGCC |
| 1079 | AGTTGGAGGAGGTGAGGTCA |
| 1080 | GAATATAGTTGGAGGAGGTG |
| 1081 | GAGAACTAGAATATAGTTGG |
| 1082 | GTGGGGTGTTGAGAACTAGA |
| 1083 | GGCAAGTGGGGTGTTGAGAA |
| 1084 | GGCCAGGCAAGTGGGGTGTT |
| 1085 | CAAGAGGCCAGGCAAGTGGG |
| 1086 | GTCAGCAAGAGGCCAGGCAA |
| 1087 | GACAAAGGTCAGCAAGAGGC |
| 1088 | GCATGGACAAAGGTCAGCAA |
| 1089 | GTTGGGCATGGACAAAGGTC |
| 1090 | GAACAGTTGGGCATGGACAA |
| 1091 | AGGTGGGAACAGTTGGGCAT |
| 1092 | CCGCACCCAGAGGTGGGAAC |
| 1093 | GGTGCCCGCACCCAGAGGTG |
| 1094 | TAGCAGGTGCCCGCACCCAG |
| 1095 | AAGGAGTAGCAGGTGCCCGC |
| 1096 | GGGGAGAAGGAGTAGCAGGT |
| 1097 | CACCTGGGGAGAAGGAGTAG |
| 1098 | AAATGCACCTGGGGAGAAGG |
| 1099 | CCTAAAGAAAATGCACCTGG |
| 1100 | TGTGGTTACCTAAAGAAAAT |
| 1101 | TGGAGCTGTCCCTGTGGTTA |
| 1102 | GGTGATGGAGCTGTCCCTGT |
| 1103 | GCGGAGGTGATGGAGCTGTC |
| 1104 | AGGTCTAGCGGAGGTGATGG |
| 1105 | TTCAAAGGAAGGTCTAGCGG |
| 1106 | GGTGATTTCAAAGGAAGGTC |
| 1107 | TGAGTAGGTGATTTCAAAGG |
| 1108 | TCTGTTGAGTAGGTGATTTC |
| 1109 | GGCAAGACTCTGTTGAGTAG |
| 1110 | TTTGGCCAGGCAAGACTCTG |
| 1111 | AATTTTAAAGACTTTGGCCA |
| 1112 | GGGGTTGAAATTTTAAAGAC |
| 1113 | CGTGGGGGGTGGGGTTGAAA |
| 1114 | AATGGTGCGTGGGGGGTGGG |
| 1115 | TATGAAATGGTGCGTGGGGG |
| 1116 | GGAGCTATGAAATGGTGCGT |
| 1117 | GAAATGGAGCTATGAAATGG |
| 1118 | AAACTGGAAATGGAGCTATG |
| 1119 | ATCACTTTCTTTCTGAGAAG |
| 1120 | AATCTATCACTTTCTTTCTG |
| 1121 | AGTGATAATCTATCACTTTC |
| 1122 | GTAGCTAGTGATAATCTATC |
| 1123 | GTATAGCATGGTAGCTAGTG |
| 1124 | CAAATGTATAGCATGGTAGC |
| 1125 | TAAATCAAATGTATAGCATG |
| 1126 | CATTGTAAATAAATCAAATG |
| 1127 | ATAAACATTGTAAATAAATC |
| 1128 | GGGAGATATAATAAACATTG |
| 1129 | TTTAGTGAGGGAGATATAAT |
| 1130 | GTTTACATTTTAGTGAGGGA |
| 1131 | GTTGGCCCTTGAGGAGTTTA |
| 1132 | ATAGGCAAAAATGTTGGCCC |
| 1133 | GCGGGTATAGAAAAGAACAA |
| 1134 | GTGCTGCGGGTATAGAAAAG |
| 1135 | TGGTATCAGACACTATTGTA |
| 1136 | CTATTTGGTATCAGACACTA |
| 1137 | TTGAGTGTCTATTTGGTATC |
| 1138 | TGCAAATATTGAGTGTCTAT |
| 1139 | CGTTTTGCAAATATTGAGTG |
| 1140 | TTAATTCATTCGTTTTGCAA |
| 1141 | TCACTCCATTAATTCATTCG |
| 1142 | CACATTCACTCCATTAATTC |
| 1143 | CTGGAGAGCACACATTCACT |
| 1144 | TTGAGCTGGAGAGCACACAT |
| 1145 | TTGCATGTTGAGCTGGAGAG |
| 1146 | AGTGTGCATTGCATGTTGAG |
| 1147 | AGAAATGAAGTGTGCATTGC |
| 1148 | TCCGCAGAGAAATGAAGTGT |
| 1149 | GAGCAATCCGCAGAGAAATG |
| 1150 | AATGGGAGCAATCCGCAGAG |
| 1151 | CTGCTGAGAAGAATGGGAGC |
| 1152 | CAGAGCTGCTGAGAAGAATG |
| 1153 | TTTTGCCAGAGCTGCTGAGA |
| 1154 | GCAAATTTTGCCAGAGCTGC |
| 1155 | GATCACAGCAAATTTTGCCA |
| 1156 | GAGAAGATCACAGCAAATTT |
| 1157 | CATGGAATGAGAAGATCACA |
| 1158 | GGTGAGCATGGAATGAGAAG |
| 1159 | TTTAGAGGAGGTGAGCATGG |
| 1160 | GCTTTGTTTTTAGAGGAGGT |
| 1161 | ATGAGTTGCTTTGTTTTTAG |
| 1162 | GTTGAATGAGTTGCTTTGTT |
| 1163 | GGATGAGTAGGAAGTTGAAT |
| 1164 | GGAGAGGATGAGTAGGAAGT |
| 1165 | TGCGTGGGAAGTTTAAAAAA |
| 1166 | CTTATTGCGTGGGAAGTTTA |
| 1167 | CGTTTCTTATTGCGTGGGAA |
| 1168 | CTCTGAGAGCCACACGTTTC |
| 1169 | GCCTGGGCCTCTGAGAGCCA |
| 1170 | AGGTCAGCCTGGGCCTCTGA |
| 1171 | GTGGTCAGGTCAGCCTGGGC |
| 1172 | TGGGAGTGGTCAGGTCAGCC |
| 1173 | TTTGGGCTGGGAGTGGTCAG |
| 1174 | GATTAGCTTTGGGCTGGGAG |
| 1175 | GTGGTAAGATTAGCTTTGGG |
| 1176 | GAGGAAGTGGGAGTGGTAAG |
| 1177 | TCACAGCAGAGGGAGGAAGT |
| 1178 | AGTAATTCACAGCAGAGGGA |
| 1179 | TTTTAAGTAATTCACAGCAG |
| 1180 | GGTATTTTAGAAATTTTAAG |
| 1181 | GGGTTAAAGGTATTTTAGAA |
| 1182 | GTGGAAACGGAAGGGTTAAA |
| 1183 | AGGTGGTGGAAACGGAAGGG |
| 1184 | ATCAGGTAAGTTCCTGAAGT |
| 1185 | GTCTAATTTAGAAGACAGAA |
| 1186 | TAGATGAGGTCTAATTTAGA |
| 1187 | GACAGAAGTGAGGCATCA |
| 1188 | GTGAGGCATCAGGTAAGT |
| 1189 | GAGGCATCAGGTAAGTTC |
| 1190 | GCATCAGGTAAGTTCCTG |
| 1191 | TCCTGAAGTGGAGGTGGT |
| 1192 | CCTGAAGTGGAGGTGGTG |
| 1193 | GTCTAATTTAGAAGACAGA |
| 1194 | GAAGACAGAAGTGAGGCAT |
| 1195 | AGACAGAAGTGAGGCATCA |
| 1196 | GACAGAAGTGAGGCATCAG |
| 1197 | AAGTGAGGCATCAGGTAAG |
| 1198 | AGTGAGGCATCAGGTAAGT |
| 1199 | GTGAGGCATCAGGTAAGTT |
| 1200 | TGAGGCATCAGGTAAGTTC |
| 1201 | GAGGCATCAGGTAAGTTCC |
| 1202 | GGCATCAGGTAAGTTCCTG |
| 1203 | GCATCAGGTAAGTTCCTGA |
| 1204 | ATCAGGTAAGTTCCTGAAG |
| 1205 | TCAGGTAAGTTCCTGAAGT |
| 1206 | GTAAGTTCCTGAAGTGGAG |
| 1207 | AAGTTCCTGAAGTGGAGGT |
| 1208 | GTTCCTGAAGTGGAGGTGG |
| 1209 | TTCCTGAAGTGGAGGTGGT |
| 1210 | TCCTGAAGTGGAGGTGGTG |
| 1211 | CCTGAAGTGGAGGTGGTGG |
| 1212 | TGAGGTCTAATTTAGAAGAC |
| 1213 | GAGGTCTAATTTAGAAGACA |
| 1214 | AGGTCTAATTTAGAAGACAG |
| 1215 | GGTCTAATTTAGAAGACAGA |
| 1216 | AGAAGACAGAAGTGAGGCAT |
| 1217 | GAAGACAGAAGTGAGGCATC |
| 1218 | AAGACAGAAGTGAGGCATCA |
| 1219 | AGACAGAAGTGAGGCATCAG |
| 1220 | GACAGAAGTGAGGCATCAGG |
| 1221 | ACAGAAGTGAGGCATCAGGT |
| 1222 | GAAGTGAGGCATCAGGTAAG |
| 1223 | AAGTGAGGCATCAGGTAAGT |
| 1224 | AGTGAGGCATCAGGTAAGTT |
| 1225 | GTGAGGCATCAGGTAAGTTC |
| 1226 | TGAGGCATCAGGTAAGTTCC |
| 1227 | GAGGCATCAGGTAAGTTCCT |
| 1228 | AGGCATCAGGTAAGTTCCTG |
| 1229 | GGCATCAGGTAAGTTCCTGA |
| 1230 | GCATCAGGTAAGTTCCTGAA |
| 1231 | CATCAGGTAAGTTCCTGAAG |
| 1232 | TCAGGTAAGTTCCTGAAGTG |
| 1233 | CAGGTAAGTTCCTGAAGTGG |
| 1234 | GGTAAGTTCCTGAAGTGGAG |
| 1235 | GTAAGTTCCTGAAGTGGAGG |
| 1236 | TAAGTTCCTGAAGTGGAGGT |
| 1237 | AAGTTCCTGAAGTGGAGGTG |
| 1238 | AGTTCCTGAAGTGGAGGTGG |
| 1239 | GTTCCTGAAGTGGAGGTGGT |
| 1240 | TTCCTGAAGTGGAGGTGGTG |
| 1241 | TCCTGAAGTGGAGGTGGTGG |
| 1242 | CCTGAAGTGGAGGTGGTGGA |
| 1243 | CTGAAGTGGAGGTGGTGGAA |
| 1244 | ATGAGGTCTAATTTAGAAGAC |
| 1245 | TGAGGTCTAATTTAGAAGACA |
| 1246 | GAGGTCTAATTTAGAAGACAG |
| 1247 | AGGTCTAATTTAGAAGACAGA |
| 1248 | GGTCTAATTTAGAAGACAGAA |
| 1249 | GTCTAATTTAGAAGACAGAAG |
| 1250 | TCTAATTTAGAAGACAGAAGT |
| 1251 | CTAATTTAGAAGACAGAAGTG |
| 1252 | TAATTTAGAAGACAGAAGTGA |
| 1253 | AATTTAGAAGACAGAAGTGAG |
| 1254 | TAGAAGACAGAAGTGAGGCAT |
| 1255 | AGAAGACAGAAGTGAGGCATC |
| 1256 | GAAGACAGAAGTGAGGCATCA |
| 1257 | AAGACAGAAGTGAGGCATCAG |
| 1258 | AGACAGAAGTGAGGCATCAGG |
| 1259 | GACAGAAGTGAGGCATCAGGT |
| 1260 | ACAGAAGTGAGGCATCAGGTA |
| 1261 | CAGAAGTGAGGCATCAGGTAA |
| 1262 | AGAAGTGAGGCATCAGGTAAG |
| 1263 | GAAGTGAGGCATCAGGTAAGT |
| 1264 | AAGTGAGGCATCAGGTAAGTT |
| 1265 | AGTGAGGCATCAGGTAAGTTC |
| 1266 | GTGAGGCATCAGGTAAGTTCC |
| 1267 | TGAGGCATCAGGTAAGTTCCT |
| 1268 | GAGGCATCAGGTAAGTTCCTG |
| 1269 | AGGCATCAGGTAAGTTCCTGA |
| 1270 | GGCATCAGGTAAGTTCCTGAA |
| 1271 | GCATCAGGTAAGTTCCTGAAG |
| 1272 | CATCAGGTAAGTTCCTGAAGT |
| 1273 | ATCAGGTAAGTTCCTGAAGTG |
| 1274 | TCAGGTAAGTTCCTGAAGTGG |
| 1275 | CAGGTAAGTTCCTGAAGTGGA |
| 1276 | AGGTAAGTTCCTGAAGTGGAG |
| 1277 | GGTAAGTTCCTGAAGTGGAGG |
| 1278 | GTAAGTTCCTGAAGTGGAGGT |
| 1279 | TAAGTTCCTGAAGTGGAGGTG |
| 1280 | AAGTTCCTGAAGTGGAGGTGG |
| 1281 | AGTTCCTGAAGTGGAGGTGGT |
| 1282 | GTTCCTGAAGTGGAGGTGGTG |
| 1283 | TTCCTGAAGTGGAGGTGGTGG |
| 1284 | TCCTGAAGTGGAGGTGGTGGA |
| 1285 | CCTGAAGTGGAGGTGGTGGAA |
| 1286 | CTGAAGTGGAGGTGGTGGAAA |
| 1287 | ATGAGGTCTAATTTAGAAGACA |
| 1288 | TGAGGTCTAATTTAGAAGACAG |
| 1289 | GAGGTCTAATTTAGAAGACAGA |
| 1290 | AGGTCTAATTTAGAAGACAGAA |
| 1291 | GGTCTAATTTAGAAGACAGAAG |
| 1292 | GTCTAATTTAGAAGACAGAAGT |
| 1293 | TCTAATTTAGAAGACAGAAGTG |
| 1294 | CTAATTTAGAAGACAGAAGTGA |
| 1295 | TAATTTAGAAGACAGAAGTGAG |
| 1296 | AATTTAGAAGACAGAAGTGAGG |
| 1297 | ATTTAGAAGACAGAAGTGAGGC |
| 1298 | TTAGAAGACAGAAGTGAGGCAT |
| 1299 | TAGAAGACAGAAGTGAGGCATC |
| 1300 | AGAAGACAGAAGTGAGGCATCA |
| 1301 | GAAGACAGAAGTGAGGCATCAG |
| 1302 | AAGACAGAAGTGAGGCATCAGG |
| 1303 | AGACAGAAGTGAGGCATCAGGT |
| 1304 | GACAGAAGTGAGGCATCAGGTA |
| 1305 | ACAGAAGTGAGGCATCAGGTAA |
| 1306 | CAGAAGTGAGGCATCAGGTAAG |
| 1307 | AGAAGTGAGGCATCAGGTAAGT |
| 1308 | GAAGTGAGGCATCAGGTAAGTT |
| 1309 | AAGTGAGGCATCAGGTAAGTTC |
| 1310 | AGTGAGGCATCAGGTAAGTTCC |
| 1311 | GTGAGGCATCAGGTAAGTTCCT |
| 1312 | TGAGGCATCAGGTAAGTTCCTG |
| 1313 | GAGGCATCAGGTAAGTTCCTGA |
| 1314 | AGGCATCAGGTAAGTTCCTGAA |
| 1315 | GGCATCAGGTAAGTTCCTGAAG |
| 1316 | GCATCAGGTAAGTTCCTGAAGT |
| 1317 | CATCAGGTAAGTTCCTGAAGTG |
| 1318 | ATCAGGTAAGTTCCTGAAGTGG |
| 1319 | TCAGGTAAGTTCCTGAAGTGGA |
| 1320 | CAGGTAAGTTCCTGAAGTGGAG |
| 1321 | AGGTAAGTTCCTGAAGTGGAGG |
| 1322 | GGTAAGTTCCTGAAGTGGAGGT |
| 1323 | GTAAGTTCCTGAAGTGGAGGTG |
| 1324 | TAAGTTCCTGAAGTGGAGGTGG |
| 1325 | AAGTTCCTGAAGTGGAGGTGGT |
| 1326 | AGTTCCTGAAGTGGAGGTGGTG |
| 1327 | GTTCCTGAAGTGGAGGTGGTGG |
| 1328 | TTCCTGAAGTGGAGGTGGTGGA |
| 1329 | TCCTGAAGTGGAGGTGGTGGAA |
| 1330 | CCTGAAGTGGAGGTGGTGGAAA |
| 1331 | CTGAAGTGGAGGTGGTGGAAAC |
| 1332 | TGAAGTGGAGGTGGTGGAAACG |
| 1333 | ATGAGGTCTAATTTAGAAGACAG |
| 1334 | TGAGGTCTAATTTAGAAGACAGA |
| 1335 | GAGGTCTAATTTAGAAGACAGAA |
| 1336 | AGGTCTAATTTAGAAGACAGAAG |
| 1337 | GGTCTAATTTAGAAGACAGAAGT |
| 1338 | GTCTAATTTAGAAGACAGAAGTG |
| 1339 | TCTAATTTAGAAGACAGAAGTGA |
| 1340 | CTAATTTAGAAGACAGAAGTGAG |
| 1341 | TAATTTAGAAGACAGAAGTGAGG |
| 1342 | AATTTAGAAGACAGAAGTGAGGC |
| 1343 | ATTTAGAAGACAGAAGTGAGGCA |
| 1344 | TTTAGAAGACAGAAGTGAGGCAT |
| 1345 | TTAGAAGACAGAAGTGAGGCATC |
| 1346 | TAGAAGACAGAAGTGAGGCATCA |
| 1347 | AGAAGACAGAAGTGAGGCATCAG |
| 1348 | GAAGACAGAAGTGAGGCATCAGG |
| 1349 | AAGACAGAAGTGAGGCATCAGGT |
| 1350 | AGACAGAAGTGAGGCATCAGGTA |
| 1351 | GACAGAAGTGAGGCATCAGGTAA |
| 1352 | ACAGAAGTGAGGCATCAGGTAAG |
| 1353 | CAGAAGTGAGGCATCAGGTAAGT |
| 1354 | AGAAGTGAGGCATCAGGTAAGTT |
| 1355 | GAAGTGAGGCATCAGGTAAGTTC |
| 1356 | AAGTGAGGCATCAGGTAAGTTCC |
| 1357 | AGTGAGGCATCAGGTAAGTTCCT |
| 1358 | GTGAGGCATCAGGTAAGTTCCTG |
| 1359 | TGAGGCATCAGGTAAGTTCCTGA |
| 1360 | GAGGCATCAGGTAAGTTCCTGAA |
| 1361 | AGGCATCAGGTAAGTTCCTGAAG |
| 1362 | GGCATCAGGTAAGTTCCTGAAGT |
| 1363 | GCATCAGGTAAGTTCCTGAAGTG |
| 1364 | CATCAGGTAAGTTCCTGAAGTGG |
| 1365 | ATCAGGTAAGTTCCTGAAGTGGA |
| 1366 | TCAGGTAAGTTCCTGAAGTGGAG |
| 1367 | CAGGTAAGTTCCTGAAGTGGAGG |
| 1368 | AGGTAAGTTCCTGAAGTGGAGGT |
| 1369 | GGTAAGTTCCTGAAGTGGAGGTG |
| 1370 | GTAAGTTCCTGAAGTGGAGGTGG |
| 1371 | TAAGTTCCTGAAGTGGAGGTGGT |
| 1372 | AAGTTCCTGAAGTGGAGGTGGTG |
| 1373 | AGTTCCTGAAGTGGAGGTGGTGG |
| 1374 | GTTCCTGAAGTGGAGGTGGTGGA |
| 1375 | TTCCTGAAGTGGAGGTGGTGGAA |
| 1376 | TCCTGAAGTGGAGGTGGTGGAAA |
| 1377 | CCTGAAGTGGAGGTGGTGGAAAC |
| 1378 | CTGAAGTGGAGGTGGTGGAAACG |
| 1379 | TGAAGTGGAGGTGGTGGAAACGG |
| 1380 | ATGAGGTCTAATTTAGAAGACAGA |
| 1381 | TGAGGTCTAATTTAGAAGACAGAA |
| 1382 | GAGGTCTAATTTAGAAGACAGAAG |
| 1383 | AGGTCTAATTTAGAAGACAGAAGT |
| 1384 | GGTCTAATTTAGAAGACAGAAGTG |
| 1385 | GTCTAATTTAGAAGACAGAAGTGA |
| 1386 | TCTAATTTAGAAGACAGAAGTGAG |
| 1387 | CTAATTTAGAAGACAGAAGTGAGG |
| 1388 | TAATTTAGAAGACAGAAGTGAGGC |
| 1389 | AATTTAGAAGACAGAAGTGAGGCA |
| 1390 | ATTTAGAAGACAGAAGTGAGGCAT |
| 1391 | TTTAGAAGACAGAAGTGAGGCATC |
| 1392 | TTAGAAGACAGAAGTGAGGCATCA |
| 1393 | TAGAAGACAGAAGTGAGGCATCAG |
| 1394 | AGAAGACAGAAGTGAGGCATCAGG |
| 1395 | GAAGACAGAAGTGAGGCATCAGGT |
| 1396 | AAGACAGAAGTGAGGCATCAGGTA |
| 1397 | AGACAGAAGTGAGGCATCAGGTAA |
| 1398 | GACAGAAGTGAGGCATCAGGTAAG |
| 1399 | ACAGAAGTGAGGCATCAGGTAAGT |
| 1400 | CAGAAGTGAGGCATCAGGTAAGTT |
| 1401 | AGAAGTGAGGCATCAGGTAAGTTC |
| 1402 | GAAGTGAGGCATCAGGTAAGTTCC |
| 1403 | AAGTGAGGCATCAGGTAAGTTCCT |
| 1404 | AGTGAGGCATCAGGTAAGTTCCTG |
| 1405 | GTGAGGCATCAGGTAAGTTCCTGA |
| 1406 | TGAGGCATCAGGTAAGTTCCTGAA |
| 1407 | GAGGCATCAGGTAAGTTCCTGAAG |
| 1408 | AGGCATCAGGTAAGTTCCTGAAGT |
| 1409 | GGCATCAGGTAAGTTCCTGAAGTG |
| 1410 | GCATCAGGTAAGTTCCTGAAGTGG |
| 1411 | CATCAGGTAAGTTCCTGAAGTGGA |
| 1412 | ATCAGGTAAGTTCCTGAAGTGGAG |
| 1413 | TCAGGTAAGTTCCTGAAGTGGAGG |
| 1414 | CAGGTAAGTTCCTGAAGTGGAGGT |
| 1415 | AGGTAAGTTCCTGAAGTGGAGGTG |
| 1416 | GGTAAGTTCCTGAAGTGGAGGTGG |
| 1417 | GTAAGTTCCTGAAGTGGAGGTGGT |
| 1418 | TAAGTTCCTGAAGTGGAGGTGGTG |
| 1419 | AAGTTCCTGAAGTGGAGGTGGTGG |
| 1420 | AGTTCCTGAAGTGGAGGTGGTGGA |
| 1421 | GTTCCTGAAGTGGAGGTGGTGGAA |
| 1422 | TTCCTGAAGTGGAGGTGGTGGAAA |
| 1423 | TCCTGAAGTGGAGGTGGTGGAAAC |
| 1424 | CCTGAAGTGGAGGTGGTGGAAACG |
| 1425 | CTGAAGTGGAGGTGGTGGAAACGG |
| 1426 | TGAAGTGGAGGTGGTGGAAACGGA |
| 1427 | ATGAGGTCTAATTTAGAAGACAGAA |
| 1428 | TGAGGTCTAATTTAGAAGACAGAAG |
| 1429 | GAGGTCTAATTTAGAAGACAGAAGT |
| 1430 | AGGTCTAATTTAGAAGACAGAAGTG |
| 1431 | GGTCTAATTTAGAAGACAGAAGTGA |
| 1432 | GTCTAATTTAGAAGACAGAAGTGAG |
| 1433 | TCTAATTTAGAAGACAGAAGTGAGG |
| 1434 | CTAATTTAGAAGACAGAAGTGAGGC |
| 1435 | TAATTTAGAAGACAGAAGTGAGGCA |
| 1436 | AATTTAGAAGACAGAAGTGAGGCAT |
| 1437 | ATTTAGAAGACAGAAGTGAGGCATC |
| 1438 | TTTAGAAGACAGAAGTGAGGCATCA |
| 1439 | TTAGAAGACAGAAGTGAGGCATCAG |
| 1440 | TAGAAGACAGAAGTGAGGCATCAGG |
| 1441 | AGAAGACAGAAGTGAGGCATCAGGT |
| 1442 | GAAGACAGAAGTGAGGCATCAGGTA |
| 1443 | AAGACAGAAGTGAGGCATCAGGTAA |
| 1444 | AGACAGAAGTGAGGCATCAGGTAAG |
| 1445 | GACAGAAGTGAGGCATCAGGTAAGT |
| 1446 | ACAGAAGTGAGGCATCAGGTAAGTT |
| 1447 | CAGAAGTGAGGCATCAGGTAAGTTC |
| 1448 | AGAAGTGAGGCATCAGGTAAGTTCC |
| 1449 | GAAGTGAGGCATCAGGTAAGTTCCT |
| 1450 | AAGTGAGGCATCAGGTAAGTTCCTG |
| 1451 | AGTGAGGCATCAGGTAAGTTCCTGA |
| 1452 | GTGAGGCATCAGGTAAGTTCCTGAA |
| 1453 | TGAGGCATCAGGTAAGTTCCTGAAG |
| 1454 | GAGGCATCAGGTAAGTTCCTGAAGT |
| 1455 | AGGCATCAGGTAAGTTCCTGAAGTG |
| 1456 | GGCATCAGGTAAGTTCCTGAAGTGG |
| 1457 | GCATCAGGTAAGTTCCTGAAGTGGA |
| 1458 | CATCAGGTAAGTTCCTGAAGTGGAG |
| 1459 | ATCAGGTAAGTTCCTGAAGTGGAGG |
| 1460 | TCAGGTAAGTTCCTGAAGTGGAGGT |
| 1461 | CAGGTAAGTTCCTGAAGTGGAGGTG |
| 1462 | AGGTAAGTTCCTGAAGTGGAGGTGG |
| 1463 | GGTAAGTTCCTGAAGTGGAGGTGGT |
| 1464 | GTAAGTTCCTGAAGTGGAGGTGGTG |
| 1465 | TAAGTTCCTGAAGTGGAGGTGGTGG |
| 1466 | AAGTTCCTGAAGTGGAGGTGGTGGA |
| 1467 | AGTTCCTGAAGTGGAGGTGGTGGAA |
| 1468 | GTTCCTGAAGTGGAGGTGGTGGAAA |
| 1469 | TTCCTGAAGTGGAGGTGGTGGAAAC |
| 1470 | TCCTGAAGTGGAGGTGGTGGAAACG |
| 1471 | CCTGAAGTGGAGGTGGTGGAAACGG |
| 1472 | CTGAAGTGGAGGTGGTGGAAACGGA |
| 1473 | ATGAGGTCTAATTTAGAAGACAGAAG |
| 1474 | TGAGGTCTAATTTAGAAGACAGAAGT |
| 1475 | GAGGTCTAATTTAGAAGACAGAAGTG |
| 1476 | AGGTCTAATTTAGAAGACAGAAGTGA |
| 1477 | GGTCTAATTTAGAAGACAGAAGTGAG |
| 1478 | GTCTAATTTAGAAGACAGAAGTGAGG |
| 1479 | TCTAATTTAGAAGACAGAAGTGAGGC |
| 1480 | CTAATTTAGAAGACAGAAGTGAGGCA |
| 1481 | TAATTTAGAAGACAGAAGTGAGGCAT |
| 1482 | AATTTAGAAGACAGAAGTGAGGCATC |
| 1483 | ATTTAGAAGACAGAAGTGAGGCATCA |
| 1484 | TTTAGAAGACAGAAGTGAGGCATCAG |
| 1485 | TTAGAAGACAGAAGTGAGGCATCAGG |
| 1486 | TAGAAGACAGAAGTGAGGCATCAGGT |
| 1487 | AGAAGACAGAAGTGAGGCATCAGGTA |
| 1488 | GAAGACAGAAGTGAGGCATCAGGTAA |
| 1489 | AAGACAGAAGTGAGGCATCAGGTAAG |
| 1490 | AGACAGAAGTGAGGCATCAGGTAAGT |
| 1491 | GACAGAAGTGAGGCATCAGGTAAGTT |
| 1492 | ACAGAAGTGAGGCATCAGGTAAGTTC |
| 1493 | CAGAAGTGAGGCATCAGGTAAGTTCC |
| 1494 | AGAAGTGAGGCATCAGGTAAGTTCCT |
| 1495 | GAAGTGAGGCATCAGGTAAGTTCCTG |
| 1496 | AAGTGAGGCATCAGGTAAGTTCCTGA |
| 1497 | AGTGAGGCATCAGGTAAGTTCCTGAA |
| 1498 | GTGAGGCATCAGGTAAGTTCCTGAAG |
| 1499 | TGAGGCATCAGGTAAGTTCCTGAAGT |
| 1500 | GAGGCATCAGGTAAGTTCCTGAAGTG |
| 1501 | AGGCATCAGGTAAGTTCCTGAAGTGG |
| 1502 | GGCATCAGGTAAGTTCCTGAAGTGGA |
| 1503 | GCATCAGGTAAGTTCCTGAAGTGGAG |
| 1504 | CATCAGGTAAGTTCCTGAAGTGGAGG |
| 1505 | ATCAGGTAAGTTCCTGAAGTGGAGGT |
| 1506 | TCAGGTAAGTTCCTGAAGTGGAGGTG |
| 1507 | CAGGTAAGTTCCTGAAGTGGAGGTGG |
| 1508 | AGGTAAGTTCCTGAAGTGGAGGTGGT |
| 1509 | GGTAAGTTCCTGAAGTGGAGGTGGTG |
| 1510 | GTAAGTTCCTGAAGTGGAGGTGGTGG |
| 1511 | TAAGTTCCTGAAGTGGAGGTGGTGGA |
| 1512 | AAGTTCCTGAAGTGGAGGTGGTGGAA |
| 1513 | AGTTCCTGAAGTGGAGGTGGTGGAAA |
| 1514 | GTTCCTGAAGTGGAGGTGGTGGAAAC |
| 1515 | TTCCTGAAGTGGAGGTGGTGGAAACG |
| 1516 | TCCTGAAGTGGAGGTGGTGGAAACGG |
| 1517 | CCTGAAGTGGAGGTGGTGGAAACGGA |

TABLE 5
Exemplary ASOs that target regRNA RR63_v1 and
RR63_v2

| SEQ ID | |
|---|---|
| NO | Sequence |
| 1518 | TTCTTAGCATATATTAAATG |
| 1519 | CTTTGTTTCTTAGCATATAT |
| 1520 | ATTTTCTTTGTTTCTTAGCA |
| 1521 | TGCCTTTCTATTTTCTTTGT |
| 1522 | AATAATGCCTTTCTATTTTC |
| 1523 | AGCATCAACCGACAGTGAGC |
| 1524 | CAGTGTTGACACACAGCATC |
| 1525 | ACCCACAGTGTTGACACACA |
| 1526 | GGGTCACCCACAGTGTTGAC |
| 1527 | CAGACTGGGTCACCCACAGT |
| 1528 | ATTCCCAGACTGGGTCACCC |
| 1529 | TAGTCTATTCCCAGACTGGG |
| 1530 | GGCCTCATTTTAGTCTATTC |
| 1531 | GGAAGGGCCTCATTTTAGTC |
| 1532 | ATAGCCCTCAGGGCTGGAAG |
| 1533 | TATTTCCCTTCCCTATCAGC |
| 1534 | CGTGTATTTATTTCCCTTCC |
| 1535 | GGGATCACCGTGTATTTATT |
| 1536 | GCCAATGGGATCACCGTGTA |
| 1537 | AGGACTTACGTGGCCAATGG |
| 1538 | GAAGAAAGGACTTACGTGGC |
| 1539 | CCTCTGAAGAAAGGACTTAC |
| 1540 | TCTACCTCAACGGTGCCTCT |
| 1541 | ATTCTTTAGTCCTCTACCTC |
| 1542 | TCCTAAGCAATTCTTTAGTC |
| 1543 | AGTTAACTTTGGTGTTACAT |
| 1544 | TTGCAGAAATCAAATACTGG |
| 1545 | GTTTCATTGCAGAAATCAAA |
| 1546 | GCCCAAGTTTCATTGCAGAA |
| 1547 | ACAATTGCCATAAAATGCCC |
| 1548 | TTAAAAGATGCTACAATTGC |
| 1549 | GTGGTTTTAAAAGATGCTAC |
| 1550 | TGCCCATTTTGTGGTTTTAA |
| 1551 | GGAAAATGCCCATTTTGTGG |
| 1552 | CTATTTAGGGGGAAAATGCC |
| 1553 | TAAGAAGCTATTTAGGGGGA |
| 1554 | ACTCCTAAAAATGGATTAAG |
| 1555 | ATATTAGGGGATGACTCCTA |
| 1556 | GTGACAAATATTAGGGGATG |
| 1557 | AGAATAATGGTGACAAATAT |
| 1558 | GTACCAAGAATAATGGTGAC |
| 1559 | TCCTAGTACCAAGAATAATG |
| 1560 | ATTTTTGTTTCCTAGTACCA |
| 1561 | GCAAGCCCCTTGAATAATTT |
| 1562 | ACTCCACCCCACTCTAGATT |
| 1563 | TTCTGTTTACACTCCACCCC |
| 1564 | GGAGCTTCTGTTTACACTCC |
| 1565 | TCCCTGTCTAGGAGCTTCTG |
| 1566 | GTTGAAGCCTCCATTTCCCT |
| 1567 | CTCTGGTTGAAGCCTCCATT |
| 1568 | TCTGTAACACTGTGATTGTC |
| 1569 | CCCCCTGCTGTGCTCTGTAA |
| 1570 | CCCTCACTGCTCACCCCCCT |
| 1571 | ACCTCGTCCCCTCACTGCTC |
| 1572 | TTTCTGTGGGCAACCTCGTC |
| 1573 | GTTCTTTTCTGTGGGCAACC |
| 1574 | GTTTAATAGAAGTTCTTTTC |
| 1575 | GAGCTGTTGTTTAATAGAAG |
| 1576 | TTTATGAGCTGTTGTTTAAT |
| 1577 | GAAGTGATAACTTATTTATG |
| 1578 | ACATTTGAAGTGATAACTTA |
| 1579 | ATTAAACATTTGAAGTGATA |
| 1580 | GGTTTTTATTAAACATTTGA |
| 1581 | AGTAGTGGTTTTTATTAAAC |
| 1582 | GGAGTTAGTAGTGGTTTTTA |
| 1583 | TTAATATTAGGAGTTAGTAG |
| 1584 | TACCTAAAACTTTTTAATAT |
| 1585 | TGGTTATACCTAAAACTTTT |
| 1586 | CAAATTATAGTGGTTATACC |
| 1587 | GTTAGAGACAAATTATAGTG |
| 1588 | TAGAGATGTTAGAGACAAAT |
| 1589 | GAGGGGGAATAGCTTTTTAG |
| 1590 | CATTTTGAGGGGGAATAGCT |
| 1591 | GTCAACATTTTGAGGGGGAA |
| 1592 | CAGTATTTGTCAACATTTTG |
| 1593 | AAAGACAGTATTTGTCAACA |
| 1594 | CTGGGAATAAAAGACAGTAT |
| 1595 | TCAGACTGGGAATAAAAGAC |
| 1596 | TTATTCACACTTCAGACTGG |
| 1597 | AAATATTATTCACACTTCAG |
| 1598 | AGATCAAATATTATTCACAC |
| 1599 | TTGAGCACAGAGATCAAATA |
| 1600 | CTAATGGCTTGAGCACAGAG |
| 1601 | TCACATATTGCTAATGGCTT |
| 1602 | CAGTGGGAGTCACATATTGC |
| 1603 | CAAGCCAGTGGGAGTCACAT |
| 1604 | TAGGTCACCCAAGCCAGTGG |
| 1605 | TTTCAGTTTTTAGGTCACCC |
| 1606 | TCTAATTTCAGTTTTTAGGT |
| 1607 | TTTGTTTCTAGGATCTAATT |
| 1608 | GAAAGTTTGTTTCTAGGATC |
| 1609 | TAGAGGGGAGAAAGTTTGTT |
| 1610 | ATGTTTAGAGGGGAGAAAGT |
| 1611 | ATGAGCTGTGGATGCATGTT |
| 1612 | AAAAAATGAGCTGTGGATGC |
| 1613 | CCCGGTTTTCAAAAATATAG |
| 1614 | CCGTACTCATTTCTTCATTG |
| 1615 | CCGACTTTATCTTTTCTCCA |
| 1616 | GCTGGTTTAATGCAGAACTA |
| 1617 | ACCCTTGAAAGCTGGTTTAA |
| 1618 | CAAATTCTACCCTTGAAAGC |
| 1619 | CAATGATCACAAATTCTACC |
| 1620 | CACAGATTCAATGATCACAA |
| 1621 | GATAATCACAGATTCAATGA |
| 1622 | GCTCTGATAATCACAGATTC |
| 1623 | TTCTGTTTGCTCTGATAATC |
| 1624 | TGAAAGCACAATTCTGTTTG |
| 1625 | GCTTTGTGAAAGCACAATTC |
| 1626 | ACTTGGCTTTGTGAAAGCAC |
| 1627 | CTTCTTTTTTAACTTGGCTT |
| 1628 | CAGTGGGCTTCTTTTTTAAC |
| 1629 | AAAAATACCAGTGGGCTTCT |
| 1630 | TGAATAATCTGCTCAAAGTA |
| 1631 | CCGAATAGATTGAATAATCT |
| 1632 | TACAAATGGCCGAATAGATT |
| 1633 | GGGTGTGTGGTACAAATGGC |
| 1634 | TGTACATGGGTGTGTGGTAC |
| 1635 | TTAGAGAGAGAGAGAGTGGT |
| 1636 | TATCAATCTCTTAGAGAGAG |
| 1637 | CTATTTCATGTATCAATCTC |
| 1638 | GCAATGCTATTTCATGTATC |
| 1639 | CTTTATAGCAATGCTATTTC |
| 1640 | GTTGCTGTCTTCTTTATAGC |
| 1641 | CTCTTGGTTGCTGTCTTCTT |
| 1642 | GTATACAATACAGATGACTC |
| 1643 | ACATTGTATACAATACAGAT |
| 1644 | GATTTCACAACATTGTATAC |
| 1645 | GCATTAATGATTTCACAACA |
| 1646 | CAGGATGCATTAATGATTTC |
| 1647 | AGAAACTTACAGGATGCATT |
| 1648 | ACAATACTTGGATAGAAACT |
| 1649 | TCCAGTGTAAACAACGCTAC |
| 1650 | GTCTCTTGTCCAGTGTAAAC |
| 1651 | CATTGGTCTCTTGTCCAGTG |
| 1652 | GGTTTATTAAACATTGGTCT |
| 1653 | GTGTTTCTGGTTTATTAAAC |
| 1654 | TACTTTGTGGAGTGGAGTGT |
| 1655 | CCCTTTAACTCTTACTTTGT |
| 1656 | GTCTCTGCAAATCCATTCCC |
| 1657 | GTAACTTATACTTGTCTCTG |
| 1658 | AGGTAAGTAACTTATACTTG |
| 1659 | ATATTCACAGGTAAGTAACT |
| 1660 | CTCAAATTCATATTCACAGG |
| 1661 | CTCTGGCTTCCTCAAATTCA |
| 1662 | ATTTCCTCTGGCTTCCTCAA |
| 1663 | CAGTGGAGTCCATTATTTCC |
| 1664 | CTCCACAGTGGAGTCCATTA |
| 1665 | CTTTTAACTCTCCACAGTGG |
| 1666 | AGGTTGTGAAACCTTTTAAC |
| 1667 | GTTGGAAAAGGTTGTGAAAC |
| 1668 | ATGAAAACCGAGGTTGGAAA |
| 1669 | CTTTAGAATGATGAAAACCG |
| 1670 | AATTCTGTTCAGTGCTTTAG |
| 1671 | GAAAAAGTTTGAATTCTGTT |
| 1672 | GTTCTTGAAAAAGTTTGAAT |
| 1673 | GATAATGTTCTTGAAAAAGT |
| 1674 | CTTAGGCAAATAGATAATGT |
| 1675 | CATTGATCCTTAGGCAAATA |
| 1676 | GCCCTTAGGGACATTGATCC |
| 1677 | TTGGGCTGCCCTTAGGGACA |
| 1678 | TGTGCTTGGGCTGCCCTTAG |
| 1679 | TGAACTGTGCTTGGGCTGCC |
| 1680 | TGTGTTTGGGGTTATTGCTC |
| 1681 | CACTCTGTGTTTGGGGTTAT |
| 1682 | CCCAAGCACACTCTGTGTTT |
| 1683 | GAGGGTTTCCCAAGCACACT |
| 1684 | CACCCTCAGAGGGTTTCCCA |
| 1685 | GTTGACACCCTCAGAGGGTT |
| 1686 | AGGCCTGTTGACACCCTCAG |
| 1687 | AATTTCCAAGGCCTGTTGAC |
| 1688 | GTGAATCAGCAATTTCCAAG |
| 1689 | CGTTCCAAGGTGAATCAGCA |
| 1690 | ATTTGCCTCTCGTTCCAAGG |
| 1691 | ACTCTGCTCAATTTGCCTCT |
| 1692 | CTGGGCCCACTCTGCTCAAT |
| 1693 | TCTTGCTGGGCCCACTCTGC |
| 1694 | AGGGCTCTTGCTGGGCCCAC |
| 1695 | CTGAATTCCAGGGCTCTTGC |
| 1696 | GGATAGCCTGAATTCCAGGG |
| 1697 | AAATAAGGGTTGGGATAGCC |
| 1698 | AGGCCTGTAAAAAATAAGGG |
| 1699 | ACTACAGGCCTGTAAAAAAT |
| 1700 | TTCACAACTACAGGCCTGTA |
| 1701 | AGTAACTTTTCACAACTACA |
| 1702 | GTTTTTAAGTAACTTTTCAC |
| 1703 | CTTGTTATGTTTTTAAGTAA |
| 1704 | GAGGGGGAAAGTTTAAAGGT |
| 1705 | TTATATTGAGGGGGAAAGTT |
| 1706 | TCCAAGAGCAATCTTTTATA |
| 1707 | GGGATTTTCCAAGAGCAATC |
| 1708 | GCAACCAGGGATTTTCCAAG |
| 1709 | ATCCTGCAACCAGGGATTTT |
| 1710 | TCTGGATCCTGCAACCAGGG |
| 1711 | GGCCAGGAGCATCTGGATCC |
| 1712 | AGCTGGGCCAGGAGCATCTG |
| 1713 | TCTATAGCTGGGCCAGGAGC |
| 1714 | CAAAACTCTATAGCTGGGCC |
| 1715 | GCTGTTTCAAATGTTCTTTT |
| 1716 | TTTGAGCAAGGCTGTTTCAA |
| 1717 | CTCAGACTTTTGAGCAAGGC |
| 1718 | GTGCACTCAGACTTTTGAGC |
| 1719 | AGTTGTCTGTGCACTCAGAC |
| 1720 | GGTGCAAGAGTTGTCTGTGC |
| 1721 | GTCCATGCCAGGGTGCAAGA |
| 1722 | TCATCTGGTGTGTCCATGCC |
| 1723 | ATACAGAGCTTCATCTGGTG |
| 1724 | CATCTTTGCTTATACAGAGC |
| 1725 | AGAAAATCGTTCATCTTTGC |
| 1726 | ACCTTTAGAAAATCGTTCAT |
| 1727 | GACCTGTTCATTTGAACCTT |
| 1728 | ATTTGACTCAAGACCTGTTC |
| 1729 | GCTCCACATGCTTATTTGAC |
| 1730 | GAGGAGCTCCACATGCTTAT |
| 1731 | TATTGGAGGAGCTCCACATG |
| 1732 | CAAATATATTGGAGGAGCTC |
| 1733 | GAGGCAGCACAAATATATTG |
| 1734 | TGGAAAGATTCACAAGAAGG |
| 1735 | GGAAAAATGGAAAGATTCAC |
| 1736 | ACATTGTAGGAAAAATGGAA |
| 1737 | AAAAATAAGAACATTGTAGG |
| 1738 | GCTCAGGCAAAGGAGAAAAA |
| 1739 | TTTAGGGAAAAGTAAGCTCA |
| 1740 | GAGGCATTTAGGGAAAAGTA |
| 1741 | GCCTATTATTATTCATTGCT |
| 1742 | ATGTTGCCTATTATTATTCA |
| 1743 | GTCTTGACCAATGTTGCCTA |
| 1744 | CCAACCAAGTCTTGACCAAT |
| 1745 | CCAGGCACCCAACCAAGTCT |
| 1746 | GTGGGCCAGGCACCCAACCA |
| 1747 | TCAAATACTGGAGTTGTG |
| 1748 | CAAATACTGGAGTTGTGT |
| 1749 | GTTTTGCTTGGTCAAGAG |
| 1750 | GGTCAAGAGGAAAGAACT |
| 1751 | GCAGAAATCAAATACTGGA |
| 1752 | AATCAAATACTGGAGTTGT |
| 1753 | ATCAAATACTGGAGTTGTG |
| 1754 | TCAAATACTGGAGTTGTGT |
| 1755 | CAAATACTGGAGTTGTGTT |
| 1756 | AAATACTGGAGTTGTGTTT |
| 1757 | AGTTGTGTTTTGCTTGGTC |
| 1758 | TGTGTTTTGCTTGGTCAAG |
| 1759 | GTGTTTTGCTTGGTCAAGA |
| 1760 | TGTTTTGCTTGGTCAAGAG |
| 1761 | GTTTTGCTTGGTCAAGAGG |
| 1762 | TTTTGCTTGGTCAAGAGGA |
| 1763 | TGCTTGGTCAAGAGGAAAG |
| 1764 | TTGGTCAAGAGGAAAGAAC |
| 1765 | TGGTCAAGAGGAAAGAACT |
| 1766 | GGTCAAGAGGAAAGAACTG |
| 1767 | AGAGGAAAGAACTGGCCGG |
| 1768 | GAGGAAAGAACTGGCCGGG |
| 1769 | TGCAGAAATCAAATACTGGA |
| 1770 | GCAGAAATCAAATACTGGAG |
| 1771 | CAGAAATCAAATACTGGAGT |
| 1772 | AGAAATCAAATACTGGAGTT |
| 1773 | GAAATCAAATACTGGAGTTG |
| 1774 | AAATCAAATACTGGAGTTGT |
| 1775 | AATCAAATACTGGAGTTGTG |
| 1776 | ATCAAATACTGGAGTTGTGT |
| 1777 | TCAAATACTGGAGTTGTGTT |
| 1778 | CAAATACTGGAGTTGTGTTT |
| 1779 | AAATACTGGAGTTGTGTTTT |
| 1780 | TACTGGAGTTGTGTTTTGCT |
| 1781 | ACTGGAGTTGTGTTTTGCTT |
| 1782 | CTGGAGTTGTGTTTTGCTTG |
| 1783 | TGGAGTTGTGTTTTGCTTGG |
| 1784 | GGAGTTGTGTTTTGCTTGGT |
| 1785 | GAGTTGTGTTTTGCTTGGTC |
| 1786 | AGTTGTGTTTTGCTTGGTCA |
| 1787 | GTTGTGTTTTGCTTGGTCAA |
| 1788 | TTGTGTTTTGCTTGGTCAAG |
| 1789 | TGTGTTTTGCTTGGTCAAGA |
| 1790 | GTGTTTTGCTTGGTCAAGAG |
| 1791 | TGTTTTGCTTGGTCAAGAGG |
| 1792 | GTTTTGCTTGGTCAAGAGGA |
| 1793 | TTTTGCTTGGTCAAGAGGAA |
| 1794 | TTTGCTTGGTCAAGAGGAAA |
| 1795 | TTGCTTGGTCAAGAGGAAAG |
| 1796 | TGCTTGGTCAAGAGGAAAGA |
| 1797 | CTTGGTCAAGAGGAAAGAAC |
| 1798 | TTGGTCAAGAGGAAAGAACT |
| 1799 | TGGTCAAGAGGAAAGAACTG |
| 1800 | GGTCAAGAGGAAAGAACTGG |
| 1801 | GTCAAGAGGAAAGAACTGGC |
| 1802 | TCAAGAGGAAAGAACTGGCC |
| 1803 | CAAGAGGAAAGAACTGGCCG |
| 1804 | AAGAGGAAAGAACTGGCCGG |
| 1805 | AGAGGAAAGAACTGGCCGGG |
| 1806 | GAGGAAAGAACTGGCCGGGC |
| 1807 | TTGCAGAAATCAAATACTGGA |
| 1808 | TGCAGAAATCAAATACTGGAG |
| 1809 | GCAGAAATCAAATACTGGAGT |
| 1810 | CAGAAATCAAATACTGGAGTT |
| 1811 | AGAAATCAAATACTGGAGTTG |
| 1812 | GAAATCAAATACTGGAGTTGT |
| 1813 | AAATCAAATACTGGAGTTGTG |
| 1814 | AATCAAATACTGGAGTTGTGT |
| 1815 | ATCAAATACTGGAGTTGTGTT |
| 1816 | TCAAATACTGGAGTTGTGTTT |
| 1817 | CAAATACTGGAGTTGTGTTTT |
| 1818 | AAATACTGGAGTTGTGTTTTG |
| 1819 | AATACTGGAGTTGTGTTTTGC |
| 1820 | ATACTGGAGTTGTGTTTTGCT |
| 1821 | TACTGGAGTTGTGTTTTGCTT |
| 1822 | ACTGGAGTTGTGTTTTGCTTG |
| 1823 | CTGGAGTTGTGTTTTGCTTGG |
| 1824 | TGGAGTTGTGTTTTGCTTGGT |
| 1825 | GGAGTTGTGTTTTGCTTGGTC |
| 1826 | GAGTTGTGTTTTGCTTGGTCA |
| 1827 | AGTTGTGTTTTGCTTGGTCAA |
| 1828 | GTTGTGTTTTGCTTGGTCAAG |
| 1829 | TTGTGTTTTGCTTGGTCAAGA |
| 1830 | TGTGTTTTGCTTGGTCAAGAG |
| 1831 | GTGTTTTGCTTGGTCAAGAGG |
| 1832 | TGTTTTGCTTGGTCAAGAGGA |
| 1833 | GTTTTGCTTGGTCAAGAGGAA |
| 1834 | TTTTGCTTGGTCAAGAGGAAA |
| 1835 | TTTGCTTGGTCAAGAGGAAAG |
| 1836 | TTGCTTGGTCAAGAGGAAAGA |
| 1837 | TGCTTGGTCAAGAGGAAAGAA |
| 1838 | GCTTGGTCAAGAGGAAAGAAC |
| 1839 | CTTGGTCAAGAGGAAAGAACT |
| 1840 | TTGGTCAAGAGGAAAGAACTG |
| 1841 | TGGTCAAGAGGAAAGAACTGG |
| 1842 | GGTCAAGAGGAAAGAACTGGC |
| 1843 | GTCAAGAGGAAAGAACTGGCC |
| 1844 | TCAAGAGGAAAGAACTGGCCG |
| 1845 | CAAGAGGAAAGAACTGGCCGG |
| 1846 | AAGAGGAAAGAACTGGCCGGG |
| 1847 | AGAGGAAAGAACTGGCCGGGC |
| 1848 | GAGGAAAGAACTGGCCGGGCG |
| 1849 | TTGCAGAAATCAAATACTGGAG |
| 1850 | TGCAGAAATCAAATACTGGAGT |
| 1851 | GCAGAAATCAAATACTGGAGTT |
| 1852 | CAGAAATCAAATACTGGAGTTG |
| 1853 | AGAAATCAAATACTGGAGTTGT |
| 1854 | GAAATCAAATACTGGAGTTGTG |
| 1855 | AAATCAAATACTGGAGTTGTGT |
| 1856 | AATCAAATACTGGAGTTGTGTT |
| 1857 | ATCAAATACTGGAGTTGTGTTT |
| 1858 | TCAAATACTGGAGTTGTGTTTT |
| 1859 | CAAATACTGGAGTTGTGTTTTG |
| 1860 | AAATACTGGAGTTGTGTTTTGC |
| 1861 | AATACTGGAGTTGTGTTTTGCT |
| 1862 | ATACTGGAGTTGTGTTTTGCTT |
| 1863 | TACTGGAGTTGTGTTTTGCTTG |
| 1864 | ACTGGAGTTGTGTTTTGCTTGG |
| 1865 | CTGGAGTTGTGTTTTGCTTGGT |
| 1866 | TGGAGTTGTGTTTTGCTTGGTC |
| 1867 | GGAGTTGTGTTTTGCTTGGTCA |
| 1868 | GAGTTGTGTTTTGCTTGGTCAA |
| 1869 | AGTTGTGTTTTGCTTGGTCAAG |
| 1870 | GTTGTGTTTTGCTTGGTCAAGA |
| 1871 | TTGTGTTTTGCTTGGTCAAGAG |
| 1872 | TGTGTTTTGCTTGGTCAAGAGG |
| 1873 | GTGTTTTGCTTGGTCAAGAGGA |
| 1874 | TGTTTTGCTTGGTCAAGAGGAA |
| 1875 | GTTTTGCTTGGTCAAGAGGAAA |
| 1876 | TTTTGCTTGGTCAAGAGGAAAG |
| 1877 | TTTGCTTGGTCAAGAGGAAAGA |
| 1878 | TTGCTTGGTCAAGAGGAAAGAA |
| 1879 | TGCTTGGTCAAGAGGAAAGAAC |
| 1880 | GCTTGGTCAAGAGGAAAGAACT |
| 1881 | CTTGGTCAAGAGGAAAGAACTG |
| 1882 | TTGGTCAAGAGGAAAGAACTGG |
| 1883 | TGGTCAAGAGGAAAGAACTGGC |
| 1884 | GGTCAAGAGGAAAGAACTGGCC |
| 1885 | GTCAAGAGGAAAGAACTGGCCG |
| 1886 | TCAAGAGGAAAGAACTGGCCGG |
| 1887 | CAAGAGGAAAGAACTGGCCGGG |
| 1888 | AAGAGGAAAGAACTGGCCGGGC |
| 1889 | AGAGGAAAGAACTGGCCGGGCG |
| 1890 | GAGGAAAGAACTGGCCGGGCGC |
| 1891 | TTGCAGAAATCAAATACTGGAGT |
| 1892 | TGCAGAAATCAAATACTGGAGTT |
| 1893 | GCAGAAATCAAATACTGGAGTTG |
| 1894 | CAGAAATCAAATACTGGAGTTGT |
| 1895 | AGAAATCAAATACTGGAGTTGTG |
| 1896 | GAAATCAAATACTGGAGTTGTGT |
| 1897 | AAATCAAATACTGGAGTTGTGTT |
| 1898 | AATCAAATACTGGAGTTGTGTTT |
| 1899 | ATCAAATACTGGAGTTGTGTTTT |
| 1900 | TCAAATACTGGAGTTGTGTTTTG |
| 1901 | CAAATACTGGAGTTGTGTTTTGC |
| 1902 | AAATACTGGAGTTGTGTTTTGCT |
| 1903 | AATACTGGAGTTGTGTTTTGCTT |
| 1904 | ATACTGGAGTTGTGTTTTGCTTG |
| 1905 | TACTGGAGTTGTGTTTTGCTTGG |
| 1906 | ACTGGAGTTGTGTTTTGCTTGGT |
| 1907 | CTGGAGTTGTGTTTTGCTTGGTC |
| 1908 | TGGAGTTGTGTTTTGCTTGGTCA |
| 1909 | GGAGTTGTGTTTTGCTTGGTCAA |
| 1910 | GAGTTGTGTTTTGCTTGGTCAAG |
| 1911 | AGTTGTGTTTTGCTTGGTCAAGA |
| 1912 | GTTGTGTTTTGCTTGGTCAAGAG |
| 1913 | TTGTGTTTTGCTTGGTCAAGAGG |
| 1914 | TGTGTTTTGCTTGGTCAAGAGGA |
| 1915 | GTGTTTTGCTTGGTCAAGAGGAA |
| 1916 | TGTTTTGCTTGGTCAAGAGGAAA |
| 1917 | GTTTTGCTTGGTCAAGAGGAAAG |
| 1918 | TTTTGCTTGGTCAAGAGGAAAGA |
| 1919 | TTTGCTTGGTCAAGAGGAAAGAA |
| 1920 | TTGCTTGGTCAAGAGGAAAGAAC |
| 1921 | TGCTTGGTCAAGAGGAAAGAACT |
| 1922 | GCTTGGTCAAGAGGAAAGAACTG |
| 1923 | CTTGGTCAAGAGGAAAGAACTGG |
| 1924 | TTGGTCAAGAGGAAAGAACTGGC |
| 1925 | TGGTCAAGAGGAAAGAACTGGCC |
| 1926 | GGTCAAGAGGAAAGAACTGGCCG |
| 1927 | GTCAAGAGGAAAGAACTGGCCGG |
| 1928 | TCAAGAGGAAAGAACTGGCCGGG |
| 1929 | CAAGAGGAAAGAACTGGCCGGGC |
| 1930 | AAGAGGAAAGAACTGGCCGGGCG |
| 1931 | AGAGGAAAGAACTGGCCGGGCGC |
| 1932 | GAGGAAAGAACTGGCCGGGCGCG |
| 1933 | TTGCAGAAATCAAATACTGGAGTT |
| 1934 | TGCAGAAATCAAATACTGGAGTTG |
| 1935 | GCAGAAATCAAATACTGGAGTTGT |
| 1936 | CAGAAATCAAATACTGGAGTTGTG |
| 1937 | AGAAATCAAATACTGGAGTTGTGT |
| 1938 | GAAATCAAATACTGGAGTTGTGTT |
| 1939 | AAATCAAATACTGGAGTTGTGTTT |
| 1940 | AATCAAATACTGGAGTTGTGTTTT |
| 1941 | ATCAAATACTGGAGTTGTGTTTTG |
| 1942 | TCAAATACTGGAGTTGTGTTTTGC |
| 1943 | CAAATACTGGAGTTGTGTTTTGCT |
| 1944 | AAATACTGGAGTTGTGTTTTGCTT |
| 1945 | AATACTGGAGTTGTGTTTTGCTTG |
| 1946 | ATACTGGAGTTGTGTTTTGCTTGG |
| 1947 | TACTGGAGTTGTGTTTTGCTTGGT |
| 1948 | ACTGGAGTTGTGTTTTGCTTGGTC |
| 1949 | CTGGAGTTGTGTTTTGCTTGGTCA |
| 1950 | TGGAGTTGTGTTTTGCTTGGTCAA |
| 1951 | GGAGTTGTGTTTTGCTTGGTCAAG |
| 1952 | GAGTTGTGTTTTGCTTGGTCAAGA |
| 1953 | AGTTGTGTTTTGCTTGGTCAAGAG |
| 1954 | GTTGTGTTTTGCTTGGTCAAGAGG |
| 1955 | TTGTGTTTTGCTTGGTCAAGAGGA |
| 1956 | TGTGTTTTGCTTGGTCAAGAGGAA |
| 1957 | GTGTTTTGCTTGGTCAAGAGGAAA |
| 1958 | TGTTTTGCTTGGTCAAGAGGAAAG |
| 1959 | GTTTTGCTTGGTCAAGAGGAAAGA |
| 1960 | TTTTGCTTGGTCAAGAGGAAAGAA |
| 1961 | TTTGCTTGGTCAAGAGGAAAGAAC |
| 1962 | TTGCTTGGTCAAGAGGAAAGAACT |
| 1963 | TGCTTGGTCAAGAGGAAAGAACTG |
| 1964 | GCTTGGTCAAGAGGAAAGAACTGG |
| 1965 | CTTGGTCAAGAGGAAAGAACTGGC |
| 1966 | TTGGTCAAGAGGAAAGAACTGGCC |
| 1967 | TGGTCAAGAGGAAAGAACTGGCCG |
| 1968 | GGTCAAGAGGAAAGAACTGGCCGG |
| 1969 | GTCAAGAGGAAAGAACTGGCCGGG |
| 1970 | TCAAGAGGAAAGAACTGGCCGGGC |
| 1971 | CAAGAGGAAAGAACTGGCCGGGCG |
| 1972 | AAGAGGAAAGAACTGGCCGGGCGC |
| 1973 | AGAGGAAAGAACTGGCCGGGCGCG |
| 1974 | GAGGAAAGAACTGGCCGGGCGCGG |
| 1975 | TTGCAGAAATCAAATACTGGAGTTG |
| 1976 | TGCAGAAATCAAATACTGGAGTTGT |
| 1977 | GCAGAAATCAAATACTGGAGTTGTG |
| 1978 | CAGAAATCAAATACTGGAGTTGTGT |
| 1979 | AGAAATCAAATACTGGAGTTGTGTT |
| 1980 | GAAATCAAATACTGGAGTTGTGTTT |
| 1981 | AAATCAAATACTGGAGTTGTGTTTT |
| 1982 | AATCAAATACTGGAGTTGTGTTTTG |
| 1983 | ATCAAATACTGGAGTTGTGTTTTGC |
| 1984 | TCAAATACTGGAGTTGTGTTTTGCT |
| 1985 | CAAATACTGGAGTTGTGTTTTGCTT |
| 1986 | AAATACTGGAGTTGTGTTTTGCTTG |
| 1987 | AATACTGGAGTTGTGTTTTGCTTGG |
| 1988 | ATACTGGAGTTGTGTTTTGCTTGGT |
| 1989 | TACTGGAGTTGTGTTTTGCTTGGTC |
| 1990 | ACTGGAGTTGTGTTTTGCTTGGTCA |
| 1991 | CTGGAGTTGTGTTTTGCTTGGTCAA |
| 1992 | TGGAGTTGTGTTTTGCTTGGTCAAG |
| 1993 | GGAGTTGTGTTTTGCTTGGTCAAGA |
| 1994 | GAGTTGTGTTTTGCTTGGTCAAGAG |
| 1995 | AGTTGTGTTTTGCTTGGTCAAGAGG |
| 1996 | GTTGTGTTTTGCTTGGTCAAGAGGA |
| 1997 | TTGTGTTTTGCTTGGTCAAGAGGAA |
| 1998 | TGTGTTTTGCTTGGTCAAGAGGAAA |
| 1999 | GTGTTTTGCTTGGTCAAGAGGAAAG |
| 2000 | TGTTTTGCTTGGTCAAGAGGAAAGA |
| 2001 | GTTTTGCTTGGTCAAGAGGAAAGAA |
| 2002 | TTTTGCTTGGTCAAGAGGAAAGAAC |
| 2003 | TTTGCTTGGTCAAGAGGAAAGAACT |
| 2004 | TTGCTTGGTCAAGAGGAAAGAACTG |
| 2005 | TGCTTGGTCAAGAGGAAAGAACTGG |
| 2006 | GCTTGGTCAAGAGGAAAGAACTGGC |
| 2007 | CTTGGTCAAGAGGAAAGAACTGGCC |
| 2008 | TTGGTCAAGAGGAAAGAACTGGCCG |
| 2009 | TGGTCAAGAGGAAAGAACTGGCCGG |
| 2010 | GGTCAAGAGGAAAGAACTGGCCGGG |
| 2011 | GTCAAGAGGAAAGAACTGGCCGGGC |
| 2012 | TCAAGAGGAAAGAACTGGCCGGGCG |
| 2013 | CAAGAGGAAAGAACTGGCCGGGCGC |
| 2014 | AAGAGGAAAGAACTGGCCGGGCGCG |
| 2015 | AGAGGAAAGAACTGGCCGGGCGCGG |
| 2016 | GAGGAAAGAACTGGCCGGGCGCGGT |
| 2017 | TTGCAGAAATCAAATACTGGAGTTGT |
| 2018 | TGCAGAAATCAAATACTGGAGTTGTG |
| 2019 | GCAGAAATCAAATACTGGAGTTGTGT |
| 2020 | CAGAAATCAAATACTGGAGTTGTGTT |
| 2021 | AGAAATCAAATACTGGAGTTGTGTTT |
| 2022 | GAAATCAAATACTGGAGTTGTGTTTT |
| 2023 | AAATCAAATACTGGAGTTGTGTTTTG |
| 2024 | AATCAAATACTGGAGTTGTGTTTTGC |
| 2025 | ATCAAATACTGGAGTTGTGTTTTGCT |
| 2026 | TCAAATACTGGAGTTGTGTTTTGCTT |
| 2027 | CAAATACTGGAGTTGTGTTTTGCTTG |
| 2028 | AAATACTGGAGTTGTGTTTTGCTTGG |
| 2029 | AATACTGGAGTTGTGTTTTGCTTGGT |
| 2030 | ATACTGGAGTTGTGTTTTGCTTGGTC |
| 2031 | TACTGGAGTTGTGTTTTGCTTGGTCA |
| 2032 | ACTGGAGTTGTGTTTTGCTTGGTCAA |
| 2033 | CTGGAGTTGTGTTTTGCTTGGTCAAG |
| 2034 | TGGAGTTGTGTTTTGCTTGGTCAAGA |
| 2035 | GGAGTTGTGTTTTGCTTGGTCAAGAG |
| 2036 | GAGTTGTGTTTTGCTTGGTCAAGAGG |
| 2037 | AGTTGTGTTTTGCTTGGTCAAGAGGA |
| 2038 | GTTGTGTTTTGCTTGGTCAAGAGGAA |
| 2039 | TTGTGTTTTGCTTGGTCAAGAGGAAA |
| 2040 | TGTGTTTTGCTTGGTCAAGAGGAAAG |
| 2041 | GTGTTTTGCTTGGTCAAGAGGAAAGA |
| 2042 | TGTTTTGCTTGGTCAAGAGGAAAGAA |
| 2043 | GTTTTGCTTGGTCAAGAGGAAAGAAC |
| 2044 | TTTTGCTTGGTCAAGAGGAAAGAACT |
| 2045 | TTTGCTTGGTCAAGAGGAAAGAACTG |
| 2046 | TTGCTTGGTCAAGAGGAAAGAACTGG |
| 2047 | TGCTTGGTCAAGAGGAAAGAACTGGC |
| 2048 | GCTTGGTCAAGAGGAAAGAACTGGCC |
| 2049 | CTTGGTCAAGAGGAAAGAACTGGCCG |
| 2050 | TTGGTCAAGAGGAAAGAACTGGCCGG |
| 2051 | TGGTCAAGAGGAAAGAACTGGCCGGG |
| 2052 | GGTCAAGAGGAAAGAACTGGCCGGGC |
| 2053 | GTCAAGAGGAAAGAACTGGCCGGGCG |
| 2054 | TCAAGAGGAAAGAACTGGCCGGGCGC |
| 2055 | CAAGAGGAAAGAACTGGCCGGGCGCG |
| 2056 | AAGAGGAAAGAACTGGCCGGGCGCGG |
| 2057 | AGAGGAAAGAACTGGCCGGGCGCGGT |

TABLE 6
Exemplary ASOs that target regRNA RR63_v2

| SEQ ID | |
|---|---|
| NO | Sequence |
| 2058 | TTCTTAGCATATATTAAATG |
| 2059 | CTTTGTTTCTTAGCATATAT |
| 2060 | ATTTTCTTTGTTTCTTAGCA |
| 2061 | TGCCTTTCTATTTTCTTTGT |
| 2062 | AATAATGCCTTTCTATTTTC |
| 2063 | AGCATCAACCGACAGTGAGC |
| 2064 | CAGTGTTGACACACAGCATC |
| 2065 | ACCCACAGTGTTGACACACA |
| 2066 | GGGTCACCCACAGTGTTGAC |
| 2067 | CAGACTGGGTCACCCACAGT |
| 2068 | ATTCCCAGACTGGGTCACCC |
| 2069 | TAGTCTATTCCCAGACTGGG |
| 2070 | GGCCTCATTTTAGTCTATTC |
| 2071 | GGAAGGGCCTCATTTTAGTC |
| 2072 | ATAGCCCTCAGGGCTGGAAG |
| 2073 | TATTTCCCTTCCCTATCAGC |
| 2074 | CGTGTATTTATTTCCCTTCC |
| 2075 | GGGATCACCGTGTATTTATT |
| 2076 | GCCAATGGGATCACCGTGTA |
| 2077 | AGGACTTACGTGGCCAATGG |
| 2078 | GAAGAAAGGACTTACGTGGC |
| 2079 | CCTCTGAAGAAAGGACTTAC |
| 2080 | TCTACCTCAACGGTGCCTCT |
| 2081 | ATTCTTTAGTCCTCTACCTC |
| 2082 | TCCTAAGCAATTCTTTAGTC |
| 2083 | AGTTAACTTTGGTGTTACAT |
| 2084 | TTGCAGAAATCAAATACTGG |
| 2085 | GTTTCATTGCAGAAATCAAA |
| 2086 | GCCCAAGTTTCATTGCAGAA |
| 2087 | ACAATTGCCATAAAATGCCC |
| 2088 | TTAAAAGATGCTACAATTGC |
| 2089 | GTGGTTTTAAAAGATGCTAC |
| 2090 | TGCCCATTTTGTGGTTTTAA |
| 2091 | GGAAAATGCCCATTTTGTGG |
| 2092 | CTATTTAGGGGGAAAATGCC |
| 2093 | TAAGAAGCTATTTAGGGGGA |
| 2094 | ACTCCTAAAAATGGATTAAG |
| 2095 | ATATTAGGGGATGACTCCTA |
| 2096 | GTGACAAATATTAGGGGATG |
| 2097 | AGAATAATGGTGACAAATAT |
| 2098 | GTACCAAGAATAATGGTGAC |
| 2099 | TCCTAGTACCAAGAATAATG |
| 2100 | ATTTTTGTTTCCTAGTACCA |
| 2101 | GCAAGCCCCTTGAATAATTT |
| 2102 | ACTCCACCCCACTCTAGATT |
| 2103 | TTCTGTTTACACTCCACCCC |
| 2104 | GGAGCTTCTGTTTACACTCC |
| 2105 | TCCCTGTCTAGGAGCTTCTG |
| 2106 | GTTGAAGCCTCCATTTCCCT |
| 2107 | CTCTGGTTGAAGCCTCCATT |
| 2108 | TCTGTAACACTGTGATTGTC |
| 2109 | CCCCCTGCTGTGCTCTGTAA |
| 2110 | CCCTCACTGCTCACCCCCCT |
| 2111 | ACCTCGTCCCCTCACTGCTC |
| 2112 | TTTCTGTGGGCAACCTCGTC |
| 2113 | GTTCTTTTCTGTGGGCAACC |
| 2114 | GTTTAATAGAAGTTCTTTTC |
| 2115 | GAGCTGTTGTTTAATAGAAG |
| 2116 | TTTATGAGCTGTTGTTTAAT |
| 2117 | GAAGTGATAACTTATTTATG |
| 2118 | ACATTTGAAGTGATAACTTA |
| 2119 | ATTAAACATTTGAAGTGATA |
| 2120 | GGTTTTTATTAAACATTTGA |
| 2121 | AGTAGTGGTTTTTATTAAAC |
| 2122 | GGAGTTAGTAGTGGTTTTTA |
| 2123 | TTAATATTAGGAGTTAGTAG |
| 2124 | TACCTAAAACTTTTTAATAT |
| 2125 | TGGTTATACCTAAAACTTTT |
| 2126 | CAAATTATAGTGGTTATACC |
| 2127 | GTTAGAGACAAATTATAGTG |
| 2128 | TAGAGATGTTAGAGACAAAT |
| 2129 | GAGGGGGAATAGCTTTTTAG |
| 2130 | CATTTTGAGGGGGAATAGCT |
| 2131 | GTCAACATTTTGAGGGGGAA |
| 2132 | CAGTATTTGTCAACATTTTG |
| 2133 | AAAGACAGTATTTGTCAACA |
| 2134 | CTGGGAATAAAAGACAGTAT |
| 2135 | TCAGACTGGGAATAAAAGAC |
| 2136 | TTATTCACACTTCAGACTGG |
| 2137 | AAATATTATTCACACTTCAG |
| 2138 | AGATCAAATATTATTCACAC |
| 2139 | TTGAGCACAGAGATCAAATA |
| 2140 | CTAATGGCTTGAGCACAGAG |
| 2141 | TCACATATTGCTAATGGCTT |
| 2142 | CAGTGGGAGTCACATATTGC |
| 2143 | CAAGCCAGTGGGAGTCACAT |
| 2144 | TTAGGTCACCCAAGCCAGTG |
| 2145 | TTTCAGTTTTTAGGTCACCC |
| 2146 | TCTAATTTCAGTTTTTAGGT |
| 2147 | TTTGTTTCTAGGATCTAATT |
| 2148 | GAAAGTTTGTTTCTAGGATC |
| 2149 | TAGAGGGGAGAAAGTTTGTT |
| 2150 | ATGTTTAGAGGGGAGAAAGT |
| 2151 | ATGAGCTGTGGATGCATGTT |
| 2152 | AAAAAATGAGCTGTGGATGC |
| 2153 | CCCGGTTTTCAAAAATATAG |
| 2154 | CCGTACTCATTTCTTCATTG |
| 2155 | CCGACTTTATCTTTTCTCCA |
| 2156 | GCTGGTTTAATGCAGAACTA |
| 2157 | ACCCTTGAAAGCTGGTTTAA |
| 2158 | CAAATTCTACCCTTGAAAGC |
| 2159 | CAATGATCACAAATTCTACC |
| 2160 | CACAGATTCAATGATCACAA |
| 2161 | GATAATCACAGATTCAATGA |
| 2162 | GCTCTGATAATCACAGATTC |
| 2163 | TTCTGTTTGCTCTGATAATC |
| 2164 | TGAAAGCACAATTCTGTTTG |
| 2165 | GCTTTGTGAAAGCACAATTC |
| 2166 | ACTTGGCTTTGTGAAAGCAC |
| 2167 | CTTCTTTTTTAACTTGGCTT |
| 2168 | CAGTGGGCTTCTTTTTTAAC |
| 2169 | AAAAATACCAGTGGGCTTCT |
| 2170 | TGAATAATCTGCTCAAAGTA |
| 2171 | CCGAATAGATTGAATAATCT |
| 2172 | TACAAATGGCCGAATAGATT |
| 2173 | GGGTGTGTGGTACAAATGGC |
| 2174 | TGTACATGGGTGTGTGGTAC |
| 2175 | TTAGAGAGAGAGAGAGTGGT |
| 2176 | TATCAATCTCTTAGAGAGAG |
| 2177 | CTATTTCATGTATCAATCTC |
| 2178 | GCAATGCTATTTCATGTATC |
| 2179 | CTTTATAGCAATGCTATTTC |
| 2180 | GTTGCTGTCTTCTTTATAGC |
| 2181 | CTCTTGGTTGCTGTCTTCTT |
| 2182 | GTATACAATACAGATGACTC |
| 2183 | ACATTGTATACAATACAGAT |
| 2184 | GATTTCACAACATTGTATAC |
| 2185 | GCATTAATGATTTCACAACA |
| 2186 | CAGGATGCATTAATGATTTC |
| 2187 | AGAAACTTACAGGATGCATT |
| 2188 | ACAATACTTGGATAGAAACT |
| 2189 | TCCAGTGTAAACAACGCTAC |
| 2190 | GTCTCTTGTCCAGTGTAAAC |
| 2191 | CATTGGTCTCTTGTCCAGTG |
| 2192 | GGTTTATTAAACATTGGTCT |
| 2193 | GTGTTTCTGGTTTATTAAAC |
| 2194 | TACTTTGTGGAGTGGAGTGT |
| 2195 | CCCTTTAACTCTTACTTTGT |
| 2196 | GTCTCTGCAAATCCATTCCC |
| 2197 | GTAACTTATACTTGTCTCTG |
| 2198 | AGGTAAGTAACTTATACTTG |
| 2199 | ATATTCACAGGTAAGTAACT |
| 2200 | CTCAAATTCATATTCACAGG |
| 2201 | CTCTGGCTTCCTCAAATTCA |
| 2202 | ATTTCCTCTGGCTTCCTCAA |
| 2203 | CAGTGGAGTCCATTATTTCC |
| 2204 | CTCCACAGTGGAGTCCATTA |
| 2205 | CTTTTAACTCTCCACAGTGG |
| 2206 | AGGTTGTGAAACCTTTTAAC |
| 2207 | GTTGGAAAAGGTTGTGAAAC |
| 2208 | ATGAAAACCGAGGTTGGAAA |
| 2209 | CTTTAGAATGATGAAAACCG |
| 2210 | AATTCTGTTCAGTGCTTTAG |
| 2211 | GAAAAAGTTTGAATTCTGTT |
| 2212 | GTTCTTGAAAAAGTTTGAAT |
| 2213 | GATAATGTTCTTGAAAAAGT |
| 2214 | CTTAGGCAAATAGATAATGT |
| 2215 | CATTGATCCTTAGGCAAATA |
| 2216 | GCCCTTAGGGACATTGATCC |
| 2217 | TTGGGCTGCCCTTAGGGACA |
| 2218 | TGTGCTTGGGCTGCCCTTAG |
| 2219 | TGAACTGTGCTTGGGCTGCC |
| 2220 | TGTGTTTGGGGTTATTGCTC |
| 2221 | CACTCTGTGTTTGGGGTTAT |
| 2222 | CCCAAGCACACTCTGTGTTT |
| 2223 | GAGGGTTTCCCAAGCACACT |
| 2224 | CACCCTCAGAGGGTTTCCCA |
| 2225 | GTTGACACCCTCAGAGGGTT |
| 2226 | AGGCCTGTTGACACCCTCAG |
| 2227 | AATTTCCAAGGCCTGTTGAC |
| 2228 | GTGAATCAGCAATTTCCAAG |
| 2229 | CGTTCCAAGGTGAATCAGCA |
| 2230 | ATTTGCCTCTCGTTCCAAGG |
| 2231 | ACTCTGCTCAATTTGCCTCT |
| 2232 | CTGGGCCCACTCTGCTCAAT |
| 2233 | TCTTGCTGGGCCCACTCTGC |
| 2234 | AGGGCTCTTGCTGGGCCCAC |
| 2235 | CTGAATTCCAGGGCTCTTGC |
| 2236 | GGATAGCCTGAATTCCAGGG |
| 2237 | AAATAAGGGTTGGGATAGCC |
| 2238 | AGGCCTGTAAAAAATAAGGG |
| 2239 | ACTACAGGCCTGTAAAAAAT |
| 2240 | TTCACAACTACAGGCCTGTA |
| 2241 | AGTAACTTTTCACAACTACA |
| 2242 | GTTTTTAAGTAACTTTTCAC |
| 2243 | CTTGTTATGTTTTTAAGTAA |
| 2244 | GAGGGGGAAAGTTTAAAGGT |
| 2245 | TTATATTGAGGGGGAAAGTT |
| 2246 | TCCAAGAGCAATCTTTTATA |
| 2247 | GGGATTTTCCAAGAGCAATC |
| 2248 | GCAACCAGGGATTTTCCAAG |
| 2249 | ATCCTGCAACCAGGGATTTT |
| 2250 | TCTGGATCCTGCAACCAGGG |
| 2251 | GGCCAGGAGCATCTGGATCC |
| 2252 | AGCTGGGCCAGGAGCATCTG |
| 2253 | TCTATAGCTGGGCCAGGAGC |
| 2254 | CAAAACTCTATAGCTGGGCC |
| 2255 | GCTGTTTCAAATGTTCTTTT |
| 2256 | TTTGAGCAAGGCTGTTTCAA |
| 2257 | CTCAGACTTTTGAGCAAGGC |
| 2258 | GTGCACTCAGACTTTTGAGC |
| 2259 | AGTTGTCTGTGCACTCAGAC |
| 2260 | GGTGCAAGAGTTGTCTGTGC |
| 2261 | GTCCATGCCAGGGTGCAAGA |
| 2262 | TCATCTGGTGTGTCCATGCC |
| 2263 | ATACAGAGCTTCATCTGGTG |
| 2264 | CATCTTTGCTTATACAGAGC |
| 2265 | AGAAAATCGTTCATCTTTGC |
| 2266 | ACCTTTAGAAAATCGTTCAT |
| 2267 | GACCTGTTCATTTGAACCTT |
| 2268 | ATTTGACTCAAGACCTGTTC |
| 2269 | GCTCCACATGCTTATTTGAC |
| 2270 | GAGGAGCTCCACATGCTTAT |
| 2271 | TATTGGAGGAGCTCCACATG |
| 2272 | CAAATATATTGGAGGAGCTC |
| 2273 | GAGGCAGCACAAATATATTG |
| 2274 | TGGAAAGATTCACAAGAAGG |
| 2275 | GGAAAAATGGAAAGATTCAC |
| 2276 | ACATTGTAGGAAAAATGGAA |
| 2277 | AAAAATAAGAACATTGTAGG |
| 2278 | GCTCAGGCAAAGGAGAAAAA |
| 2279 | TTTAGGGAAAAGTAAGCTCA |
| 2280 | GAGGCATTTAGGGAAAAGTA |
| 2281 | GCCTATTATTATTCATTGCT |
| 2282 | ATGTTGCCTATTATTATTCA |
| 2283 | GTCTTGACCAATGTTGCCTA |
| 2284 | CCAACCAAGTCTTGACCAAT |
| 2285 | CCAGGCACCCAACCAAGTCT |
| 2286 | AAGTGGGCCAGGCACCCAAC |
| 2287 | TTTTTGAAGTGGGCCAGGCA |
| 2288 | TAACATTTTTGAAGTGGGCC |
| 2289 | GAGGTGATACAAATAACATT |
| 2290 | TAGGGCTGTTGAGAGGCCCA |
| 2291 | ACTCCTAGGGCTGTTGAGAG |
| 2292 | CACCCAACTCCTAGGGCTGT |
| 2293 | TAGAAATGACATGATAGCAC |
| 2294 | CCCAACTATAAAATAGAAAT |
| 2295 | ACATCTACTAGTTGTTACAC |
| 2296 | GCATGTGGCTTCACATCTAC |
| 2297 | TAGCACAGACTGGCATGTGG |
| 2298 | GAAGGTTAGCACAGACTGGC |
| 2299 | GAGTGGAAGAAGGTTAGCAC |
| 2300 | TCAGAGCACAGAGTGGAAGA |
| 2301 | CAATTGCATTTCAGAGCACA |
| 2302 | GCCCATCAATTGCATTTCAG |
| 2303 | AATTTCTGCAAATTAAGAAG |
| 2304 | ACAGTTTAATTTCTGCAAAT |
| 2305 | ATTTGATCTTACAGTTTAAT |
| 2306 | GAGAAAATTTGATCTTACAG |
| 2307 | TTATGACAGAGAAACCTAAA |
| 2308 | ATAAAAAATTTATGACAGAG |

TABLE 7
Exemplary ASOs that target regRNA
RR64_v1 and RR64 v2

| SEQ ID | |
|---|---|
| NO | Sequence |
| 2309 | CAATGTTGCCTGTGTGCACT |
| 2310 | GTCCTTTCAATGTTGCCTGT |
| 2311 | GCCATCTTGGTCCTTTCAAT |
| 2312 | TCAGCAGCCATCTTGGTCCT |
| 2313 | TTCTCTCAGCAGCCATCTTG |
| 2314 | GCCTCTTCTCTCAGCAGCCA |
| 2315 | TTTTTGCCTCTTCTCTCAGC |
| 2316 | TACAAGCCACTTTTTGCCTC |
| 2317 | AAGCAAATTTACAAGCCACT |
| 2318 | CCTACTTAAGCAAATTTACA |
| 2319 | CTTCACCTACTTAAGCAAAT |
| 2320 | GTCTTTCTTCACCTACTTAA |
| 2321 | TCTTTGTCTTTCTTCACCTA |
| 2322 | GCACTGAAAGCCCTTCTTTG |
| 2323 | CTACTGGCACTGAAAGCCCT |
| 2324 | TCATCCTACTGGCACTGAAA |
| 2325 | GCAACTCATCCTACTGGCAC |
| 2326 | ACTGCATGCAACTCATCCTA |
| 2327 | TATCTTAGACTGCATGCAAC |
| 2328 | GTTCTTATCTTAGACTGCAT |
| 2329 | CAGAAGTTCTTATCTTAGAC |
| 2330 | CGTGAACAGAAGTTCTTATC |
| 2331 | GGTGGCCGTGAACAGAAGTT |
| 2332 | CCCTGGGTGGCCGTGAACAG |
| 2333 | AACACTGGCCCTGGGTGGCC |
| 2334 | TTGTAAACACTGGCCCTGGG |
| 2335 | GCTCAGCTTTGTAAACACTG |
| 2336 | CTTCTTGTGGCTCAGCTTTG |
| 2337 | CCCATCCTTCTTGTGGCTCA |
| 2338 | CCTAACCCATCCTTCTTGTG |
| 2339 | TTCCTGCCTAACCCATCCTT |
| 2340 | CAAGACTTCCTGCCTAACCC |
| 2341 | GCATGTGCAAGACTTCCTGC |
| 2342 | TTGGTGTGCATGTGCAAGAC |
| 2343 | TGTGATTGGTGTGCATGTGC |
| 2344 | GCTTCTGTGATTGGTGTGCA |
| 2345 | TAACCAGCTTCTGTGATTGG |
| 2346 | GTGGTAATAACCAGCTTCTG |
| 2347 | TCAGGGTGGTAATAACCAGC |
| 2348 | GATTTTCAGGGTGGTAATAA |
| 2349 | TATATTGATTTTCAGGGTGG |
| 2350 | CAATTTATATTGATTTTCAG |
| 2351 | CTTTACACAATTTATATTGA |
| 2352 | GCTGGCTTTACACAATTTAT |
| 2353 | GACTCATGCTGGCTTTACAC |
| 2354 | GTTAACTGACTCATGCTGGC |
| 2355 | TATATTGTGTTAACTGACTC |
| 2356 | GCCAATTATATTGTGTTAAC |
| 2357 | ACATAGCCAATTATATTGTG |
| 2358 | CAAAAAAACATAGCCAATTA |
| 2359 | TGCCACAAAAAAACATAGCC |
| 2360 | TGCTAGTCTCTATGCCACAA |
| 2361 | TCTCTCTGCTAGTCTCTATG |
| 2362 | GACATTTTTCTCTCTGCTAG |
| 2363 | TCTAATTTAGACATTTTTCT |
| 2364 | GAAAGCTCTAATTTAGACAT |
| 2365 | GCTACCTAAGAAAGCTCTAA |
| 2366 | AATACATAAGCTACCTAAGA |
| 2367 | CACAGCTTGCTTTAAAAATA |
| 2368 | CTTCCACACAGCTTGCTTTA |
| 2369 | GTGTCCTTCCACACAGCTTG |
| 2370 | GAAATTGTGTCCTTCCACAC |
| 2371 | AAACTACAGAAATTGTGTCC |
| 2372 | TGCAGTCAACTAAACTACAG |
| 2373 | CCCTCTGCAGTCAACTAAAC |
| 2374 | TGCCTCCCTCTGCAGTCAAC |
| 2375 | TACCATTCTGCCTCCCTCTG |
| 2376 | CACAAGCCTGTGCTCTTTTC |
| 2377 | GTGACTTCACAAGCCTGTGC |
| 2378 | TTCTTTGTGACTTCACAAGC |
| 2379 | GCCAGTTCTTTGTGACTTCA |
| 2380 | TCTAAAGCCAGTTCTTTGTG |
| 2381 | CCAGGATCTAAAGCCAGTTC |
| 2382 | AATCATCCAACAAGTAAGTG |
| 2383 | TAACCAAATCATCCAACAAG |
| 2384 | ATCACTACCTTAACCAAATC |
| 2385 | GCAAGTATCACTACCTTAAC |
| 2386 | AATTAGCAAGTATCACTACC |
| 2387 | CATGAGGGGAAATAATTAGC |
| 2388 | ACTTCACATGAGGGGAAATA |
| 2389 | ACTGGACTTCACATGAGGGG |
| 2390 | GGCCTACTGGACTTCACATG |
| 2391 | TTCAAGGCCTACTGGACTTC |
| 2392 | CCCTTATTCAAGGCCTACTG |
| 2393 | GATTTCCCCTTATTCAAGGC |
| 2394 | ACACCGATTTCCCCTTATTC |
| 2395 | TTGCCCAACACCGATTTCCC |
| 2396 | GCACCTTTGCCCAACACCGA |
| 2397 | GCAGAGCACCTTTGCCCAAC |
| 2398 | CATGGAGCAGAGCACCTTTG |
| 2399 | TGGTAGAGCTTCTGTCAGCC |
| 2400 | GTTGAGGATGGTAGAGCTTC |
| 2401 | TTAAAAGCCACATGTTGAGG |
| 2402 | CCAAGGAGACCTTAAAAGCC |
| 2403 | CATGTCCAAGGAGACCTTAA |
| 2404 | ATGTCCATGTCCAAGGAGAC |
| 2405 | GATACATGTCCATGTCCAAG |
| 2406 | GTGTGATGGATACATGTCCA |
| 2407 | CTCTTCCCTGTGTGATGGAT |
| 2408 | CTCCATATTTTCTCTTCCCT |
| 2409 | ACAAAATGCTCCATATTTTC |
| 2410 | TCCATACAAAATGCTCCATA |
| 2411 | GAACTTTCCATACAAAATGC |
| 2412 | CCCACAGGAACTTTCCATAC |
| 2413 | GATTGGCCCACAGGAACTTT |
| 2414 | TTCCAGGATTGGCCCACAGG |
| 2415 | GATGCCACTTCCAGGATTGG |
| 2416 | GAGTAGAAGTAATGGATGCC |
| 2417 | AATCTGAGTAGAAGTAATGG |
| 2418 | CTCCAGTGGAATCTGAGTAG |
| 2419 | CAGTCCTCTCCAGTGGAATC |
| 2420 | GTGGCACAGTCCTCTCCAGT |
| 2421 | GGTTATGTGGCACAGTCCTC |
| 2422 | TTAGGTGTGGTTATGTGGCA |
| 2423 | TCTTGCAGTTAGGTGTGGTT |
| 2424 | TCCAATCTTTCTTGCAGTTA |
| 2425 | TGACCTTATCTTCCAATCTT |
| 2426 | ACAGCCTGACCTTATCTTCC |
| 2427 | CCAGACACACAGCCTGACCT |
| 2428 | AAACCTCCCAGACACACAGC |
| 2429 | TTTAACTAAACCTCCCAGAC |
| 2430 | GGTTAACTTTTAACTAAACC |
| 2431 | GAGATTGGTTAACTTTTAAC |
| 2432 | CATCTCTTGAGATTGGTTAA |
| 2433 | AAGACCATCTCTTGAGATTG |
| 2434 | GTGATTTCAAAGACCATCTC |
| 2435 | GTGAACACAAACCTTTGTGA |
| 2436 | CTAGTAAGTGAACACAAACC |
| 2437 | GATAGCTAGTAAGTGAACAC |
| 2438 | GCTAATGGAGATTAAGTAAC |
| 2439 | CTGAGGCTAATGGAGATTAA |
| 2440 | GGTGGTATTCTCACTCCCAT |
| 2441 | CTGTGCAGGTGGTATTCTCA |
| 2442 | ATCATCCTGTGCAGGTGGTA |
| 2443 | AGGCCTCATCATCCTGTGCA |
| 2444 | AAGTTCAGGCAGGCCTCATC |
| 2445 | GGCACAGGGAAGTTCAGGCA |
| 2446 | TCTATGGCACAGGGAAGTTC |
| 2447 | CCCAATCCAGAATCTATGGC |
| 2448 | GGAGGAACTCCCAATCCAGA |
| 2449 | TTGGGGGAGGAACTCCCAAT |
| 2450 | GCTACTTGGGGGAGGAACTC |
| 2451 | TAAATGGCTACTTGGGGGAG |
| 2452 | TCAATTAAATGGCTACTTGG |
| 2453 | ACAATTCAATTAAATGGCTA |
| 2454 | TGGTAGGTAAAAAAATATAC |
| 2455 | TACACTAATTTTGGTAGGTA |
| 2456 | TAGGAATACACTAATTTTGG |
| 2457 | GACAAAATTAGGAATACACT |
| 2458 | GTGTACATGACAAAATTAGG |
| 2459 | CTACATGTGTACATGACAAA |
| 2460 | AGTCTTTTCTACATGTGTAC |
| 2461 | CTTCCAGTCTTTTCTACATG |
| 2462 | GTACTTCTTCCAGTCTTTTC |
| 2463 | ATATTGTACTTCTTCCAGTC |
| 2464 | TTTTTATATTGTACTTCTTC |
| 2465 | AGCACTTTTTATATTGTACT |
| 2466 | CTACAGATAAGCACTTTTTA |
| 2467 | CATAACCTACAGATAAGCAC |
| 2468 | CACATTTGCATAACCTACAG |
| 2469 | CCCTCATTCACATTTGCATA |
| 2470 | TGAAAACCACCCTCATTCAC |
| 2471 | AAAGCATGAGGAAGAAAATG |
| 2472 | GATGCAGAAAAGCATGAGGA |
| 2473 | TTTAGAAGATGCAGAAAAGC |
| 2474 | GTATATAATTTAGAAGATGC |
| 2475 | TACCCTTATTGTATATAATT |
| 2476 | TAGTTTACCCTTATTGTATA |
| 2477 | TAAAAAATAGTTTACCCTTA |
| 2478 | CTTATATGTTTAAAAAATAG |
| 2479 | CATTTAATAACTTATATGTT |
| 2480 | CATTATTTTCATTTAATAAC |
| 2481 | TTCCTTTGTCATATTCATTA |
| 2482 | TTGATATTTCCTTTGTCATA |
| 2483 | GCATTATTATTGATATTTCC |
| 2484 | ATTATGCATTATTATTGATA |
| 2485 | TAATAAAATTTTATTATGCA |
| 2486 | GTATTTCTAAAAATCATTAA |
| 2487 | GTACTCTCTATGTATTTCTA |
| 2488 | ATAGTTGTACTCTCTATGTA |
| 2489 | GGAGAACACCCACATAGTTG |
| 2490 | TATTTGGAGAACACCCACAT |
| 2491 | GCTAATATTTGGAGAACACC |
| 2492 | ACTACAGCTAATATTTGGAG |
| 2493 | GACTGAACTACAGCTAATAT |
| 2494 | GCAATGACTGAACTACAGCT |
| 2495 | TCTGAGTAGAAGTAATGG |
| 2496 | ATCTGAGTAGAAGTAATGG |
| 2497 | TCTGAGTAGAAGTAATGGA |
| 2498 | GAGTAGAAGTAATGGATGC |
| 2499 | AGTAGAAGTAATGGATGCC |
| 2500 | GTAGAAGTAATGGATGCCA |
| 2501 | GATGCCACTTCCAGGATTG |
| 2502 | CACTTCCAGGATTGGCCCA |
| 2503 | ACTTCCAGGATTGGCCCAC |
| 2504 | TTCCAGGATTGGCCCACAG |
| 2505 | AGGATTGGCCCACAGGAAC |
| 2506 | GATTGGCCCACAGGAACTT |
| 2507 | GCCCACAGGAACTTTCCAT |
| 2508 | CCACAGGAACTTTCCATAC |
| 2509 | CACAGGAACTTTCCATACA |
| 2510 | ATCTGAGTAGAAGTAATGGA |
| 2511 | TCTGAGTAGAAGTAATGGAT |
| 2512 | CTGAGTAGAAGTAATGGATG |
| 2513 | TGAGTAGAAGTAATGGATGC |
| 2514 | AGTAGAAGTAATGGATGCCA |
| 2515 | GTAGAAGTAATGGATGCCAC |
| 2516 | TAGAAGTAATGGATGCCACT |
| 2517 | GGATGCCACTTCCAGGATTG |
| 2518 | TGCCACTTCCAGGATTGGCC |
| 2519 | CCACTTCCAGGATTGGCCCA |
| 2520 | CACTTCCAGGATTGGCCCAC |
| 2521 | ACTTCCAGGATTGGCCCACA |
| 2522 | CTTCCAGGATTGGCCCACAG |
| 2523 | CAGGATTGGCCCACAGGAAC |
| 2524 | AGGATTGGCCCACAGGAACT |
| 2525 | GGATTGGCCCACAGGAACTT |
| 2526 | TGGCCCACAGGAACTTTCCA |
| 2527 | GGCCCACAGGAACTTTCCAT |
| 2528 | GCCCACAGGAACTTTCCATA |
| 2529 | CCACAGGAACTTTCCATACA |
| 2530 | CACAGGAACTTTCCATACAA |
| 2531 | ACAGGAACTTTCCATACAAA |
| 2532 | CAGGAACTTTCCATACAAAA |
| 2533 | GGAACTTTCCATACAAAATG |
| 2534 | CTTTCCATACAAAATGCTCC |
| 2535 | GAATCTGAGTAGAAGTAATGG |
| 2536 | AATCTGAGTAGAAGTAATGGA |
| 2537 | ATCTGAGTAGAAGTAATGGAT |
| 2538 | TCTGAGTAGAAGTAATGGATG |
| 2539 | CTGAGTAGAAGTAATGGATGC |
| 2540 | TGAGTAGAAGTAATGGATGCC |
| 2541 | GAGTAGAAGTAATGGATGCCA |
| 2542 | AGTAGAAGTAATGGATGCCAC |
| 2543 | GTAGAAGTAATGGATGCCACT |
| 2544 | TAGAAGTAATGGATGCCACTT |
| 2545 | TAATGGATGCCACTTCCAGGA |
| 2546 | TGGATGCCACTTCCAGGATTG |
| 2547 | GGATGCCACTTCCAGGATTGG |
| 2548 | GATGCCACTTCCAGGATTGGC |
| 2549 | ATGCCACTTCCAGGATTGGCC |
| 2550 | TGCCACTTCCAGGATTGGCCC |
| 2551 | GCCACTTCCAGGATTGGCCCA |
| 2552 | CCACTTCCAGGATTGGCCCAC |
| 2553 | CACTTCCAGGATTGGCCCACA |
| 2554 | ACTTCCAGGATTGGCCCACAG |
| 2555 | CTTCCAGGATTGGCCCACAGG |
| 2556 | TTCCAGGATTGGCCCACAGGA |
| 2557 | CCAGGATTGGCCCACAGGAAC |
| 2558 | CAGGATTGGCCCACAGGAACT |
| 2559 | AGGATTGGCCCACAGGAACTT |
| 2560 | GGATTGGCCCACAGGAACTTT |
| 2561 | GATTGGCCCACAGGAACTTTC |
| 2562 | ATTGGCCCACAGGAACTTTCC |
| 2563 | TTGGCCCACAGGAACTTTCCA |
| 2564 | TGGCCCACAGGAACTTTCCAT |
| 2565 | GGCCCACAGGAACTTTCCATA |
| 2566 | GCCCACAGGAACTTTCCATAC |
| 2567 | CCCACAGGAACTTTCCATACA |
| 2568 | CCACAGGAACTTTCCATACAA |
| 2569 | CACAGGAACTTTCCATACAAA |
| 2570 | ACAGGAACTTTCCATACAAAA |
| 2571 | CAGGAACTTTCCATACAAAAT |
| 2572 | AGGAACTTTCCATACAAAATG |
| 2573 | GGAACTTTCCATACAAAATGC |
| 2574 | GAACTTTCCATACAAAATGCT |
| 2575 | ACTTTCCATACAAAATGCTCC |
| 2576 | GAATCTGAGTAGAAGTAATGGA |
| 2577 | AATCTGAGTAGAAGTAATGGAT |
| 2578 | ATCTGAGTAGAAGTAATGGATG |
| 2579 | TCTGAGTAGAAGTAATGGATGC |
| 2580 | CTGAGTAGAAGTAATGGATGCC |
| 2581 | TGAGTAGAAGTAATGGATGCCA |
| 2582 | GAGTAGAAGTAATGGATGCCAC |
| 2583 | AGTAGAAGTAATGGATGCCACT |
| 2584 | GTAGAAGTAATGGATGCCACTT |
| 2585 | TAGAAGTAATGGATGCCACTTC |
| 2586 | AAGTAATGGATGCCACTTCCAG |
| 2587 | AGTAATGGATGCCACTTCCAGG |
| 2588 | GTAATGGATGCCACTTCCAGGA |
| 2589 | TAATGGATGCCACTTCCAGGAT |
| 2590 | ATGGATGCCACTTCCAGGATTG |
| 2591 | TGGATGCCACTTCCAGGATTGG |
| 2592 | GGATGCCACTTCCAGGATTGGC |
| 2593 | GATGCCACTTCCAGGATTGGCC |
| 2594 | ATGCCACTTCCAGGATTGGCCC |
| 2595 | TGCCACTTCCAGGATTGGCCCA |
| 2596 | GCCACTTCCAGGATTGGCCCAC |
| 2597 | CCACTTCCAGGATTGGCCCACA |
| 2598 | CACTTCCAGGATTGGCCCACAG |
| 2599 | ACTTCCAGGATTGGCCCACAGG |
| 2600 | CTTCCAGGATTGGCCCACAGGA |
| 2601 | TTCCAGGATTGGCCCACAGGAA |
| 2602 | TCCAGGATTGGCCCACAGGAAC |
| 2603 | CCAGGATTGGCCCACAGGAACT |
| 2604 | CAGGATTGGCCCACAGGAACTT |
| 2605 | AGGATTGGCCCACAGGAACTTT |
| 2606 | GGATTGGCCCACAGGAACTTTC |
| 2607 | GATTGGCCCACAGGAACTTTCC |
| 2608 | ATTGGCCCACAGGAACTTTCCA |
| 2609 | TTGGCCCACAGGAACTTTCCAT |
| 2610 | TGGCCCACAGGAACTTTCCATA |
| 2611 | GGCCCACAGGAACTTTCCATAC |
| 2612 | GCCCACAGGAACTTTCCATACA |
| 2613 | CCCACAGGAACTTTCCATACAA |
| 2614 | CCACAGGAACTTTCCATACAAA |
| 2615 | CACAGGAACTTTCCATACAAAA |
| 2616 | ACAGGAACTTTCCATACAAAAT |
| 2617 | CAGGAACTTTCCATACAAAATG |
| 2618 | AGGAACTTTCCATACAAAATGC |
| 2619 | GGAACTTTCCATACAAAATGCT |
| 2620 | GAACTTTCCATACAAAATGCTC |
| 2621 | AACTTTCCATACAAAATGCTCC |
| 2622 | GAATCTGAGTAGAAGTAATGGAT |
| 2623 | AATCTGAGTAGAAGTAATGGATG |
| 2624 | ATCTGAGTAGAAGTAATGGATGC |
| 2625 | TCTGAGTAGAAGTAATGGATGCC |
| 2626 | CTGAGTAGAAGTAATGGATGCCA |
| 2627 | TGAGTAGAAGTAATGGATGCCAC |
| 2628 | GAGTAGAAGTAATGGATGCCACT |
| 2629 | AGTAGAAGTAATGGATGCCACTT |
| 2630 | GTAGAAGTAATGGATGCCACTTC |
| 2631 | TAGAAGTAATGGATGCCACTTCC |
| 2632 | GAAGTAATGGATGCCACTTCCAG |
| 2633 | AAGTAATGGATGCCACTTCCAGG |
| 2634 | AGTAATGGATGCCACTTCCAGGA |
| 2635 | GTAATGGATGCCACTTCCAGGAT |
| 2636 | TAATGGATGCCACTTCCAGGATT |
| 2637 | AATGGATGCCACTTCCAGGATTG |
| 2638 | ATGGATGCCACTTCCAGGATTGG |
| 2639 | TGGATGCCACTTCCAGGATTGGC |
| 2640 | GGATGCCACTTCCAGGATTGGCC |
| 2641 | GATGCCACTTCCAGGATTGGCCC |
| 2642 | ATGCCACTTCCAGGATTGGCCCA |
| 2643 | TGCCACTTCCAGGATTGGCCCAC |
| 2644 | GCCACTTCCAGGATTGGCCCACA |
| 2645 | CCACTTCCAGGATTGGCCCACAG |
| 2646 | CACTTCCAGGATTGGCCCACAGG |
| 2647 | ACTTCCAGGATTGGCCCACAGGA |
| 2648 | CTTCCAGGATTGGCCCACAGGAA |
| 2649 | TTCCAGGATTGGCCCACAGGAAC |
| 2650 | TCCAGGATTGGCCCACAGGAACT |
| 2651 | CCAGGATTGGCCCACAGGAACTT |
| 2652 | CAGGATTGGCCCACAGGAACTTT |
| 2653 | AGGATTGGCCCACAGGAACTTTC |
| 2654 | GGATTGGCCCACAGGAACTTTCC |
| 2655 | GATTGGCCCACAGGAACTTTCCA |
| 2656 | ATTGGCCCACAGGAACTTTCCAT |
| 2657 | TTGGCCCACAGGAACTTTCCATA |
| 2658 | TGGCCCACAGGAACTTTCCATAC |
| 2659 | GGCCCACAGGAACTTTCCATACA |
| 2660 | GCCCACAGGAACTTTCCATACAA |
| 2661 | CCCACAGGAACTTTCCATACAAA |
| 2662 | CCACAGGAACTTTCCATACAAAA |
| 2663 | CACAGGAACTTTCCATACAAAAT |
| 2664 | ACAGGAACTTTCCATACAAAATG |
| 2665 | CAGGAACTTTCCATACAAAATGC |
| 2666 | AGGAACTTTCCATACAAAATGCT |
| 2667 | GGAACTTTCCATACAAAATGCTC |
| 2668 | GAACTTTCCATACAAAATGCTCC |
| 2669 | GAATCTGAGTAGAAGTAATGGATG |
| 2670 | AATCTGAGTAGAAGTAATGGATGC |
| 2671 | ATCTGAGTAGAAGTAATGGATGCC |
| 2672 | TCTGAGTAGAAGTAATGGATGCCA |
| 2673 | CTGAGTAGAAGTAATGGATGCCAC |
| 2674 | TGAGTAGAAGTAATGGATGCCACT |
| 2675 | GAGTAGAAGTAATGGATGCCACTT |
| 2676 | AGTAGAAGTAATGGATGCCACTTC |
| 2677 | GTAGAAGTAATGGATGCCACTTCC |
| 2678 | TAGAAGTAATGGATGCCACTTCCA |
| 2679 | AGAAGTAATGGATGCCACTTCCAG |
| 2680 | GAAGTAATGGATGCCACTTCCAGG |
| 2681 | AAGTAATGGATGCCACTTCCAGGA |
| 2682 | AGTAATGGATGCCACTTCCAGGAT |
| 2683 | GTAATGGATGCCACTTCCAGGATT |
| 2684 | TAATGGATGCCACTTCCAGGATTG |
| 2685 | AATGGATGCCACTTCCAGGATTGG |
| 2686 | ATGGATGCCACTTCCAGGATTGGC |
| 2687 | TGGATGCCACTTCCAGGATTGGCC |
| 2688 | GGATGCCACTTCCAGGATTGGCCC |
| 2689 | GATGCCACTTCCAGGATTGGCCCA |
| 2690 | ATGCCACTTCCAGGATTGGCCCAC |
| 2691 | TGCCACTTCCAGGATTGGCCCACA |
| 2692 | GCCACTTCCAGGATTGGCCCACAG |
| 2693 | CCACTTCCAGGATTGGCCCACAGG |
| 2694 | CACTTCCAGGATTGGCCCACAGGA |
| 2695 | ACTTCCAGGATTGGCCCACAGGAA |
| 2696 | CTTCCAGGATTGGCCCACAGGAAC |
| 2697 | TTCCAGGATTGGCCCACAGGAACT |
| 2698 | TCCAGGATTGGCCCACAGGAACTT |
| 2699 | CCAGGATTGGCCCACAGGAACTTT |
| 2700 | CAGGATTGGCCCACAGGAACTTTC |
| 2701 | AGGATTGGCCCACAGGAACTTTCC |
| 2702 | GGATTGGCCCACAGGAACTTTCCA |
| 2703 | GATTGGCCCACAGGAACTTTCCAT |
| 2704 | ATTGGCCCACAGGAACTTTCCATA |
| 2705 | TTGGCCCACAGGAACTTTCCATAC |
| 2706 | TGGCCCACAGGAACTTTCCATACA |
| 2707 | GGCCCACAGGAACTTTCCATACAA |
| 2708 | GCCCACAGGAACTTTCCATACAAA |
| 2709 | CCCACAGGAACTTTCCATACAAAA |
| 2710 | CCACAGGAACTTTCCATACAAAAT |
| 2711 | CACAGGAACTTTCCATACAAAATG |
| 2712 | ACAGGAACTTTCCATACAAAATGC |
| 2713 | CAGGAACTTTCCATACAAAATGCT |
| 2714 | AGGAACTTTCCATACAAAATGCTC |
| 2715 | GGAACTTTCCATACAAAATGCTCC |
| 2716 | GAATCTGAGTAGAAGTAATGGATGC |
| 2717 | AATCTGAGTAGAAGTAATGGATGCC |
| 2718 | ATCTGAGTAGAAGTAATGGATGCCA |
| 2719 | TCTGAGTAGAAGTAATGGATGCCAC |
| 2720 | CTGAGTAGAAGTAATGGATGCCACT |
| 2721 | TGAGTAGAAGTAATGGATGCCACTT |
| 2722 | GAGTAGAAGTAATGGATGCCACTTC |
| 2723 | AGTAGAAGTAATGGATGCCACTTCC |
| 2724 | GTAGAAGTAATGGATGCCACTTCCA |
| 2725 | TAGAAGTAATGGATGCCACTTCCAG |
| 2726 | AGAAGTAATGGATGCCACTTCCAGG |
| 2727 | GAAGTAATGGATGCCACTTCCAGGA |
| 2728 | AAGTAATGGATGCCACTTCCAGGAT |
| 2729 | AGTAATGGATGCCACTTCCAGGATT |
| 2730 | GTAATGGATGCCACTTCCAGGATTG |
| 2731 | TAATGGATGCCACTTCCAGGATTGG |
| 2732 | AATGGATGCCACTTCCAGGATTGGC |
| 2733 | ATGGATGCCACTTCCAGGATTGGCC |
| 2734 | TGGATGCCACTTCCAGGATTGGCCC |
| 2735 | GGATGCCACTTCCAGGATTGGCCCA |
| 2736 | GATGCCACTTCCAGGATTGGCCCAC |
| 2737 | ATGCCACTTCCAGGATTGGCCCACA |
| 2738 | TGCCACTTCCAGGATTGGCCCACAG |
| 2739 | GCCACTTCCAGGATTGGCCCACAGG |
| 2740 | CCACTTCCAGGATTGGCCCACAGGA |
| 2741 | CACTTCCAGGATTGGCCCACAGGAA |
| 2742 | ACTTCCAGGATTGGCCCACAGGAAC |
| 2743 | CTTCCAGGATTGGCCCACAGGAACT |
| 2744 | TTCCAGGATTGGCCCACAGGAACTT |
| 2745 | TCCAGGATTGGCCCACAGGAACTTT |
| 2746 | CCAGGATTGGCCCACAGGAACTTTC |
| 2747 | CAGGATTGGCCCACAGGAACTTTCC |
| 2748 | AGGATTGGCCCACAGGAACTTTCCA |
| 2749 | GGATTGGCCCACAGGAACTTTCCAT |
| 2750 | GATTGGCCCACAGGAACTTTCCATA |
| 2751 | ATTGGCCCACAGGAACTTTCCATAC |
| 2752 | TTGGCCCACAGGAACTTTCCATACA |
| 2753 | TGGCCCACAGGAACTTTCCATACAA |
| 2754 | GGCCCACAGGAACTTTCCATACAAA |
| 2755 | GCCCACAGGAACTTTCCATACAAAA |
| 2756 | CCCACAGGAACTTTCCATACAAAAT |
| 2757 | CCACAGGAACTTTCCATACAAAATG |
| 2758 | CACAGGAACTTTCCATACAAAATGC |
| 2759 | ACAGGAACTTTCCATACAAAATGCT |
| 2760 | CAGGAACTTTCCATACAAAATGCTC |
| 2761 | AGGAACTTTCCATACAAAATGCTCC |
| 2762 | GAATCTGAGTAGAAGTAATGGATGCC |
| 2763 | AATCTGAGTAGAAGTAATGGATGCCA |
| 2764 | ATCTGAGTAGAAGTAATGGATGCCAC |
| 2765 | TCTGAGTAGAAGTAATGGATGCCACT |
| 2766 | CTGAGTAGAAGTAATGGATGCCACTT |
| 2767 | TGAGTAGAAGTAATGGATGCCACTTC |
| 2768 | GAGTAGAAGTAATGGATGCCACTTCC |
| 2769 | AGTAGAAGTAATGGATGCCACTTCCA |
| 2770 | GTAGAAGTAATGGATGCCACTTCCAG |
| 2771 | TAGAAGTAATGGATGCCACTTCCAGG |
| 2772 | AGAAGTAATGGATGCCACTTCCAGGA |
| 2773 | GAAGTAATGGATGCCACTTCCAGGAT |
| 2774 | AAGTAATGGATGCCACTTCCAGGATT |
| 2775 | AGTAATGGATGCCACTTCCAGGATTG |
| 2776 | GTAATGGATGCCACTTCCAGGATTGG |
| 2777 | TAATGGATGCCACTTCCAGGATTGGC |
| 2778 | AATGGATGCCACTTCCAGGATTGGCC |
| 2779 | ATGGATGCCACTTCCAGGATTGGCCC |
| 2780 | TGGATGCCACTTCCAGGATTGGCCCA |
| 2781 | GGATGCCACTTCCAGGATTGGCCCAC |
| 2782 | GATGCCACTTCCAGGATTGGCCCACA |
| 2783 | ATGCCACTTCCAGGATTGGCCCACAG |
| 2784 | TGCCACTTCCAGGATTGGCCCACAGG |
| 2785 | GCCACTTCCAGGATTGGCCCACAGGA |
| 2786 | CCACTTCCAGGATTGGCCCACAGGAA |
| 2787 | CACTTCCAGGATTGGCCCACAGGAAC |
| 2788 | ACTTCCAGGATTGGCCCACAGGAACT |
| 2789 | CTTCCAGGATTGGCCCACAGGAACTT |
| 2790 | TTCCAGGATTGGCCCACAGGAACTTT |
| 2791 | TCCAGGATTGGCCCACAGGAACTTTC |
| 2792 | CCAGGATTGGCCCACAGGAACTTTCC |
| 2793 | CAGGATTGGCCCACAGGAACTTTCCA |
| 2794 | AGGATTGGCCCACAGGAACTTTCCAT |
| 2795 | GGATTGGCCCACAGGAACTTTCCATA |
| 2796 | GATTGGCCCACAGGAACTTTCCATAC |
| 2797 | ATTGGCCCACAGGAACTTTCCATACA |
| 2798 | TTGGCCCACAGGAACTTTCCATACAA |
| 2799 | TGGCCCACAGGAACTTTCCATACAAA |
| 2800 | GGCCCACAGGAACTTTCCATACAAAA |
| 2801 | GCCCACAGGAACTTTCCATACAAAAT |
| 2802 | CCCACAGGAACTTTCCATACAAAATG |
| 2803 | CCACAGGAACTTTCCATACAAAATGC |
| 2804 | CACAGGAACTTTCCATACAAAATGCT |
| 2805 | ACAGGAACTTTCCATACAAAATGCTC |
| 2806 | CAGGAACTTTCCATACAAAATGCTCC |

TABLE 8

| Exemplary ASOs that target regRNA RR64_v2 | |
|---|---|
| SEQ | |
| ID NO | Sequence |
| 2807 | AAAAAGCAATGACTGAACTA |
| 2808 | TTCCCAGAAAAAAAGCAATG |
| 2809 | GAGTTTTCCCAGAAAAAAAG |
| 2810 | GCAGAAGTCAGAGTTTTCCC |
| 2811 | ATCCCTTCTGCAGAAGTCAG |
| 2812 | GCCCCATCCCTTCTGCAGAA |
| 2813 | ATGACGGCCCCATCCCTTCT |
| 2814 | ATGTCCAGATGACGGCCCCA |
| 2815 | ACTTTGCTTGTGATGTCCAG |
| 2816 | CAGAAACTTTGCTTGTGATG |
| 2817 | TTGAATCCACAGAAACTTTG |
| 2818 | TAATGGGCTTTGAATCCACA |
| 2819 | GTTCTTAATGGGCTTTGAAT |
| 2820 | AGCAAGTTCTTAATGGGCTT |
| 2821 | GTTAGGATAAAGCAAGTTCT |
| 2822 | GGTCTGTGTTAGGATAAAGC |
| 2823 | AGGGCTGGTCTGTGTTAGGA |
| 2824 | CATGACACAGGGCTGGTCTG |
| 2825 | AGGTACCATGACACAGGGCT |
| 2826 | GCAGGAGCATCAGGTACCAT |
| 2827 | ACTGAGCAGGAGCATCAGGT |
| 2828 | GGCACACCACTGAGCAGGAG |
| 2829 | GACATTCAGGGCACACCACT |
| 2830 | GGTTCAGGACATTCAGGGCA |
| 2831 | TACCTCCAGGTTCAGGACAT |
| 2832 | GTGGATCTACCTCCAGGTTC |
| 2833 | GATAGGGTGGATCTACCTCC |
| 2834 | AGAGCTGGGATAGGGTGGAT |
| 2835 | AGCCAGATGAGGCAGAGAGC |
| 2836 | TTGGGAGAGCCAGATGAGGC |
| 2837 | AGCCAGCTTTTGGGAGAGCC |
| 2838 | GTGATAGCCAGCTTTTGGGA |
| 2839 | GGGATCAGTGATAGCCAGCT |
| 2840 | CAAAGGGGATCAGTGATAGC |
| 2841 | GCACTGAGCCAAAGGGGATC |
| 2842 | AACTAGGCACTGAGCCAAAG |
| 2843 | TACCCAGACATGATGTGGAG |
| 2844 | TCCAGGAGCCCTACCCAGAC |
| 2845 | GGAATTCCAGGAGCCCTACC |
| 2846 | CTGAAGGAATTCCAGGAGCC |
| 2847 | GTGAGGCCTGAAGGAATTCC |
| 2848 | CCCACAAGTGAGGCCTGAAG |
| 2849 | TTTTACTCTAAATGGCCCAC |
| 2850 | GCCCACTATTTTACTCTAAA |
| 2851 | GTTGTATGTGCCCACTATTT |
| 2852 | GACCATAATGAAGTGTTGTA |
| 2853 | TTGAAGACCATAATGAAGTG |
| 2854 | CATGGTTTGAAGACCATAAT |
| 2855 | GGAAACATGGTTTGAAGACC |
| 2856 | ATCTTAAAGGAAACATGGTT |
| 2857 | CTTAAGATCATCTTAAAGGA |
| 2858 | TCGTCCTTAAGATCATCTTA |
| 2859 | TCTTACTTTCGTCCTTAAGA |
| 2860 | CACTATTTCTTACTTTCGTC |
| 2861 | CTCAACTCACTATTTCTTAC |
| 2862 | GGACTTACTCAACTCACTAT |
| 2863 | ATAATACATTGGACTTACTC |
| 2864 | AACATTTAATAATACATTGG |
| 2865 | CAGAGGGAGCATAAAACATT |
| 2866 | GACAACTTGCTAGGATTGTA |
| 2867 | ATAAATGACAACTTGCTAGG |
| 2868 | TAACTATAAATGACAACTTG |
| 2869 | TTTAGATAACTATAAATGAC |
| 2870 | GTATTTCATTTAGATAACTA |
| 2871 | TCAATGTATTTCATTTAGAT |
| 2872 | GGAGATCAATGTATTTCATT |
| 2873 | GCTGTGGAGATCAATGTATT |
| 2874 | TACTGAGTGCTGTGGAGATC |
| 2875 | AACCAAGTACTGAGTGCTGT |
| 2876 | ATTCTAACCAAGTACTGAGT |
| 2877 | TATAAATTCTAACCAAGTAC |
| 2878 | AGAACCAAAAATATAAATTC |
| 2879 | ATTAGAGGGAGAACCAAAAA |
| 2880 | TCCTCATTAGAGGGAGAACC |
| 2881 | GCTGGTTTTCCTCATTAGAG |
| 2882 | TATTTTTTGTGCTGGTTTTC |
| 2883 | GTCCATATTTTTTGTGCTGG |
| 2884 | AATGAAGTCCATATTTTTTG |
| 2885 | AGATTTGCTTATGAATGAAG |
| 2886 | AACATCAGAAAAGATTTGCT |
| 2887 | CCGCACTCAAAAACATCAGA |
| 2888 | GAACTACCGCACTCAAAAAC |
| 2889 | GGAAAGACAAGAACTACCGC |
| 2890 | TTCAGGGAAAGACAAGAACT |
| 2891 | GAGCCTTCAGGGAAAGACAA |
| 2892 | AATAATGAGCCTTCAGGGAA |
| 2893 | TGCACAATAATGAGCCTTCA |
| 2894 | TAACTTTATGCACAATAATG |
| 2895 | ATTTTTATAACTTTATGCAC |
| 2896 | GGTACCCTTATAATTTTTAT |
| 2897 | ATTATAAAGGTACCCTTATA |
| 2898 | TCATTTGGCAATTATAAAGG |
| 2899 | TTCTTTTCATTTGGCAATTA |
| 2900 | CCTATTTCTTTTCATTTGGC |
| 2901 | CTTTATTTATTTCACCTATT |
| 2902 | GATTCCTCTTCTTTATTTAT |
| 2903 | CTCTCTGATTCCTCTTCTTT |
| 2904 | GAATTCTCTCTGATTCCTCT |
| 2905 | GTAGCAGAATTCTCTCTGAT |
| 2906 | TTGTTCTGTAGCAGAATTCT |
| 2907 | TTGACTTTTGTTCTGTAGCA |
| 2908 | CTTATAATTGACTTTTGTTC |
| 2909 | GCACAATTACTTATAATTGA |
| 2910 | TGCAGGCACAATTACTTATA |
| 2911 | TCAACACTGCAGGCACAATT |
| 2912 | TGAGTATTCAACACTGCAGG |
| 2913 | AGTTCAAATGAGTATTCAAC |
| 2914 | TCTACAGTTCAAATGAGTAT |
| 2915 | GATAAATTCTACAGTTCAAA |
| 2916 | CGAGTGATAAATTCTACAGT |
| 2917 | GACTTGCCCCTTGCGAGTGA |
| 2918 | AGGCAGACTTGCCCCTTGCG |
| 2919 | CCCCCAGGCAGACTTGCCCC |
| 2920 | CCATACCCCCAGGCAGACTT |
| 2921 | AATCTACCCATACCCCCAGG |
| 2922 | CATGAAAATCCAATCTACCC |
| 2923 | CAAGACATGAAAATCCAATC |
| 2924 | TAAAGCCAAGACATGAAAAT |
| 2925 | CTGGATAAAGCCAAGACATG |
| 2926 | GGAGGTGTCCTGGATAAAGC |
| 2927 | TAAGAAGGGAGGTGTCCTGG |
| 2928 | ACATGACTTTAAGAAGGGAG |
| 2929 | CGACTGAAACATGACTTTAA |
| 2930 | ATAGTTTCCACGACTGAAAC |
| 2931 | AAGGAATAGTTTCCACGACT |
| 2932 | AGCCCTACCCAGACATGA |
| 2933 | GCCCTACCCAGACATGAT |
| 2934 | CCCTACCCAGACATGATG |
| 2935 | CCTACCCAGACATGATGT |
| 2936 | TACCCAGACATGATGTGG |
| 2937 | TGGAGGTGGAGCAAACTA |
| 2938 | GAGGTGGAGCAAACTAGG |
| 2939 | AGGTGGAGCAAACTAGGC |
| 2940 | GGTGGAGCAAACTAGGCA |
| 2941 | GTGGAGCAAACTAGGCAC |
| 2942 | TGGAGCAAACTAGGCACT |
| 2943 | GGAGCAAACTAGGCACTG |
| 2944 | AACTAGGCACTGAGCCAA |
| 2945 | GGCACTGAGCCAAAGGGG |
| 2946 | AGCCAAAGGGGATCAGTG |
| 2947 | GCCAAAGGGGATCAGTGA |
| 2948 | AGGGGATCAGTGATAGCC |
| 2949 | AGCCCTACCCAGACATGAT |
| 2950 | GCCCTACCCAGACATGATG |
| 2951 | CCCTACCCAGACATGATGT |
| 2952 | CCTACCCAGACATGATGTG |
| 2953 | CTACCCAGACATGATGTGG |
| 2954 | TACCCAGACATGATGTGGA |
| 2955 | ATGTGGAGGTGGAGCAAAC |
| 2956 | GTGGAGGTGGAGCAAACTA |
| 2957 | TGGAGGTGGAGCAAACTAG |
| 2958 | GGAGGTGGAGCAAACTAGG |
| 2959 | GAGGTGGAGCAAACTAGGC |
| 2960 | AGGTGGAGCAAACTAGGCA |
| 2961 | GGTGGAGCAAACTAGGCAC |
| 2962 | GTGGAGCAAACTAGGCACT |
| 2963 | TGGAGCAAACTAGGCACTG |
| 2964 | GGAGCAAACTAGGCACTGA |
| 2965 | GAGCAAACTAGGCACTGAG |
| 2966 | AGCAAACTAGGCACTGAGC |
| 2967 | GCAAACTAGGCACTGAGCC |
| 2968 | CAAACTAGGCACTGAGCCA |
| 2969 | AAACTAGGCACTGAGCCAA |
| 2970 | AACTAGGCACTGAGCCAAA |
| 2971 | AGGCACTGAGCCAAAGGGG |
| 2972 | GGCACTGAGCCAAAGGGGA |
| 2973 | GCACTGAGCCAAAGGGGAT |
| 2974 | CTGAGCCAAAGGGGATCAG |
| 2975 | TGAGCCAAAGGGGATCAGT |
| 2976 | GAGCCAAAGGGGATCAGTG |
| 2977 | AGCCAAAGGGGATCAGTGA |
| 2978 | GCCAAAGGGGATCAGTGAT |
| 2979 | CAAAGGGGATCAGTGATAG |
| 2980 | AAAGGGGATCAGTGATAGC |
| 2981 | AAGGGGATCAGTGATAGCC |
| 2982 | AGGGGATCAGTGATAGCCA |
| 2983 | AGCCCTACCCAGACATGATG |
| 2984 | GCCCTACCCAGACATGATGT |
| 2985 | CCCTACCCAGACATGATGTG |
| 2986 | CCTACCCAGACATGATGTGG |
| 2987 | CTACCCAGACATGATGTGGA |
| 2988 | CCCAGACATGATGTGGAGGT |
| 2989 | CAGACATGATGTGGAGGTGG |
| 2990 | ATGATGTGGAGGTGGAGCAA |
| 2991 | TGATGTGGAGGTGGAGCAAA |
| 2992 | GATGTGGAGGTGGAGCAAAC |
| 2993 | ATGTGGAGGTGGAGCAAACT |
| 2994 | TGTGGAGGTGGAGCAAACTA |
| 2995 | GTGGAGGTGGAGCAAACTAG |
| 2996 | TGGAGGTGGAGCAAACTAGG |
| 2997 | GGAGGTGGAGCAAACTAGGC |
| 2998 | GAGGTGGAGCAAACTAGGCA |
| 2999 | AGGTGGAGCAAACTAGGCAC |
| 3000 | GGTGGAGCAAACTAGGCACT |
| 3001 | GTGGAGCAAACTAGGCACTG |
| 3002 | TGGAGCAAACTAGGCACTGA |
| 3003 | GGAGCAAACTAGGCACTGAG |
| 3004 | GAGCAAACTAGGCACTGAGC |
| 3005 | AGCAAACTAGGCACTGAGCC |
| 3006 | GCAAACTAGGCACTGAGCCA |
| 3007 | CAAACTAGGCACTGAGCCAA |
| 3008 | AAACTAGGCACTGAGCCAAA |
| 3009 | TAGGCACTGAGCCAAAGGGG |
| 3010 | AGGCACTGAGCCAAAGGGGA |
| 3011 | GGCACTGAGCCAAAGGGGAT |
| 3012 | CACTGAGCCAAAGGGGATCA |
| 3013 | ACTGAGCCAAAGGGGATCAG |
| 3014 | CTGAGCCAAAGGGGATCAGT |
| 3015 | TGAGCCAAAGGGGATCAGTG |
| 3016 | GAGCCAAAGGGGATCAGTGA |
| 3017 | AGCCAAAGGGGATCAGTGAT |
| 3018 | GCCAAAGGGGATCAGTGATA |
| 3019 | CCAAAGGGGATCAGTGATAG |
| 3020 | AAAGGGGATCAGTGATAGCC |
| 3021 | AAGGGGATCAGTGATAGCCA |
| 3022 | AGCCCTACCCAGACATGATGT |
| 3023 | GCCCTACCCAGACATGATGTG |
| 3024 | CCCTACCCAGACATGATGTGG |
| 3025 | CCTACCCAGACATGATGTGGA |
| 3026 | CTACCCAGACATGATGTGGAG |
| 3027 | TACCCAGACATGATGTGGAGG |
| 3028 | ACCCAGACATGATGTGGAGGT |
| 3029 | CCCAGACATGATGTGGAGGTG |
| 3030 | CCAGACATGATGTGGAGGTGG |
| 3031 | CAGACATGATGTGGAGGTGGA |
| 3032 | CATGATGTGGAGGTGGAGCAA |
| 3033 | ATGATGTGGAGGTGGAGCAAA |
| 3034 | TGATGTGGAGGTGGAGCAAAC |
| 3035 | GATGTGGAGGTGGAGCAAACT |
| 3036 | ATGTGGAGGTGGAGCAAACTA |
| 3037 | TGTGGAGGTGGAGCAAACTAG |
| 3038 | GTGGAGGTGGAGCAAACTAGG |
| 3039 | TGGAGGTGGAGCAAACTAGGC |
| 3040 | GGAGGTGGAGCAAACTAGGCA |
| 3041 | GAGGTGGAGCAAACTAGGCAC |
| 3042 | AGGTGGAGCAAACTAGGCACT |
| 3043 | GGTGGAGCAAACTAGGCACTG |
| 3044 | GTGGAGCAAACTAGGCACTGA |
| 3045 | TGGAGCAAACTAGGCACTGAG |
| 3046 | GGAGCAAACTAGGCACTGAGC |
| 3047 | GAGCAAACTAGGCACTGAGCC |
| 3048 | AGCAAACTAGGCACTGAGCCA |
| 3049 | GCAAACTAGGCACTGAGCCAA |
| 3050 | CAAACTAGGCACTGAGCCAAA |
| 3051 | AAACTAGGCACTGAGCCAAAG |
| 3052 | AACTAGGCACTGAGCCAAAGG |
| 3053 | ACTAGGCACTGAGCCAAAGGG |
| 3054 | CTAGGCACTGAGCCAAAGGGG |
| 3055 | TAGGCACTGAGCCAAAGGGGA |
| 3056 | AGGCACTGAGCCAAAGGGGAT |
| 3057 | GGCACTGAGCCAAAGGGGATC |
| 3058 | GCACTGAGCCAAAGGGGATCA |
| 3059 | CACTGAGCCAAAGGGGATCAG |
| 3060 | ACTGAGCCAAAGGGGATCAGT |
| 3061 | CTGAGCCAAAGGGGATCAGTG |
| 3062 | TGAGCCAAAGGGGATCAGTGA |
| 3063 | GAGCCAAAGGGGATCAGTGAT |
| 3064 | AGCCAAAGGGGATCAGTGATA |
| 3065 | GCCAAAGGGGATCAGTGATAG |
| 3066 | CCAAAGGGGATCAGTGATAGC |
| 3067 | CAAAGGGGATCAGTGATAGCC |
| 3068 | AAAGGGGATCAGTGATAGCCA |
| 3069 | AGCCCTACCCAGACATGATGTG |
| 3070 | GCCCTACCCAGACATGATGTGG |
| 3071 | CCCTACCCAGACATGATGTGGA |
| 3072 | CCTACCCAGACATGATGTGGAG |
| 3073 | CTACCCAGACATGATGTGGAGG |
| 3074 | TACCCAGACATGATGTGGAGGT |
| 3075 | ACCCAGACATGATGTGGAGGTG |
| 3076 | CCCAGACATGATGTGGAGGTGG |
| 3077 | CCAGACATGATGTGGAGGTGGA |
| 3078 | CAGACATGATGTGGAGGTGGAG |
| 3079 | AGACATGATGTGGAGGTGGAGC |
| 3080 | ACATGATGTGGAGGTGGAGCAA |
| 3081 | CATGATGTGGAGGTGGAGCAAA |
| 3082 | ATGATGTGGAGGTGGAGCAAAC |
| 3083 | TGATGTGGAGGTGGAGCAAACT |
| 3084 | GATGTGGAGGTGGAGCAAACTA |
| 3085 | ATGTGGAGGTGGAGCAAACTAG |
| 3086 | TGTGGAGGTGGAGCAAACTAGG |
| 3087 | GTGGAGGTGGAGCAAACTAGGC |
| 3088 | TGGAGGTGGAGCAAACTAGGCA |
| 3089 | GGAGGTGGAGCAAACTAGGCAC |
| 3090 | GAGGTGGAGCAAACTAGGCACT |
| 3091 | AGGTGGAGCAAACTAGGCACTG |
| 3092 | GGTGGAGCAAACTAGGCACTGA |
| 3093 | GTGGAGCAAACTAGGCACTGAG |
| 3094 | TGGAGCAAACTAGGCACTGAGC |
| 3095 | GGAGCAAACTAGGCACTGAGCC |
| 3096 | GAGCAAACTAGGCACTGAGCCA |
| 3097 | AGCAAACTAGGCACTGAGCCAA |
| 3098 | GCAAACTAGGCACTGAGCCAAA |
| 3099 | CAAACTAGGCACTGAGCCAAAG |
| 3100 | AAACTAGGCACTGAGCCAAAGG |
| 3101 | AACTAGGCACTGAGCCAAAGGG |
| 3102 | ACTAGGCACTGAGCCAAAGGGG |
| 3103 | CTAGGCACTGAGCCAAAGGGGA |
| 3104 | TAGGCACTGAGCCAAAGGGGAT |
| 3105 | AGGCACTGAGCCAAAGGGGATC |
| 3106 | GGCACTGAGCCAAAGGGGATCA |
| 3107 | GCACTGAGCCAAAGGGGATCAG |
| 3108 | CACTGAGCCAAAGGGGATCAGT |
| 3109 | ACTGAGCCAAAGGGGATCAGTG |
| 3110 | CTGAGCCAAAGGGGATCAGTGA |
| 3111 | TGAGCCAAAGGGGATCAGTGAT |
| 3112 | GAGCCAAAGGGGATCAGTGATA |
| 3113 | AGCCAAAGGGGATCAGTGATAG |
| 3114 | GCCAAAGGGGATCAGTGATAGC |
| 3115 | CCAAAGGGGATCAGTGATAGCC |
| 3116 | CAAAGGGGATCAGTGATAGCCA |
| 3117 | AGCCCTACCCAGACATGATGTGG |
| 3118 | GCCCTACCCAGACATGATGTGGA |
| 3119 | CCCTACCCAGACATGATGTGGAG |
| 3120 | CCTACCCAGACATGATGTGGAGG |
| 3121 | CTACCCAGACATGATGTGGAGGT |
| 3122 | TACCCAGACATGATGTGGAGGTG |
| 3123 | ACCCAGACATGATGTGGAGGTGG |
| 3124 | CCCAGACATGATGTGGAGGTGGA |
| 3125 | CCAGACATGATGTGGAGGTGGAG |
| 3126 | CAGACATGATGTGGAGGTGGAGC |
| 3127 | AGACATGATGTGGAGGTGGAGCA |
| 3128 | GACATGATGTGGAGGTGGAGCAA |
| 3129 | ACATGATGTGGAGGTGGAGCAAA |
| 3130 | CATGATGTGGAGGTGGAGCAAAC |
| 3131 | ATGATGTGGAGGTGGAGCAAACT |
| 3132 | TGATGTGGAGGTGGAGCAAACTA |
| 3133 | GATGTGGAGGTGGAGCAAACTAG |
| 3134 | ATGTGGAGGTGGAGCAAACTAGG |
| 3135 | TGTGGAGGTGGAGCAAACTAGGC |
| 3136 | GTGGAGGTGGAGCAAACTAGGCA |
| 3137 | TGGAGGTGGAGCAAACTAGGCAC |
| 3138 | GGAGGTGGAGCAAACTAGGCACT |
| 3139 | GAGGTGGAGCAAACTAGGCACTG |
| 3140 | AGGTGGAGCAAACTAGGCACTGA |
| 3141 | GGTGGAGCAAACTAGGCACTGAG |
| 3142 | GTGGAGCAAACTAGGCACTGAGC |
| 3143 | TGGAGCAAACTAGGCACTGAGCC |
| 3144 | GGAGCAAACTAGGCACTGAGCCA |
| 3145 | GAGCAAACTAGGCACTGAGCCAA |
| 3146 | AGCAAACTAGGCACTGAGCCAAA |
| 3147 | GCAAACTAGGCACTGAGCCAAAG |
| 3148 | CAAACTAGGCACTGAGCCAAAGG |
| 3149 | AAACTAGGCACTGAGCCAAAGGG |
| 3150 | AACTAGGCACTGAGCCAAAGGGG |
| 3151 | ACTAGGCACTGAGCCAAAGGGGA |
| 3152 | CTAGGCACTGAGCCAAAGGGGAT |
| 3153 | TAGGCACTGAGCCAAAGGGGATC |
| 3154 | AGGCACTGAGCCAAAGGGGATCA |
| 3155 | GGCACTGAGCCAAAGGGGATCAG |
| 3156 | GCACTGAGCCAAAGGGGATCAGT |
| 3157 | CACTGAGCCAAAGGGGATCAGTG |
| 3158 | ACTGAGCCAAAGGGGATCAGTGA |
| 3159 | CTGAGCCAAAGGGGATCAGTGAT |
| 3160 | TGAGCCAAAGGGGATCAGTGATA |
| 3161 | GAGCCAAAGGGGATCAGTGATAG |
| 3162 | AGCCAAAGGGGATCAGTGATAGC |
| 3163 | GCCAAAGGGGATCAGTGATAGCC |
| 3164 | CCAAAGGGGATCAGTGATAGCCA |
| 3165 | AGCCCTACCCAGACATGATGTGGA |
| 3166 | GCCCTACCCAGACATGATGTGGAG |
| 3167 | CCCTACCCAGACATGATGTGGAGG |
| 3168 | CCTACCCAGACATGATGTGGAGGT |
| 3169 | CTACCCAGACATGATGTGGAGGTG |
| 3170 | TACCCAGACATGATGTGGAGGTGG |
| 3171 | ACCCAGACATGATGTGGAGGTGGA |
| 3172 | CCCAGACATGATGTGGAGGTGGAG |
| 3173 | CCAGACATGATGTGGAGGTGGAGC |
| 3174 | CAGACATGATGTGGAGGTGGAGCA |
| 3175 | AGACATGATGTGGAGGTGGAGCAA |
| 3176 | GACATGATGTGGAGGTGGAGCAAA |
| 3177 | ACATGATGTGGAGGTGGAGCAAAC |
| 3178 | CATGATGTGGAGGTGGAGCAAACT |
| 3179 | ATGATGTGGAGGTGGAGCAAACTA |
| 3180 | TGATGTGGAGGTGGAGCAAACTAG |
| 3181 | GATGTGGAGGTGGAGCAAACTAGG |
| 3182 | ATGTGGAGGTGGAGCAAACTAGGC |
| 3183 | TGTGGAGGTGGAGCAAACTAGGCA |
| 3184 | GTGGAGGTGGAGCAAACTAGGCAC |
| 3185 | TGGAGGTGGAGCAAACTAGGCACT |
| 3186 | GGAGGTGGAGCAAACTAGGCACTG |
| 3187 | GAGGTGGAGCAAACTAGGCACTGA |
| 3188 | AGGTGGAGCAAACTAGGCACTGAG |
| 3189 | GGTGGAGCAAACTAGGCACTGAGC |
| 3190 | GTGGAGCAAACTAGGCACTGAGCC |
| 3191 | TGGAGCAAACTAGGCACTGAGCCA |
| 3192 | GGAGCAAACTAGGCACTGAGCCAA |
| 3193 | GAGCAAACTAGGCACTGAGCCAAA |
| 3194 | AGCAAACTAGGCACTGAGCCAAAG |
| 3195 | GCAAACTAGGCACTGAGCCAAAGG |
| 3196 | CAAACTAGGCACTGAGCCAAAGGG |
| 3197 | AAACTAGGCACTGAGCCAAAGGGG |
| 3198 | AACTAGGCACTGAGCCAAAGGGGA |
| 3199 | ACTAGGCACTGAGCCAAAGGGGAT |
| 3200 | CTAGGCACTGAGCCAAAGGGGATC |
| 3201 | TAGGCACTGAGCCAAAGGGGATCA |
| 3202 | AGGCACTGAGCCAAAGGGGATCAG |
| 3203 | GGCACTGAGCCAAAGGGGATCAGT |
| 3204 | GCACTGAGCCAAAGGGGATCAGTG |
| 3205 | CACTGAGCCAAAGGGGATCAGTGA |
| 3206 | ACTGAGCCAAAGGGGATCAGTGAT |
| 3207 | CTGAGCCAAAGGGGATCAGTGATA |
| 3208 | TGAGCCAAAGGGGATCAGTGATAG |
| 3209 | GAGCCAAAGGGGATCAGTGATAGC |
| 3210 | AGCCAAAGGGGATCAGTGATAGCC |
| 3211 | GCCAAAGGGGATCAGTGATAGCCA |
| 3212 | AGCCCTACCCAGACATGATGTGGAG |
| 3213 | GCCCTACCCAGACATGATGTGGAGG |
| 3214 | CCCTACCCAGACATGATGTGGAGGT |
| 3215 | CCTACCCAGACATGATGTGGAGGTG |
| 3216 | CTACCCAGACATGATGTGGAGGTGG |
| 3217 | TACCCAGACATGATGTGGAGGTGGA |
| 3218 | ACCCAGACATGATGTGGAGGTGGAG |
| 3219 | CCCAGACATGATGTGGAGGTGGAGC |
| 3220 | CCAGACATGATGTGGAGGTGGAGCA |
| 3221 | CAGACATGATGTGGAGGTGGAGCAA |
| 3222 | AGACATGATGTGGAGGTGGAGCAAA |
| 3223 | GACATGATGTGGAGGTGGAGCAAAC |
| 3224 | ACATGATGTGGAGGTGGAGCAAACT |
| 3225 | CATGATGTGGAGGTGGAGCAAACTA |
| 3226 | ATGATGTGGAGGTGGAGCAAACTAG |
| 3227 | TGATGTGGAGGTGGAGCAAACTAGG |
| 3228 | GATGTGGAGGTGGAGCAAACTAGGC |
| 3229 | ATGTGGAGGTGGAGCAAACTAGGCA |
| 3230 | TGTGGAGGTGGAGCAAACTAGGCAC |
| 3231 | GTGGAGGTGGAGCAAACTAGGCACT |
| 3232 | TGGAGGTGGAGCAAACTAGGCACTG |
| 3233 | GGAGGTGGAGCAAACTAGGCACTGA |
| 3234 | GAGGTGGAGCAAACTAGGCACTGAG |
| 3235 | AGGTGGAGCAAACTAGGCACTGAGC |
| 3236 | GGTGGAGCAAACTAGGCACTGAGCC |
| 3237 | GTGGAGCAAACTAGGCACTGAGCCA |
| 3238 | TGGAGCAAACTAGGCACTGAGCCAA |
| 3239 | GGAGCAAACTAGGCACTGAGCCAAA |
| 3240 | GAGCAAACTAGGCACTGAGCCAAAG |
| 3241 | AGCAAACTAGGCACTGAGCCAAAGG |
| 3242 | GCAAACTAGGCACTGAGCCAAAGGG |
| 3243 | CAAACTAGGCACTGAGCCAAAGGGG |
| 3244 | AAACTAGGCACTGAGCCAAAGGGGA |
| 3245 | AACTAGGCACTGAGCCAAAGGGGAT |
| 3246 | ACTAGGCACTGAGCCAAAGGGGATC |
| 3247 | CTAGGCACTGAGCCAAAGGGGATCA |
| 3248 | TAGGCACTGAGCCAAAGGGGATCAG |
| 3249 | AGGCACTGAGCCAAAGGGGATCAGT |
| 3250 | GGCACTGAGCCAAAGGGGATCAGTG |
| 3251 | GCACTGAGCCAAAGGGGATCAGTGA |
| 3252 | CACTGAGCCAAAGGGGATCAGTGAT |
| 3253 | ACTGAGCCAAAGGGGATCAGTGATA |
| 3254 | CTGAGCCAAAGGGGATCAGTGATAG |
| 3255 | TGAGCCAAAGGGGATCAGTGATAGC |
| 3256 | GAGCCAAAGGGGATCAGTGATAGCC |
| 3257 | AGCCAAAGGGGATCAGTGATAGCCA |
| 3258 | AGCCCTACCCAGACATGATGTGGAGG |
| 3259 | GCCCTACCCAGACATGATGTGGAGGT |
| 3260 | CCCTACCCAGACATGATGTGGAGGTG |
| 3261 | CCTACCCAGACATGATGTGGAGGTGG |
| 3262 | CTACCCAGACATGATGTGGAGGTGGA |
| 3263 | TACCCAGACATGATGTGGAGGTGGAG |
| 3264 | ACCCAGACATGATGTGGAGGTGGAGC |
| 3265 | CCCAGACATGATGTGGAGGTGGAGCA |
| 3266 | CCAGACATGATGTGGAGGTGGAGCAA |
| 3267 | CAGACATGATGTGGAGGTGGAGCAAA |
| 3268 | AGACATGATGTGGAGGTGGAGCAAAC |
| 3269 | GACATGATGTGGAGGTGGAGCAAACT |
| 3270 | ACATGATGTGGAGGTGGAGCAAACTA |
| 3271 | CATGATGTGGAGGTGGAGCAAACTAG |
| 3272 | ATGATGTGGAGGTGGAGCAAACTAGG |
| 3273 | TGATGTGGAGGTGGAGCAAACTAGGC |
| 3274 | GATGTGGAGGTGGAGCAAACTAGGCA |
| 3275 | ATGTGGAGGTGGAGCAAACTAGGCAC |
| 3276 | TGTGGAGGTGGAGCAAACTAGGCACT |
| 3277 | GTGGAGGTGGAGCAAACTAGGCACTG |
| 3278 | TGGAGGTGGAGCAAACTAGGCACTGA |
| 3279 | GGAGGTGGAGCAAACTAGGCACTGAG |
| 3280 | GAGGTGGAGCAAACTAGGCACTGAGC |
| 3281 | AGGTGGAGCAAACTAGGCACTGAGCC |
| 3282 | GGTGGAGCAAACTAGGCACTGAGCCA |
| 3283 | GTGGAGCAAACTAGGCACTGAGCCAA |
| 3284 | TGGAGCAAACTAGGCACTGAGCCAAA |
| 3285 | GGAGCAAACTAGGCACTGAGCCAAAG |
| 3286 | GAGCAAACTAGGCACTGAGCCAAAGG |
| 3287 | AGCAAACTAGGCACTGAGCCAAAGGG |
| 3288 | GCAAACTAGGCACTGAGCCAAAGGGG |
| 3289 | CAAACTAGGCACTGAGCCAAAGGGGA |
| 3290 | AAACTAGGCACTGAGCCAAAGGGGAT |
| 3291 | AACTAGGCACTGAGCCAAAGGGGATC |
| 3292 | ACTAGGCACTGAGCCAAAGGGGATCA |
| 3293 | CTAGGCACTGAGCCAAAGGGGATCAG |
| 3294 | TAGGCACTGAGCCAAAGGGGATCAGT |
| 3295 | AGGCACTGAGCCAAAGGGGATCAGTG |
| 3296 | GGCACTGAGCCAAAGGGGATCAGTGA |
| 3297 | GCACTGAGCCAAAGGGGATCAGTGAT |
| 3298 | CACTGAGCCAAAGGGGATCAGTGATA |
| 3299 | ACTGAGCCAAAGGGGATCAGTGATAG |
| 3300 | CTGAGCCAAAGGGGATCAGTGATAGC |
| 3301 | TGAGCCAAAGGGGATCAGTGATAGCC |
| 3302 | GAGCCAAAGGGGATCAGTGATAGCCA |
| 3303 | ATTAGTCTTATTTGGCGT |
| 3304 | TTAGTCTTATTTGGCGTT |
| 3305 | TAGTCTTATTTGGCGTTA |
| 3306 | AGTCTTATTTGGCGTTAT |
| 3307 | GTCTTATTTGGCGTTATT |
| 3308 | TCTTATTTGGCGTTATTA |
| 3309 | CTTATTTGGCGTTATTAA |
| 3310 | GCGTTATTAATTCTACAG |
| 3311 | CGTTATTAATTCTACAGA |
| 3312 | TAATTCTACAGAGGGAGC |
| 3313 | TTCTACAGAGGGAGCATA |
| 3314 | CTACAGAGGGAGCATAAA |
| 3315 | TGCTAGGATTGTAATTAGT |
| 3316 | CTAGGATTGTAATTAGTCT |
| 3317 | GTAATTAGTCTTATTTGGC |
| 3318 | TAATTAGTCTTATTTGGCG |
| 3319 | AATTAGTCTTATTTGGCGT |
| 3320 | ATTAGTCTTATTTGGCGTT |
| 3321 | TTAGTCTTATTTGGCGTTA |
| 3322 | TAGTCTTATTTGGCGTTAT |
| 3323 | AGTCTTATTTGGCGTTATT |
| 3324 | GTCTTATTTGGCGTTATTA |
| 3325 | TCTTATTTGGCGTTATTAA |
| 3326 | CTTATTTGGCGTTATTAAT |
| 3327 | TTATTTGGCGTTATTAATT |
| 3328 | GGCGTTATTAATTCTACAG |
| 3329 | GCGTTATTAATTCTACAGA |
| 3330 | CGTTATTAATTCTACAGAG |
| 3331 | ATTAATTCTACAGAGGGAG |
| 3332 | TTAATTCTACAGAGGGAGC |
| 3333 | TAATTCTACAGAGGGAGCA |
| 3334 | ATTCTACAGAGGGAGCATA |
| 3335 | TTCTACAGAGGGAGCATAA |
| 3336 | TCTACAGAGGGAGCATAAA |
| 3337 | CTACAGAGGGAGCATAAAA |
| 3338 | TACAGAGGGAGCATAAAAC |
| 3339 | TGCTAGGATTGTAATTAGTC |
| 3340 | GCTAGGATTGTAATTAGTCT |
| 3341 | CTAGGATTGTAATTAGTCTT |
| 3342 | TAGGATTGTAATTAGTCTTA |
| 3343 | TGTAATTAGTCTTATTTGGC |
| 3344 | GTAATTAGTCTTATTTGGCG |
| 3345 | TAATTAGTCTTATTTGGCGT |
| 3346 | AATTAGTCTTATTTGGCGTT |
| 3347 | ATTAGTCTTATTTGGCGTTA |
| 3348 | TTAGTCTTATTTGGCGTTAT |
| 3349 | TAGTCTTATTTGGCGTTATT |
| 3350 | AGTCTTATTTGGCGTTATTA |
| 3351 | GTCTTATTTGGCGTTATTAA |
| 3352 | TCTTATTTGGCGTTATTAAT |
| 3353 | CTTATTTGGCGTTATTAATT |
| 3354 | TTATTTGGCGTTATTAATTC |
| 3355 | TTTGGCGTTATTAATTCTAC |
| 3356 | TGGCGTTATTAATTCTACAG |
| 3357 | GGCGTTATTAATTCTACAGA |
| 3358 | GCGTTATTAATTCTACAGAG |
| 3359 | CGTTATTAATTCTACAGAGG |
| 3360 | GTTATTAATTCTACAGAGGG |
| 3361 | TATTAATTCTACAGAGGGAG |
| 3362 | ATTAATTCTACAGAGGGAGC |
| 3363 | TTAATTCTACAGAGGGAGCA |
| 3364 | TAATTCTACAGAGGGAGCAT |
| 3365 | AATTCTACAGAGGGAGCATA |
| 3366 | ATTCTACAGAGGGAGCATAA |
| 3367 | TTCTACAGAGGGAGCATAAA |
| 3368 | TCTACAGAGGGAGCATAAAA |
| 3369 | CTACAGAGGGAGCATAAAAC |
| 3370 | TACAGAGGGAGCATAAAACA |
| 3371 | AGAGGGAGCATAAAACATTT |
| 3372 | GGGAGCATAAAACATTTAAT |
| 3373 | TGCTAGGATTGTAATTAGTCT |
| 3374 | GCTAGGATTGTAATTAGTCTT |
| 3375 | CTAGGATTGTAATTAGTCTTA |
| 3376 | TAGGATTGTAATTAGTCTTAT |
| 3377 | ATTGTAATTAGTCTTATTTGG |
| 3378 | TTGTAATTAGTCTTATTTGGC |
| 3379 | TGTAATTAGTCTTATTTGGCG |
| 3380 | GTAATTAGTCTTATTTGGCGT |
| 3381 | TAATTAGTCTTATTTGGCGTT |
| 3382 | AATTAGTCTTATTTGGCGTTA |
| 3383 | ATTAGTCTTATTTGGCGTTAT |
| 3384 | TTAGTCTTATTTGGCGTTATT |
| 3385 | TAGTCTTATTTGGCGTTATTA |
| 3386 | AGTCTTATTTGGCGTTATTAA |
| 3387 | GTCTTATTTGGCGTTATTAAT |
| 3388 | TCTTATTTGGCGTTATTAATT |
| 3389 | CTTATTTGGCGTTATTAATTC |
| 3390 | TTATTTGGCGTTATTAATTCT |
| 3391 | ATTTGGCGTTATTAATTCTAC |
| 3392 | TTTGGCGTTATTAATTCTACA |
| 3393 | TTGGCGTTATTAATTCTACAG |
| 3394 | TGGCGTTATTAATTCTACAGA |
| 3395 | GGCGTTATTAATTCTACAGAG |
| 3396 | GCGTTATTAATTCTACAGAGG |
| 3397 | CGTTATTAATTCTACAGAGGG |
| 3398 | GTTATTAATTCTACAGAGGGA |
| 3399 | TTATTAATTCTACAGAGGGAG |
| 3400 | TATTAATTCTACAGAGGGAGC |
| 3401 | ATTAATTCTACAGAGGGAGCA |
| 3402 | TTAATTCTACAGAGGGAGCAT |
| 3403 | TAATTCTACAGAGGGAGCATA |
| 3404 | AATTCTACAGAGGGAGCATAA |
| 3405 | ATTCTACAGAGGGAGCATAAA |
| 3406 | TTCTACAGAGGGAGCATAAAA |
| 3407 | TCTACAGAGGGAGCATAAAAC |
| 3408 | CTACAGAGGGAGCATAAAACA |
| 3409 | TACAGAGGGAGCATAAAACAT |
| 3410 | ACAGAGGGAGCATAAAACATT |
| 3411 | CAGAGGGAGCATAAAACATTT |
| 3412 | AGAGGGAGCATAAAACATTTA |
| 3413 | AGGGAGCATAAAACATTTAAT |
| 3414 | GGGAGCATAAAACATTTAATA |
| 3415 | GGAGCATAAAACATTTAATAA |
| 3416 | GAGCATAAAACATTTAATAAT |
| 3417 | TGCTAGGATTGTAATTAGTCTT |
| 3418 | GCTAGGATTGTAATTAGTCTTA |
| 3419 | CTAGGATTGTAATTAGTCTTAT |
| 3420 | TAGGATTGTAATTAGTCTTATT |
| 3421 | GATTGTAATTAGTCTTATTTGG |
| 3422 | ATTGTAATTAGTCTTATTTGGC |
| 3423 | TTGTAATTAGTCTTATTTGGCG |
| 3424 | TGTAATTAGTCTTATTTGGCGT |
| 3425 | GTAATTAGTCTTATTTGGCGTT |
| 3426 | TAATTAGTCTTATTTGGCGTTA |
| 3427 | AATTAGTCTTATTTGGCGTTAT |
| 3428 | ATTAGTCTTATTTGGCGTTATT |
| 3429 | TTAGTCTTATTTGGCGTTATTA |
| 3430 | TAGTCTTATTTGGCGTTATTAA |
| 3431 | AGTCTTATTTGGCGTTATTAAT |
| 3432 | GTCTTATTTGGCGTTATTAATT |
| 3433 | TCTTATTTGGCGTTATTAATTC |
| 3434 | CTTATTTGGCGTTATTAATTCT |
| 3435 | TTATTTGGCGTTATTAATTCTA |
| 3436 | TATTTGGCGTTATTAATTCTAC |
| 3437 | ATTTGGCGTTATTAATTCTACA |
| 3438 | TTTGGCGTTATTAATTCTACAG |
| 3439 | TTGGCGTTATTAATTCTACAGA |
| 3440 | TGGCGTTATTAATTCTACAGAG |
| 3441 | GGCGTTATTAATTCTACAGAGG |
| 3442 | GCGTTATTAATTCTACAGAGGG |
| 3443 | CGTTATTAATTCTACAGAGGGA |
| 3444 | GTTATTAATTCTACAGAGGGAG |
| 3445 | TTATTAATTCTACAGAGGGAGC |
| 3446 | TATTAATTCTACAGAGGGAGCA |
| 3447 | ATTAATTCTACAGAGGGAGCAT |
| 3448 | TTAATTCTACAGAGGGAGCATA |
| 3449 | TAATTCTACAGAGGGAGCATAA |
| 3450 | AATTCTACAGAGGGAGCATAAA |
| 3451 | ATTCTACAGAGGGAGCATAAAA |
| 3452 | TTCTACAGAGGGAGCATAAAAC |
| 3453 | TCTACAGAGGGAGCATAAAACA |
| 3454 | CTACAGAGGGAGCATAAAACAT |
| 3455 | TACAGAGGGAGCATAAAACATT |
| 3456 | ACAGAGGGAGCATAAAACATTT |
| 3457 | CAGAGGGAGCATAAAACATTTA |
| 3458 | AGAGGGAGCATAAAACATTTAA |
| 3459 | GAGGGAGCATAAAACATTTAAT |
| 3460 | AGGGAGCATAAAACATTTAATA |
| 3461 | GGGAGCATAAAACATTTAATAA |
| 3462 | GGAGCATAAAACATTTAATAAT |
| 3463 | GAGCATAAAACATTTAATAATA |
| 3464 | TGCTAGGATTGTAATTAGTCTTA |
| 3465 | GCTAGGATTGTAATTAGTCTTAT |
| 3466 | CTAGGATTGTAATTAGTCTTATT |
| 3467 | TAGGATTGTAATTAGTCTTATTT |
| 3468 | AGGATTGTAATTAGTCTTATTTG |
| 3469 | GGATTGTAATTAGTCTTATTTGG |
| 3470 | GATTGTAATTAGTCTTATTTGGC |
| 3471 | ATTGTAATTAGTCTTATTTGGCG |
| 3472 | TTGTAATTAGTCTTATTTGGCGT |
| 3473 | TGTAATTAGTCTTATTTGGCGTT |
| 3474 | GTAATTAGTCTTATTTGGCGTTA |
| 3475 | TAATTAGTCTTATTTGGCGTTAT |
| 3476 | AATTAGTCTTATTTGGCGTTATT |
| 3477 | ATTAGTCTTATTTGGCGTTATTA |
| 3478 | TTAGTCTTATTTGGCGTTATTAA |
| 3479 | TAGTCTTATTTGGCGTTATTAAT |
| 3480 | AGTCTTATTTGGCGTTATTAATT |
| 3481 | GTCTTATTTGGCGTTATTAATTC |
| 3482 | TCTTATTTGGCGTTATTAATTCT |
| 3483 | CTTATTTGGCGTTATTAATTCTA |
| 3484 | TTATTTGGCGTTATTAATTCTAC |
| 3485 | TATTTGGCGTTATTAATTCTACA |
| 3486 | ATTTGGCGTTATTAATTCTACAG |
| 3487 | TTTGGCGTTATTAATTCTACAGA |
| 3488 | TTGGCGTTATTAATTCTACAGAG |
| 3489 | TGGCGTTATTAATTCTACAGAGG |
| 3490 | GGCGTTATTAATTCTACAGAGGG |
| 3491 | GCGTTATTAATTCTACAGAGGGA |
| 3492 | CGTTATTAATTCTACAGAGGGAG |
| 3493 | GTTATTAATTCTACAGAGGGAGC |
| 3494 | TTATTAATTCTACAGAGGGAGCA |
| 3495 | TATTAATTCTACAGAGGGAGCAT |
| 3496 | ATTAATTCTACAGAGGGAGCATA |
| 3497 | TTAATTCTACAGAGGGAGCATAA |
| 3498 | TAATTCTACAGAGGGAGCATAAA |
| 3499 | AATTCTACAGAGGGAGCATAAAA |
| 3500 | ATTCTACAGAGGGAGCATAAAAC |
| 3501 | TTCTACAGAGGGAGCATAAAACA |
| 3502 | TCTACAGAGGGAGCATAAAACAT |
| 3503 | CTACAGAGGGAGCATAAAACATT |
| 3504 | TACAGAGGGAGCATAAAACATTT |
| 3505 | ACAGAGGGAGCATAAAACATTTA |
| 3506 | CAGAGGGAGCATAAAACATTTAA |
| 3507 | AGAGGGAGCATAAAACATTTAAT |
| 3508 | GAGGGAGCATAAAACATTTAATA |
| 3509 | AGGGAGCATAAAACATTTAATAA |
| 3510 | GGGAGCATAAAACATTTAATAAT |
| 3511 | GGAGCATAAAACATTTAATAATA |
| 3512 | TGCTAGGATTGTAATTAGTCTTAT |
| 3513 | GCTAGGATTGTAATTAGTCTTATT |
| 3514 | CTAGGATTGTAATTAGTCTTATTT |
| 3515 | TAGGATTGTAATTAGTCTTATTTG |
| 3516 | AGGATTGTAATTAGTCTTATTTGG |
| 3517 | GGATTGTAATTAGTCTTATTTGGC |
| 3518 | GATTGTAATTAGTCTTATTTGGCG |
| 3519 | ATTGTAATTAGTCTTATTTGGCGT |
| 3520 | TTGTAATTAGTCTTATTTGGCGTT |
| 3521 | TGTAATTAGTCTTATTTGGCGTTA |
| 3522 | GTAATTAGTCTTATTTGGCGTTAT |
| 3523 | TAATTAGTCTTATTTGGCGTTATT |
| 3524 | AATTAGTCTTATTTGGCGTTATTA |
| 3525 | ATTAGTCTTATTTGGCGTTATTAA |
| 3526 | TTAGTCTTATTTGGCGTTATTAAT |
| 3527 | TAGTCTTATTTGGCGTTATTAATT |
| 3528 | AGTCTTATTTGGCGTTATTAATTC |
| 3529 | GTCTTATTTGGCGTTATTAATTCT |
| 3530 | TCTTATTTGGCGTTATTAATTCTA |
| 3531 | CTTATTTGGCGTTATTAATTCTAC |
| 3532 | TTATTTGGCGTTATTAATTCTACA |
| 3533 | TATTTGGCGTTATTAATTCTACAG |
| 3534 | ATTTGGCGTTATTAATTCTACAGA |
| 3535 | TTTGGCGTTATTAATTCTACAGAG |
| 3536 | TTGGCGTTATTAATTCTACAGAGG |
| 3537 | TGGCGTTATTAATTCTACAGAGGG |
| 3538 | GGCGTTATTAATTCTACAGAGGGA |
| 3539 | GCGTTATTAATTCTACAGAGGGAG |
| 3540 | CGTTATTAATTCTACAGAGGGAGC |
| 3541 | GTTATTAATTCTACAGAGGGAGCA |
| 3542 | TTATTAATTCTACAGAGGGAGCAT |
| 3543 | TATTAATTCTACAGAGGGAGCATA |
| 3544 | ATTAATTCTACAGAGGGAGCATAA |
| 3545 | TTAATTCTACAGAGGGAGCATAAA |
| 3546 | TAATTCTACAGAGGGAGCATAAAA |
| 3547 | AATTCTACAGAGGGAGCATAAAAC |
| 3548 | ATTCTACAGAGGGAGCATAAAACA |
| 3549 | TTCTACAGAGGGAGCATAAAACAT |
| 3550 | TCTACAGAGGGAGCATAAAACATT |
| 3551 | CTACAGAGGGAGCATAAAACATTT |
| 3552 | TACAGAGGGAGCATAAAACATTTA |
| 3553 | ACAGAGGGAGCATAAAACATTTAA |
| 3554 | CAGAGGGAGCATAAAACATTTAAT |
| 3555 | AGAGGGAGCATAAAACATTTAATA |
| 3556 | GAGGGAGCATAAAACATTTAATAA |
| 3557 | AGGGAGCATAAAACATTTAATAAT |
| 3558 | GGGAGCATAAAACATTTAATAATA |
| 3559 | TGCTAGGATTGTAATTAGTCTTATT |
| 3560 | GCTAGGATTGTAATTAGTCTTATTT |
| 3561 | CTAGGATTGTAATTAGTCTTATTTG |
| 3562 | TAGGATTGTAATTAGTCTTATTTGG |
| 3563 | AGGATTGTAATTAGTCTTATTTGGC |
| 3564 | GGATTGTAATTAGTCTTATTTGGCG |
| 3565 | GATTGTAATTAGTCTTATTTGGCGT |
| 3566 | ATTGTAATTAGTCTTATTTGGCGTT |
| 3567 | TTGTAATTAGTCTTATTTGGCGTTA |
| 3568 | TGTAATTAGTCTTATTTGGCGTTAT |
| 3569 | GTAATTAGTCTTATTTGGCGTTATT |
| 3570 | TAATTAGTCTTATTTGGCGTTATTA |
| 3571 | AATTAGTCTTATTTGGCGTTATTAA |
| 3572 | ATTAGTCTTATTTGGCGTTATTAAT |
| 3573 | TTAGTCTTATTTGGCGTTATTAATT |
| 3574 | TAGTCTTATTTGGCGTTATTAATTC |
| 3575 | AGTCTTATTTGGCGTTATTAATTCT |
| 3576 | GTCTTATTTGGCGTTATTAATTCTA |
| 3577 | TCTTATTTGGCGTTATTAATTCTAC |
| 3578 | CTTATTTGGCGTTATTAATTCTACA |
| 3579 | TTATTTGGCGTTATTAATTCTACAG |
| 3580 | TATTTGGCGTTATTAATTCTACAGA |
| 3581 | ATTTGGCGTTATTAATTCTACAGAG |
| 3582 | TTTGGCGTTATTAATTCTACAGAGG |
| 3583 | TTGGCGTTATTAATTCTACAGAGGG |
| 3584 | TGGCGTTATTAATTCTACAGAGGGA |
| 3585 | GGCGTTATTAATTCTACAGAGGGAG |
| 3586 | GCGTTATTAATTCTACAGAGGGAGC |
| 3587 | CGTTATTAATTCTACAGAGGGAGCA |
| 3588 | GTTATTAATTCTACAGAGGGAGCAT |
| 3589 | TTATTAATTCTACAGAGGGAGCATA |
| 3590 | TATTAATTCTACAGAGGGAGCATAA |
| 3591 | ATTAATTCTACAGAGGGAGCATAAA |
| 3592 | TTAATTCTACAGAGGGAGCATAAAA |
| 3593 | TAATTCTACAGAGGGAGCATAAAAC |
| 3594 | AATTCTACAGAGGGAGCATAAAACA |
| 3595 | ATTCTACAGAGGGAGCATAAAACAT |
| 3596 | TTCTACAGAGGGAGCATAAAACATT |
| 3597 | TCTACAGAGGGAGCATAAAACATTT |
| 3598 | CTACAGAGGGAGCATAAAACATTTA |
| 3599 | TACAGAGGGAGCATAAAACATTTAA |
| 3600 | ACAGAGGGAGCATAAAACATTTAAT |
| 3601 | CAGAGGGAGCATAAAACATTTAATA |
| 3602 | AGAGGGAGCATAAAACATTTAATAA |
| 3603 | GAGGGAGCATAAAACATTTAATAAT |
| 3604 | AGGGAGCATAAAACATTTAATAATA |
| 3605 | TGCTAGGATTGTAATTAGTCTTATTT |
| 3606 | GCTAGGATTGTAATTAGTCTTATTTG |
| 3607 | CTAGGATTGTAATTAGTCTTATTTGG |
| 3608 | TAGGATTGTAATTAGTCTTATTTGGC |
| 3609 | AGGATTGTAATTAGTCTTATTTGGCG |
| 3610 | GGATTGTAATTAGTCTTATTTGGCGT |
| 3611 | GATTGTAATTAGTCTTATTTGGCGTT |
| 3612 | ATTGTAATTAGTCTTATTTGGCGTTA |
| 3613 | TTGTAATTAGTCTTATTTGGCGTTAT |
| 3614 | TGTAATTAGTCTTATTTGGCGTTATT |
| 3615 | GTAATTAGTCTTATTTGGCGTTATTA |
| 3616 | TAATTAGTCTTATTTGGCGTTATTAA |
| 3617 | AATTAGTCTTATTTGGCGTTATTAAT |
| 3618 | ATTAGTCTTATTTGGCGTTATTAATT |
| 3619 | TTAGTCTTATTTGGCGTTATTAATTC |
| 3620 | TAGTCTTATTTGGCGTTATTAATTCT |
| 3621 | AGTCTTATTTGGCGTTATTAATTCTA |
| 3622 | GTCTTATTTGGCGTTATTAATTCTAC |
| 3623 | TCTTATTTGGCGTTATTAATTCTACA |
| 3624 | CTTATTTGGCGTTATTAATTCTACAG |
| 3625 | TTATTTGGCGTTATTAATTCTACAGA |
| 3626 | TATTTGGCGTTATTAATTCTACAGAG |
| 3627 | ATTTGGCGTTATTAATTCTACAGAGG |
| 3628 | TTTGGCGTTATTAATTCTACAGAGGG |
| 3629 | TTGGCGTTATTAATTCTACAGAGGGA |
| 3630 | TGGCGTTATTAATTCTACAGAGGGAG |
| 3631 | GGCGTTATTAATTCTACAGAGGGAGC |
| 3632 | GCGTTATTAATTCTACAGAGGGAGCA |
| 3633 | CGTTATTAATTCTACAGAGGGAGCAT |
| 3634 | GTTATTAATTCTACAGAGGGAGCATA |
| 3635 | TTATTAATTCTACAGAGGGAGCATAA |
| 3636 | TATTAATTCTACAGAGGGAGCATAAA |
| 3637 | ATTAATTCTACAGAGGGAGCATAAAA |
| 3638 | TTAATTCTACAGAGGGAGCATAAAAC |
| 3639 | TAATTCTACAGAGGGAGCATAAAACA |
| 3640 | AATTCTACAGAGGGAGCATAAAACAT |
| 3641 | ATTCTACAGAGGGAGCATAAAACATT |
| 3642 | TTCTACAGAGGGAGCATAAAACATTT |
| 3643 | TCTACAGAGGGAGCATAAAACATTTA |
| 3644 | CTACAGAGGGAGCATAAAACATTTAA |
| 3645 | TACAGAGGGAGCATAAAACATTTAAT |
| 3646 | ACAGAGGGAGCATAAAACATTTAATA |
| 3647 | CAGAGGGAGCATAAAACATTTAATAA |
| 3648 | AGAGGGAGCATAAAACATTTAATAAT |
| 3649 | GAGGGAGCATAAAACATTTAATAATA |
| 3650 | CATTGGACTTACTCAACT |
| 3651 | ATTGGACTTACTCAACTC |
| 3652 | TTGGACTTACTCAACTCA |
| 3653 | GACTTACTCAACTCACTA |
| 3654 | ACTTACTCAACTCACTAT |
| 3655 | TATTTCTTACTTTCGTCC |
| 3656 | CTTACTTTCGTCCTTAAG |
| 3657 | ATAATACATTGGACTTACT |
| 3658 | TAATACATTGGACTTACTC |
| 3659 | TACATTGGACTTACTCAAC |
| 3660 | ACATTGGACTTACTCAACT |
| 3661 | CATTGGACTTACTCAACTC |
| 3662 | ATTGGACTTACTCAACTCA |
| 3663 | TTGGACTTACTCAACTCAC |
| 3664 | GGACTTACTCAACTCACTA |
| 3665 | GACTTACTCAACTCACTAT |
| 3666 | ACTTACTCAACTCACTATT |
| 3667 | TTACTCAACTCACTATTTC |
| 3668 | AACTCACTATTTCTTACTT |
| 3669 | TCACTATTTCTTACTTTCG |
| 3670 | ACTATTTCTTACTTTCGTC |
| 3671 | CTATTTCTTACTTTCGTCC |
| 3672 | TATTTCTTACTTTCGTCCT |
| 3673 | ATTTCTTACTTTCGTCCTT |
| 3674 | TCTTACTTTCGTCCTTAAG |
| 3675 | TTAATAATACATTGGACTTA |
| 3676 | AATAATACATTGGACTTACT |
| 3677 | TAATACATTGGACTTACTCA |
| 3678 | AATACATTGGACTTACTCAA |
| 3679 | ATACATTGGACTTACTCAAC |
| 3680 | TACATTGGACTTACTCAACT |
| 3681 | ACATTGGACTTACTCAACTC |
| 3682 | CATTGGACTTACTCAACTCA |
| 3683 | ATTGGACTTACTCAACTCAC |
| 3684 | TTGGACTTACTCAACTCACT |
| 3685 | TGGACTTACTCAACTCACTA |
| 3686 | GACTTACTCAACTCACTATT |
| 3687 | ACTTACTCAACTCACTATTT |
| 3688 | CTTACTCAACTCACTATTTC |
| 3689 | TTACTCAACTCACTATTTCT |
| 3690 | TACTCAACTCACTATTTCTT |
| 3691 | CAACTCACTATTTCTTACTT |
| 3692 | AACTCACTATTTCTTACTTT |
| 3693 | CTCACTATTTCTTACTTTCG |
| 3694 | TCACTATTTCTTACTTTCGT |
| 3695 | ACTATTTCTTACTTTCGTCC |
| 3696 | CTATTTCTTACTTTCGTCCT |
| 3697 | TATTTCTTACTTTCGTCCTT |
| 3698 | ATTTCTTACTTTCGTCCTTA |
| 3699 | TTCTTACTTTCGTCCTTAAG |
| 3700 | AAAACATTTAATAATACATTG |
| 3701 | AAACATTTAATAATACATTGG |
| 3702 | AACATTTAATAATACATTGGA |
| 3703 | ACATTTAATAATACATTGGAC |
| 3704 | ATTTAATAATACATTGGACTT |
| 3705 | TTTAATAATACATTGGACTTA |
| 3706 | TTAATAATACATTGGACTTAC |
| 3707 | TAATAATACATTGGACTTACT |
| 3708 | AATAATACATTGGACTTACTC |
| 3709 | ATAATACATTGGACTTACTCA |
| 3710 | TAATACATTGGACTTACTCAA |
| 3711 | AATACATTGGACTTACTCAAC |
| 3712 | ATACATTGGACTTACTCAACT |
| 3713 | TACATTGGACTTACTCAACTC |
| 3714 | ACATTGGACTTACTCAACTCA |
| 3715 | CATTGGACTTACTCAACTCAC |
| 3716 | ATTGGACTTACTCAACTCACT |
| 3717 | TTGGACTTACTCAACTCACTA |
| 3718 | TGGACTTACTCAACTCACTAT |
| 3719 | GGACTTACTCAACTCACTATT |
| 3720 | GACTTACTCAACTCACTATTT |
| 3721 | ACTTACTCAACTCACTATTTC |
| 3722 | CTTACTCAACTCACTATTTCT |
| 3723 | TTACTCAACTCACTATTTCTT |
| 3724 | TACTCAACTCACTATTTCTTA |
| 3725 | ACTCAACTCACTATTTCTTAC |
| 3726 | CTCAACTCACTATTTCTTACT |
| 3727 | TCAACTCACTATTTCTTACTT |
| 3728 | CAACTCACTATTTCTTACTTT |
| 3729 | AACTCACTATTTCTTACTTTC |
| 3730 | ACTCACTATTTCTTACTTTCG |
| 3731 | CTCACTATTTCTTACTTTCGT |
| 3732 | TCACTATTTCTTACTTTCGTC |
| 3733 | CACTATTTCTTACTTTCGTCC |
| 3734 | ACTATTTCTTACTTTCGTCCT |
| 3735 | CTATTTCTTACTTTCGTCCTT |
| 3736 | TATTTCTTACTTTCGTCCTTA |
| 3737 | ATTTCTTACTTTCGTCCTTAA |
| 3738 | TTTCTTACTTTCGTCCTTAAG |
| 3739 | GCATAAAACATTTAATAATACA |
| 3740 | CATAAAACATTTAATAATACAT |
| 3741 | TAAAACATTTAATAATACATTG |
| 3742 | AAAACATTTAATAATACATTGG |
| 3743 | AAACATTTAATAATACATTGGA |
| 3744 | AACATTTAATAATACATTGGAC |
| 3745 | ACATTTAATAATACATTGGACT |
| 3746 | CATTTAATAATACATTGGACTT |
| 3747 | ATTTAATAATACATTGGACTTA |
| 3748 | TTTAATAATACATTGGACTTAC |
| 3749 | TTAATAATACATTGGACTTACT |
| 3750 | TAATAATACATTGGACTTACTC |
| 3751 | AATAATACATTGGACTTACTCA |
| 3752 | ATAATACATTGGACTTACTCAA |
| 3753 | TAATACATTGGACTTACTCAAC |
| 3754 | AATACATTGGACTTACTCAACT |
| 3755 | ATACATTGGACTTACTCAACTC |
| 3756 | TACATTGGACTTACTCAACTCA |
| 3757 | ACATTGGACTTACTCAACTCAC |
| 3758 | CATTGGACTTACTCAACTCACT |
| 3759 | ATTGGACTTACTCAACTCACTA |
| 3760 | TTGGACTTACTCAACTCACTAT |
| 3761 | TGGACTTACTCAACTCACTATT |
| 3762 | GGACTTACTCAACTCACTATTT |
| 3763 | GACTTACTCAACTCACTATTTC |
| 3764 | ACTTACTCAACTCACTATTTCT |
| 3765 | CTTACTCAACTCACTATTTCTT |
| 3766 | TTACTCAACTCACTATTTCTTA |
| 3767 | TACTCAACTCACTATTTCTTAC |
| 3768 | ACTCAACTCACTATTTCTTACT |
| 3769 | CTCAACTCACTATTTCTTACTT |
| 3770 | TCAACTCACTATTTCTTACTTT |
| 3771 | CAACTCACTATTTCTTACTTTC |
| 3772 | AACTCACTATTTCTTACTTTCG |
| 3773 | ACTCACTATTTCTTACTTTCGT |
| 3774 | CTCACTATTTCTTACTTTCGTC |
| 3775 | TCACTATTTCTTACTTTCGTCC |
| 3776 | CACTATTTCTTACTTTCGTCCT |
| 3777 | ACTATTTCTTACTTTCGTCCTT |
| 3778 | CTATTTCTTACTTTCGTCCTTA |
| 3779 | TATTTCTTACTTTCGTCCTTAA |
| 3780 | ATTTCTTACTTTCGTCCTTAAG |
| 3781 | GAGCATAAAACATTTAATAATAC |
| 3782 | AGCATAAAACATTTAATAATACA |
| 3783 | GCATAAAACATTTAATAATACAT |
| 3784 | CATAAAACATTTAATAATACATT |
| 3785 | ATAAAACATTTAATAATACATTG |
| 3786 | TAAAACATTTAATAATACATTGG |
| 3787 | AAAACATTTAATAATACATTGGA |
| 3788 | AAACATTTAATAATACATTGGAC |
| 3789 | AACATTTAATAATACATTGGACT |
| 3790 | ACATTTAATAATACATTGGACTT |
| 3791 | CATTTAATAATACATTGGACTTA |
| 3792 | ATTTAATAATACATTGGACTTAC |
| 3793 | TTTAATAATACATTGGACTTACT |
| 3794 | TTAATAATACATTGGACTTACTC |
| 3795 | TAATAATACATTGGACTTACTCA |
| 3796 | AATAATACATTGGACTTACTCAA |
| 3797 | ATAATACATTGGACTTACTCAAC |
| 3798 | TAATACATTGGACTTACTCAACT |
| 3799 | AATACATTGGACTTACTCAACTC |
| 3800 | ATACATTGGACTTACTCAACTCA |
| 3801 | TACATTGGACTTACTCAACTCAC |
| 3802 | ACATTGGACTTACTCAACTCACT |
| 3803 | CATTGGACTTACTCAACTCACTA |
| 3804 | ATTGGACTTACTCAACTCACTAT |
| 3805 | TTGGACTTACTCAACTCACTATT |
| 3806 | TGGACTTACTCAACTCACTATTT |
| 3807 | GGACTTACTCAACTCACTATTTC |
| 3808 | GACTTACTCAACTCACTATTTCT |
| 3809 | ACTTACTCAACTCACTATTTCTT |
| 3810 | CTTACTCAACTCACTATTTCTTA |
| 3811 | TTACTCAACTCACTATTTCTTAC |
| 3812 | TACTCAACTCACTATTTCTTACT |
| 3813 | ACTCAACTCACTATTTCTTACTT |
| 3814 | CTCAACTCACTATTTCTTACTTT |
| 3815 | TCAACTCACTATTTCTTACTTTC |
| 3816 | CAACTCACTATTTCTTACTTTCG |
| 3817 | AACTCACTATTTCTTACTTTCGT |
| 3818 | ACTCACTATTTCTTACTTTCGTC |
| 3819 | CTCACTATTTCTTACTTTCGTCC |
| 3820 | TCACTATTTCTTACTTTCGTCCT |
| 3821 | CACTATTTCTTACTTTCGTCCTT |
| 3822 | ACTATTTCTTACTTTCGTCCTTA |
| 3823 | CTATTTCTTACTTTCGTCCTTAA |
| 3824 | TATTTCTTACTTTCGTCCTTAAG |
| 3825 | GGAGCATAAAACATTTAATAATAC |
| 3826 | GAGCATAAAACATTTAATAATACA |
| 3827 | AGCATAAAACATTTAATAATACAT |
| 3828 | GCATAAAACATTTAATAATACATT |
| 3829 | CATAAAACATTTAATAATACATTG |
| 3830 | ATAAAACATTTAATAATACATTGG |
| 3831 | TAAAACATTTAATAATACATTGGA |
| 3832 | AAAACATTTAATAATACATTGGAC |
| 3833 | AAACATTTAATAATACATTGGACT |
| 3834 | AACATTTAATAATACATTGGACTT |
| 3835 | ACATTTAATAATACATTGGACTTA |
| 3836 | CATTTAATAATACATTGGACTTAC |
| 3837 | ATTTAATAATACATTGGACTTACT |
| 3838 | TTTAATAATACATTGGACTTACTC |
| 3839 | TTAATAATACATTGGACTTACTCA |
| 3840 | TAATAATACATTGGACTTACTCAA |
| 3841 | AATAATACATTGGACTTACTCAAC |
| 3842 | ATAATACATTGGACTTACTCAACT |
| 3843 | TAATACATTGGACTTACTCAACTC |
| 3844 | AATACATTGGACTTACTCAACTCA |
| 3845 | ATACATTGGACTTACTCAACTCAC |
| 3846 | TACATTGGACTTACTCAACTCACT |
| 3847 | ACATTGGACTTACTCAACTCACTA |
| 3848 | CATTGGACTTACTCAACTCACTAT |
| 3849 | ATTGGACTTACTCAACTCACTATT |
| 3850 | TTGGACTTACTCAACTCACTATTT |
| 3851 | TGGACTTACTCAACTCACTATTTC |
| 3852 | GGACTTACTCAACTCACTATTTCT |
| 3853 | GACTTACTCAACTCACTATTTCTT |
| 3854 | ACTTACTCAACTCACTATTTCTTA |
| 3855 | CTTACTCAACTCACTATTTCTTAC |
| 3856 | TTACTCAACTCACTATTTCTTACT |
| 3857 | TACTCAACTCACTATTTCTTACTT |
| 3858 | ACTCAACTCACTATTTCTTACTTT |
| 3859 | CTCAACTCACTATTTCTTACTTTC |
| 3860 | TCAACTCACTATTTCTTACTTTCG |
| 3861 | CAACTCACTATTTCTTACTTTCGT |
| 3862 | AACTCACTATTTCTTACTTTCGTC |
| 3863 | ACTCACTATTTCTTACTTTCGTCC |
| 3864 | CTCACTATTTCTTACTTTCGTCCT |
| 3865 | TCACTATTTCTTACTTTCGTCCTT |
| 3866 | CACTATTTCTTACTTTCGTCCTTA |
| 3867 | ACTATTTCTTACTTTCGTCCTTAA |
| 3868 | CTATTTCTTACTTTCGTCCTTAAG |
| 3869 | GGGAGCATAAAACATTTAATAATAC |
| 3870 | GGAGCATAAAACATTTAATAATACA |
| 3871 | GAGCATAAAACATTTAATAATACAT |
| 3872 | AGCATAAAACATTTAATAATACATT |
| 3873 | GCATAAAACATTTAATAATACATTG |
| 3874 | CATAAAACATTTAATAATACATTGG |
| 3875 | ATAAAACATTTAATAATACATTGGA |
| 3876 | TAAAACATTTAATAATACATTGGAC |
| 3877 | AAAACATTTAATAATACATTGGACT |
| 3878 | AAACATTTAATAATACATTGGACTT |
| 3879 | AACATTTAATAATACATTGGACTTA |
| 3880 | ACATTTAATAATACATTGGACTTAC |
| 3881 | CATTTAATAATACATTGGACTTACT |
| 3882 | ATTTAATAATACATTGGACTTACTC |
| 3883 | TTTAATAATACATTGGACTTACTCA |
| 3884 | TTAATAATACATTGGACTTACTCAA |
| 3885 | TAATAATACATTGGACTTACTCAAC |
| 3886 | AATAATACATTGGACTTACTCAACT |
| 3887 | ATAATACATTGGACTTACTCAACTC |
| 3888 | TAATACATTGGACTTACTCAACTCA |
| 3889 | AATACATTGGACTTACTCAACTCAC |
| 3890 | ATACATTGGACTTACTCAACTCACT |
| 3891 | TACATTGGACTTACTCAACTCACTA |
| 3892 | ACATTGGACTTACTCAACTCACTAT |
| 3893 | CATTGGACTTACTCAACTCACTATT |
| 3894 | ATTGGACTTACTCAACTCACTATTT |
| 3895 | TTGGACTTACTCAACTCACTATTTC |
| 3896 | TGGACTTACTCAACTCACTATTTCT |
| 3897 | GGACTTACTCAACTCACTATTTCTT |
| 3898 | GACTTACTCAACTCACTATTTCTTA |
| 3899 | ACTTACTCAACTCACTATTTCTTAC |
| 3900 | CTTACTCAACTCACTATTTCTTACT |
| 3901 | TTACTCAACTCACTATTTCTTACTT |
| 3902 | TACTCAACTCACTATTTCTTACTTT |
| 3903 | ACTCAACTCACTATTTCTTACTTTC |
| 3904 | CTCAACTCACTATTTCTTACTTTCG |
| 3905 | TCAACTCACTATTTCTTACTTTCGT |
| 3906 | CAACTCACTATTTCTTACTTTCGTC |
| 3907 | AACTCACTATTTCTTACTTTCGTCC |
| 3908 | ACTCACTATTTCTTACTTTCGTCCT |
| 3909 | CTCACTATTTCTTACTTTCGTCCTT |
| 3910 | TCACTATTTCTTACTTTCGTCCTTA |
| 3911 | CACTATTTCTTACTTTCGTCCTTAA |
| 3912 | ACTATTTCTTACTTTCGTCCTTAAG |
| 3913 | AGGGAGCATAAAACATTTAATAATAC |
| 3914 | GGGAGCATAAAACATTTAATAATACA |
| 3915 | GGAGCATAAAACATTTAATAATACAT |
| 3916 | GAGCATAAAACATTTAATAATACATT |
| 3917 | AGCATAAAACATTTAATAATACATTG |
| 3918 | GCATAAAACATTTAATAATACATTGG |
| 3919 | CATAAAACATTTAATAATACATTGGA |
| 3920 | ATAAAACATTTAATAATACATTGGAC |
| 3921 | TAAAACATTTAATAATACATTGGACT |
| 3922 | AAAACATTTAATAATACATTGGACTT |
| 3923 | AAACATTTAATAATACATTGGACTTA |
| 3924 | AACATTTAATAATACATTGGACTTAC |
| 3925 | ACATTTAATAATACATTGGACTTACT |
| 3926 | CATTTAATAATACATTGGACTTACTC |
| 3927 | ATTTAATAATACATTGGACTTACTCA |
| 3928 | TTTAATAATACATTGGACTTACTCAA |
| 3929 | TTAATAATACATTGGACTTACTCAAC |
| 3930 | TAATAATACATTGGACTTACTCAACT |
| 3931 | AATAATACATTGGACTTACTCAACTC |
| 3932 | ATAATACATTGGACTTACTCAACTCA |
| 3933 | TAATACATTGGACTTACTCAACTCAC |
| 3934 | AATACATTGGACTTACTCAACTCACT |
| 3935 | ATACATTGGACTTACTCAACTCACTA |
| 3936 | TACATTGGACTTACTCAACTCACTAT |
| 3937 | ACATTGGACTTACTCAACTCACTATT |
| 3938 | CATTGGACTTACTCAACTCACTATTT |
| 3939 | ATTGGACTTACTCAACTCACTATTTC |
| 3940 | TTGGACTTACTCAACTCACTATTTCT |
| 3941 | TGGACTTACTCAACTCACTATTTCTT |
| 3942 | GGACTTACTCAACTCACTATTTCTTA |
| 3943 | GACTTACTCAACTCACTATTTCTTAC |
| 3944 | ACTTACTCAACTCACTATTTCTTACT |
| 3945 | CTTACTCAACTCACTATTTCTTACTT |
| 3946 | TTACTCAACTCACTATTTCTTACTTT |
| 3947 | TACTCAACTCACTATTTCTTACTTTC |
| 3948 | ACTCAACTCACTATTTCTTACTTTCG |
| 3949 | CTCAACTCACTATTTCTTACTTTCGT |
| 3950 | TCAACTCACTATTTCTTACTTTCGTC |
| 3951 | CAACTCACTATTTCTTACTTTCGTCC |
| 3952 | AACTCACTATTTCTTACTTTCGTCCT |
| 3953 | ACTCACTATTTCTTACTTTCGTCCTT |
| 3954 | CTCACTATTTCTTACTTTCGTCCTTA |
| 3955 | TCACTATTTCTTACTTTCGTCCTTAA |
| 3956 | CACTATTTCTTACTTTCGTCCTTAAG |

TABLE 9
Exemplary ASOs that target regRNA RR65_v1

| SEQ ID | |
|---|---|
| NO | Sequence |
| 3957 | GAATCACTTAGTTGTACTGT |
| 3958 | GTCATTGAAGAATCACTTAG |
| 3959 | GAGATGAGTCATTGAAGAAT |
| 3960 | TGAAGATTTGAGATGAGTCA |
| 3961 | TCGGAATGAAGATTTGAGAT |
| 3962 | GAAGTCATCGGAATGAAGAT |
| 3963 | CTTGAGAAGTCATCGGAATG |
| 3964 | GCGAAGCCTTGAGAAGTCAT |
| 3965 | ACTCAGCGAAGCCTTGAGAA |
| 3966 | GGATCACTCAGCGAAGCCTT |
| 3967 | GTGAGGGATCACTCAGCGAA |
| 3968 | TTTGGGTGAGGGATCACTCA |
| 3969 | AATCATTTGGGTGAGGGATC |
| 3970 | AGTCAATAATCATTTGGGTG |
| 3971 | GGAGAGGGAAGTCAATAATC |
| 3972 | TGAGGGAGGAGAGGGAAGTC |
| 3973 | AAATCTGGGGTGAGGGAGGA |
| 3974 | CATGAGAAATCTGGGGTGAG |
| 3975 | GCAGTGACACATGAGAAATC |
| 3976 | AATTGGCAGTGACACATGAG |
| 3977 | ATTTGAATTGGCAGTGACAC |
| 3978 | AGCCATGAATTTGAATTGGC |
| 3979 | GTGCTTTAGCCATGAATTTG |
| 3980 | AAGAGATTAGTGCTTTAGCC |
| 3981 | GTGTGGAAAGAGATTAGTGC |
| 3982 | TTCCAGGAAGTAGAGGTGTG |
| 3983 | AGAAAGGATTCCAGGAAGTA |
| 3984 | TAACAAGAAAGGATTCCAGG |
| 3985 | GACTGATAACAAGAAAGGAT |
| 3986 | GATATAAGACTGATAACAAG |
| 3987 | GGCATAGATATAAGACTGAT |
| 3988 | GAATTGGGCATAGATATAAG |
| 3989 | GCTGGCTGAATTGGGCATAG |
| 3990 | CCCAATGGCTGGCTGAATTG |
| 3991 | CATTACCCCAATGGCTGGCT |
| 3992 | GAAAGTTCATTACCCCAATG |
| 3993 | GAATGGAAAGTTCATTACCC |
| 3994 | GTTCTAGAATGGAAAGTTCA |
| 3995 | GGACTGTGGTTCTAGAATGG |
| 3996 | GGCAAATTGGACTGTGGTTC |
| 3997 | GGTAACAGGCAAATTGGACT |
| 3998 | AGTGAGGTAACAGGCAAATT |
| 3999 | TTCTCAGTGAGGTAACAGGC |
| 4000 | AGAGCCTTCTCAGTGAGGTA |
| 4001 | GCCCCAGAGCCTTCTCAGTG |
| 4002 | TGTCAGAGCCCCAGAGCCTT |
| 4003 | GTGTTTGTCAGAGCCCCAGA |
| 4004 | CAGCAGTGTTTGTCAGAGCC |
| 4005 | AACTCACAGCAGTGTTTGTC |
| 4006 | TTCAGGAAACTCACAGCAGT |
| 4007 | AAGTAGGTTTCAGGAAACTC |
| 4008 | GGTACTCCTGAGCCTGAAAT |
| 4009 | ATTGAGTTACTAGGCTTAGT |
| 4010 | TAACTATTGAGTTACTAGGC |
| 4011 | AAAATTAACTATTGAGTTAC |
| 4012 | TGGGGGCCCATTGGAGGGTG |
| 4013 | GACACTGGGGGCCCATTGGA |
| 4014 | CCAGTAAGTGTGAACTAAAT |
| 4015 | GAGGTTTCCAGTAAGTGTGA |
| 4016 | GCCAAAGGAGGTTTCCAGTA |
| 4017 | ATGTGGCCAAAGGAGGTTTC |
| 4018 | TGTCAAAATGTGGCCAAAGG |
| 4019 | CATAGTGTCAAAATGTGGCC |
| 4020 | CCTGACATAGTGTCAAAATG |
| 4021 | CAACCTCTCCTGACATAGTG |
| 4022 | AGGATCCAACCTCTCCTGAC |
| 4023 | GTTGGAGGATCCAACCTCTC |
| 4024 | TCGGCAGGGGTTGGAGGATC |
| 4025 | TGATCTCGGCAGGGGTTGGA |
| 4026 | TGCAGTGATCTCGGCAGGGG |
| 4027 | GTATCTGCAGTGATCTCGGC |
| 4028 | TCTAAGTATCTGCAGTGATC |
| 4029 | ATCCAGGCTCTAAGTATCTG |
| 4030 | ATCTTATCCAGGCTCTAAGT |
| 4031 | GTTGATCATCTTATCCAGGC |
| 4032 | AAGCACTGTTGATCATCTTA |
| 4033 | AGCTATTTAAAGCACTGTTG |
| 4034 | ATATAAGCTATTTAAAGCAC |
| 4035 | TCAGCACAGCTTTTTAAAAT |
| 4036 | GCAAAACATTCAGCACAGCT |
| 4037 | CAGTGGCCTGCAAAACATTC |
| 4038 | CACCTCAGTGGCCTGCAAAA |
| 4039 | GTCCGCACCTCAGTGGCCTG |
| 4040 | ACCATGTCCGCACCTCAGTG |
| 4041 | TATAAAACCATGTCCGCACC |
| 4042 | ACTGATATAAAACCATGTCC |
| 4043 | CACCTGTGATCTCACTGATA |
| 4044 | ACCTTGCTCACCTGTGATCT |
| 4045 | CAATAACACCTTGCTCACCT |
| 4046 | TATAGCCAATAACACCTTGC |
| 4047 | CAAACATTATAGCCAATAAC |
| 4048 | AGGCTGATTTTCAAACATTA |
| 4049 | TGTTAAAGGCTGATTTTCAA |
| 4050 | TCAGATGTTAAAGGCTGATT |
| 4051 | TATAATCAGATGTTAAAGGC |
| 4052 | ATAAGAAGTCAAATATAATC |
| 4053 | GGACACTCAAATAAGAAGTC |
| 4054 | TCCAAGGACACTCAAATAAG |
| 4055 | ACATGGTCCAAGGACACTCA |
| 4056 | TAGAAAATTTATACATTATC |
| 4057 | GAAGTTATAGAAAATTTATA |
| 4058 | TTATGCATGAAGAAGTTATA |
| 4059 | GGATTTTATGCATGAAGAAG |
| 4060 | ATGTTAATAGGATTTTATGC |
| 4061 | TCAGCAATGTTAATAGGATT |

TABLE 10
Exemplary ASOs that target regRNA RR74_v1

| SEQ ID | |
|---|---|
| NO | Sequence |
| 4062 | GGTTTGTGTTCACTTACTAG |
| 4063 | TCACAAAGGTTTGTGTTCAC |
| 4064 | TGAAATCACAAAGGTTTGTG |
| 4065 | GATGGTCTTTGAAATCACAA |
| 4066 | ATCTCAAGAGATGGTCTTTG |
| 4067 | TTAACCAATCTCAAGAGATG |
| 4068 | GTTAAAAGTTAACCAATCTC |
| 4069 | GGTTTAGTTAAAAGTTAACC |
| 4070 | TGGGAGGTTTAGTTAAAAGT |
| 4071 | CTGTGTGTCTGGGAGGTTTA |
| 4072 | TCAGGCTGTGTGTCTGGGAG |
| 4073 | TAAGGTCAGGCTGTGTGTCT |
| 4074 | ATTGGAAGATAAGGTCAGGC |
| 4075 | TGCAAGAAAGATTGGAAGAT |
| 4076 | CCTAACTGCAAGAAAGATTG |
| 4077 | ACATAACCACACCTAACTGC |
| 4078 | GTGCCACATAACCACACCTA |
| 4079 | TCCACTGGAGAGGACTGTGC |
| 4080 | CAGATTCCACTGGAGAGGAC |
| 4081 | TACTTCTACTCAGATTCCAC |
| 4082 | GTGGCATCCATTACTTCTAC |
| 4083 | CCTGGAAGTGGCATCCATTA |
| 4084 | TGTGGGCCAATCCTGGAAGT |
| 4085 | GAAAGTTCCTGTGGGCCAAT |
| 4086 | TTGTATGGAAAGTTCCTGTG |
| 4087 | GGAGCATTTTGTATGGAAAG |
| 4088 | GAAAATATGGAGCATTTTGT |
| 4089 | GAAGAGAAAATATGGAGCAT |
| 4090 | TCACACAGGGAAGAGAAAAT |
| 4091 | ATCCATCACACAGGGAAGAG |
| 4092 | CATGTATCCATCACACAGGG |
| 4093 | CATGGACATGTATCCATCAC |
| 4094 | CTTGGACATGGACATGTATC |
| 4095 | GGTCTCCTTGGACATGGACA |
| 4096 | GGCTTTTAAGGTCTCCTTGG |
| 4097 | CATGTGGCTTTTAAGGTCTC |
| 4098 | CCTCAACATGTGGCTTTTAA |
| 4099 | GAAGCTCTACCATCCTCAAC |
| 4100 | AGGCTGACAGAAGCTCTACC |
| 4101 | AGAGACCCAGGCTGACAGAA |
| 4102 | GCAAAGGTGCTCTGCTCCAT |
| 4103 | GTTGGGCAAAGGTGCTCTGC |
| 4104 | TCGGTGTTGGGCAAAGGTGC |
| 4105 | GGAAATCGGTGTTGGGCAAA |
| 4106 | TAAGGGGAAATCGGTGTTGG |
| 4107 | CTTGAATAAGGGGAAATCGG |
| 4108 | GTAGGCCTTGAATAAGGGGA |
| 4109 | AGTCCAGTAGGCCTTGAATA |
| 4110 | ATGTGAAGTCCAGTAGGCCT |
| 4111 | ATTTCCCCTCATGTGAAGTC |
| 4112 | GCTAATTATTTCCCCTCATG |
| 4113 | GTGATACTTGCTAATTATTT |
| 4114 | TTAAGGTAGTGATACTTGCT |
| 4115 | TTTGGTTAAGGTAGTGATAC |
| 4116 | GATGATTTGGTTAAGGTAGT |
| 4117 | CTTACTTGTTGGATGATTTG |
| 4118 | TTGCCACTTACTTGTTGGAT |
| 4119 | CTTTAGATCCTGGTTTTGCC |
| 4120 | ACTGGCTTTAGATCCTGGTT |
| 4121 | CAAAGAACTGGCTTTAGATC |
| 4122 | TGAAGTCACAAAGAACTGGC |
| 4123 | GCACAGGCTTGTGAAGTCAC |
| 4124 | GAAAAGAGCACAGGCTTGTG |
| 4125 | AATGGTATAGTGAAAAGAGC |
| 4126 | GGGAGGCAGAATGGTATAGT |
| 4127 | GCAGAGGGAGGCAGAATGGT |
| 4128 | TTGACTGCAGAGGGAGGCAG |
| 4129 | CAATTTCTGTAGTTTAGTTG |
| 4130 | CTGTGTGGAAGGACACAATT |
| 4131 | TTTTTAAAGCAAGCTGTGTG |
| 4132 | GCTTATGTATTTTTAAAGCA |
| 4133 | TCTTAGGTAGCTTATGTATT |
| 4134 | TTAGAGCTTTCTTAGGTAGC |
| 4135 | GTCTAAATTAGAGCTTTCTT |
| 4136 | AAAATGTCTAAATTAGAGCT |
| 4137 | CTAGCAGAGAGAAAAATGTC |
| 4138 | TAGAGACTAGCAGAGAGAAA |
| 4139 | TGGCATAGAGACTAGCAGAG |
| 4140 | GTTTTTTTGTGGCATAGAGA |
| 4141 | GGCTATGTTTTTTTGTGGCA |
| 4142 | TAATTGGCTATGTTTTTTTG |
| 4143 | CAATATAATTGGCTATGTTT |
| 4144 | GTTAACACAATATAATTGGC |
| 4145 | GAGTCAGTTAACACAATATA |
| 4146 | GCCAGCATGAGTCAGTTAAC |
| 4147 | ATAAATTGTGTAAAGCCAGC |
| 4148 | TGAAAATCAATATAAATTGT |
| 4149 | CCACCCTGAAAATCAATATA |
| 4150 | GCTGGTTATTACCACCCTGA |
| 4151 | CACAGAAGCTGGTTATTACC |
| 4152 | CCAATCACAGAAGCTGGTTA |
| 4153 | TGCACATGCACACCAATCAC |
| 4154 | CAGGAAGTCTTGCACATGCA |
| 4155 | GGTTAGGCAGGAAGTCTTGC |
| 4156 | ACAAGAAGGATGGGTTAGGC |
| 4157 | AGCTGAGCCACAAGAAGGAT |
| 4158 | TACAAAGCTGAGCCACAAGA |
| 4159 | AGTGTTTACAAAGCTGAGCC |
| 4160 | GGGCCAGTGTTTACAAAGCT |
| 4161 | CACCCAGGGCCAGTGTTTAC |
| 4162 | TCACGGCCACCCAGGGCCAG |
| 4163 | TCTGTTCACGGCCACCCAGG |
| 4164 | TAAGAACTTCTGTTCACGGC |
| 4165 | CTAAGATAAGAACTTCTGTT |
| 4166 | TGCAGTCTAAGATAAGAACT |
| 4167 | GTTGCATGCAGTCTAAGATA |
| 4168 | GATGAGTTGCATGCAGTCTA |
| 4169 | CAGTAGGATGAGTTGCATGC |
| 4170 | GCTTTCAGTGCCAGTAGGAT |
| 4171 | GAAGGGCTTTCAGTGCCAGT |
| 4172 | ACAAAGAAGGGCTTTCAGTG |
| 4173 | GTAGGTGAAGAAAGACAAAG |
| 4174 | TTGCTTAAGTAGGTGAAGAA |
| 4175 | GTAAATTTGCTTAAGTAGGT |
| 4176 | AAAGTGGCTTGTAAATTTGC |
| 4177 | GGCAAAAAGTGGCTTGTAAA |
| 4178 | GAAGAGGCAAAAAGTGGCTT |
| 4179 | TGAGAGAAGAGGCAAAAAGT |
| 4180 | GGCTGCTGAGAGAAGAGGCA |
| 4181 | AAGATGGCTGCTGAGAGAAG |
| 4182 | AAGGACCAAGATGGCTGCTG |
| 4183 | ATTGAAAGGACCAAGATGGC |
| 4184 | CAGGCAACATTGAAAGGACC |
| 4185 | GTGCACACAGGCAACATTGA |
| 4186 | TACAGCATGAGTGCACACAG |
| 4187 | ATAAATACAGCATGAGTGCA |
| 4188 | AGGTCATAAATACAGCATGA |
| 4189 | GAGGGACCAGGTCATAAATA |
| 4190 | TATGAGAGGGACCAGGTCAT |
| 4191 | CTCAAAGTATGAGAGGGACC |
| 4192 | GTTATATCTCAAAGTATGAG |
| 4193 | CTTGAGTTATATCTCAAAGT |
| 4194 | AGAAGGAAGGACTTGAGTTA |
| 4195 | TTCTCAAAGAAGGAAGGACT |
| 4196 | GTATGGATTCTCAAAGAAGG |
| 4197 | CCATCAGTATGGATTCTCAA |
| 4198 | GTGACCCATCAGTATGGATT |
| 4199 | GTATTTAAAAAGTGACCCAT |
| 4200 | GACCTTAAGTATTTAAAAAG |
| 4201 | TGATGCAGACCTTAAGTATT |
| 4202 | TATAATGTTTGATGCAGACC |
| 4203 | AATGTTATAATGTTTGATGC |
| 4204 | GAAGAACAGCAAATGTTATA |
| 4205 | AAAAAGAAGAACAGCAAATG |
| 4206 | GAATAAAAAAGAAGAACAGC |
| 4207 | GTATATAAACCAAGTAAGAA |
| 4208 | AGCTTAGTATATAAACCAAG |
| 4209 | GGATAATAAAGCTTAGTATA |
| 4210 | CTTAAAAGGATAATAAAGCT |
| 4211 | GAGAAAGTTTTAACCTTAAA |
| 4212 | TCCTTGAGAAAGTTTTAACC |
| 4213 | GTTAGTGTCCTTGAGAAAGT |
| 4214 | ACACATTACAGTTAGTGTCC |
| 4215 | CAAGTTGTACACATTACAGT |
| 4216 | ACGCTGGCTACCAAGTTGTA |
| 4217 | CTATGTCAAATGTTACGCTG |
| 4218 | CAAGACACTATGTCAAATGT |
| 4219 | TCTTTTTACAAGACACTATG |
| 4220 | GTATCTTCTTTTTACAAGAC |
| 4221 | AACTGATTGAGTATCTTCTT |
| 4222 | TCAAAAACTGATTGAGTATC |
| 4223 | AATCATTCAAAAACTGATTG |
| 4224 | TACTCTTTGAATCATTCAAA |
| 4225 | GCCATTATTTTACTCTTTGA |
| 4226 | AGAAGTTGCCATTATTTTAC |
| 4227 | GATAAAAGAAGTTGCCATTA |
| 4228 | CACTATGGAAGAGATAAAAG |
| 4229 | GATTGCACTATGGAAGAGAT |
| 4230 | AAAATATTGAAGATTGCACT |
| 4231 | GTTTAAAAAATATTGAAGAT |
| 4232 | CCAATAGTTGTTTAAAAAAT |
| 4233 | AATTCAGCCAATAGTTGTTT |
| 4234 | CACAATGGTAGAATTCAGCC |
| 4235 | CTAAACCACAATGGTAGAAT |
| 4236 | AAGCTCTAAACCACAATGGT |
| 4237 | TCTCAGAGAAAGCTCTAAAC |
| 4238 | CCTTAGCAAAAATGTCTCAG |
| 4239 | TCAAAACCTTAGCAAAAATG |
| 4240 | CCTGTTTCAAAACCTTAGCA |
| 4241 | TGGAACCTGTTTCAAAACCT |
| 4242 | GGACCAATTTGGAACCTGTT |
| 4243 | GTCACTGTGGACCAATTTGG |
| 4244 | CTTGGAGGTCACTGTGGACC |
| 4245 | TAGAGCCTTGGAGGTCACTG |
| 4246 | TCCTGTAGAGCCTTGGAGGT |
| 4247 | ACCACTCCTGTAGAGCCTTG |
| 4248 | CTTGAGGACCACTCCTGTAG |
| 4249 | TGAAGCTTGAGGACCACTCC |
| 4250 | ATGAGCTGAAGCTTGAGGAC |
| 4251 | GGCCCAATGAGCTGAAGCTT |
| 4252 | AGTGGGGCCCAATGAGCTGA |
| 4253 | CTGGGAGTGGGGCCCAATGA |
| 4254 | TTTCCTGAGACCTACTGAAT |
| 4255 | GGTCCTTTCCTGAGACCTAC |
| 4256 | GTTCTTGGTCCTTTCCTGAG |
| 4257 | GGAATGTATGTTCTTGGTCC |
| 4258 | CCAGTAGGAATGTATGTTCT |
| 4259 | ATTACCCAGTAGGAATGTAT |
| 4260 | TCCTCATTACCCAGTAGGAA |
| 4261 | AGCAGCAGCCTCCTCATTAC |
| 4262 | CTACTGGACCAGCAGCAGCC |
| 4263 | TGTGACTACTGGACCAGCAG |
| 4264 | GAAAGTGTGACTACTGGACC |
| 4265 | AGTGGCTCTGAAAGTGTGAC |
| 4266 | TCACAGGGCAGTGGCTCTGA |
| 4267 | CCATGAGCTCACAGGGCAGT |
| 4268 | GGAGTGCCATGAGCTCACAG |
| 4269 | ATCATGGAGTGCCATGAGCT |
| 4270 | TCAGTGTGGCGGGAATCATG |
| 4271 | GGGATTATGTCAGTGTGGCG |
| 4272 | AGCTGGGGATTATGTCAGTG |
| 4273 | ACTGAAGTCAGCTGGGGATT |
| 4274 | GCAAGACTGAAGTCAGCTGG |
| 4275 | ACCTGGAGTGGAGTAAGCAA |
| 4276 | GCTGAACCTGGAGTGGAGTA |
| 4277 | AAGGAAGCTGAACCTGGAGT |
| 4278 | GTATGTAAGGAAGCTGAACC |
| 4279 | ATGCCTGGTGTATGTAAGGA |
| 4280 | AGGAGCATGCCTGGTGTATG |
| 4281 | TGGGCAGGAGCATGCCTGGT |
| 4282 | AAAGCTCTGGGCAGGAGCAT |
| 4283 | AAACCACATGGGCAAAGCTC |
| 4284 | TGGGGAAACCACATGGGCAA |
| 4285 | GTATCTGGGGAAACCACATG |
| 4286 | ACATGGGTATCTGGGGAAAC |
| 4287 | CAAGTACATGGGTATCTGGG |
| 4288 | GTAAAGGAACAAGTACATGG |
| 4289 | GGGAGGTAAAGGAACAAGTA |
| 4290 | CCTGAGGGAGGTAAAGGAAC |
| 4291 | GGAATGACCTGAGGGAGGTA |
| 4292 | GGTGACATATGAGGAATGAC |
| 4293 | GAGATGGTGACATATGAGGA |
| 4294 | CCCACTGAGATGGTGACATA |
| 4295 | AAGGGCCCACTGAGATGGTG |
| 4296 | TATGGAAGGGCCCACTGAGA |
| 4297 | TGGGTTTTATGGAAGGGCCC |
| 4298 | TTTTTAACTGGGTTTTATGG |
| 4299 | TGTTGCCATTTTTAACTGGG |
| 4300 | GAGAAGGATGTTGCCATTTT |
| 4301 | TGAGGGGAGAAGGATGTTGC |
| 4302 | GCGTGTGAGGGGAGAAGGAT |
| 4303 | AGTTAGCGTGTGAGGGGAGA |
| 4304 | TGTGTGTCAGTTAGCGTGTG |
| 4305 | ATGTGTGTGTGTCAGTTAGC |
| 4306 | TGCTGTGTGAGAGAGAGAGT |
| 4307 | GTGTACGTATGCTGTGTGAG |
| 4308 | GGAGTGTGTGTACGTATGCT |
| 4309 | TGATAGGGAGTGTGTGTACG |
| 4310 | GCAGGAAAGGGATATGATAG |
| 4311 | GCATTGCAGGAAAGGGATAT |
| 4312 | TGGAGAAAAGCATTGCAGGA |
| 4313 | TGGTATGGAGAAAAGCATTG |
| 4314 | TCAGATGGTGTAAATGGTAT |
| 4315 | GTGTGGAAGGACACAATT |
| 4316 | GGACACAATTTCTGTAGT |
| 4317 | TGTAGTTTAGTTGACTGC |
| 4318 | AGTTTAGTTGACTGCAGA |
| 4319 | TTAGTTGACTGCAGAGGG |
| 4320 | TAGTTGACTGCAGAGGGA |
| 4321 | TAAAGCAAGCTGTGTGGAA |
| 4322 | GCTGTGTGGAAGGACACAA |
| 4323 | CTGTGTGGAAGGACACAAT |
| 4324 | TGTGTGGAAGGACACAATT |
| 4325 | GTGTGGAAGGACACAATTT |
| 4326 | TGGAAGGACACAATTTCTG |
| 4327 | GGAAGGACACAATTTCTGT |
| 4328 | GAAGGACACAATTTCTGTA |
| 4329 | AAGGACACAATTTCTGTAG |
| 4330 | AGGACACAATTTCTGTAGT |
| 4331 | GGACACAATTTCTGTAGTT |
| 4332 | CACAATTTCTGTAGTTTAG |
| 4333 | ACAATTTCTGTAGTTTAGT |
| 4334 | TTTCTGTAGTTTAGTTGAC |
| 4335 | TCTGTAGTTTAGTTGACTG |
| 4336 | CTGTAGTTTAGTTGACTGC |
| 4337 | TGTAGTTTAGTTGACTGCA |
| 4338 | TAGTTTAGTTGACTGCAGA |
| 4339 | AGTTTAGTTGACTGCAGAG |
| 4340 | GTTTAGTTGACTGCAGAGG |
| 4341 | TTTAGTTGACTGCAGAGGG |
| 4342 | TTAGTTGACTGCAGAGGGA |
| 4343 | TAGTTGACTGCAGAGGGAG |
| 4344 | AGTTGACTGCAGAGGGAGG |
| 4345 | TATTTTTAAAGCAAGCTGTG |
| 4346 | ATTTTTAAAGCAAGCTGTGT |
| 4347 | TTAAAGCAAGCTGTGTGGAA |
| 4348 | TAAAGCAAGCTGTGTGGAAG |
| 4349 | AAGCAAGCTGTGTGGAAGGA |
| 4350 | AGCAAGCTGTGTGGAAGGAC |
| 4351 | AGCTGTGTGGAAGGACACAA |
| 4352 | GCTGTGTGGAAGGACACAAT |
| 4353 | TGTGTGGAAGGACACAATTT |
| 4354 | GTGTGGAAGGACACAATTTC |
| 4355 | TGTGGAAGGACACAATTTCT |
| 4356 | GTGGAAGGACACAATTTCTG |
| 4357 | TGGAAGGACACAATTTCTGT |
| 4358 | GGAAGGACACAATTTCTGTA |
| 4359 | GAAGGACACAATTTCTGTAG |
| 4360 | AAGGACACAATTTCTGTAGT |
| 4361 | AGGACACAATTTCTGTAGTT |
| 4362 | GGACACAATTTCTGTAGTTT |
| 4363 | GACACAATTTCTGTAGTTTA |
| 4364 | ACACAATTTCTGTAGTTTAG |
| 4365 | CACAATTTCTGTAGTTTAGT |
| 4366 | ACAATTTCTGTAGTTTAGTT |
| 4367 | AATTTCTGTAGTTTAGTTGA |
| 4368 | ATTTCTGTAGTTTAGTTGAC |
| 4369 | TTTCTGTAGTTTAGTTGACT |
| 4370 | TTCTGTAGTTTAGTTGACTG |
| 4371 | TCTGTAGTTTAGTTGACTGC |
| 4372 | CTGTAGTTTAGTTGACTGCA |
| 4373 | TGTAGTTTAGTTGACTGCAG |
| 4374 | GTAGTTTAGTTGACTGCAGA |
| 4375 | TAGTTTAGTTGACTGCAGAG |
| 4376 | AGTTTAGTTGACTGCAGAGG |
| 4377 | GTTTAGTTGACTGCAGAGGG |
| 4378 | TTTAGTTGACTGCAGAGGGA |
| 4379 | TTAGTTGACTGCAGAGGGAG |
| 4380 | TAGTTGACTGCAGAGGGAGG |
| 4381 | AGTTGACTGCAGAGGGAGGC |
| 4382 | ATGTATTTTTAAAGCAAGCTG |
| 4383 | GTATTTTTAAAGCAAGCTGTG |
| 4384 | TATTTTTAAAGCAAGCTGTGT |
| 4385 | ATTTTTAAAGCAAGCTGTGTG |
| 4386 | TTTTTAAAGCAAGCTGTGTGG |
| 4387 | TTTAAAGCAAGCTGTGTGGAA |
| 4388 | TTAAAGCAAGCTGTGTGGAAG |
| 4389 | TAAAGCAAGCTGTGTGGAAGG |
| 4390 | AAAGCAAGCTGTGTGGAAGGA |
| 4391 | AAGCAAGCTGTGTGGAAGGAC |
| 4392 | AGCAAGCTGTGTGGAAGGACA |
| 4393 | AAGCTGTGTGGAAGGACACAA |
| 4394 | AGCTGTGTGGAAGGACACAAT |
| 4395 | GCTGTGTGGAAGGACACAATT |
| 4396 | CTGTGTGGAAGGACACAATTT |
| 4397 | TGTGTGGAAGGACACAATTTC |
| 4398 | GTGTGGAAGGACACAATTTCT |
| 4399 | TGTGGAAGGACACAATTTCTG |
| 4400 | GTGGAAGGACACAATTTCTGT |
| 4401 | TGGAAGGACACAATTTCTGTA |
| 4402 | GGAAGGACACAATTTCTGTAG |
| 4403 | GAAGGACACAATTTCTGTAGT |
| 4404 | AAGGACACAATTTCTGTAGTT |
| 4405 | AGGACACAATTTCTGTAGTTT |
| 4406 | GGACACAATTTCTGTAGTTTA |
| 4407 | GACACAATTTCTGTAGTTTAG |
| 4408 | ACACAATTTCTGTAGTTTAGT |
| 4409 | CACAATTTCTGTAGTTTAGTT |
| 4410 | ACAATTTCTGTAGTTTAGTTG |
| 4411 | CAATTTCTGTAGTTTAGTTGA |
| 4412 | AATTTCTGTAGTTTAGTTGAC |
| 4413 | ATTTCTGTAGTTTAGTTGACT |
| 4414 | TTTCTGTAGTTTAGTTGACTG |
| 4415 | TTCTGTAGTTTAGTTGACTGC |
| 4416 | TCTGTAGTTTAGTTGACTGCA |
| 4417 | CTGTAGTTTAGTTGACTGCAG |
| 4418 | TGTAGTTTAGTTGACTGCAGA |
| 4419 | GTAGTTTAGTTGACTGCAGAG |
| 4420 | TAGTTTAGTTGACTGCAGAGG |
| 4421 | AGTTTAGTTGACTGCAGAGGG |
| 4422 | GTTTAGTTGACTGCAGAGGGA |
| 4423 | TTTAGTTGACTGCAGAGGGAG |
| 4424 | TTAGTTGACTGCAGAGGGAGG |
| 4425 | TAGTTGACTGCAGAGGGAGGC |
| 4426 | AGTTGACTGCAGAGGGAGGCA |
| 4427 | GTTGACTGCAGAGGGAGGCAG |
| 4428 | ATGTATTTTTAAAGCAAGCTGT |
| 4429 | TGTATTTTTAAAGCAAGCTGTG |
| 4430 | GTATTTTTAAAGCAAGCTGTGT |
| 4431 | TATTTTTAAAGCAAGCTGTGTG |
| 4432 | ATTTTTAAAGCAAGCTGTGTGG |
| 4433 | TTTTTAAAGCAAGCTGTGTGGA |
| 4434 | TTTTAAAGCAAGCTGTGTGGAA |
| 4435 | TTTAAAGCAAGCTGTGTGGAAG |
| 4436 | TTAAAGCAAGCTGTGTGGAAGG |
| 4437 | TAAAGCAAGCTGTGTGGAAGGA |
| 4438 | AAAGCAAGCTGTGTGGAAGGAC |
| 4439 | AAGCAAGCTGTGTGGAAGGACA |
| 4440 | AGCAAGCTGTGTGGAAGGACAC |
| 4441 | CAAGCTGTGTGGAAGGACACAA |
| 4442 | AAGCTGTGTGGAAGGACACAAT |
| 4443 | AGCTGTGTGGAAGGACACAATT |
| 4444 | GCTGTGTGGAAGGACACAATTT |
| 4445 | CTGTGTGGAAGGACACAATTTC |
| 4446 | TGTGTGGAAGGACACAATTTCT |
| 4447 | GTGTGGAAGGACACAATTTCTG |
| 4448 | TGTGGAAGGACACAATTTCTGT |
| 4449 | GTGGAAGGACACAATTTCTGTA |
| 4450 | TGGAAGGACACAATTTCTGTAG |
| 4451 | GGAAGGACACAATTTCTGTAGT |
| 4452 | GAAGGACACAATTTCTGTAGTT |
| 4453 | AAGGACACAATTTCTGTAGTTT |
| 4454 | AGGACACAATTTCTGTAGTTTA |
| 4455 | GGACACAATTTCTGTAGTTTAG |
| 4456 | GACACAATTTCTGTAGTTTAGT |
| 4457 | ACACAATTTCTGTAGTTTAGTT |
| 4458 | CACAATTTCTGTAGTTTAGTTG |
| 4459 | ACAATTTCTGTAGTTTAGTTGA |
| 4460 | CAATTTCTGTAGTTTAGTTGAC |
| 4461 | AATTTCTGTAGTTTAGTTGACT |
| 4462 | ATTTCTGTAGTTTAGTTGACTG |
| 4463 | TTTCTGTAGTTTAGTTGACTGC |
| 4464 | TTCTGTAGTTTAGTTGACTGCA |
| 4465 | TCTGTAGTTTAGTTGACTGCAG |
| 4466 | CTGTAGTTTAGTTGACTGCAGA |
| 4467 | TGTAGTTTAGTTGACTGCAGAG |
| 4468 | GTAGTTTAGTTGACTGCAGAGG |
| 4469 | TAGTTTAGTTGACTGCAGAGGG |
| 4470 | AGTTTAGTTGACTGCAGAGGGA |
| 4471 | GTTTAGTTGACTGCAGAGGGAG |
| 4472 | TTTAGTTGACTGCAGAGGGAGG |
| 4473 | TTAGTTGACTGCAGAGGGAGGC |
| 4474 | TAGTTGACTGCAGAGGGAGGCA |
| 4475 | AGTTGACTGCAGAGGGAGGCAG |
| 4476 | ATGTATTTTTAAAGCAAGCTGTG |
| 4477 | TGTATTTTTAAAGCAAGCTGTGT |
| 4478 | GTATTTTTAAAGCAAGCTGTGTG |
| 4479 | TATTTTTAAAGCAAGCTGTGTGG |
| 4480 | ATTTTTAAAGCAAGCTGTGTGGA |
| 4481 | TTTTTAAAGCAAGCTGTGTGGAA |
| 4482 | TTTTAAAGCAAGCTGTGTGGAAG |
| 4483 | TTTAAAGCAAGCTGTGTGGAAGG |
| 4484 | TTAAAGCAAGCTGTGTGGAAGGA |
| 4485 | TAAAGCAAGCTGTGTGGAAGGAC |
| 4486 | AAAGCAAGCTGTGTGGAAGGACA |
| 4487 | AAGCAAGCTGTGTGGAAGGACAC |
| 4488 | AGCAAGCTGTGTGGAAGGACACA |
| 4489 | GCAAGCTGTGTGGAAGGACACAA |
| 4490 | CAAGCTGTGTGGAAGGACACAAT |
| 4491 | AAGCTGTGTGGAAGGACACAATT |
| 4492 | AGCTGTGTGGAAGGACACAATTT |
| 4493 | GCTGTGTGGAAGGACACAATTTC |
| 4494 | CTGTGTGGAAGGACACAATTTCT |
| 4495 | TGTGTGGAAGGACACAATTTCTG |
| 4496 | GTGTGGAAGGACACAATTTCTGT |
| 4497 | TGTGGAAGGACACAATTTCTGTA |
| 4498 | GTGGAAGGACACAATTTCTGTAG |
| 4499 | TGGAAGGACACAATTTCTGTAGT |
| 4500 | GGAAGGACACAATTTCTGTAGTT |
| 4501 | GAAGGACACAATTTCTGTAGTTT |
| 4502 | AAGGACACAATTTCTGTAGTTTA |
| 4503 | AGGACACAATTTCTGTAGTTTAG |
| 4504 | GGACACAATTTCTGTAGTTTAGT |
| 4505 | GACACAATTTCTGTAGTTTAGTT |
| 4506 | ACACAATTTCTGTAGTTTAGTTG |
| 4507 | CACAATTTCTGTAGTTTAGTTGA |
| 4508 | ACAATTTCTGTAGTTTAGTTGAC |
| 4509 | CAATTTCTGTAGTTTAGTTGACT |
| 4510 | AATTTCTGTAGTTTAGTTGACTG |
| 4511 | ATTTCTGTAGTTTAGTTGACTGC |
| 4512 | TTTCTGTAGTTTAGTTGACTGCA |
| 4513 | TTCTGTAGTTTAGTTGACTGCAG |
| 4514 | TCTGTAGTTTAGTTGACTGCAGA |
| 4515 | CTGTAGTTTAGTTGACTGCAGAG |
| 4516 | TGTAGTTTAGTTGACTGCAGAGG |
| 4517 | GTAGTTTAGTTGACTGCAGAGGG |
| 4518 | TAGTTTAGTTGACTGCAGAGGGA |
| 4519 | AGTTTAGTTGACTGCAGAGGGAG |
| 4520 | GTTTAGTTGACTGCAGAGGGAGG |
| 4521 | TTTAGTTGACTGCAGAGGGAGGC |
| 4522 | TTAGTTGACTGCAGAGGGAGGCA |
| 4523 | TAGTTGACTGCAGAGGGAGGCAG |
| 4524 | ATGTATTTTTAAAGCAAGCTGTGT |
| 4525 | TGTATTTTTAAAGCAAGCTGTGTG |
| 4526 | GTATTTTTAAAGCAAGCTGTGTGG |
| 4527 | TATTTTTAAAGCAAGCTGTGTGGA |
| 4528 | ATTTTTAAAGCAAGCTGTGTGGAA |
| 4529 | TTTTTAAAGCAAGCTGTGTGGAAG |
| 4530 | TTTTAAAGCAAGCTGTGTGGAAGG |
| 4531 | TTTAAAGCAAGCTGTGTGGAAGGA |
| 4532 | TTAAAGCAAGCTGTGTGGAAGGAC |
| 4533 | TAAAGCAAGCTGTGTGGAAGGACA |
| 4534 | AAAGCAAGCTGTGTGGAAGGACAC |
| 4535 | AAGCAAGCTGTGTGGAAGGACACA |
| 4536 | AGCAAGCTGTGTGGAAGGACACAA |
| 4537 | GCAAGCTGTGTGGAAGGACACAAT |
| 4538 | CAAGCTGTGTGGAAGGACACAATT |
| 4539 | AAGCTGTGTGGAAGGACACAATTT |
| 4540 | AGCTGTGTGGAAGGACACAATTTC |
| 4541 | GCTGTGTGGAAGGACACAATTTCT |
| 4542 | CTGTGTGGAAGGACACAATTTCTG |
| 4543 | TGTGTGGAAGGACACAATTTCTGT |
| 4544 | GTGTGGAAGGACACAATTTCTGTA |
| 4545 | TGTGGAAGGACACAATTTCTGTAG |
| 4546 | GTGGAAGGACACAATTTCTGTAGT |
| 4547 | TGGAAGGACACAATTTCTGTAGTT |
| 4548 | GGAAGGACACAATTTCTGTAGTTT |
| 4549 | GAAGGACACAATTTCTGTAGTTTA |
| 4550 | AAGGACACAATTTCTGTAGTTTAG |
| 4551 | AGGACACAATTTCTGTAGTTTAGT |
| 4552 | GGACACAATTTCTGTAGTTTAGTT |
| 4553 | GACACAATTTCTGTAGTTTAGTTG |
| 4554 | ACACAATTTCTGTAGTTTAGTTGA |
| 4555 | CACAATTTCTGTAGTTTAGTTGAC |
| 4556 | ACAATTTCTGTAGTTTAGTTGACT |
| 4557 | CAATTTCTGTAGTTTAGTTGACTG |
| 4558 | AATTTCTGTAGTTTAGTTGACTGC |
| 4559 | ATTTCTGTAGTTTAGTTGACTGCA |
| 4560 | TTTCTGTAGTTTAGTTGACTGCAG |
| 4561 | TTCTGTAGTTTAGTTGACTGCAGA |
| 4562 | TCTGTAGTTTAGTTGACTGCAGAG |
| 4563 | CTGTAGTTTAGTTGACTGCAGAGG |
| 4564 | TGTAGTTTAGTTGACTGCAGAGGG |
| 4565 | GTAGTTTAGTTGACTGCAGAGGGA |
| 4566 | TAGTTTAGTTGACTGCAGAGGGAG |
| 4567 | AGTTTAGTTGACTGCAGAGGGAGG |
| 4568 | GTTTAGTTGACTGCAGAGGGAGGC |
| 4569 | TTTAGTTGACTGCAGAGGGAGGCA |
| 4570 | TTAGTTGACTGCAGAGGGAGGCAG |
| 4571 | ATGTATTTTTAAAGCAAGCTGTGTG |
| 4572 | TGTATTTTTAAAGCAAGCTGTGTGG |
| 4573 | GTATTTTTAAAGCAAGCTGTGTGGA |
| 4574 | TATTTTTAAAGCAAGCTGTGTGGAA |
| 4575 | ATTTTTAAAGCAAGCTGTGTGGAAG |
| 4576 | TTTTTAAAGCAAGCTGTGTGGAAGG |
| 4577 | TTTTAAAGCAAGCTGTGTGGAAGGA |
| 4578 | TTTAAAGCAAGCTGTGTGGAAGGAC |
| 4579 | TTAAAGCAAGCTGTGTGGAAGGACA |
| 4580 | TAAAGCAAGCTGTGTGGAAGGACAC |
| 4581 | AAAGCAAGCTGTGTGGAAGGACACA |
| 4582 | AAGCAAGCTGTGTGGAAGGACACAA |
| 4583 | AGCAAGCTGTGTGGAAGGACACAAT |
| 4584 | GCAAGCTGTGTGGAAGGACACAATT |
| 4585 | CAAGCTGTGTGGAAGGACACAATTT |
| 4586 | AAGCTGTGTGGAAGGACACAATTTC |
| 4587 | AGCTGTGTGGAAGGACACAATTTCT |
| 4588 | GCTGTGTGGAAGGACACAATTTCTG |
| 4589 | CTGTGTGGAAGGACACAATTTCTGT |
| 4590 | TGTGTGGAAGGACACAATTTCTGTA |
| 4591 | GTGTGGAAGGACACAATTTCTGTAG |
| 4592 | TGTGGAAGGACACAATTTCTGTAGT |
| 4593 | GTGGAAGGACACAATTTCTGTAGTT |
| 4594 | TGGAAGGACACAATTTCTGTAGTTT |
| 4595 | GGAAGGACACAATTTCTGTAGTTTA |
| 4596 | GAAGGACACAATTTCTGTAGTTTAG |
| 4597 | AAGGACACAATTTCTGTAGTTTAGT |
| 4598 | AGGACACAATTTCTGTAGTTTAGTT |
| 4599 | GGACACAATTTCTGTAGTTTAGTTG |
| 4600 | GACACAATTTCTGTAGTTTAGTTGA |
| 4601 | ACACAATTTCTGTAGTTTAGTTGAC |
| 4602 | CACAATTTCTGTAGTTTAGTTGACT |
| 4603 | ACAATTTCTGTAGTTTAGTTGACTG |
| 4604 | CAATTTCTGTAGTTTAGTTGACTGC |
| 4605 | AATTTCTGTAGTTTAGTTGACTGCA |
| 4606 | ATTTCTGTAGTTTAGTTGACTGCAG |
| 4607 | TTTCTGTAGTTTAGTTGACTGCAGA |
| 4608 | TTCTGTAGTTTAGTTGACTGCAGAG |
| 4609 | TCTGTAGTTTAGTTGACTGCAGAGG |
| 4610 | CTGTAGTTTAGTTGACTGCAGAGGG |
| 4611 | TGTAGTTTAGTTGACTGCAGAGGGA |
| 4612 | GTAGTTTAGTTGACTGCAGAGGGAG |
| 4613 | TAGTTTAGTTGACTGCAGAGGGAGG |
| 4614 | AGTTTAGTTGACTGCAGAGGGAGGC |
| 4615 | GTTTAGTTGACTGCAGAGGGAGGCA |
| 4616 | TTTAGTTGACTGCAGAGGGAGGCAG |
| 4617 | ATGTATTTTTAAAGCAAGCTGTGTGG |
| 4618 | TGTATTTTTAAAGCAAGCTGTGTGGA |
| 4619 | GTATTTTTAAAGCAAGCTGTGTGGAA |
| 4620 | TATTTTTAAAGCAAGCTGTGTGGAAG |
| 4621 | ATTTTTAAAGCAAGCTGTGTGGAAGG |
| 4622 | TTTTTAAAGCAAGCTGTGTGGAAGGA |
| 4623 | TTTTAAAGCAAGCTGTGTGGAAGGAC |
| 4624 | TTTAAAGCAAGCTGTGTGGAAGGACA |
| 4625 | TTAAAGCAAGCTGTGTGGAAGGACAC |
| 4626 | TAAAGCAAGCTGTGTGGAAGGACACA |
| 4627 | AAAGCAAGCTGTGTGGAAGGACACAA |
| 4628 | AAGCAAGCTGTGTGGAAGGACACAAT |
| 4629 | AGCAAGCTGTGTGGAAGGACACAATT |
| 4630 | GCAAGCTGTGTGGAAGGACACAATTT |
| 4631 | CAAGCTGTGTGGAAGGACACAATTTC |
| 4632 | AAGCTGTGTGGAAGGACACAATTTCT |
| 4633 | AGCTGTGTGGAAGGACACAATTTCTG |
| 4634 | GCTGTGTGGAAGGACACAATTTCTGT |
| 4635 | CTGTGTGGAAGGACACAATTTCTGTA |
| 4636 | TGTGTGGAAGGACACAATTTCTGTAG |
| 4637 | GTGTGGAAGGACACAATTTCTGTAGT |
| 4638 | TGTGGAAGGACACAATTTCTGTAGTT |
| 4639 | GTGGAAGGACACAATTTCTGTAGTTT |
| 4640 | TGGAAGGACACAATTTCTGTAGTTTA |
| 4641 | GGAAGGACACAATTTCTGTAGTTTAG |
| 4642 | GAAGGACACAATTTCTGTAGTTTAGT |
| 4643 | AAGGACACAATTTCTGTAGTTTAGTT |
| 4644 | AGGACACAATTTCTGTAGTTTAGTTG |
| 4645 | GGACACAATTTCTGTAGTTTAGTTGA |
| 4646 | GACACAATTTCTGTAGTTTAGTTGAC |
| 4647 | ACACAATTTCTGTAGTTTAGTTGACT |
| 4648 | CACAATTTCTGTAGTTTAGTTGACTG |
| 4649 | ACAATTTCTGTAGTTTAGTTGACTGC |
| 4650 | CAATTTCTGTAGTTTAGTTGACTGCA |
| 4651 | AATTTCTGTAGTTTAGTTGACTGCAG |
| 4652 | ATTTCTGTAGTTTAGTTGACTGCAGA |
| 4653 | TTTCTGTAGTTTAGTTGACTGCAGAG |
| 4654 | TTCTGTAGTTTAGTTGACTGCAGAGG |
| 4655 | TCTGTAGTTTAGTTGACTGCAGAGGG |
| 4656 | CTGTAGTTTAGTTGACTGCAGAGGGA |
| 4657 | TGTAGTTTAGTTGACTGCAGAGGGAG |
| 4658 | GTAGTTTAGTTGACTGCAGAGGGAGG |
| 4659 | TAGTTTAGTTGACTGCAGAGGGAGGC |
| 4660 | AGTTTAGTTGACTGCAGAGGGAGGCA |
| 4661 | GTTTAGTTGACTGCAGAGGGAGGCAG |
| 4662 | AGCTCTACCATCCTCAAC |
| 4663 | CAGGCTGACAGAAGCTCTA |
| 4664 | AGGCTGACAGAAGCTCTAC |
| 4665 | GACAGAAGCTCTACCATCC |
| 4666 | ACAGAAGCTCTACCATCCT |
| 4667 | AGAAGCTCTACCATCCTCA |
| 4668 | AAGCTCTACCATCCTCAAC |
| 4669 | AGCTCTACCATCCTCAACA |
| 4670 | CATGTGGCTTTTAAGGTCT |
| 4671 | TGTGGCTTTTAAGGTCTCC |
| 4672 | CCCAGGCTGACAGAAGCTCT |
| 4673 | CCAGGCTGACAGAAGCTCTA |
| 4674 | CAGGCTGACAGAAGCTCTAC |
| 4675 | GGCTGACAGAAGCTCTACCA |
| 4676 | GCTGACAGAAGCTCTACCAT |
| 4677 | TGACAGAAGCTCTACCATCC |
| 4678 | GACAGAAGCTCTACCATCCT |
| 4679 | ACAGAAGCTCTACCATCCTC |
| 4680 | CAGAAGCTCTACCATCCTCA |
| 4681 | AGAAGCTCTACCATCCTCAA |
| 4682 | AAGCTCTACCATCCTCAACA |
| 4683 | AGCTCTACCATCCTCAACAT |
| 4684 | GCTCTACCATCCTCAACATG |
| 4685 | ACATGTGGCTTTTAAGGTCT |
| 4686 | ATGTGGCTTTTAAGGTCTCC |
| 4687 | CAGAGACCCAGGCTGACAGAA |
| 4688 | AGAGACCCAGGCTGACAGAAG |
| 4689 | GAGACCCAGGCTGACAGAAGC |
| 4690 | ACCCAGGCTGACAGAAGCTCT |
| 4691 | CCCAGGCTGACAGAAGCTCTA |
| 4692 | CCAGGCTGACAGAAGCTCTAC |
| 4693 | CAGGCTGACAGAAGCTCTACC |
| 4694 | AGGCTGACAGAAGCTCTACCA |
| 4695 | GGCTGACAGAAGCTCTACCAT |
| 4696 | GCTGACAGAAGCTCTACCATC |
| 4697 | CTGACAGAAGCTCTACCATCC |
| 4698 | TGACAGAAGCTCTACCATCCT |
| 4699 | GACAGAAGCTCTACCATCCTC |
| 4700 | ACAGAAGCTCTACCATCCTCA |
| 4701 | CAGAAGCTCTACCATCCTCAA |
| 4702 | AGAAGCTCTACCATCCTCAAC |
| 4703 | GAAGCTCTACCATCCTCAACA |
| 4704 | AAGCTCTACCATCCTCAACAT |
| 4705 | AGCTCTACCATCCTCAACATG |
| 4706 | GCTCTACCATCCTCAACATGT |
| 4707 | TCCTCAACATGTGGCTTTTAA |
| 4708 | CCTCAACATGTGGCTTTTAAG |
| 4709 | AACATGTGGCTTTTAAGGTCT |
| 4710 | ACATGTGGCTTTTAAGGTCTC |
| 4711 | CATGTGGCTTTTAAGGTCTCC |
| 4712 | TTCAGAGACCCAGGCTGACAGA |
| 4713 | TCAGAGACCCAGGCTGACAGAA |
| 4714 | CAGAGACCCAGGCTGACAGAAG |
| 4715 | AGAGACCCAGGCTGACAGAAGC |
| 4716 | GAGACCCAGGCTGACAGAAGCT |
| 4717 | AGACCCAGGCTGACAGAAGCTC |
| 4718 | GACCCAGGCTGACAGAAGCTCT |
| 4719 | ACCCAGGCTGACAGAAGCTCTA |
| 4720 | CCCAGGCTGACAGAAGCTCTAC |
| 4721 | CCAGGCTGACAGAAGCTCTACC |
| 4722 | CAGGCTGACAGAAGCTCTACCA |
| 4723 | AGGCTGACAGAAGCTCTACCAT |
| 4724 | GGCTGACAGAAGCTCTACCATC |
| 4725 | GCTGACAGAAGCTCTACCATCC |
| 4726 | CTGACAGAAGCTCTACCATCCT |
| 4727 | TGACAGAAGCTCTACCATCCTC |
| 4728 | GACAGAAGCTCTACCATCCTCA |
| 4729 | ACAGAAGCTCTACCATCCTCAA |
| 4730 | CAGAAGCTCTACCATCCTCAAC |
| 4731 | AGAAGCTCTACCATCCTCAACA |
| 4732 | GAAGCTCTACCATCCTCAACAT |
| 4733 | AAGCTCTACCATCCTCAACATG |
| 4734 | AGCTCTACCATCCTCAACATGT |
| 4735 | GCTCTACCATCCTCAACATGTG |
| 4736 | ATCCTCAACATGTGGCTTTTAA |
| 4737 | TCCTCAACATGTGGCTTTTAAG |
| 4738 | CCTCAACATGTGGCTTTTAAGG |
| 4739 | CAACATGTGGCTTTTAAGGTCT |
| 4740 | AACATGTGGCTTTTAAGGTCTC |
| 4741 | ACATGTGGCTTTTAAGGTCTCC |
| 4742 | GTCATTCAGAGACCCAGGCTGAC |
| 4743 | ATTCAGAGACCCAGGCTGACAGA |
| 4744 | TTCAGAGACCCAGGCTGACAGAA |
| 4745 | TCAGAGACCCAGGCTGACAGAAG |
| 4746 | CAGAGACCCAGGCTGACAGAAGC |
| 4747 | AGAGACCCAGGCTGACAGAAGCT |
| 4748 | GAGACCCAGGCTGACAGAAGCTC |
| 4749 | AGACCCAGGCTGACAGAAGCTCT |
| 4750 | GACCCAGGCTGACAGAAGCTCTA |
| 4751 | ACCCAGGCTGACAGAAGCTCTAC |
| 4752 | CCCAGGCTGACAGAAGCTCTACC |
| 4753 | CCAGGCTGACAGAAGCTCTACCA |
| 4754 | CAGGCTGACAGAAGCTCTACCAT |
| 4755 | AGGCTGACAGAAGCTCTACCATC |
| 4756 | GGCTGACAGAAGCTCTACCATCC |
| 4757 | GCTGACAGAAGCTCTACCATCCT |
| 4758 | CTGACAGAAGCTCTACCATCCTC |
| 4759 | TGACAGAAGCTCTACCATCCTCA |
| 4760 | GACAGAAGCTCTACCATCCTCAA |
| 4761 | ACAGAAGCTCTACCATCCTCAAC |
| 4762 | CAGAAGCTCTACCATCCTCAACA |
| 4763 | AGAAGCTCTACCATCCTCAACAT |
| 4764 | GAAGCTCTACCATCCTCAACATG |
| 4765 | AAGCTCTACCATCCTCAACATGT |
| 4766 | AGCTCTACCATCCTCAACATGTG |
| 4767 | GCTCTACCATCCTCAACATGTGG |
| 4768 | CATCCTCAACATGTGGCTTTTAA |
| 4769 | ATCCTCAACATGTGGCTTTTAAG |
| 4770 | TCCTCAACATGTGGCTTTTAAGG |
| 4771 | CCTCAACATGTGGCTTTTAAGGT |
| 4772 | TCAACATGTGGCTTTTAAGGTCT |
| 4773 | CAACATGTGGCTTTTAAGGTCTC |
| 4774 | AACATGTGGCTTTTAAGGTCTCC |
| 4775 | TGCAGTCATTCAGAGACCCAGGCT |
| 4776 | GCAGTCATTCAGAGACCCAGGCTG |
| 4777 | AGTCATTCAGAGACCCAGGCTGAC |
| 4778 | GTCATTCAGAGACCCAGGCTGACA |
| 4779 | CATTCAGAGACCCAGGCTGACAGA |
| 4780 | ATTCAGAGACCCAGGCTGACAGAA |
| 4781 | TTCAGAGACCCAGGCTGACAGAAG |
| 4782 | TCAGAGACCCAGGCTGACAGAAGC |
| 4783 | CAGAGACCCAGGCTGACAGAAGCT |
| 4784 | AGAGACCCAGGCTGACAGAAGCTC |
| 4785 | GAGACCCAGGCTGACAGAAGCTCT |
| 4786 | AGACCCAGGCTGACAGAAGCTCTA |
| 4787 | GACCCAGGCTGACAGAAGCTCTAC |
| 4788 | ACCCAGGCTGACAGAAGCTCTACC |
| 4789 | CCCAGGCTGACAGAAGCTCTACCA |
| 4790 | CCAGGCTGACAGAAGCTCTACCAT |
| 4791 | CAGGCTGACAGAAGCTCTACCATC |
| 4792 | AGGCTGACAGAAGCTCTACCATCC |
| 4793 | GGCTGACAGAAGCTCTACCATCCT |
| 4794 | GCTGACAGAAGCTCTACCATCCTC |
| 4795 | CTGACAGAAGCTCTACCATCCTCA |
| 4796 | TGACAGAAGCTCTACCATCCTCAA |
| 4797 | GACAGAAGCTCTACCATCCTCAAC |
| 4798 | ACAGAAGCTCTACCATCCTCAACA |
| 4799 | CAGAAGCTCTACCATCCTCAACAT |
| 4800 | AGAAGCTCTACCATCCTCAACATG |
| 4801 | GAAGCTCTACCATCCTCAACATGT |
| 4802 | AAGCTCTACCATCCTCAACATGTG |
| 4803 | AGCTCTACCATCCTCAACATGTGG |
| 4804 | GCTCTACCATCCTCAACATGTGGC |
| 4805 | ACCATCCTCAACATGTGGCTTTTA |
| 4806 | CCATCCTCAACATGTGGCTTTTAA |
| 4807 | CATCCTCAACATGTGGCTTTTAAG |
| 4808 | ATCCTCAACATGTGGCTTTTAAGG |
| 4809 | TCCTCAACATGTGGCTTTTAAGGT |
| 4810 | CCTCAACATGTGGCTTTTAAGGTC |
| 4811 | CTCAACATGTGGCTTTTAAGGTCT |
| 4812 | TCAACATGTGGCTTTTAAGGTCTC |
| 4813 | CAACATGTGGCTTTTAAGGTCTCC |
| 4814 | ATGCAGTCATTCAGAGACCCAGGCT |
| 4815 | TGCAGTCATTCAGAGACCCAGGCTG |
| 4816 | GCAGTCATTCAGAGACCCAGGCTGA |
| 4817 | CAGTCATTCAGAGACCCAGGCTGAC |
| 4818 | AGTCATTCAGAGACCCAGGCTGACA |
| 4819 | GTCATTCAGAGACCCAGGCTGACAG |
| 4820 | TCATTCAGAGACCCAGGCTGACAGA |
| 4821 | CATTCAGAGACCCAGGCTGACAGAA |
| 4822 | ATTCAGAGACCCAGGCTGACAGAAG |
| 4823 | TTCAGAGACCCAGGCTGACAGAAGC |
| 4824 | TCAGAGACCCAGGCTGACAGAAGCT |
| 4825 | CAGAGACCCAGGCTGACAGAAGCTC |
| 4826 | AGAGACCCAGGCTGACAGAAGCTCT |
| 4827 | GAGACCCAGGCTGACAGAAGCTCTA |
| 4828 | AGACCCAGGCTGACAGAAGCTCTAC |
| 4829 | GACCCAGGCTGACAGAAGCTCTACC |
| 4830 | ACCCAGGCTGACAGAAGCTCTACCA |
| 4831 | CCCAGGCTGACAGAAGCTCTACCAT |
| 4832 | CCAGGCTGACAGAAGCTCTACCATC |
| 4833 | CAGGCTGACAGAAGCTCTACCATCC |
| 4834 | AGGCTGACAGAAGCTCTACCATCCT |
| 4835 | GGCTGACAGAAGCTCTACCATCCTC |
| 4836 | GCTGACAGAAGCTCTACCATCCTCA |
| 4837 | CTGACAGAAGCTCTACCATCCTCAA |
| 4838 | TGACAGAAGCTCTACCATCCTCAAC |
| 4839 | GACAGAAGCTCTACCATCCTCAACA |
| 4840 | ACAGAAGCTCTACCATCCTCAACAT |
| 4841 | CAGAAGCTCTACCATCCTCAACATG |
| 4842 | AGAAGCTCTACCATCCTCAACATGT |
| 4843 | GAAGCTCTACCATCCTCAACATGTG |
| 4844 | AAGCTCTACCATCCTCAACATGTGG |
| 4845 | AGCTCTACCATCCTCAACATGTGGC |
| 4846 | GCTCTACCATCCTCAACATGTGGCT |
| 4847 | TACCATCCTCAACATGTGGCTTTTA |
| 4848 | ACCATCCTCAACATGTGGCTTTTAA |
| 4849 | CCATCCTCAACATGTGGCTTTTAAG |
| 4850 | CATCCTCAACATGTGGCTTTTAAGG |
| 4851 | ATCCTCAACATGTGGCTTTTAAGGT |
| 4852 | TCCTCAACATGTGGCTTTTAAGGTC |
| 4853 | CCTCAACATGTGGCTTTTAAGGTCT |
| 4854 | CTCAACATGTGGCTTTTAAGGTCTC |
| 4855 | TCAACATGTGGCTTTTAAGGTCTCC |
| 4856 | CATGCAGTCATTCAGAGACCCAGGCT |
| 4857 | ATGCAGTCATTCAGAGACCCAGGCTG |
| 4858 | TGCAGTCATTCAGAGACCCAGGCTGA |
| 4859 | GCAGTCATTCAGAGACCCAGGCTGAC |
| 4860 | CAGTCATTCAGAGACCCAGGCTGACA |
| 4861 | AGTCATTCAGAGACCCAGGCTGACAG |
| 4862 | GTCATTCAGAGACCCAGGCTGACAGA |
| 4863 | TCATTCAGAGACCCAGGCTGACAGAA |
| 4864 | CATTCAGAGACCCAGGCTGACAGAAG |
| 4865 | ATTCAGAGACCCAGGCTGACAGAAGC |
| 4866 | TTCAGAGACCCAGGCTGACAGAAGCT |
| 4867 | TCAGAGACCCAGGCTGACAGAAGCTC |
| 4868 | CAGAGACCCAGGCTGACAGAAGCTCT |
| 4869 | AGAGACCCAGGCTGACAGAAGCTCTA |
| 4870 | GAGACCCAGGCTGACAGAAGCTCTAC |
| 4871 | AGACCCAGGCTGACAGAAGCTCTACC |
| 4872 | GACCCAGGCTGACAGAAGCTCTACCA |
| 4873 | ACCCAGGCTGACAGAAGCTCTACCAT |
| 4874 | CCCAGGCTGACAGAAGCTCTACCATC |
| 4875 | CCAGGCTGACAGAAGCTCTACCATCC |
| 4876 | CAGGCTGACAGAAGCTCTACCATCCT |
| 4877 | AGGCTGACAGAAGCTCTACCATCCTC |
| 4878 | GGCTGACAGAAGCTCTACCATCCTCA |
| 4879 | GCTGACAGAAGCTCTACCATCCTCAA |
| 4880 | CTGACAGAAGCTCTACCATCCTCAAC |
| 4881 | TGACAGAAGCTCTACCATCCTCAACA |
| 4882 | GACAGAAGCTCTACCATCCTCAACAT |
| 4883 | ACAGAAGCTCTACCATCCTCAACATG |
| 4884 | CAGAAGCTCTACCATCCTCAACATGT |
| 4885 | AGAAGCTCTACCATCCTCAACATGTG |
| 4886 | GAAGCTCTACCATCCTCAACATGTGG |
| 4887 | AAGCTCTACCATCCTCAACATGTGGC |
| 4888 | AGCTCTACCATCCTCAACATGTGGCT |
| 4889 | GCTCTACCATCCTCAACATGTGGCTT |
| 4890 | CTACCATCCTCAACATGTGGCTTTTA |
| 4891 | TACCATCCTCAACATGTGGCTTTTAA |
| 4892 | ACCATCCTCAACATGTGGCTTTTAAG |
| 4893 | CCATCCTCAACATGTGGCTTTTAAGG |
| 4894 | CATCCTCAACATGTGGCTTTTAAGGT |
| 4895 | ATCCTCAACATGTGGCTTTTAAGGTC |
| 4896 | TCCTCAACATGTGGCTTTTAAGGTCT |
| 4897 | CCTCAACATGTGGCTTTTAAGGTCTC |
| 4898 | CTCAACATGTGGCTTTTAAGGTCTCC |
| 4899 | GGCTCTGAAAGTGTGACT |
| 4900 | CTCTGAAAGTGTGACTAC |
| 4901 | TCTGAAAGTGTGACTACT |
| 4902 | CTGAAAGTGTGACTACTG |
| 4903 | AAGTGTGACTACTGGACC |
| 4904 | GTGTGACTACTGGACCAG |
| 4905 | TGACTACTGGACCAGCAG |
| 4906 | ACTACTGGACCAGCAGCA |
| 4907 | GCCATGAGCTCACAGGGCA |
| 4908 | CCATGAGCTCACAGGGCAG |
| 4909 | CATGAGCTCACAGGGCAGT |
| 4910 | ATGAGCTCACAGGGCAGTG |
| 4911 | GTGGCTCTGAAAGTGTGAC |
| 4912 | TGGCTCTGAAAGTGTGACT |
| 4913 | GGCTCTGAAAGTGTGACTA |
| 4914 | GCTCTGAAAGTGTGACTAC |
| 4915 | CTCTGAAAGTGTGACTACT |
| 4916 | TCTGAAAGTGTGACTACTG |
| 4917 | CTGAAAGTGTGACTACTGG |
| 4918 | GAAAGTGTGACTACTGGAC |
| 4919 | AAAGTGTGACTACTGGACC |
| 4920 | AAGTGTGACTACTGGACCA |
| 4921 | AGTGTGACTACTGGACCAG |
| 4922 | GTGTGACTACTGGACCAGC |
| 4923 | GTGACTACTGGACCAGCAG |
| 4924 | TGACTACTGGACCAGCAGC |
| 4925 | GACTACTGGACCAGCAGCA |
| 4926 | ACTACTGGACCAGCAGCAG |
| 4927 | ACTGGACCAGCAGCAGCCT |
| 4928 | AGCAGCAGCCTCCTCATTA |
| 4929 | TGCCATGAGCTCACAGGGCA |
| 4930 | GCCATGAGCTCACAGGGCAG |
| 4931 | CATGAGCTCACAGGGCAGTG |
| 4932 | ATGAGCTCACAGGGCAGTGG |
| 4933 | TGAGCTCACAGGGCAGTGGC |
| 4934 | GAGCTCACAGGGCAGTGGCT |
| 4935 | CTCACAGGGCAGTGGCTCTG |
| 4936 | CACAGGGCAGTGGCTCTGAA |
| 4937 | GTGGCTCTGAAAGTGTGACT |
| 4938 | TGGCTCTGAAAGTGTGACTA |
| 4939 | GGCTCTGAAAGTGTGACTAC |
| 4940 | GCTCTGAAAGTGTGACTACT |
| 4941 | CTCTGAAAGTGTGACTACTG |
| 4942 | TCTGAAAGTGTGACTACTGG |
| 4943 | CTGAAAGTGTGACTACTGGA |
| 4944 | TGAAAGTGTGACTACTGGAC |
| 4945 | AAAGTGTGACTACTGGACCA |
| 4946 | AAGTGTGACTACTGGACCAG |
| 4947 | AGTGTGACTACTGGACCAGC |
| 4948 | GTGTGACTACTGGACCAGCA |
| 4949 | GTGACTACTGGACCAGCAGC |
| 4950 | TGACTACTGGACCAGCAGCA |
| 4951 | GACTACTGGACCAGCAGCAG |
| 4952 | ACTACTGGACCAGCAGCAGC |
| 4953 | TACTGGACCAGCAGCAGCCT |
| 4954 | ACTGGACCAGCAGCAGCCTC |
| 4955 | ACCAGCAGCAGCCTCCTCAT |
| 4956 | CAGCAGCAGCCTCCTCATTA |
| 4957 | AGTGCCATGAGCTCACAGGGC |
| 4958 | GTGCCATGAGCTCACAGGGCA |
| 4959 | TGCCATGAGCTCACAGGGCAG |
| 4960 | GCCATGAGCTCACAGGGCAGT |
| 4961 | CCATGAGCTCACAGGGCAGTG |
| 4962 | CATGAGCTCACAGGGCAGTGG |
| 4963 | ATGAGCTCACAGGGCAGTGGC |
| 4964 | TGAGCTCACAGGGCAGTGGCT |
| 4965 | GAGCTCACAGGGCAGTGGCTC |
| 4966 | AGCTCACAGGGCAGTGGCTCT |
| 4967 | GCTCACAGGGCAGTGGCTCTG |
| 4968 | CTCACAGGGCAGTGGCTCTGA |
| 4969 | TCACAGGGCAGTGGCTCTGAA |
| 4970 | CACAGGGCAGTGGCTCTGAAA |
| 4971 | ACAGGGCAGTGGCTCTGAAAG |
| 4972 | CAGGGCAGTGGCTCTGAAAGT |
| 4973 | CAGTGGCTCTGAAAGTGTGAC |
| 4974 | AGTGGCTCTGAAAGTGTGACT |
| 4975 | GTGGCTCTGAAAGTGTGACTA |
| 4976 | TGGCTCTGAAAGTGTGACTAC |
| 4977 | GGCTCTGAAAGTGTGACTACT |
| 4978 | GCTCTGAAAGTGTGACTACTG |
| 4979 | CTCTGAAAGTGTGACTACTGG |
| 4980 | TCTGAAAGTGTGACTACTGGA |
| 4981 | CTGAAAGTGTGACTACTGGAC |
| 4982 | TGAAAGTGTGACTACTGGACC |
| 4983 | GAAAGTGTGACTACTGGACCA |
| 4984 | AAAGTGTGACTACTGGACCAG |
| 4985 | AAGTGTGACTACTGGACCAGC |
| 4986 | AGTGTGACTACTGGACCAGCA |
| 4987 | GTGTGACTACTGGACCAGCAG |
| 4988 | TGTGACTACTGGACCAGCAGC |
| 4989 | GTGACTACTGGACCAGCAGCA |
| 4990 | TGACTACTGGACCAGCAGCAG |
| 4991 | GACTACTGGACCAGCAGCAGC |
| 4992 | ACTACTGGACCAGCAGCAGCC |
| 4993 | CTACTGGACCAGCAGCAGCCT |
| 4994 | TACTGGACCAGCAGCAGCCTC |
| 4995 | ACTGGACCAGCAGCAGCCTCC |
| 4996 | GACCAGCAGCAGCCTCCTCAT |
| 4997 | ACCAGCAGCAGCCTCCTCATT |
| 4998 | CCAGCAGCAGCCTCCTCATTA |
| 4999 | CAGCAGCAGCCTCCTCATTAC |
| 5000 | AGTGCCATGAGCTCACAGGGCA |
| 5001 | GTGCCATGAGCTCACAGGGCAG |
| 5002 | TGCCATGAGCTCACAGGGCAGT |
| 5003 | GCCATGAGCTCACAGGGCAGTG |
| 5004 | CCATGAGCTCACAGGGCAGTGG |
| 5005 | CATGAGCTCACAGGGCAGTGGC |
| 5006 | ATGAGCTCACAGGGCAGTGGCT |
| 5007 | TGAGCTCACAGGGCAGTGGCTC |
| 5008 | GAGCTCACAGGGCAGTGGCTCT |
| 5009 | AGCTCACAGGGCAGTGGCTCTG |
| 5010 | GCTCACAGGGCAGTGGCTCTGA |
| 5011 | CTCACAGGGCAGTGGCTCTGAA |
| 5012 | TCACAGGGCAGTGGCTCTGAAA |
| 5013 | CACAGGGCAGTGGCTCTGAAAG |
| 5014 | ACAGGGCAGTGGCTCTGAAAGT |
| 5015 | CAGGGCAGTGGCTCTGAAAGTG |
| 5016 | GGCAGTGGCTCTGAAAGTGTGA |
| 5017 | GCAGTGGCTCTGAAAGTGTGAC |
| 5018 | CAGTGGCTCTGAAAGTGTGACT |
| 5019 | AGTGGCTCTGAAAGTGTGACTA |
| 5020 | GTGGCTCTGAAAGTGTGACTAC |
| 5021 | TGGCTCTGAAAGTGTGACTACT |
| 5022 | GGCTCTGAAAGTGTGACTACTG |
| 5023 | GCTCTGAAAGTGTGACTACTGG |
| 5024 | CTCTGAAAGTGTGACTACTGGA |
| 5025 | TCTGAAAGTGTGACTACTGGAC |
| 5026 | CTGAAAGTGTGACTACTGGACC |
| 5027 | TGAAAGTGTGACTACTGGACCA |
| 5028 | GAAAGTGTGACTACTGGACCAG |
| 5029 | AAAGTGTGACTACTGGACCAGC |
| 5030 | AAGTGTGACTACTGGACCAGCA |
| 5031 | AGTGTGACTACTGGACCAGCAG |
| 5032 | GTGTGACTACTGGACCAGCAGC |
| 5033 | TGTGACTACTGGACCAGCAGCA |
| 5034 | GTGACTACTGGACCAGCAGCAG |
| 5035 | TGACTACTGGACCAGCAGCAGC |
| 5036 | GACTACTGGACCAGCAGCAGCC |
| 5037 | ACTACTGGACCAGCAGCAGCCT |
| 5038 | CTACTGGACCAGCAGCAGCCTC |
| 5039 | TACTGGACCAGCAGCAGCCTCC |
| 5040 | ACTGGACCAGCAGCAGCCTCCT |
| 5041 | GGACCAGCAGCAGCCTCCTCAT |
| 5042 | GACCAGCAGCAGCCTCCTCATT |
| 5043 | ACCAGCAGCAGCCTCCTCATTA |
| 5044 | CCAGCAGCAGCCTCCTCATTAC |
| 5045 | AGTGCCATGAGCTCACAGGGCAG |
| 5046 | GTGCCATGAGCTCACAGGGCAGT |
| 5047 | TGCCATGAGCTCACAGGGCAGTG |
| 5048 | GCCATGAGCTCACAGGGCAGTGG |
| 5049 | CCATGAGCTCACAGGGCAGTGGC |
| 5050 | CATGAGCTCACAGGGCAGTGGCT |
| 5051 | ATGAGCTCACAGGGCAGTGGCTC |
| 5052 | TGAGCTCACAGGGCAGTGGCTCT |
| 5053 | GAGCTCACAGGGCAGTGGCTCTG |
| 5054 | AGCTCACAGGGCAGTGGCTCTGA |
| 5055 | GCTCACAGGGCAGTGGCTCTGAA |
| 5056 | CTCACAGGGCAGTGGCTCTGAAA |
| 5057 | TCACAGGGCAGTGGCTCTGAAAG |
| 5058 | CACAGGGCAGTGGCTCTGAAAGT |
| 5059 | ACAGGGCAGTGGCTCTGAAAGTG |
| 5060 | CAGGGCAGTGGCTCTGAAAGTGT |
| 5061 | GGGCAGTGGCTCTGAAAGTGTGA |
| 5062 | GGCAGTGGCTCTGAAAGTGTGAC |
| 5063 | GCAGTGGCTCTGAAAGTGTGACT |
| 5064 | CAGTGGCTCTGAAAGTGTGACTA |
| 5065 | AGTGGCTCTGAAAGTGTGACTAC |
| 5066 | GTGGCTCTGAAAGTGTGACTACT |
| 5067 | TGGCTCTGAAAGTGTGACTACTG |
| 5068 | GGCTCTGAAAGTGTGACTACTGG |
| 5069 | GCTCTGAAAGTGTGACTACTGGA |
| 5070 | CTCTGAAAGTGTGACTACTGGAC |
| 5071 | TCTGAAAGTGTGACTACTGGACC |
| 5072 | CTGAAAGTGTGACTACTGGACCA |
| 5073 | TGAAAGTGTGACTACTGGACCAG |
| 5074 | GAAAGTGTGACTACTGGACCAGC |
| 5075 | AAAGTGTGACTACTGGACCAGCA |
| 5076 | AAGTGTGACTACTGGACCAGCAG |
| 5077 | AGTGTGACTACTGGACCAGCAGC |
| 5078 | GTGTGACTACTGGACCAGCAGCA |
| 5079 | TGTGACTACTGGACCAGCAGCAG |
| 5080 | GTGACTACTGGACCAGCAGCAGC |
| 5081 | TGACTACTGGACCAGCAGCAGCC |
| 5082 | GACTACTGGACCAGCAGCAGCCT |
| 5083 | ACTACTGGACCAGCAGCAGCCTC |
| 5084 | CTACTGGACCAGCAGCAGCCTCC |
| 5085 | TACTGGACCAGCAGCAGCCTCCT |
| 5086 | ACTGGACCAGCAGCAGCCTCCTC |
| 5087 | TGGACCAGCAGCAGCCTCCTCAT |
| 5088 | GGACCAGCAGCAGCCTCCTCATT |
| 5089 | GACCAGCAGCAGCCTCCTCATTA |
| 5090 | ACCAGCAGCAGCCTCCTCATTAC |
| 5091 | AGTGCCATGAGCTCACAGGGCAGT |
| 5092 | GTGCCATGAGCTCACAGGGCAGTG |
| 5093 | TGCCATGAGCTCACAGGGCAGTGG |
| 5094 | GCCATGAGCTCACAGGGCAGTGGC |
| 5095 | CCATGAGCTCACAGGGCAGTGGCT |
| 5096 | CATGAGCTCACAGGGCAGTGGCTC |
| 5097 | ATGAGCTCACAGGGCAGTGGCTCT |
| 5098 | TGAGCTCACAGGGCAGTGGCTCTG |
| 5099 | GAGCTCACAGGGCAGTGGCTCTGA |
| 5100 | AGCTCACAGGGCAGTGGCTCTGAA |
| 5101 | GCTCACAGGGCAGTGGCTCTGAAA |
| 5102 | CTCACAGGGCAGTGGCTCTGAAAG |
| 5103 | TCACAGGGCAGTGGCTCTGAAAGT |
| 5104 | CACAGGGCAGTGGCTCTGAAAGTG |
| 5105 | ACAGGGCAGTGGCTCTGAAAGTGT |
| 5106 | CAGGGCAGTGGCTCTGAAAGTGTG |
| 5107 | AGGGCAGTGGCTCTGAAAGTGTGA |
| 5108 | GGGCAGTGGCTCTGAAAGTGTGAC |
| 5109 | GGCAGTGGCTCTGAAAGTGTGACT |
| 5110 | GCAGTGGCTCTGAAAGTGTGACTA |
| 5111 | CAGTGGCTCTGAAAGTGTGACTAC |
| 5112 | AGTGGCTCTGAAAGTGTGACTACT |
| 5113 | GTGGCTCTGAAAGTGTGACTACTG |
| 5114 | TGGCTCTGAAAGTGTGACTACTGG |
| 5115 | GGCTCTGAAAGTGTGACTACTGGA |
| 5116 | GCTCTGAAAGTGTGACTACTGGAC |
| 5117 | CTCTGAAAGTGTGACTACTGGACC |
| 5118 | TCTGAAAGTGTGACTACTGGACCA |
| 5119 | CTGAAAGTGTGACTACTGGACCAG |
| 5120 | TGAAAGTGTGACTACTGGACCAGC |
| 5121 | GAAAGTGTGACTACTGGACCAGCA |
| 5122 | AAAGTGTGACTACTGGACCAGCAG |
| 5123 | AAGTGTGACTACTGGACCAGCAGC |
| 5124 | AGTGTGACTACTGGACCAGCAGCA |
| 5125 | GTGTGACTACTGGACCAGCAGCAG |
| 5126 | TGTGACTACTGGACCAGCAGCAGC |
| 5127 | GTGACTACTGGACCAGCAGCAGCC |
| 5128 | TGACTACTGGACCAGCAGCAGCCT |
| 5129 | GACTACTGGACCAGCAGCAGCCTC |
| 5130 | ACTACTGGACCAGCAGCAGCCTCC |
| 5131 | CTACTGGACCAGCAGCAGCCTCCT |
| 5132 | TACTGGACCAGCAGCAGCCTCCTC |
| 5133 | ACTGGACCAGCAGCAGCCTCCTCA |
| 5134 | CTGGACCAGCAGCAGCCTCCTCAT |
| 5135 | TGGACCAGCAGCAGCCTCCTCATT |
| 5136 | GGACCAGCAGCAGCCTCCTCATTA |
| 5137 | GACCAGCAGCAGCCTCCTCATTAC |
| 5138 | AGTGCCATGAGCTCACAGGGCAGTG |
| 5139 | GTGCCATGAGCTCACAGGGCAGTGG |
| 5140 | TGCCATGAGCTCACAGGGCAGTGGC |
| 5141 | GCCATGAGCTCACAGGGCAGTGGCT |
| 5142 | CCATGAGCTCACAGGGCAGTGGCTC |
| 5143 | CATGAGCTCACAGGGCAGTGGCTCT |
| 5144 | ATGAGCTCACAGGGCAGTGGCTCTG |
| 5145 | TGAGCTCACAGGGCAGTGGCTCTGA |
| 5146 | GAGCTCACAGGGCAGTGGCTCTGAA |
| 5147 | AGCTCACAGGGCAGTGGCTCTGAAA |
| 5148 | GCTCACAGGGCAGTGGCTCTGAAAG |
| 5149 | CTCACAGGGCAGTGGCTCTGAAAGT |
| 5150 | TCACAGGGCAGTGGCTCTGAAAGTG |
| 5151 | CACAGGGCAGTGGCTCTGAAAGTGT |
| 5152 | ACAGGGCAGTGGCTCTGAAAGTGTG |
| 5153 | CAGGGCAGTGGCTCTGAAAGTGTGA |
| 5154 | AGGGCAGTGGCTCTGAAAGTGTGAC |
| 5155 | GGGCAGTGGCTCTGAAAGTGTGACT |
| 5156 | GGCAGTGGCTCTGAAAGTGTGACTA |
| 5157 | GCAGTGGCTCTGAAAGTGTGACTAC |
| 5158 | CAGTGGCTCTGAAAGTGTGACTACT |
| 5159 | AGTGGCTCTGAAAGTGTGACTACTG |
| 5160 | GTGGCTCTGAAAGTGTGACTACTGG |
| 5161 | TGGCTCTGAAAGTGTGACTACTGGA |
| 5162 | GGCTCTGAAAGTGTGACTACTGGAC |
| 5163 | GCTCTGAAAGTGTGACTACTGGACC |
| 5164 | CTCTGAAAGTGTGACTACTGGACCA |
| 5165 | TCTGAAAGTGTGACTACTGGACCAG |
| 5166 | CTGAAAGTGTGACTACTGGACCAGC |
| 5167 | TGAAAGTGTGACTACTGGACCAGCA |
| 5168 | GAAAGTGTGACTACTGGACCAGCAG |
| 5169 | AAAGTGTGACTACTGGACCAGCAGC |
| 5170 | AAGTGTGACTACTGGACCAGCAGCA |
| 5171 | AGTGTGACTACTGGACCAGCAGCAG |
| 5172 | GTGTGACTACTGGACCAGCAGCAGC |
| 5173 | TGTGACTACTGGACCAGCAGCAGCC |
| 5174 | GTGACTACTGGACCAGCAGCAGCCT |
| 5175 | TGACTACTGGACCAGCAGCAGCCTC |
| 5176 | GACTACTGGACCAGCAGCAGCCTCC |
| 5177 | ACTACTGGACCAGCAGCAGCCTCCT |
| 5178 | CTACTGGACCAGCAGCAGCCTCCTC |
| 5179 | TACTGGACCAGCAGCAGCCTCCTCA |
| 5180 | ACTGGACCAGCAGCAGCCTCCTCAT |
| 5181 | CTGGACCAGCAGCAGCCTCCTCATT |
| 5182 | TGGACCAGCAGCAGCCTCCTCATTA |
| 5183 | GGACCAGCAGCAGCCTCCTCATTAC |
| 5184 | AGTGCCATGAGCTCACAGGGCAGTGG |
| 5185 | GTGCCATGAGCTCACAGGGCAGTGGC |
| 5186 | TGCCATGAGCTCACAGGGCAGTGGCT |
| 5187 | GCCATGAGCTCACAGGGCAGTGGCTC |
| 5188 | CCATGAGCTCACAGGGCAGTGGCTCT |
| 5189 | CATGAGCTCACAGGGCAGTGGCTCTG |
| 5190 | ATGAGCTCACAGGGCAGTGGCTCTGA |
| 5191 | TGAGCTCACAGGGCAGTGGCTCTGAA |
| 5192 | GAGCTCACAGGGCAGTGGCTCTGAAA |
| 5193 | AGCTCACAGGGCAGTGGCTCTGAAAG |
| 5194 | GCTCACAGGGCAGTGGCTCTGAAAGT |
| 5195 | CTCACAGGGCAGTGGCTCTGAAAGTG |
| 5196 | TCACAGGGCAGTGGCTCTGAAAGTGT |
| 5197 | CACAGGGCAGTGGCTCTGAAAGTGTG |
| 5198 | ACAGGGCAGTGGCTCTGAAAGTGTGA |
| 5199 | CAGGGCAGTGGCTCTGAAAGTGTGAC |
| 5200 | AGGGCAGTGGCTCTGAAAGTGTGACT |
| 5201 | GGGCAGTGGCTCTGAAAGTGTGACTA |
| 5202 | GGCAGTGGCTCTGAAAGTGTGACTAC |
| 5203 | GCAGTGGCTCTGAAAGTGTGACTACT |
| 5204 | CAGTGGCTCTGAAAGTGTGACTACTG |
| 5205 | AGTGGCTCTGAAAGTGTGACTACTGG |
| 5206 | GTGGCTCTGAAAGTGTGACTACTGGA |
| 5207 | TGGCTCTGAAAGTGTGACTACTGGAC |
| 5208 | GGCTCTGAAAGTGTGACTACTGGACC |
| 5209 | GCTCTGAAAGTGTGACTACTGGACCA |
| 5210 | CTCTGAAAGTGTGACTACTGGACCAG |
| 5211 | TCTGAAAGTGTGACTACTGGACCAGC |
| 5212 | CTGAAAGTGTGACTACTGGACCAGCA |
| 5213 | TGAAAGTGTGACTACTGGACCAGCAG |
| 5214 | GAAAGTGTGACTACTGGACCAGCAGC |
| 5215 | AAAGTGTGACTACTGGACCAGCAGCA |
| 5216 | AAGTGTGACTACTGGACCAGCAGCAG |
| 5217 | AGTGTGACTACTGGACCAGCAGCAGC |
| 5218 | GTGTGACTACTGGACCAGCAGCAGCC |
| 5219 | TGTGACTACTGGACCAGCAGCAGCCT |
| 5220 | GTGACTACTGGACCAGCAGCAGCCTC |
| 5221 | TGACTACTGGACCAGCAGCAGCCTCC |
| 5222 | GACTACTGGACCAGCAGCAGCCTCCT |
| 5223 | ACTACTGGACCAGCAGCAGCCTCCTC |
| 5224 | CTACTGGACCAGCAGCAGCCTCCTCA |
| 5225 | TACTGGACCAGCAGCAGCCTCCTCAT |
| 5226 | ACTGGACCAGCAGCAGCCTCCTCATT |
| 5227 | CTGGACCAGCAGCAGCCTCCTCATTA |
| 5228 | TGGACCAGCAGCAGCCTCCTCATTAC |

TABLE 11
Exemplary ASOs that target regRNA RR84_v1

| SEQ ID | |
|---|---|
| NO | Sequence |
| 5229 | GAATTTAACTGAAAAACTGC |
| 5230 | TGAGAATAGAATTTAACTGA |
| 5231 | GTGCTATAAATGAGAATAGA |
| 5232 | AAACTGTGCTATAAATGAGA |
| 5233 | ATACTAAAACTGTGCTATAA |
| 5234 | CAGGCATACTAAAACTGTGC |
| 5235 | TTATGGAATCAGGCATACTA |
| 5236 | GCACGTTATGGAATCAGGCA |
| 5237 | CATTAGATAGCACGTTATGG |
| 5238 | AGCCACATTAGATAGCACGT |
| 5239 | AGAGCAGCCACATTAGATAG |
| 5240 | TTATTAGAGCAGCCACATTA |
| 5241 | GAAATTATTATTAGAGCAGC |
| 5242 | GACTGAGAAATTATTATTAG |
| 5243 | CCCAAGGACTGAGAAATTAT |
| 5244 | ACACTCCCAAGGACTGAGAA |
| 5245 | GCTAAAAACACTCCCAAGGA |
| 5246 | CATCAGCTAAAAACACTCCC |
| 5247 | TCGGGTCACATCAGCTAAAA |
| 5248 | GGAATTTCGGGTCACATCAG |
| 5249 | GAATAGGAATTTCGGGTCAC |
| 5250 | TATAGGAATAGGAATTTCGG |
| 5251 | GACAACATATAGGAATAGGA |
| 5252 | GAGGCCTGGACAACATATAG |
| 5253 | GGAATTGAGGCCTGGACAAC |
| 5254 | GTTCCCCAGGAATTGAGGCC |
| 5255 | TCTCGGTTCCCCAGGAATTG |
| 5256 | AGTCACTGTCTCGGTTCCCC |
| 5257 | TATCCCAGTCACTGTCTCGG |
| 5258 | AGAGTTATCCCAGTCACTGT |
| 5259 | TTCCAAGAGTTATCCCAGTC |
| 5260 | TGACCCATTCCAAGAGTTAT |
| 5261 | GTGATCTGACCCATTCCAAG |
| 5262 | AGAGAGAGGTGATCTGACCC |
| 5263 | ACCTCTTTCAGAGAGAGGTG |
| 5264 | TCAGATAACCTCTTTCAGAG |
| 5265 | CTTATCTTTCAGATAACCTC |
| 5266 | TTCATCTTATCTTTCAGATA |
| 5267 | GTTCTCCATTTCATCTTATC |
| 5268 | CAACTGTGTTCTCCATTTCA |
| 5269 | CTGCAATCACAACTGTGTTC |
| 5270 | GGATTTCTGCAATCACAACT |
| 5271 | ACAAGAAGGATTTCTGCAAT |
| 5272 | AAGCACACAAGAAGGATTTC |
| 5273 | GCATTAACAAGCACACAAGA |
| 5274 | ACAAAGAGCATTAACAAGCA |
| 5275 | AACAGACAAAGAGCATTAAC |
| 5276 | AGGCACAACAGACAAAGAGC |
| 5277 | CCTGAAAAGGCACAACAGAC |
| 5278 | GCACTTCCCTGAAAAGGCAC |
| 5279 | AAGCTTGGGCACTTCCCTGA |
| 5280 | CTCCCAAGCTTGGGCACTTC |
| 5281 | TCCATGTCCTCCCAAGCTTG |
| 5282 | TGTTCTCCATGTCCTCCCAA |
| 5283 | GCCTATTGTTCTCCATGTCC |
| 5284 | CAATATGCCTATTGTTCTCC |
| 5285 | ACTCTCCAATATGCCTATTG |
| 5286 | CACTCACTCTCCAATATGCC |
| 5287 | AACCCCACTCACTCTCCAAT |
| 5288 | GACTCCAAACCCCACTCACT |
| 5289 | GGCTCTGACTCCAAACCCCA |
| 5290 | CTAAGGGGCTCTGACTCCAA |
| 5291 | CAAGTCTAAGGGGCTCTGAC |
| 5292 | GTAGGATTCAAGTCTAAGGG |
| 5293 | GCAGAGTAGGATTCAAGTCT |
| 5294 | ACAGCTGGCAGAGTAGGATT |
| 5295 | GACTGCACAGCTGGCAGAGT |
| 5296 | TGTCTGGGACTGCACAGCTG |
| 5297 | GGATTTGTCTGGGACTGCAC |
| 5298 | TAAGGGGATTTGTCTGGGAC |
| 5299 | CAGAAAGGTAAGGGGATTTG |
| 5300 | ACTAGGCTCAGAAAGGTAAG |
| 5301 | ATATAAGAAACTAGGCTCAG |
| 5302 | GTGAGCACTAACATCATTTT |
| 5303 | ATAAAGTGAGCACTAACATC |
| 5304 | CAATCTTATAAAGTGAGCAC |
| 5305 | TAGTCATTATCTCTAATCTT |
| 5306 | GGAGCTTAATAGTCATTATC |
| 5307 | CACTACTAGGAGCTTAATAG |
| 5308 | TCTAGGCACTACTAGGAGCT |
| 5309 | GTTTCTCTAGGCACTACTAG |
| 5310 | GCATTCACTGTTTCTCTAGG |
| 5311 | ATTGAGCATTCACTGTTTCT |
| 5312 | GTTTTATTAACAGCAATTAC |
| 5313 | GTTTTAATAGGTAAATATTG |
| 5314 | GATTAATGATGTTTTAATAG |
| 5315 | GAATAGGGATTAATGATGTT |
| 5316 | TCATCGAATAGGGATTAATG |
| 5317 | TGACATTTCATCGAATAGGG |
| 5318 | TCAAATGACATTTCATCGAA |
| 5319 | CCAGTGTCAAATGACATTTC |
| 5320 | ACTCCCCAGTGTCAAATGAC |
| 5321 | TAGATTACTCCCCAGTGTCA |
| 5322 | AGTTTGTTAGATTACTCCCC |
| 5323 | CATGTAGTTTGTTAGATTAC |
| 5324 | ATCTTCATGTAGTTTGTTAG |
| 5325 | TATCACTATCTTCATGTAGT |
| 5326 | ACATAATTATCACTATCTTC |
| 5327 | GTTAGCAACATAATTATCAC |
| 5328 | CTGAGTACAGTTAGCAACAT |
| 5329 | CTTTTCCTGAGTACAGTTAG |
| 5330 | GAGTCCCTTTTCCTGAGTAC |
| 5331 | AGCTTGAGTCCCTTTTCCTG |
| 5332 | ATGTGAAGCTTGAGTCCCTT |
| 5333 | CCAAAAATGTGAAGCTTGAG |
| 5334 | AACAGCCAAAAATGTGAAGC |
| 5335 | TTCATAAACAGCCAAAAATG |
| 5336 | TTGCCTTCATAAACAGCCAA |
| 5337 | AGACATTTGCCTTCATAAAC |
| 5338 | ATGGGAAGACATTTGCCTTC |
| 5339 | TCTCCATGGGAAGACATTTG |
| 5340 | GCATCTCTCCATGGGAAGAC |
| 5341 | CTTTTTGCATCTCTCCATGG |
| 5342 | TAAGAATGTCTTTTTGCATC |
| 5343 | GGTCTGGTAAGAATGTCTTT |
| 5344 | CCACCATTGGTCTGGTAAGA |
| 5345 | AGCAGCCACCATTGGTCTGG |
| 5346 | ATTGGAGCAGCCACCATTGG |
| 5347 | CTCATTGGGATTGGAGCAGC |
| 5348 | AGCCCTCTCATTGGGATTGG |
| 5349 | TCTGTCAGCCCTCTCATTGG |
| 5350 | TTATTTCTGTCAGCCCTCTC |
| 5351 | GGTTATTATTTCTGTCAGCC |
| 5352 | GACCTTGGTTATTATTTCTG |
| 5353 | AAACTGACCTTGGTTATTAT |
| 5354 | GTTGGAAACTGACCTTGGTT |
| 5355 | TTTTCAGGGGTTGGAAACTG |
| 5356 | GGGATTTTTCAGGGGTTGGA |
| 5357 | CTGTTGGGATTTTTCAGGGG |
| 5358 | GTTTCCTGTTGGGATTTTTC |
| 5359 | CATCTGTGGTTTCCTGTTGG |
| 5360 | TCGACCATCTGTGGTTTCCT |
| 5361 | ATGACCTCGACCATCTGTGG |
| 5362 | ATTCAGCGCATGACCTCGAC |
| 5363 | CCCTTTTCATTCAGCGCATG |
| 5364 | GACCAAATGGCCCTTTTCAT |
| 5365 | TAGCTGACCAAATGGCCCTT |
| 5366 | TGCCATAGCTGACCAAATGG |
| 5367 | CAATGATGCCATAGCTGACC |
| 5368 | AAAATCAATGATGCCATAGC |
| 5369 | ATTAATAAAATCAATGATGC |
| 5370 | GGTTTCTAGTGATATTAATA |
| 5371 | ATCTTTCTGGTTTCTAGTGA |
| 5372 | GTGACCAAAATCTTTCTGGT |
| 5373 | AGTTTTAGTGACCAAAATCT |
| 5374 | CAAATTCAGTTTTAGTGACC |
| 5375 | TTGAATGATCAAATTCAGTT |
| 5376 | GATATTTGAATGATCAAATT |
| 5377 | TCAGCAAGATATTTGAATGA |
| 5378 | GAGACTTTCAGCAAGATATT |
| 5379 | GAAAGACTGAGACTTTCAGC |
| 5380 | TGTTTAAAAGGGAAAGACTG |
| 5381 | GATGGGAATGTTTAAAAGGG |
| 5382 | ATAGAGAGATGGGAATGTTT |
| 5383 | TAGGGATAGAGAGATGGGAA |
| 5384 | GGTGATAGGGATAGAGAGAT |
| 5385 | TCCAAGGGTGATAGGGATAG |
| 5386 | TGCTATCCAAGGGTGATAGG |
| 5387 | GTTAATGCTATCCAAGGGTG |
| 5388 | AGGCTGTTAATGCTATCCAA |
| 5389 | ATCTGAGAGGCTGTTAATGC |
| 5390 | CCAAGATCTGAGAGGCTGTT |
| 5391 | ATTTTCCAAGATCTGAGAGG |
| 5392 | AGCAAATTTTCCAAGATCTG |
| 5393 | GGTGCAGCAAATTTTCCAAG |
| 5394 | CCCAAGGTGCAGCAAATTTT |
| 5395 | ATAACATCCCAAGGTGCAGC |
| 5396 | GCTTGATAACATCCCAAGGT |
| 5397 | AAGAGGCTTGATAACATCCC |
| 5398 | CAGGAAAGAGGCTTGATAAC |
| 5399 | ATTGGCTATCAGGAAAGAGG |
| 5400 | AAATTGATTGGCTATCAGGA |
| 5401 | AGAACAAAATTGATTGGCTA |
| 5402 | CTAGATTAAGAACAAAATTG |
| 5403 | GCTGGGAAACCTAGATTAAG |
| 5404 | GAGTACCAGCTGGGAAACCT |
| 5405 | ATCAACACTGAGTACCAGCT |
| 5406 | AAGCCTTTATCAACACTGAG |
| 5407 | GGTTTCCAAAAGCCTTTATC |
| 5408 | ATAAGATGGTTTCCAAAAGC |
| 5409 | GACAAATAAGATGGTTTCCA |
| 5410 | ATATTAGCTATGTAAAAGAA |
| 5411 | GAGCAAGGATATTAGCTATG |
| 5412 | CAAGACAGAGCAAGGATATT |
| 5413 | AAAGCTTTGCAAGACAGAGC |
| 5414 | GGCATGAAGAAAGAAAAGCT |
| 5415 | TTCAGCAGGCATGAAGAAAG |
| 5416 | GAAAGGATTCAGCAGGCATG |
| 5417 | CTTGAGGCTGAAAGGATTCA |
| 5418 | TAGAACCTTGAGGCTGAAAG |
| 5419 | GTGACTAGAACCTTGAGGCT |
| 5420 | GGTATGTGACTAGAACCTTG |
| 5421 | ACGAGAAGGTATGTGACTAG |
| 5422 | GATTGATGCACGAGAAGGTA |
| 5423 | TACAAGATTGATGCACGAGA |
| 5424 | GGATTATACAAGATTGATGC |
| 5425 | CTTGGGGGATTATACAAGAT |
| 5426 | TTCTTCTTGGGGGATTATAC |
| 5427 | GTTACTTCTTCTTGGGGGAT |
| 5428 | AAGGTGTTACTTCTTCTTGG |
| 5429 | AGGGAGAAAGGTGTTACTTC |
| 5430 | AAACACAGGGAGAAAGGTGT |
| 5431 | GTTACGGAAACACAGGGAGA |
| 5432 | ATTACAGTGTTACGGAAACA |
| 5433 | GATTAATTACAGTGTTACGG |
| 5434 | TGCACAAGGATTAATTACAG |
| 5435 | ACAAATGCACAAGGATTAAT |
| 5436 | CACATACAAATGCACAAGGA |
| 5437 | AATTCCACACACATACAAAT |
| 5438 | TGCAATAATTCCACACACAT |
| 5439 | GCAGAATGCAATAATTCCAC |
| 5440 | CTATATGCAGAATGCAATAA |
| 5441 | ATACTGCCTATATGCAGAAT |
| 5442 | ACTCTATACTGCCTATATGC |
| 5443 | TGCACTCTTCACCACTACTC |
| 5444 | TTCCTAGTCTGGCTCCAGAA |
| 5445 | TATTCAAATTCCTAGTCTGG |
| 5446 | GTCTGTATTCAAATTCCTAG |
| 5447 | CAAGAGTCTGTATTCAAATT |
| 5448 | GCCCAACAAGAGTCTGTATT |
| 5449 | GTAACTTGCCCAACAAGAGT |
| 5450 | AGACAAGAAAATTGAGCCAC |
| 5451 | GTTCTATTATTGTTCTTATT |
| 5452 | GGGGTAGGTTCTATTATTGT |
| 5453 | CCATTGGGGTAGGTTCTATT |
| 5454 | ATTACCCATTGGGGTAGGTT |
| 5455 | CCTCATTATTACCCATTGGG |
| 5456 | CGCTACTTTAATCCTCATTA |
| 5457 | TATATTCGCTACTTTAATCC |
| 5458 | TTTACATATATTCGCTACTT |
| 5459 | TAGGCTTTACATATATTCGC |
| 5460 | CGCTGTCCTATAGGCTTTAC |
| 5461 | ATGATAGGCGCTGTCCTATA |
| 5462 | ATTATATGATAGGCGCTGTC |
| 5463 | TGTTTATTATATGATAGGCG |
| 5464 | CATATTGTTTATTATATGAT |
| 5465 | TCAGAACTAACACATATTGT |
| 5466 | GCAAATAATCAGAACTAACA |
| 5467 | TGCAAGCAAATAATCAGAAC |
| 5468 | AGAAATGCAAGCAAATAATC |
| 5469 | GGGAAATAAGAAATGCAAGC |
| 5470 | CGTAGGGGAAATAAGAAATG |
| 5471 | AGCCTCGTAGGGGAAATAAG |
| 5472 | CTTACAGCCTCGTAGGGGAA |
| 5473 | GAGGAACTTACAGCCTCGTA |
| 5474 | GCTCTTGAGGAACTTACAGC |
| 5475 | CTTGTGCTCTTGAGGAACTT |
| 5476 | TTGCACTTGTGCTCTTGAGG |
| 5477 | ATTGCCTTGCACTTGTGCTC |
| 5478 | GGAGCAATTGCCTTGCACTT |
| 5479 | ATTCTGGAGCAATTGCCTTG |
| 5480 | TAAATATTCTGGAGCAATTG |
| 5481 | TTCAATAAATATTCTGGAGC |
| 5482 | AGGAATATGAATTTAATATA |
| 5483 | GGAAATGTTCTCAGGAATAT |
| 5484 | GATTAAGGAAATGTTCTCAG |
| 5485 | CTGGACAAGAGATTAAGGAA |
| 5486 | TCCAACTGGACAAGAGATTA |
| 5487 | TTCACTCCAACTGGACAAGA |
| 5488 | GCTCTCTTCACTCCAACTGG |
| 5489 | GTTCCAAGCTCTCTTCACTC |
| 5490 | GTTTAGTTCCAAGCTCTCTT |
| 5491 | CTTGCTGTTTAGTTCCAAGC |
| 5492 | GGTTCTTTCTTGCTGTTTAG |
| 5493 | TGTCTTAGGTTCTTTCTTGC |
| 5494 | GGGTGATGTCTTAGGTTCTT |
| 5495 | TACCTGGGTGATGTCTTAGG |
| 5496 | ACAATGGCCTAATACCTGGG |
| 5497 | GGAACACAATGGCCTAATAC |
| 5498 | CTGCTGGAACACAATGGCCT |
| 5499 | TAACCCTGCTGGAACACAAT |
| 5500 | TTATCTAACCCTGCTGGAAC |
| 5501 | TGAGCAGTTATCTAACCCTG |
| 5502 | GACCTTGAGCAGTTATCTAA |
| 5503 | TTACTGTGACCTTGAGCAGT |
| 5504 | GCTCCTTACTGTGACCTTGA |
| 5505 | TACTCTGCTCCTTACTGTGA |
| 5506 | GCACTTACTCTGCTCCTTAC |
| 5507 | TTCCGGCACTTACTCTGCTC |
| 5508 | CCTGACTTCCGGCACTTACT |
| 5509 | GGACCCCTGACTTCCGGCAC |
| 5510 | TGAAATTCAGGACCCCTGAC |
| 5511 | GCACACTGAAATTCAGGACC |
| 5512 | GATAAAGCACACTGAAATTC |
| 5513 | GTGTAGATAAAGCACACTGA |
| 5514 | CTACAGTGTAGATAAAGCAC |
| 5515 | GTCACTCTACAGTGTAGATA |
| 5516 | AAGACAGTCACTCTACAGTG |
| 5517 | CCCCTGAAAGACAGTCACTC |
| 5518 | GAGGTCCCCTGAAAGACAGT |
| 5519 | TGAATTTTGAGGTCCCCTGA |

In some embodiments, an ASO provided herein comprises 16, 17, 18, 19, 20, 21, 22, 23, 24, 26, or 26 of the nucleotide sequence of an ASO provided in Table 2, 3, 4, 5, 6, 7, 8, 9, 10, or 11. For example, an ASO can comprise the first (from 5′ to 3′) 16, 17, 18, 19, 20, 21, 22, 23, 24, 25, or 26 nucleotides of any one of SEQ ID NOs: 14-644 or 645-6021, e.g., nucleotides at positions 1 to any one of positions 16, 17, 18, 19, 21, 22, 23, 24, 25, or 26 of any one of SEQ ID NOs: 14-6021. Alternatively, an ASO can comprise the last 16, 17, 18, 19, 20, 21, 22, 23, 24, or 25, nucleotides of any one of SEQ ID NOs: 14-6021, e.g., nucleotides at positions 2, 3, 4, 5, 6, 7, 8, 9, or 10 to positions 16, 17, 18, 19, 20, 21, 22, 23, 24, 25, or 26 of any one of SEQ ID NOs: 14-644 or 645-6021. For example, an ASO provided herein comprises the nucleotide sequence of nucleotides at positions 1 to 16, 1 to 17, 1 to 18, 1 to 19, 1 to 20, 1 to 21, 1 to 22, 1 to 23, 1 to 24, 1 to 25, 1 to 26, 2 to 17, 2 to 18, 2 to 19, 2 to 20, 2 to 21, 2 to 22, 2 to 23, 2 to 24, 2 to 25, 2 to 26, 3 to 18, 3 to 19, 3 to 20, 3 to 21, 3 to 22, 3 to 23, 3 to 24, 3 to 25, 3 to 26, 4 to 19, 4 to 20, 4 to 21, 4 to 22, 4 to 23, 4 to 24, 4 to 25, 4 to 26, 5 to 20, 5 to 21, 5 to 22, 5 to 23, 5 to 24, 5 to 25, 5 to 26, 6 to 21, 6 to 22, 6 to 23, 6 to 24, 6 to 25, 6 to 26, 7 to 22, 7 to 23, 7 to 24, 7 to 25, 7 to 26, 8 to 23, 8 to 24, 8 to 25, 8 to 26, 9 to 24, 9 to 25, 9 to 26, 10 to 25, or 10 to 26 of any one of SEQ ID NOs: 14-644 or 645-6021. In some embodiments, an ASO provided herein comprises the nucleotide sequence of nucleotides at positions 1 to 16, 2 to 17, 3 to 18, 4 to 19, 5 to 20, 6 to 21, 7 to 23, 8 to 24, 9 to 25, or 10-26 of any one of SEQ ID NOs: 14-644 or 645-6021. In such embodiments, the ASO is at least 16, 17, 18, 19, or 20 nucleotides in length.

In some aspects, an ASO provided herein can comprise the nucleotide sequence of any one of SEQ ID NOs: 14-6021 plus up to four additional nucleotides at the 5′ end of said nucleotide sequence that are complementary to the target ABCB11 regRNA. For example, the ASO can comprise one, two, three, or four additional nucleotides at the 5′ end of any one of SEQ ID NOs: 14-6021 that are complementary to the target ABCB11 regRNA (e.g., any one of the regRNAs described in Table 1, including RR62 v2 (SEQ ID NO: 1), RR63 v1 (SEQ ID NO: 2), RR63 v2 (SEQ ID NO: 3), RR64 v2 (SEQ ID NO: 4) or RR74 v1 (SEQ ID NO: 5), RR54 v2 (SEQ ID NO: 6), RR62 v1 (SEQ ID NO: 7), RR64 v1 (SEQ ID NO: 8), RR65 v1 (SEQ ID NO: 9), RR84 v1 (SEQ ID NO: 10), RR54 v1 (SEQ ID NO: 11), RR75 v1 (SEQ ID NO: 12), and RR76 v1 (SEQ ID NO: 13)). In some embodiments, if the ASO includes up to four (e.g., 1, 2, 3, or 4) additional nucleotides at the 5′ end of the nucleotide sequence any one of SEQ ID NOs: 14-6021 that are complementary to the target ABCB11 regRNA, the ASO also can exclude up to four (e.g., 1, 2, 3 or 4) nucleotides from the 3′ end of the nucleotide sequence of any one of SEQ ID NOs: 14-6021. For example, the ASO can exclude one, two, three, or four 3′ end nucleotides from the nucleotide sequence of any one of SEQ ID NOs: 14-6021 if it includes one, two, three or four 5′ nucleotides that are complementary to the target ABCB11 regRNA.

In some aspects, an ASO provided herein can comprise the nucleotide sequence of any one of SEQ ID NOs: 14-6021, plus up to four additional nucleotides at the 3′ end of said nucleotide sequence that are complementary to the target ABCB11 regRNA. For example, the ASO can comprise one, two, three, or four additional nucleotides at the 3′ end of any one of SEQ ID NOs: 14-6021 that are complementary to the target ABCB11 regRNA (e.g., any one of the regRNAs described in Table 1, including RR62 v2 (SEQ ID NO: 1), RR63 v1 (SEQ ID NO: 2), RR63 v2 (SEQ ID NO: 3), RR64 v2 (SEQ ID NO: 4) or RR74 v1 (SEQ ID NO: 5), RR54 v2 (SEQ ID NO: 6), RR62 v1 (SEQ ID NO: 7), RR64 v1 (SEQ ID NO: 8), RR65 v1 (SEQ ID NO: 9), RR84 v1 (SEQ ID NO: 10), RR54 v1 (SEQ ID NO: 11), RR75 v1 (SEQ ID NO: 12), and RR76 v1 (SEQ ID NO: 13)). In some embodiments, if the ASO includes up to four (e.g., 1, 2, 3, or 4) additional nucleotides at the 3′ end of the nucleotide sequence of any one of SEQ ID NOs: 14-6021 that are complementary to the target ABCB11 regRNA, the ASO also can exclude up to four (e.g., 1, 2, 3, or 4) nucleotides from the 5′ end of the nucleotide sequence any one of SEQ ID NOs: 14-6021. For example, the ASO can exclude one, two, three, or four 5′ end nucleotides from the nucleotide sequence of any one of SEQ ID NOs: 14-6021 if it includes one, two, three or four 3′ nucleotides that are complementary to the target ABCB11 regRNA.

In some embodiments, the regulatory RNA has a nucleotide sequence of SEQ ID NO: 1, and the ASO comprises a nucleotide sequence selected from the group consisting of SEQ ID NOs: 104-138, 217-272, 516-528, 645-1517, 5610-5644, 5723-5778, and 6018-6021.

In some embodiments, the regulatory RNA has a nucleotide sequence of SEQ ID NO: 2, and the ASO comprises a nucleotide sequence selected from the group consisting of SEQ ID NOs: 14-103, 273-381, 390-407, 463, 467-478, 515, 644, 1518-2057, 5520-5609, 5779-5887, 5896-5913, 5969, 5971-5980, and 6017.

In some embodiments, the regulatory RNA has a nucleotide sequence of SEQ ID NO: 3, and the ASO comprises a nucleotide sequence selected from the group consisting of SEQ ID NOs: 14-103, 139-148, 273-407, 463, 467-478, 515, 530-623, 625-644, 1518-2308, 5520-5609, 5645-5654, 5779-5913, 5969, 5971-5980, and 6017.

In some embodiments, the regulatory RNA has a nucleotide sequence of SEQ ID NO: 4, and the ASO comprises a nucleotide sequence selected from the group consisting of SEQ ID NOs: 149-168, 408-462, 464, 479-514, 2309-3956, 5655-5674, 5914-5968, 5970, and 5981-6016.

In some embodiments, the regulatory RNA has a nucleotide sequence of SEQ ID NO: 5, and the ASO comprises a nucleotide sequence selected from the group consisting of SEQ ID NOs: 169-216, 4062-5228, and 5675-5722.

In some embodiments, the regulatory RNA has a nucleotide sequence of SEQ ID NO: 7, and the ASO comprises a nucleotide sequence selected from the group consisting of SEQ ID NOs: 645-954.

In some embodiments, the regulatory RNA has a nucleotide sequence of SEQ ID NO: 8, and the ASO comprises a nucleotide sequence selected from the group consisting of SEQ ID NOs: 2309-2806.

In some embodiments, the regulatory RNA has a nucleotide sequence of SEQ ID NO: 9, and the ASO comprises a nucleotide sequence selected from the group consisting of SEQ ID NOs: 3957-4061.

In some embodiments, the regulatory RNA has a nucleotide sequence of SEQ ID NO: 10, and the ASO comprises a nucleotide sequence selected from the group consisting of SEQ ID NOs: 5229-5519.

### Hybridization and ΔG

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm, is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn (K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the free energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem, Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Aced Sci USA. 95:1460-1465 using appropriately derived thermodynamic parameters described by Sugimoto et al., 1995, Biochemistry 34:11211-11216 and McTigue et al., 2004, Biochemistry 43:5388-5405. To have the possibility of modulating its intended nucleic acid target by hybridization, oligonucleotides of the present disclosure hybridize to a target nucleic acid with estimated ΔG° values below −10 kcal/mol for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal/mol, such as below −15 kcal/mol, such as below −20 kcal/mol and such as below −25 kcal/mol for oligonucleotides that are 8-30 nucleotides in length. In some embodiments the oligonucleotides hybridize to a target nucleic acid with an estimated ΔG° value of −10 to −60 kcal/mol, such as −12 to −40 kcal/mol, −15 to −30 kcal/mol, −16 to −27 kcal/mol, or −18 to −25 kcal/mol.

### Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 70% or greater complementarity. For example, a mismatch in a duplex region consisting of 19 base pairs results in 94.7% complementarity, rendering the duplex region substantially complementary. Duplex regions can be formed by two separate oligonucleotide strands, as well as by single oligonucleotide strands that can form hairpin structures comprising a duplex region.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an ABCB11 regRNA, such as an eRNA or paRNA. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides. Generally, the duplex structure is between 5 and 50 base pairs in length, e.g., between, 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 10-10, 10-9, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 base pairs in length. Ranges and lengths intermediate to the above recited ranges and lengths are also contemplated to be part of the disclosure.

Similarly, the region of complementarity to the target sequence can be between 5 and 50 nucleotides in length, e.g., between 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 10-10, 10-9, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 nucleotides in length. Ranges and lengths intermediate to the above recited ranges and lengths are also contemplated to be part of the disclosure.

### Chemical Modifications of ASOs

In certain embodiments, the ASO does not consist of only DNA. In certain embodiments, the ASO comprises at least one chemical modification relative to a natural nucleotide (e.g., ribonucleotide, e.g., 2′-deoxy-2′-ribonucleotide). Various chemical modifications can be included in the ASOs of the present disclosure. The modifications can include one or more modifications in a sugar group (e.g., ribose), one or more modifications in a phosphate group, one or more modifications in a nucleobase, one or more terminal modifications, or a combination thereof. In some embodiments, an exemplary ASO sequence targeting a regRNA as shown in Table 2 is chemically modified. Such modifications can be, but are not limited to, 2′-O-(2-methoxyethyl) (2′-MOE), locked nucleic acid (LNA), 5-methyl on the cytidine, constrained ethyl (CET), phosphorothioate (PS) linkage, and/or a phosphodiester (PO) linkage, or any combination thereof. Chemical modifications of RNA are known in the art and described in, for example, PCT Application Publication No. WO2013/177248, incorporated herein by reference. In certain embodiments, each cytidine in an ASO provided herein is modified by 5-methyl.

Various chemical modifications for use with ASOs of the present disclosure include, but are not limited to: 3′-terminal deoxy-thymine (dT) nucleotides, 2′-O-methyl modified nucleotides, 2′-fluoro modified nucleotides, 2′-deoxy-modified nucleotides, locked nucleotides, unlocked nucleotides, conformationally restricted nucleotides, constrained ethyl nucleotides, abasic nucleotides, 2′-amino-modified nucleotides, 2′-O-allyl-modified nucleotides, 2′-C-alkyl-modified nucleotides, 2′-hydroxyl-modified nucleotides, 2′-methoxyethyl modified nucleotides, 2′-O-alkyl-modified nucleotides, morpholino nucleotides, phosphoramidates, non-natural base comprising nucleotides, tetrahydropyran modified nucleotides, 1,5-anhydrohexitol modified nucleotides, cyclohexenyl modified nucleotides, nucleotides comprising a phosphorothioate group, nucleotides comprising a methylphosphonate group, nucleotides comprising a 5′-phosphate, and nucleotides comprising a 5′-phosphate mimic.

In certain embodiments, the ASO comprises an RNA polynucleotide chemically modified to be resistant to one or more nucleases (e.g., nuclear RNases (e.g., the exosome complex or RNaseH)). In some embodiments, all nucleotide bases are modified in the ASO. In certain embodiments, the chemical modifications comprises β-D-ribonucleotides nucleotides, 2′-modified nucleotides (e.g., 2′-O-(2-Methoxyethyl) (2′-MOE), 2′-O—CH₃, or 2′-fluoro-arabino (FANA)), bicyclic sugar modified nucleotides (e.g., having a constrained ethyl or locked nucleic acid (LNA)), and/or one or more modified internucleotide bonds (e.g., phosphorothioate internucleotide linkage). In certain embodiments, the chemical modification comprises 2′-MOE and a phosphorothioate internucleotide bond. In certain embodiments, at least 8, 9, 10, 11, 12, 13, 14, 15, 16, 17, 18, 19, 20, or more consecutive nucleotides of the ASO are modified by 2′-MOE. In certain embodiments, each nucleotide of the ASO is modified by 2′-MOE. In certain embodiments, at least 7, 8, 9, 10, 11, 12, 13, 14, 15, 16, 17, 18, 19, 20, or more consecutive internucleotide bonds of the ASO are phosphorothioate internucleotide bonds. In certain embodiments, each internucleotide bond of the ASO is a phosphorothioate internucleotide bond.

Internucleotide linkage modifications that can be used with the ASOs of the present disclosure include, but are not limited to, phosphorothioate “PS” (P(S), phosphoramidate (P(NR₁R₂) such as dimethylaminophosphoramidate (P(N(CH₁)₂)), phospbonocarboxylate (P(CH₂)ₙCOOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂COO⁻)), alkylphosphonate (P(C₁₋₃alkyl) such as methylphosphonate —P(CH₃), boranophosphonate (P(BH3)), and phosphorodithioate (P(S)₂).

In some embodiments, an ASO provided herein comprises at least 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PO bonds. In some embodiments, all internucleotide bonds of an ASO provided herein are PO internucleotide bonds. In some embodiments, an ASO provided herein does not comprise PO internucleotide bonds. In some embodiments, an ASO provided herein comprises at least 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS internucleotide bonds. In some embodiments, all internucleotide bonds of an ASO provided herein are PS bonds. In some embodiments, an ASO provided herein does not comprise PS internucleotide bonds.

In some embodiments, an ASO provided herein comprises at least 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS bonds. In some embodiments, all internucleotide bonds of an ASO provided herein are PS internucleotide bonds. In some embodiments, an ASO provided herein does not comprise PS internucleotide bonds. In some embodiments, an ASO provided herein comprises at least 1, 2, 3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS internucleotide bonds. In some embodiments, all internucleotide bonds of an ASO provided herein are PO bonds. In some embodiments, an ASO provided herein does not comprise PO internucleotide bonds.

In certain embodiments, the ASO comprises one or more chemical modifications at the 5′ end, the 3′ end, or both. Without wishing to be bound by theory, chemical modifications at one or both termini of a polynucleotide (e.g., polyribonucleotide) may stabilize the polynucleotide. In certain embodiments, the ASO comprises one or more chemical modifications in at least 1, 2, 3, 4, or 5 nucleotides at the 5′ end of the ASO. In certain embodiments, the ASO comprises one or more chemical modifications in at least 1, 2, 3, 4, or 5 nucleotides at the 3′ end of the ASO. In certain embodiments, the ASO comprises one or more chemical modifications in at least 1, 2, 3, 4, or 5 nucleotides at the 5′ end of the ASO and one or more chemical modifications in at least 1, 2, 3, 4, or 5 nucleotides at the 3′ end of the ASO.

The chemical structures can also be described in writing. In such cases, ‘M’ indicates MOE; ‘d’ indicates DNA, ‘L’ indicates LNA, “m” indicates 2′ OMethyl, ‘=’ indicates a phosphorothioate (PS) linkage, ‘-’ indicates a phosphodiester (PO) linkage; ‘*’ or ‘5C’ indicates 5-MethylCytosine, ‘ag’ indicates GalNAc, ‘tg’ or “teg” indicates Teg-GalNAc, ‘\hat{ }’ indicates FANA, “BioTeg” indicates Biotin; “Palm” indicates Palmitic acid; and “C18” indicates a Spacer 18 moiety.

To avoid ambiguity, this LNA has the formula:

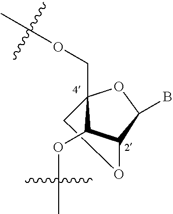

wherein B is the particular designated base.

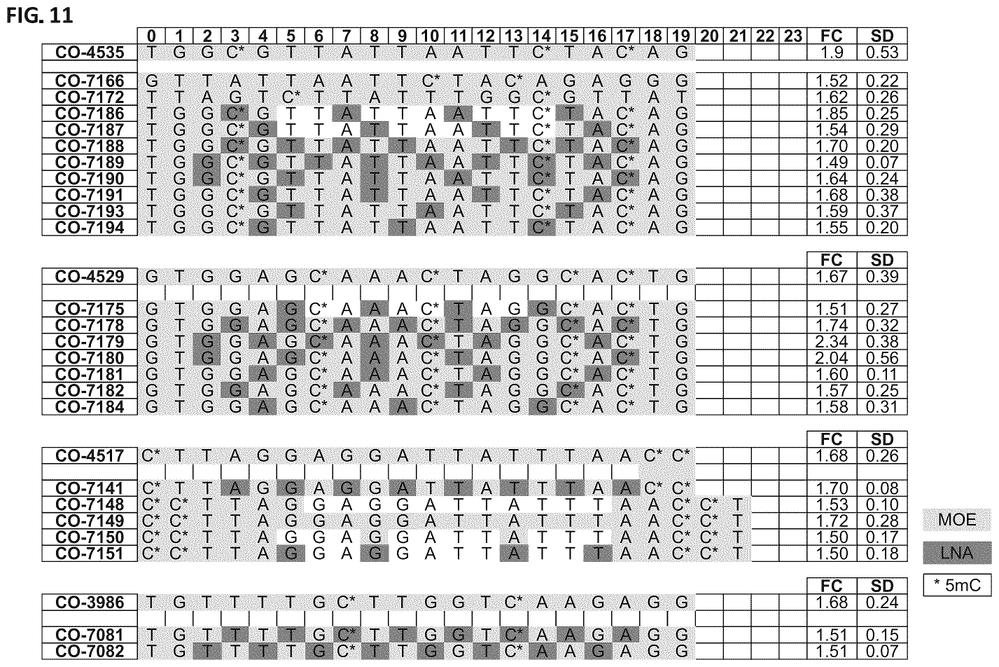

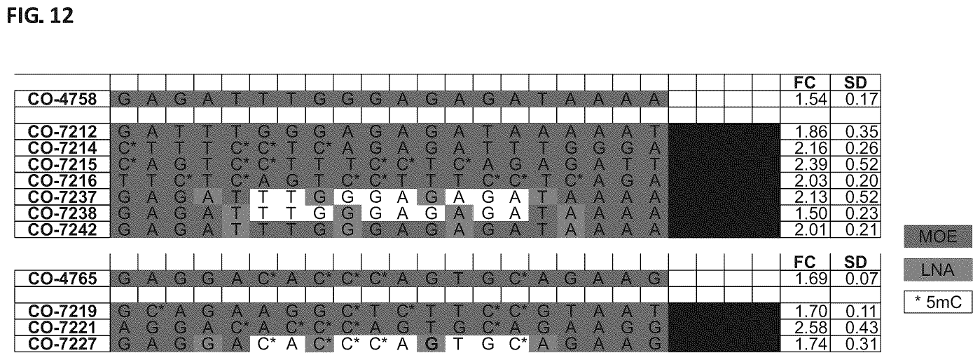

Exemplary visual representation of ASOs with chemical modifications are provided in FIGS. 11 and 12. Additional exemplary ASOs with chemical modifications are provided in Table 2. In some embodiments, an ASO provided herein comprises a nucleotide sequence and/or a chemical modification of any one of the ASOs provided in Table 2. In some embodiments, the ASO comprises a sequence and chemical modification selected from the group consisting of SEQ ID NOs: 14-644. In some embodiments, the ASO comprises a sequence and chemical modification selected from the group consisting of SEQ ID NOs: 14-103, 104-138, 139-148, 149-168, 169-216, 217-272, 273-381, 382-389, 390-407, 408-462, 464, 467-478, 479-514, 515, 516-528, 530-623, 625-644.

In some embodiments, an ASO provided herein comprises a nucleotide sequence of any one of the ASOs provided in Tables 3, 4, 5, 6, 7, 8, 9, 10, or 11. In some embodiments, the ASO comprises a sequence selected from the group consisting of SEQ ID NOs: 645-954, 955-1517, 1518-2057, 2058-2308, 2309-2806, 2807-3956, 3957-4061, 4062-5228, 5229-5519, and 5520-6021. In some embodiments, the ASO comprising a sequence selected from the group consisting of SEQ ID NOs: 645-954, 955-1517, 1518-2057, 2058-2308, 2309-2806, 2807-3956, 3957-4061, 4062-5228, 5229-5519, and 5520-6021 further comprises any chemical modification as disclosed herein.

### High Affinity Modified Nucleotides

A high affinity modified nucleotide is a modified nucleotide which, when incorporated into the oligonucleotide enhances the affinity of the oligonucleotide for its complementary target, for example as measured by the melting temperature (Tm). A high affinity modified nucleotide of the present disclosure preferably result in an increase in melting temperature between +0.5 to +12° C., such as between +1.5 to +10° C. or +3 to +8° C. per modified nucleotide. Numerous high affinity modified nucleotides are known in the art and include for example, many 2′ substituted nucleotides as well as locked nucleic acids (LNA) (see e.g., Freier & Altmann (1997) Nucl. Acid Res., 25:4429-4443 and Uhlmann (2000) Curr. Opinion in Drug Development, 3 (2), 203-213), each of which are hereby incorporated by reference.

### Sugar Modifications

The ASOs described herein may comprise one or more nucleotides which have a modified sugar moiety, i.e. a modification of the sugar moiety when compared to the ribose sugar moiety found in DNA and RNA. Numerous nucleotides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleotides include, for example, bicyclohexose nucleic acids (WO2011/017521) or tricyclic nucleic acids (WO2013/154798), both of which are hereby incorporated by reference. Modified nucleotides also include nucleotides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in RNA nucleotides. Substituents may, for example be introduced at the 2′, 3′, 4′ or 5′ positions.

In some embodiments, oligonucleotides comprise modified sugar moieties, such as any one of a 2′-O-methyl (2′OMe) moiety, a 2′-O-methoxyethyl moiety, a bicyclic sugar moiety, PNA (e.g., an oligonucleotide comprising one or more N-(2-aminoethyl)-glycine units linked by amide bonds or carbonyl methylene linkage as repeating units in place of a sugar-phosphate backbone), locked nucleotide (LNA) (e.g., an oligonucleotide comprising one or more locked ribose, and can be a mixture of 2′-deoxy nucleotides or 2′OMe nucleotides), cET (e.g., an oligonucleotide comprising one or more cET sugar), cMOE (e.g., an oligonucleotide comprising one or more cMOE sugar), morpholino oligomer (e.g., an oligonucleotide comprising a backbone comprising one or more phosphorodiamidate morpholiono oligomers), 2′-deoxy-2′-fluoro nucleotide (e.g., an oligonucleotide comprising one or more 2′-fluoro-β-D-arabinonucleotide), tcDNA (e.g., an oligonucleotide comprising one or more tcDNA modified sugar), constrained ethyl 2′-4′-bridged nucleic acid (cEt), S-cEt, ethylene bridged nucleic acid (ENA) (e.g., an oligonucleotide comprising one or more ENA modified sugar), hexitol nucleic acids (HNA) (e.g., an oligonucleotide comprising one or more HNA modified sugar), or tricyclic analog (tcDNA) (e.g., an oligonucleotide comprising one or more tcDNA modified sugar).

In some embodiments, oligonucleotides comprise nucleobase modifications selected from the group consisting of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallyIC”), an abasic nucleotide, Z base, P base, Unstructured Nucleic Acid (“UNA”), isoguanine (“isoG”), and isocytosine (“isoC”), glycerol nucleic acid (GNA), thiomorpholino (C4H9NS) or thiophosphoramidate morpholinos (TMOs). Synthesis of glycerol nucleic acid (GNA) (also known as glycol nucleic acids) is described in Zhang et al, Current Protocols in Nucleic Acid Chemistry 4.40.1-4.40.18, September 2010, hereby incorporated by reference. Synthesis of thiophosphoramidate Morpholino Oligonucleotides is described in Langer et al, J. Am. Chem. Soc. 2020, 142 (38): 16240-53

### 2′ Sugar Modified Nucleotides

A 2′ sugar modified nucleotide is a nucleotide which has a substituent other than H or —OH at the 2′ position (2′ substituted nucleotide) or comprises a 2′ linked biradical capable of forming a bridge between the 2′ carbon and a second carbon in the ribose ring, such as LNA (2′-4′ biradical bridged) nucleotides.

Without wishing to be bound by theory, the 2′ modified sugar may provide enhanced binding affinity and/or increased nuclease resistance to the oligonucleotide. Examples of 2′ substituted modified nucleotides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleotide. For further examples, see e.g., Freier & Altmann 1997, supra; Uhlmann 2000, supra, and Deleavey and Damha (2012) Chemistry and Biology 19:937, each of which are hereby incorporated by reference., each of which are hereby incorporated by reference.

Locked Nucleic Acid nucleotides (LNA Nucleotide)

A “LNA nucleotide” is a 2′-sugar modified nucleotide which comprises a biradical linking the C2′ and C4′ of the ribose sugar ring of said nucleotide (also referred to as a “2′-4′ bridge”), which restricts or locks the conformation of the ribose ring. In other words, a locked nucleotide is a nucleotide comprising a bicyclic sugar moiety comprising a 4′-CH₂—O-2′ bridge. This structure effectively “locks” the ribose in the 3′-endo structural conformation. The addition of locked nucleotides to oligonucleotides has been shown to increase oligonucleotide stability in serum, and to reduce off-target effects (Grunweller, A. et al., (2003) Nucleic Acids Research 31 (12): 3185-3193). These nucleotides are also sometimes termed bridged nucleic acid or bicyclic nucleic acid (BNA). The locking of the conformation of the ribose is associated with an enhanced affinity of hybridization (duplex stabilization) when the LNA is incorporated into an oligonucleotide with complementarity to an RNA or a DNA molecule. This can be routinely determined by measuring the melting temperature of the oligonucleotide/complement duplex. Exemplary LNA nucleotides include beta-D-oxy-LNA, 6′-methyl-beta-D-oxy LNA such as (S)-6′-methyl-beta-D-oxy-LNA (ScET) and ENA.

Examples of bicyclic nucleotides for use in the polynucleotides of the disclosure include without limitation nucleotides comprising a bridge between the 4′ and the 2′ ribosyl ring atoms. In certain embodiments, the polynucleotide agents of the disclosure include one or more bicyclic nucleotides comprising a 4′ to 2′ bridge. Examples of such 4′ to 2′ bridged bicyclic nucleotides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleotides can be prepared having one or more stereochemical sugar configurations including for example α-L-ribofuranose and β-D-ribofuranose (see PCT Application Publication No. WO 99/14226, contents of which are incorporated by reference herein).

An oligonucleotide of the disclosure can also be modified to include one or more constrained ethyl nucleotides. As used herein, a “constrained ethyl nucleotide” or “cEt” is a locked nucleotide comprising a bicyclic sugar moiety comprising a 4′-CH(CH₃)—O-2′ bridge. In one embodiment, a constrained ethyl nucleotide is in the S conformation referred to herein as “S-cEt.”

An oligonucleotide of the disclosure may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to an RNA (e.g., a regRNA or a mRNA). The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide of the disclosure comprises one or more monomers that are UNA (unlocked nucleotide) nucleotides. UNA is unlocked acyclic nucleotide, wherein any of the bonds of the sugar has been removed, forming an unlocked “sugar” residue. In one example, UNA also encompasses monomers with bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose sugar may also be modified with a cyclopropane ring to produce a tricyclodeoxynucleic acid (tricyclo DNA). The ribose moiety may be substituted for another sugar such as 1,5,-anhydrohexitol, threose to produce a threose nucleotide (TNA), or arabinose to produce an arabino nucleotide. The ribose molecule can also be replaced with non-sugars such as cyclohexene to produce cyclohexene nucleotide or glycol to produce glycol nucleotides.

Potentially stabilizing modifications to the ends of nucleotide molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT (idT) and others. Disclosure of this modification can be found in PCT Publication No. WO 2011/005861.

Other alternatives chemistries of an oligonucleotide of the disclosure include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Additional non-limiting, exemplary LNA nucleotides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75 (5) pp. 1569-81, Mitsuoka et al., Nucleic Acids Research 2009, 37 (4), 1225-1238, and Wan and Seth, J. Medical Chemistry 2016, 59, 9645-9667, each of which are hereby incorporated by reference.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the nucleotide sugar modification is 2′-O—C1-4alkyl such as 2′-O-methyl (2′-OMe), 2′-deoxy (2′-H), 2′-O—C1-3alkyl-O—C1-3alkyl such as 2′-methoxyethyl (‘2’-MOE″), 2′-fluoro (“2′-F”), 2′-amino (“2′—NH2”), 2′-arabinosyl (“2′-arabino”) nucleotide, 2′-F-arabinosyl (“2′-F-arabino”) nucleotide, 2′ locked nucleic acid (“LNA”) nucleotide, 2′-amido bridge nucleic acid (AmNA), 2′-unlocked nucleic acid (“ULNA”) nucleotide, a sugar in L form (“L-sugar”), or 4′-thioribosyl nucleotide.

### Mixmers and Gapmers

The ASO can have a mixmer and/or gapmer structure, for example, in a pattern disclosed by the ASOs in FIGS. 11 and 12.

In certain embodiments, the ASO is a mixmer. As used herein, the term “mixmer” refers to an oligonucleotide comprising an alternating composition of DNA monomers and nucleotide analogue monomers across at least a portion of the oligonucleotide sequence. In certain embodiments, the ASO is a mixmer based on the gapmer structure, comprising a mixture of DNA nucleotides and 2′-MOE nucleotides in the gap, flanked by RNA sequences (e.g., 2′-modified RNA sequences) in the wings. Mixmers may be designed to comprise a mixture of affinity enhancing nucleotide analogues, such as in non-limiting example 2′-O-alkyl-RNA monomers, 2′-amino-DNA monomers, 2′-fluoro-DNA monomers, LNA monomers, arabino nucleic acid (ANA) monomers, 2′-fluoro-ANA monomers, HNA monomers, INA monomers, 2′-MOE-RNA (2′-O-methoxyethyl-RNA), 2′-Fluoro-DNA, and LNA. In some embodiments, the mixmer is incapable of recruiting RNase H. In some embodiments, the mixmer comprises one type of affinity enhancing nucleotide analogue together with DNA and/or RNA.

Multiple different modifications can be interspaced in a mixmer. For example, the ASO can comprise LNA modification in a plurality of nucleotides and a different modification in some or all of the rest of the nucleotides. In some embodiments, any two adjacent LNA-modified nucleotides are separated by at least 1, 2, 3, 4, or 5 nucleotides. Throughout the ASO, the distance between adjacent LNA-modified nucleotides can either be constant (e.g., any two adjacent LNA-modified nucleotides are separated by 1, 2, 3, 4, or 5 nucleotides) or variable. In some embodiments, the length of the ASO is 3×n, 3×n−1, or 3×n−2 nucleotides (n is an integer of 6 or greater), wherein (a) (i) the nucleotides at positions 3×m−2 (m is an integer from 1 to n) are nucleotides (e.g., ribonucleotides or deoxyribonucleotides) comprising a first modification (e.g., LNA), (ii) the nucleotides at positions 3×m−1 (m is an integer from 1 to n) are nucleotides (e.g., ribonucleotides or deoxyribonucleotides) comprising a first modification (e.g., LNA), or (iii) the nucleotides at positions 3×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). In some embodiments, the length of the ASO is 2×n or 2×n−1 nucleotides (n is an integer of 9 or greater), wherein (a) (i) the nucleotides at positions 2×m−1 (m is an integer from 1 to n) are nucleotides (e.g., ribonucleotides or deoxyribonucleotides) comprising a first modification (e.g., LNA), or (ii) the nucleotides at positions 2×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). Similar modification patterns, for example, where the first modification is repeated very 4, 5, or more nucleotides, are also contemplated. In some embodiments, the length of the ASO is 4×n, 4×n−1, or 4×n−2 nucleotides (n is an integer of 6 or greater), wherein (a) (i) the nucleotides at positions 4×m−2 (m is an integer from 1 to n) are nucleotides (e.g., ribonucleotides or deoxyribonucleotides) comprising a first modification (e.g., LNA), (ii) the nucleotides at positions 4×m−1 (m is an integer from 1 to n) are nucleotides (e.g., ribonucleotides or deoxyribonucleotides) comprising a first modification (e.g., LNA), or (iii) the nucleotides at positions 3×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). In some embodiments, the length of the ASO is 5×n, 5×n−1, or 5×n−2 nucleotides (n is an integer of 6 or greater), wherein (a) (i) the nucleotides at positions 5×m−2 (m is an integer from 1 to n) are nucleotides (e.g., ribonucleotides or deoxyribonucleotides) comprising a first modification (e.g., LNA), (ii) the nucleotides at positions 5×m−1 (m is an integer from 1 to n) are nucleotides (e.g., ribonucleotides or deoxyribonucleotides) comprising a first modification (e.g., LNA), or (iii) the nucleotides at positions 5×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl).

In some embodiments, the ASO further comprises a GalNAc or Teg-GalNAc moiety at the 5′ or 3′ end of the ASO.

In certain embodiments, the ASO comprises a DNA sequence (e.g., having at least 1, 2, 3, 4, 5, 6, 7, 8, 9, 10, 11, 12, 13, 14, or 15 contiguous nucleotides of unmodified DNA) flanked on both sides by RNA sequences. Such structure is known as “gapmer,” in which the DNA region is referred to as the “gap” and the RNA regions is referred to as the “wings” (see, e.g., PCT Application Publication No. WO2013/177248). Gapmers were known to facilitate degradation of the target RNA by recruiting nucleases (e.g., nuclear RNAses (e.g., RNase H)). Surprisingly, in some embodiments of the present disclosure, it has been discovered that a gapmer that binds to a regRNA having the same sequence as having a parent ASO but having different chemical modifications, can also increase target gene expression. In certain embodiments, the ASO comprises a DNA sequence flanked by RNA sequences and does not induce RNAse-or RNAse H-mediated degradation.

In some embodiments, the ASO gapmer comprises an internal DNA region flanked by two external RNA “wings.” For example, the internal DNA gap can comprise at least 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s), while each of the external RNA wing(s) can independently comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Exemplary gapmer structures include, but are not limited to a 1-10-9, 2-10-8, 3-10-7, 4-10-6, 6-10-4, 7-10-3, 8-10-2, 9-10-1, 1-18-1, 2-16-2, 3-14-3, 4-12-4, 5-10-5, 6-8-6, 7-6-7, 8-5-7, 7-5-8, 8-4-8, or 9-2-9 structure where the first and third number indicate the number of external RNA nucleotides and the second number indicates the number of internal DNA nucleotides.

The ASO can also be a mixmer comprising one DNA region linked to one RNA region. In some embodiments, the mixmer comprises at least 10 DNA nucleotides linked to at least 10 RNA nucleotides, wherein the DNA nucleotides are at the 5′ end of the mixmer or the 3′ end of the mixmer. In some embodiments, the mixmer comprises at least 1, 2, 3, 4, 5, 6, 7, 8, 9, 10, 11, 12, 13, 14, 15, 16, 17, 18, or 19 DNA nucleotide(s) linked to at least 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 RNA nucleotide(s), wherein the DNA nucleotides are at the 5′ end of the mixmer or the 3′ end of the mixmer. In some embodiment, the RNA regions of the gapmer or mixmer can comprise any additional chemical modification as disclosed herein.

In certain embodiments, the ASO (e.g., the gapmer or mixmer) is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides in length. In certain embodiments, the gap is about 1, 2, 3, 4, 5, 6, 7, 8, 9, 10, 11, 12, 13, 14, 15, 16, 17, 18, 19, or more nucleotides in length. In certain embodiments, one or both wings are about 2, 3, 4, 5, 6, 7, 8, 9, 10, 11, 12, 13, 14, 15, 16, 17, 18, or 19 or more nucleotides in length. In certain embodiments, one or both wings comprises RNA modifications, for example, β-D-ribo nucleotides, 2′-modified nucleotides (e.g., 2′-O-(2-Methoxyethyl) (2′-MOE), 2′-O—CH₃, or 2′-fluoro-arabino (FANA)), and bicyclic sugar modified nucleotides (e.g., having a constrained ethyl or locked nucleic acid (LNA)). In certain embodiments, each ribonucleotide in the mixmer or gapmer is modified by 2′-MOE. In certain embodiments, the mixmer or gapmer comprises one or more modified internucleotide bonds, e.g., phosphorothioate (PS) internucleotide linkage. In certain embodiments, each two adjacent nucleotides in the mixmer or gapmer are linked by a phosphorothioate internucleotide bond.

In certain embodiments, the ASO does not comprise 1 or more, 2 or more, 3 or more, 4 or more, 5 or more, 6 or more, 7 or more, 8 or more, 9 or more, 10 or more, 10 or more, 11 or more, 12 or more, 13 or more, 14 or more, 15 or more, 20 or more, 25 or more, 30 or more, 35 or more, 40 or more, or 45 or more contiguous nucleotides of unmodified DNA. In some embodiments, such a DNA sequence is disrupted by modified (e.g., 2′-MOE modified) ribonucleotides every 2, 3, 4, 5, or more nucleotides. In some embodiments, the ASO comprises only ribonucleotides and no deoxyribonucleotides.

The structural features of mixmer and gapmer can be combined. In certain embodiments, the ASO has a structure similar to that of a mixmer disclosed herein (e.g., one having interspaced modifications), except that the second modification in the gap is changed to a third modification (e.g., deoxyribonucleotide). In certain embodiments, the ASO has a structure similar to that of a gapmer disclosed herein, except that in the gap the nucleotides are modified in a mixmer pattern.

In certain embodiments, the ASO further comprises a ligand moiety, e.g., a ligand moiety that specifically targets a tissue or organ in a subject. For example, N-Acetylgalactosamine (GalNAc) specifically targets liver. In certain embodiments, the ligand moiety comprises GalNAc. In certain embodiments, the ligand moiety comprises a three-cluster GalNAc moiety, commonly denoted GAINAc3. Other types of GalNAc moieties are one-cluster, two cluster or four cluster GAINAc, denoted as GAINAc1, GAINAc2, or GAINAc4. In certain embodiments, the ligand moiety comprises GalNAcl, GALNAc2, GAINAc3, or GalNAc4.

In certain embodiments, the ligand moiety comprises biotin. In certain embodiments, the ligand moiety comprises palmitic acid. In certain embodiments, the ligand moiety comprises a Spacer 18 moiety (C18).

### III. Pharmaceutical Compositions

In certain embodiments, an ASO disclosed herein can be present in pharmaceutical compositions. The pharmaceutical composition can be formulated for use in a variety of drug delivery systems. One or more pharmaceutically acceptable excipients or carriers can also be included in the composition for proper formulation. In some embodiments, the pharmaceutical acceptable carrier comprises sterile saline, sterile water, or phosphate buffered saline (PBS). Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

Exemplary carriers and pharmaceutical formulations suitable for delivering nucleic acids are described in Durymanov and Reineke (2018) Front. Pharmacol. 9:971; Barba et al. (2019) Pharmaceutics 11 (8): 360; Ni et al. (2019) Life (Basel) 9 (3): 59, each of which is incorporated herein by reference. It is understood that the presence of a ligand moiety conjugated to the ASO may circumvent the need for a carrier for delivery to a tissue or organ targeted by the ligand moiety.

The delivery of an oligonucleotide of the disclosure to a cell e.g., a cell within a subject, such as a human subject e.g., a subject in need thereof, such as a subject having or at risk of developing cholestasis or a cholestatic liver disease can be achieved in a number of different ways. For example, delivery may be performed by contacting a cell with an oligonucleotide of the disclosure either in vitro or in vivo. In vivo delivery may also be performed directly by administering a composition comprising an oligonucleotide to a subject. These alternatives are discussed further below.

In general, any method of delivering a nucleic acid molecule (in vitro or in vivo) can be adapted for use with an oligonucleotide of the disclosure (see e.g., Akhtar S. and Julian R L., (1992) Trends Cell. Biol. 2 (5): 139-144 and WO 94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleotide linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the disclosure. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the disclosure are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

### Membranous Molecular Assembly Delivery Methods

Oligonucleotides of the disclosure can also be delivered using a variety of membranous molecular assembly delivery methods including polymeric, biodegradable microparticle, or microcapsule delivery devices known in the art. For example, a colloidal dispersion system may be used for targeted delivery of an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75:4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4 (6): 466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside Gun or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis (oleoyloxy)-3-(trimethylammonia) propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with oligonucleotides are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present disclosure are described in PCT Publication Nos. WO 2009/088891, WO 2009/132131, and WO 2008/042973, which are hereby incorporated by reference in their entirety.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the disclosure can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Oligonucleotides of in the disclosure may be fully encapsulated in a lipid formulation, e.g., a lipid nanoparticle (LNP), or other nucleic acid-lipid particle. LNPs are useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

Non-limiting examples of cationic lipids include N,N-dioleyl-N,N-dimethylammonium chloride (DODAC), N,N-distearyl-N,N-dimethylammonium bromide (DDAB), N—(I-(2,3-dioleoyloxy) propyl)-N,N,N-trimethylammonium chloride (DOTAP), N—(I-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.C1), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z, 12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino) bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (C₁₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

The ASO may also be deliver in a lipidoid. The synthesis of lipidoids has been extensively described and formulations containing these compounds are particularly suited for delivery of modified nucleic acid molecules or ASOs (see Mahon et al, Bioconjug Chem. 2010 21:1448-1454; Schroeder et al, J Intern Med. 2010 267:9-21; Akinc et al, Nat Biotechnol. 2008 26:561-569; Love et al, Proc Natl Acad Sci USA. 2010 107:1864-1869; Siegwart et al, Proc Natl Acad Sci USA. 2011 108:12996-3001; all of which are incorporated herein in their entireties).

Lipid compositions for RNA delivery are disclosed in WO2012170930A1, WO2013149141A1, and WO2014152211A1, each of which are hereby incorporated by reference.

### IV. Therapeutic Applications

The present disclosure provides methods for treating an ABCB11-related diseases and disorders. In some embodiments, the ABCB11-related diseases and disorders is associated with decreased ABCB11 gene expression. In some embodiments, the ABCB11-related diseases and disorders is associated with accumulation of bile acid in cells (e.g., hepatocytes).

The present disclosure provides methods for treating or preventing cholestasis in a subject in need thereof. The method includes administering an ASO provided herein or a pharmaceutical composition including an ASO provided herein to the subject. In some embodiments, the subject has or is at risk of developing a cholestatic liver disease. In some embodiments, the cholestatic liver disease is progressive familial intrahepatic cholestasis (PFIC) (e.g., PFIC type 1 (PFIC1), PFIC type 2 (PFIC2) and PFIC type 3 (PFIC3)), Alagille Syndrome (ALGS), primary sclerosing cholangitis (PSC) (e.g., pediatric PSC), neonatal sclerosing cholangitis, pediatric intrahepatic cholestasis (e.g., pediatric primary intrahepatic cholestasis or pediatric secondary intrahepatic cholestasis), benign recurrent intrahepatic cholestasis (BRIC) (e.g., BRIC type 1, BRIC type 2, BRIC type 3), total parenteral nutrition associated cholestasis, paraneoplastic cholestasis, Stauffer syndrome, drug-associated cholestasis, infection-associated cholestasis, biliary atresia, intrahepatic cholestasis of pregnancy, primary biliary cholangitis (PBC), Dubin-Johnson syndrome, post-Kasai biliary atresia, post-liver transplantation biliary atresia, post-liver transplantation cholestasis, post-liver transplantation associated liver disease, intestinal failure associated liver disease, bile acid mediated liver injury, MRP3 deficiency syndrome, or gallstone disease. In some embodiments, the subject has or is at risk of developing PFIC2.

In some embodiments, the cholestatic liver disease is characterized by one or more symptoms selected from increased serum concentration of bile acids, increased hepatic concentration of bile acids, increased serum concentration of bilirubin, hepatocellular injury, liver scarring, liver failure, hepatomegaly, jaundice, pruritis, hypercholemia, neonatal respiratory distress syndrome, cirrhosis, lung pneumonia, xanthomas, malabsorption, hepatocellular carcinoma, splenomegaly, diarrhea, hearing loss, pancreatitis, hepatocellular necrosis, giant cell formation, gastrointestinal bleeding, portal hypertension, fatigue, loss of appetite, anorexia, steatorrhea, failure to thrive, and/or renal failure.

In some embodiments, the methods described herein include administering in combination with one or more additional therapeutics. Examples of additional therapeutics include obeticholic acid (OCA), ursodeoxycholic acid (UDCA), a PPARα agonist (e.g., lofibrate, gemfibrozil, ciprofibrate, bezafibrate, and fenofibrate), a PPARδ agonist (e.g., seladelpar), a dual α, δ PPAR agonist (e.g., elafibranor), a corticosteroid (e.g., budesonide), and an apical sodium-dependent bile acid transporter inhibitor (e.g., maralixibat, volixibat, odevixibat, elobixibat, and linerixibat).

The oligonucleotide compositions described herein are useful in the methods of the disclosure and, while not bound by theory, are believed to exert their desirable effects through their ability to modulate the levels of ABCB11 protein, and/or ABCB11 mRNA, and/or the ABCB11 activity within a cell, e.g., by increasing the level of the ABCB11 protein in a cell in a subject (e.g., a mammal, a mouse, a hamster, a non-human a primate (e.g., a monkey), or a human).

Still another aspect of the present disclosure includes methods of increasing expression of ABCB11 in a cell of a subject, comprising contacting the cell with an ASO of the disclosure (or a pharmaceutical composition including the ASO), thereby treating the disease or disorder in the subject.

In yet another aspect, the disclosure provides an ASO of the disclosure (or a pharmaceutical composition including the ASO) for use as a medicament. Further, the disclosure provides for an ASO of the disclosure (or a pharmaceutical composition including the ASO) for use in therapy.

Yet another aspect of the disclosure includes methods of modulating (e.g., increasing or reducing) expression of an ABCB11 gene in a cell (e.g., in vivo, ex vivo, or in vitro) including contacting the cell with an ASO of the disclosure (or a pharmaceutical composition including the ASO), thereby increasing the expression of a ABCB11 gene in the cell. In some embodiments, the cell is a mammalian cell (e.g., a human cell such as a human hepatocyte). The methods may include contacting a cell with an ASO of the disclosure (or a pharmaceutical composition including the ASO), in an amount effective to increase expression of an ABCB11 gene in the cell, thereby increasing expression of an ABCB11 gene in the cell. In some embodiments, contacting the cell with the ASO (or a pharmaceutical composition including the ASO) modulates (e.g., increases) the amount of ABCB11 mRNA in the cell. In some embodiments, contacting the cell with the ASO (or a pharmaceutical composition including the ASO) modulates (e.g., increases) the amount of ABCB11 protein in the cell.

Contacting of a cell with an ASO may be performed in vitro, ex vivo, or in vivo. Contacting a cell in vivo with the ASO includes contacting a cell or group of cells within a subject, e.g., a human subject, with the ASO. Combinations of in vitro, ex vivo, and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc3 ligand, or any other ligand that directs the oligonucleotide to a site of interest. The cell can be a liver cell (e.g., a hepatocyte).

Administration of the ASOs or pharmaceutical compositions disclosed herein to a subject can be intravenous, intraarterial, intraperitoneal, intramuscular, subcutaneous, intrapleural, intrathecal, intracavitary, by perfusion through a catheter or by direct intralesional injection. In certain embodiments, the ASO or pharmaceutical composition is administered systemically. In certain embodiments, the ASO or pharmaceutical composition is administered by a parenteral route. For example, in certain embodiments, the ASO or pharmaceutical composition is administered by intravenously (e.g., by intravenous infusion), for example, with a prefilled bag, a prefilled pen, or a prefilled syringe. In other embodiments, the ASO or pharmaceutical composition is administered locally to an organ or tissue in which an increase in the target gene expression is desirable (e.g., liver).

In some embodiments, the oligonucleotide is administered to a subject such that the oligonucleotide is delivered to a specific site within the subject. Such targeted delivery can be achieved by either systemic administration or local administration. An increase in the expression of ABCB11 may be assessed using measuring the level or change in the level of ABCB11 mRNA or ABCB11 protein in a sample (e.g., blood, tissue or liver sample) derived from a specific site within the subject. In certain embodiments, the methods include a clinically relevant increase of expression of ABCB11, e.g., as demonstrated by a clinically relevant outcome after treatment of a subject with an agent to reduce the expression of ABCB11.

In other embodiments of the methods provided herein, the oligonucleotide is administered in an amount and for a time effective to result in a reduction (e.g., by 10%, 20%, 30%, 40%, 50%, 60%, 70%, 80%, 90%, or 100%) of one or more symptoms of cholestatic liver disease provided herein.

In some embodiments of the methods provided herein, administration of an ASO of the disclosure to a subject results in a reduction in a symptom or a change in a disease-relevant laboratory measure of a cholestatic liver disease in the subject. In some embodiments, the reduction in a symptom or change in the disease-relevant laboratory measure of cholestatic liver disease comprises a reduction in serum bile acid concentration, an increase in serum concentration of 7α-hydroxy-4-cholesten-3-one (7αC4), an increase in the ratio of serum 7αC4: serum bile acid, an increase in fecal bile acid excretion, a reduction in pruritis, a reduction in serum alanine aminotransferase (ALT) concentration, or a combination thereof. In some embodiments, the reduction in the symptom or disease relevant laboratory measure is determined relevant to a baseline level, concentration, or ratio, e.g., prior to administration of the ASO to the subject or a level determined to be within a normal range as determined from a similar subject that does not have the cholestatic liver disease.

In some embodiments, administration of an ASO or a pharmaceutical composition of the disclosure to a subject results in a reduction in serum bile acid concentration (e.g., as determined using liquid chromatography tandem mass spectrometry (LC-MS/MS)). In some embodiments, the serum bile acid concentration of the subject is reduced by at least about 5%, 10%, 20%, 30%, 40%, 50%, 60%, 70%, 80%, or 90% relative to a baseline concentration.

In some embodiments, administration of an ASO or a pharmaceutical composition of the disclosure to a subject results in an increase in serum 7αC4 concentration (e.g., as determined using LC-MS/MS; see e.g., Camilleri et al. (2009) Neurogastroenterol. Motil. 21:734-e-43, incorporated herein by reference). In some embodiments, the serum 7αC4 concentration of the subject is increased by at least about 10%, 20%, 30%, 40%, 50%, 60%, 70%, 80%, 90%, 100%, 150%, 200%, 300%, 400%, 500%, 600%, 700%, 800%, 900%, 1000% relative to a baseline concentration. In some embodiments, the serum 7αC4 concentration of the subject is increased by at least about 1-fold, 2-fold, 3-fold, 4-fold, 5-fold, 6-fold, 7-fold, 8-fold, 9-fold, or 10-fold, 20-fold, 30-fold, 40-fold, 50-fold, 60-fold, 70-fold, 80-fold, 90-fold, 100-fold, 200-fold, 300-fold, 400-fold, 500-fold or more relative to a baseline concentration.

In some embodiments, administration of an ASO or a pharmaceutical composition of the disclosure to a subject results in an increase in the ratio of serum 7αC4: serum bile acid in the subject by at least about 10%, 20%, 30%, 40%, 50%, 60%, 70%, 80%, 90%, 100%, 150%, 200%, 300%, 400%, 500%, 600%, 700%, 800%, 900%, 1000% relative to a baseline ratio. In some embodiments, administration of an ASO of the disclosure to a subject results in an increased ratio of serum 7αC4: serum bile acid in the subject by at least about 1-fold, 1.5-fold, 1.75-fold, 2-fold, 2.5-fold, 3-fold, 4-fold, 5-fold, 6-fold, 7-fold, 8-fold, 9-fold, or 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 3000-fold, 4000-fold, 5000-fold, 10000-fold, or more relative to a baseline ratio.

In some embodiments, administration of an ASO or a pharmaceutical composition of the disclosure to a subject results in an increase in fecal bile acid excretion (as determined using LC-MS/MS) by at least about 10%, 20%, 30%, 40%, 50%, 60%, 70%, 80%, 90%, 100%, 150%, 200%, 300%, 400%, 500%, 600%, 700%, 800%, 900%, 1000% relative to a baseline level. In some embodiments, administration of an ASO or a pharmaceutical composition of the disclosure to a subject results in increased fecal bile acid excretion by at least about 1-fold, 2-fold, 3-fold, 4-fold, 5-fold, 6-fold, 7-fold, 8-fold, 9-fold, or 10-fold, 20-fold, 30-fold, 40-fold, 50-fold, 60-fold, 70-fold, 80-fold, 90-fold, 100-fold, or more relative to a baseline level.

In some embodiments, administration of an ASO of the disclosure to a subject results in an reduction in the severity of pruritis in the subject (e.g., as determined using an ItchRO (Observer) score, an ItchRO (Patient) score, or a combination thereof (see e.g., Kamath et al. (2018) Patient 11 (1): 69-82, incorporated herein by reference). In some embodiments, administration of an ASO of the disclosure results in a reduction in the ItchRO (Observer) score on a scale of 1 to 4 of about or of at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.25, 2.5, or 3 or more relative to baseline. In some embodiments, the administration of an ASO of the disclosure results in a reduction in the ItchRO (Patient) score on a scale of 1 to 10 of about or of at least about 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, or 10. In some embodiments, administration of an ASO of the disclosure results in a reduction of the ItchRO (Observer) score, the ItchRO (Patient) score, or both to zero.

In some embodiments, administration of an ASO of the disclosure to a subject results in a reduction in serum alanine aminotransferase (ALT) concentration (e.g., as determined using a spectrophotometric method as described in Knight et al. (1968) J. Med. Lab Technol. 25:106-111) of at least about 1%, 2%, 3%, 4%, 5%, 6%, 7%, 8%, 9%, 10%, 11%, 12%, 13%, 14%, 15%, 16%, 17%, 18%, 19%, 20%, 25%, or 30% relative to a baseline concentration.

### Increase of ABCB11 Expression

In some aspects, the therapeutic methods disclosed herein, using an ASO that targets an ABCB11 regRNA, result in increased ABCB11 expression in a subject. Increasing the expression of an ABCB11 gene includes any level of increasing expression of a ABCB11 gene, e.g., at least partial increase of the expression of a ABCB11 gene. Increased expression may be assessed by an increase in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only (vehicle) control or inactive agent control). In certain embodiments, the method causes a clinically relevant increase of expression of ABCB11, e.g., as demonstrated by a clinically relevant outcome after treatment of a subject with an ASO provided herein to increase the expression of ABCB11.

In certain embodiments, the methods disclosed herein result in increased ABCB11 gene expression in a cell, tissue, or subject by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, relative to the pre-dose, pre-administration, or pre-exposure baseline level. In certain embodiments, the methods disclosed herein result in increased ABCB11 gene expression in a cell, tissue, or subject by at least 1-fold, at least 1.5-fold, at least 2-fold, at least 3-fold, at least 4-fold, at least 5-fold, at least 6-fold, at least 7-fold, at least 8-fold, at least 9-fold, or at least 10-fold or more relative to the pre-dose, pre-administration, or pre-exposure baseline level. In certain embodiments, the cell, tissue, or subject has a deficiency in ABCB11 expression, and the method disclosed herein restores the ABCB11 expression level (e.g., ABCB11 protein level or ABCB11 mRNA level) or ABCB11 protein activity to at least 20%, at least 30%, at least 40%, at least 50%, at least 60%, at least 70%, at least 80%, at least 90%, or at least 100% of the average ABCB11 expression level (e.g., ABCB11 protein level or ABCB11 mRNA level) or ABCB11 protein activity in similar cells, tissues or subjects (e.g., of the same species, of the like age, and/or of the same sex) that do not have a deficiency in ABCB11 expression.

In some embodiments, an ASO of the disclosure may enhance the production of ABCB11 mRNA (e.g., in a cell or in a cell, tissue, or sample of a subject) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, an ASO of the disclosure may enhance the production of ABCB11 mRNA (e.g., in a cell or in a cell, tissue, or sample of a subject) by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

In some embodiments, an ASO of the disclosure may enhance the production of ABCB11 protein (e.g., in a cell or in a cell, tissue, or sample of a subject) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, an ASO of the disclosure may enhance the production of ABCB11 protein (e.g., in a cell or in a cell, tissue, or sample of a subject) by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

The expression of an ABCB11 gene may be assessed based on the level of any variable associated with ABCB11 gene expression, e.g., ABCB11 mRNA level or ABCB11 protein level. In certain embodiments, the expression level or activity of ABCB11 herein refers to the average expression level or activity of ABCB11 in the liver (e.g., of a mammal or human subject).

In certain embodiments, surrogate markers can be used to detect an increase of ABCB11 expression. For example, effective treatment of a ABCB11-related disorder (e.g., a cholestatic liver disease provided herein), as demonstrated by acceptable diagnostic and monitoring criteria with an agent to increase ABCB11 expression can be understood to demonstrate a clinically relevant increase in ABCB11.

Increase of the expression of a ABCB11 gene may be manifested by an increase of the amount of mRNA expressed by a first cell or group of cells (such cells may be present, for example, in a sample derived from a subject) in which a ABCB11 gene is transcribed and which has or have been treated (e.g., by contacting the cell or cells with an oligonucleotide of the disclosure, or by administering an oligonucleotide of the disclosure to a subject in which the cells are or were present) such that the expression of a ABCB11 gene is increased, as compared to a second cell or group of cells substantially identical to the first cell or group of cells but which has not or have not been so treated (control cell(s) not treated with an oligonucleotide or not treated with an oligonucleotide targeted to the gene of interest).

In other embodiments, increase of the expression of a ABCB11 gene may be assessed in terms of an increase of a parameter that is functionally linked to ABCB11 gene expression, e.g., bile acids, ABCB11 protein expression or ABCB11 activity or as otherwise provided herein. An increase in ABCB11 expression may be determined in any cell expressing ABCB11 mRNA or protein, either endogenous or heterologous from an expression construct, and by any assay known in the art.

An increase of ABCB11 mRNA or protein expression may be manifested by an increase in the level of the ABCB11 protein that is expressed by a cell or group of cells (e.g., the level of protein expressed in a sample derived from a subject), relative to a control cell or a control group of cells. An increase of ABCB11 expression may also be manifested by an increase in the level of the ABCB11 mRNA level in a treated cell or group of cells, relative to a control cell or a control group of cells.

A control cell or group of cells that may be used to assess the increase of the expression of a ABCB11 gene includes a cell or group of cells that has not yet been contacted with an oligonucleotide of the disclosure. For example, the control cell or group of cells may be derived from an individual subject (e.g., a human or animal subject) prior to treatment of the subject with an oligonucleotide.

The level of ABCB11 mRNA that is expressed by a cell or group of cells may be determined using any method known in the art for assessing mRNA expression. In one embodiment, the level of expression of ABCB11 in a sample is determined by detecting a transcribed polynucleotide, or portion thereof, e.g., ABCB11 mRNA. RNA may be extracted from cells using RNA extraction techniques including, for example, using acid phenol/guanidine isothiocyanate extraction (RNAzol B; Biogenesis), RNEasy™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating ABCB11 mRNA may be detected using methods the described in PCT Publication WO 2012/177906, the entire contents of which are hereby incorporated herein by reference. In some embodiments, the level of expression of ABCB11 is determined using a nucleic acid probe. The term “probe,” as used herein, refers to any molecule that is capable of selectively binding to a specific ABCB11 sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR) analyses, and probe arrays. One method for the determination of mRNA levels involves contacting the isolated mRNA with a nucleic acid molecule (probe) that can hybridize to ABCB11 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of ABCB11 mRNA.

An alternative method for determining the level of expression of ABCB11 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of ABCB11 is determined by quantitative fluorogenic RT-PCR (i.e., the TaqMan™ System) or the DUAL-GLOR Luciferase assay.

The expression levels of ABCB11 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of ABCB11 expression levels may also comprise using nucleic acid probes in solution.

In some embodiments, the level of mRNA expression is assessed using branched DNA (bDNA) assays, quantitative PCR (qPCR), real-time quantitative PCR (RT-qPCR), multiplex qPCR or RT-qPCR, RNA-seq, or microarray analysis. Such methods can also be used for the detection of ABCB11 nucleic acids.

The level of ABCB11 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, FACS, immunodiffusion (single or double), immunoelectrophoresis, western blotting, radioimmunoassay (RIA), enzyme-linked immunosorbent assays (ELISAs), immunofluorescent assays, electrochemiluminescence assays, Luminex, MSD, FISH, and the like. Such assays can also be used for the detection of proteins indicative of the presence or replication of ABCB11 proteins.

### Examples

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Modulation of ABCB11 expression using regRNA-targeting ASOs

### Materials and Methods

### Aso Treatment of Primary Human Hepatocytes.

Primary human hepatocytes (PHH) were thawed using Lonza Human Cryopreserved Hepatocyte Thawing Medium and plated in 96-well collagen-coated plates (5.5-6.0×10⁴ cells per well). Four hours later, the media was replaced with sterile-filtered QualGro™ Hepatocyte Culture Medium (BioIVT) containing antibiotic supplement, 0.3 mg/ml Matrigel® matrix (Corning®), and ASO at the desired concentration (e.g., 1.25 μM, 2.5 μM, 5 μM, or 10 μM). After 72 hrs., cells were harvested by aspirating media, washing once with phosphate-buffered saline (PBS), and lysed with 150 μl of Buffer RLT lysis buffer (Qiagen®). Plates were frozen at-80° C. during cell lysis. After thawing, total RNA was isolated using the Qiagen RNEasy kit as instructed by manufacturer. cDNA (random-primed) was synthesized from 10 μl of RNA using the LifeTech High Capacity cDNA Reverse Transcription kit. 2 μl of cDNA was used for reverse transcription-quantitative PCR (RT-qPCR) analyses using standard Taqman” reagents from Applied Biosystems™ to measure ABCB11 and CYP7A1 gene expression. Gene expression data was normalized to the housekeeper gene, beta-2-microglobulin (B2M).

### In Vitro Biliary Clearance Assay

The ACCULIVER™ Human Biliary Clearance Kit (also known as the B-CLEAR® assay; BioIVT) was used to assess bile acid efflux in PHHs after ASO treatment. On Day 0, 4×10⁵ cells were plated in 24-well collagen-coated plates and QualGro medium containing 0.3% Matrigel matrix and ASO was added 4 hrs. later. On Day 2, medium was refreshed. On Day 3, cells from some wells were harvested for RNA analysis using 350 μl Buffer RLT lysis buffer, and lysates frozen at −80° C. for later RNA extraction. On Day 4, cells in the remaining wells had medium refreshed. On Day 6, the assay was performed according to the manufacturer's instructions. Briefly, cells were conditioned with Plus or Minus Buffers provided with the assay for 10 minutes, followed by addition of deuterium-labelled sodium taurocholate (d8-TCA) for 10 mins. Cells were washed 3 times, and frozen after the final wash. Analysis of d8-TCA was performed using liquid chromatography-mass spectrometry (LC-MS) (BioIVT). Calculations of bile acid clearance were made from the resulting LC-MS data according to the manufacturer's instructions.

### Results

1. Synthesis and In Vitro Characterization of Human ABCB11 regRNA-Targeting ASOs

Four human ABCB11 regRNAs were identified in the human genome (referred to herein as RR62 v2, RR63 v1, RR64 v2, and RR74 v1). An extended version of RR63 (referred to herein as RR63 v2) was also identified. FIG. 1C provides a diagram of the identified regRNAs aligned with the ABCB11 gene and active transcription sites.

To assess the ability of ASOs targeting ABCB11 regRNAs to modulate the expression of human ABCB11, 195 ASOs targeting the human ABCB11 regRNAs were synthesized. These initial ASOs were screened in PHHs at 5 μM to determine their efficacy in increasing human ABCB11 mRNA using quantitative PCR. This initial screen identified 10 ASOs that induced increased expression of ABCB11 mRNA by more than 1.5-fold (as compared to untreated PHHs).

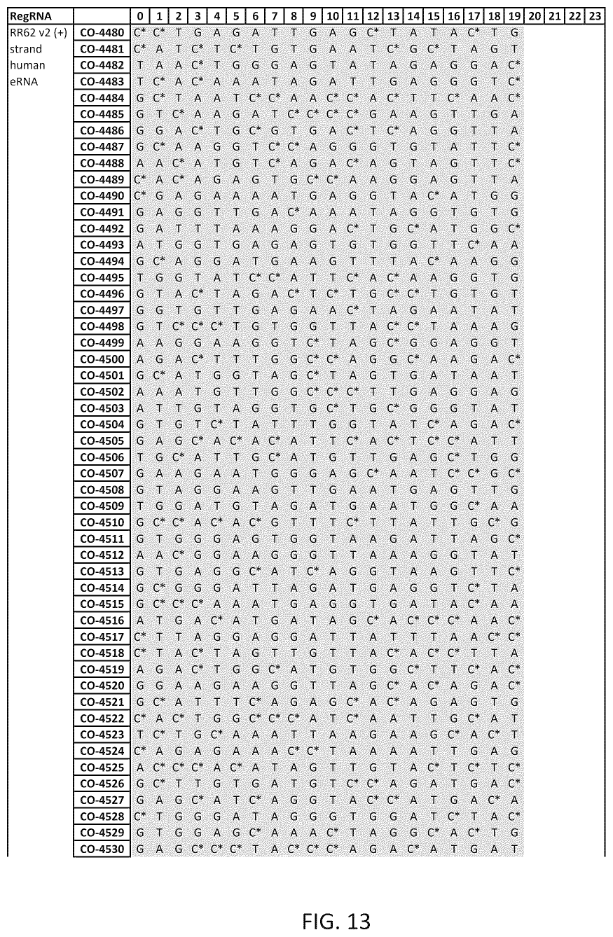

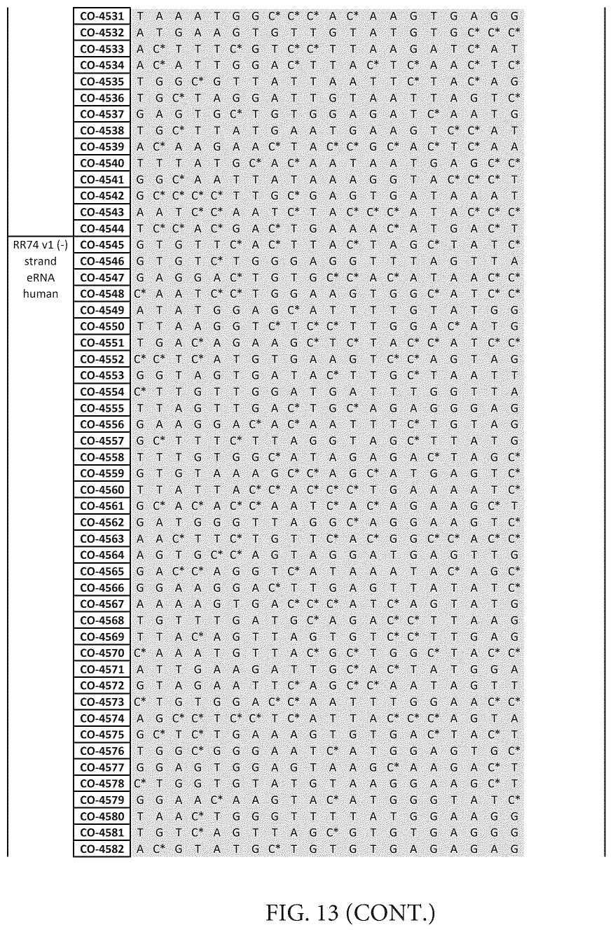

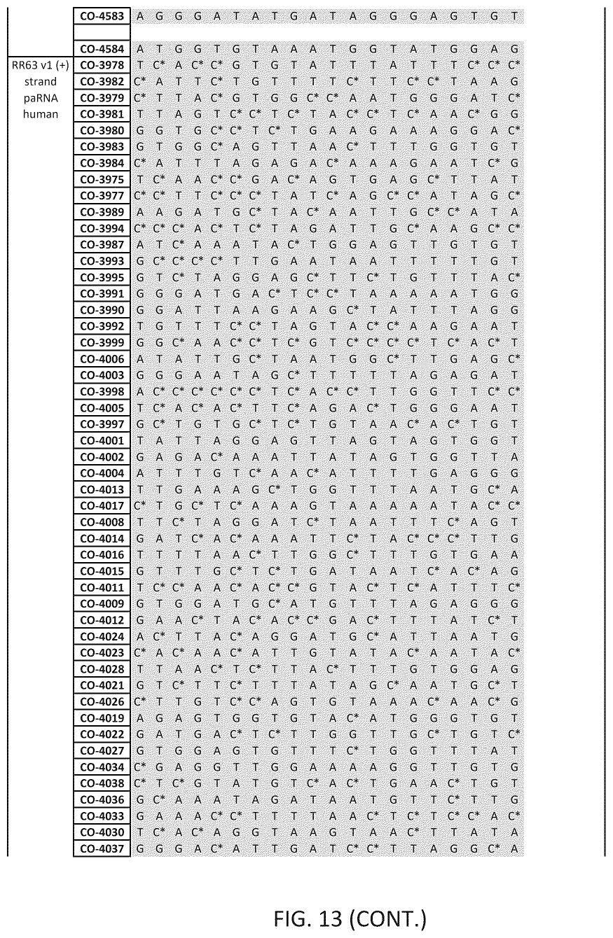

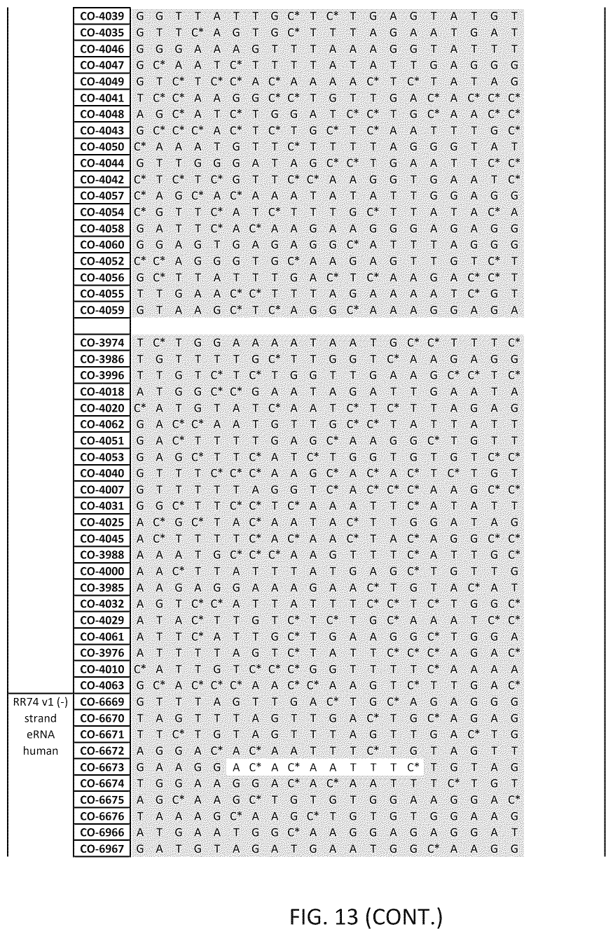

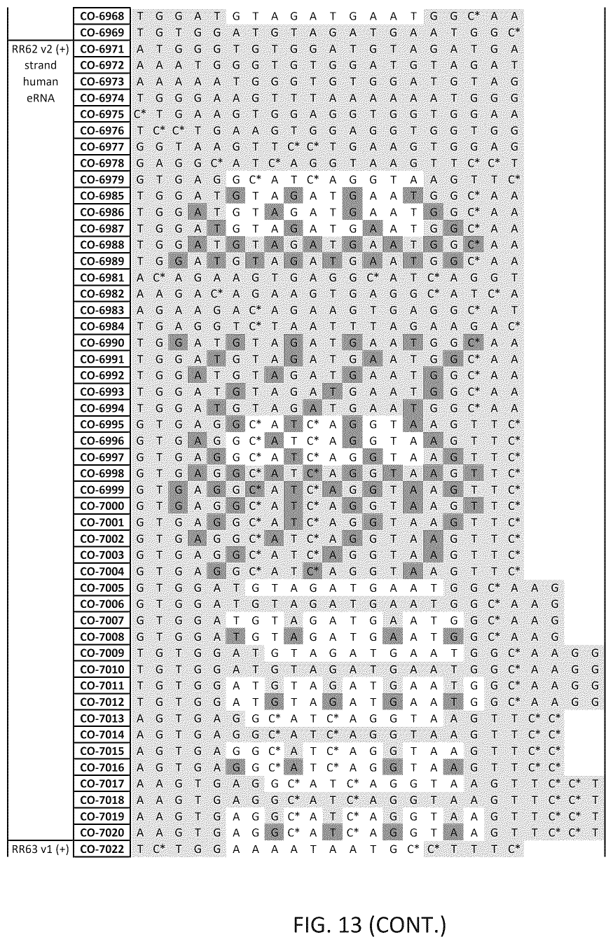

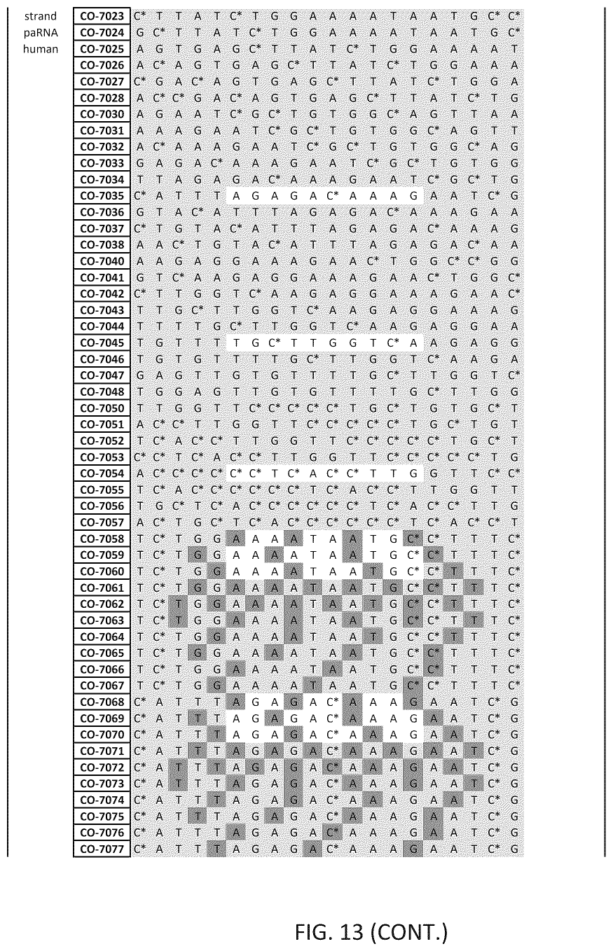

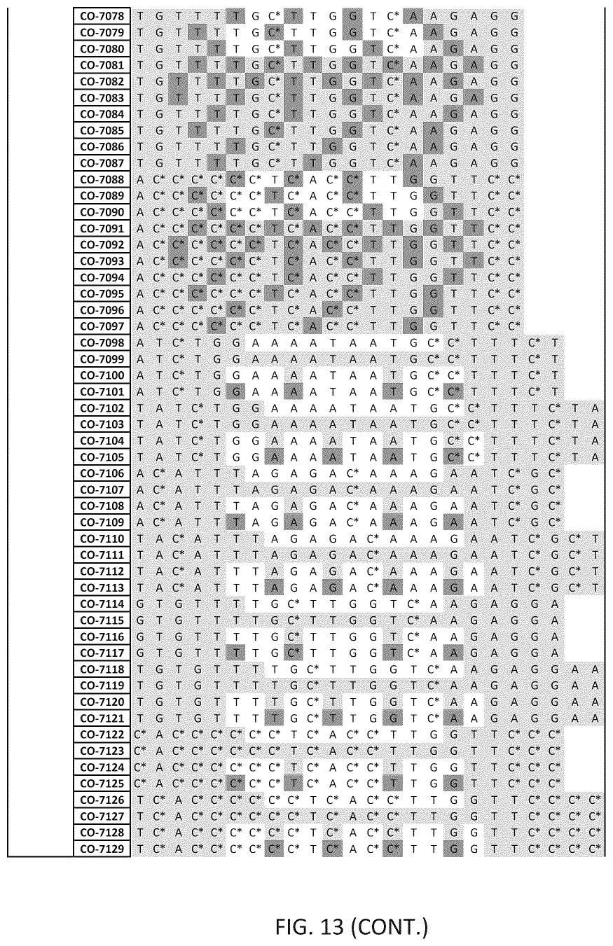

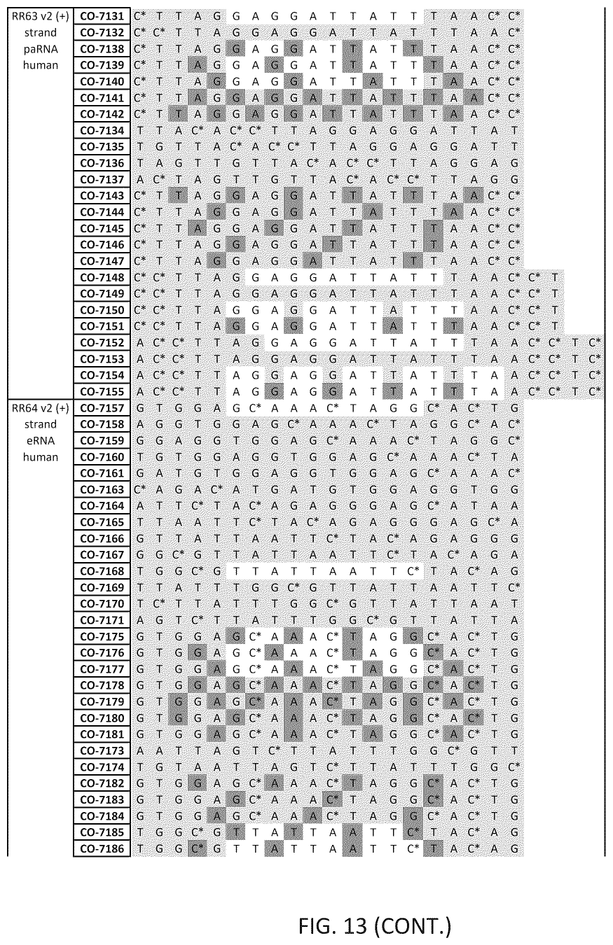

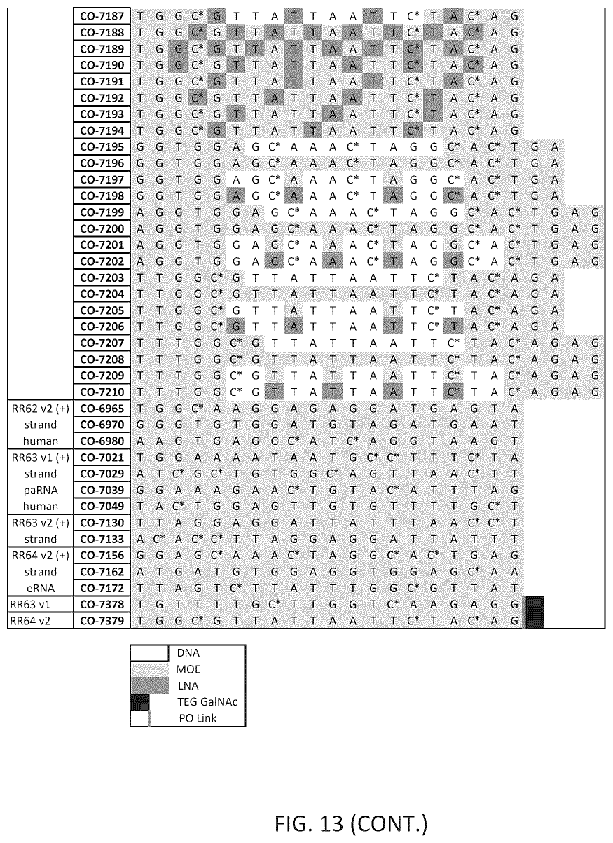

ASOs that showed efficacy were selected for basewalking and tiling around their targeted regRNA hit areas. 254 additional ASOs synthesized from the basewalking and tiling of the identified ASOs. Of these, 26 ASOs were selected for chemistry fine tuning by altering the chemistry, type, and position of chemical modification of the selected ASOs. ASO sequences and chemistries are provided in Tables 2, 3, 4, 5, 6, 7, 8, 9, 10, and 11, and FIGS. 11 and 13. ABCB11 mRNA fold change (FC) and standard deviation (SD) after treatment with the ASO, normalized to untreated PHHs (UNT), is provided in Table 12. In particular, ASOs CO-3974, CO-3984, CO-3986, and CO-3998 showed an approximately 1.5-fold upregulation of ABCB11 mRNA in PHHs on Day 3. Each of these ASOs targets the paRNA regRNA RR63.

TABLE 12
ASO sequences and ABCB11 mRNA fold change.

| | FC | | | FC | | | FC | | | FC | |
|---|---|---|---|---|---|---|---|---|---|---|---|
| | mRNA | | | mRNA | | | mRNA | | | mRNA | |
| | (5 μM | | | (5 μM | | | (5 μM | | | (5 μM | |
| | ASO | | | ASO | | | ASO | | | ASO | |
| | vs. | | | vs. | | | vs. | | | vs. | |
| Name | UNT) | SD | Name | UNT) | SD | Name | UNT) | SD | Name | UNT) | SD |
| CO-4480 | 0.931 | 0.23 | CO-4026 | 1.234 | 0.43 | CO-6990 | 1.16 | 0.14 | CO-7090 | 1.203 | 0.16 |
| CO-4481 | 1.18 | 0.16 | CO-4019 | 1.028 | 0.22 | CO-6991 | 1.193 | 0.21 | CO-7091 | 1.164 | 0.04 |
| CO-4482 | 1.001 | 0.24 | CO-4022 | 1.168 | 0.29 | CO-6992 | 0.992 | 0.14 | CO-7093 | 1.269 | 0.09 |
| CO-4483 | 1.041 | 0.16 | CO-4027 | 1.095 | 0.43 | CO-6993 | 1.209 | 0.17 | CO-7094 | 0.993 | 0.08 |
| CO-4484 | 1.113 | 0.06 | CO-4034 | 0.792 | 0.1 | CO-6994 | 1.131 | 0.19 | CO-7095 | 0.925 | 0.45 |
| CO-4485 | 1.194 | 0.24 | CO-4038 | 1.701 | 0.52 | CO-6995 | 1.277 | 0.09 | CO-7096 | 1.157 | 0.12 |
| CO-4486 | 1.151 | 0.27 | CO-4036 | 1.235 | 0.69 | CO-6996 | 1.314 | 0.21 | CO-7097 | 1.028 | 0.07 |
| CO-4487 | 1.052 | 0.18 | CO-4033 | 1.472 | 0.22 | CO-6997 | 1.058 | 0.11 | CO-7098 | 1.04 | 0.17 |
| CO-4488 | 1.084 | 0.15 | CO-4030 | 1.106 | 0.24 | CO-6998 | 1.246 | 0.07 | CO-7099 | 0.882 | 0.05 |
| CO-4489 | 1.083 | 0.24 | CO-4037 | 0.697 | 0.12 | CO-6999 | 1.24 | 0.09 | CO-7100 | 1.406 | 0.64 |
| CO-4490 | 1.158 | 0.18 | CO-4039 | 1.022 | 0.34 | CO-7000 | 1.203 | 0.09 | CO-7101 | 1.165 | 0.06 |
| CO-4491 | 1.101 | 0.09 | CO-4035 | 0.926 | 0.41 | CO-7001 | 1.237 | 0.14 | CO-7102 | 1.068 | 0.07 |
| CO-4492 | 1.192 | 0.14 | CO-4046 | 1.035 | 0.12 | CO-7002 | 1.367 | 0.17 | CO-7103 | 1.006 | 0.2 |
| CO-4493 | 1.178 | 0.17 | CO-4047 | 0.676 | 0.14 | CO-7003 | 1.066 | 0.07 | CO-7104 | 0.918 | 0.13 |
| CO-4494 | 1.12 | 0.19 | CO-4049 | 0.889 | 0.19 | CO-7004 | 1.285 | 0.1 | CO-7105 | 1.002 | 0.12 |
| CO-4495 | 1.07 | 0.14 | CO-4041 | 0.963 | 0.2 | CO-7005 | 1.257 | 0.18 | CO-7106 | 1.246 | 0.17 |
| CO-4496 | 0.984 | 0.15 | CO-4048 | 1.448 | 0.28 | CO-7006 | 1.279 | 0.18 | CO-7107 | 1.148 | 0.18 |
| CO-4497 | 1.024 | 0.13 | CO-4043 | 1.934 | 0.21 | CO-7007 | 0.963 | 0.12 | CO-7108 | 1.101 | 0.24 |
| CO-4498 | 0.934 | 0.15 | CO-4050 | 0.968 | 0.11 | CO-7008 | 1.175 | 0.13 | CO-7109 | 1.043 | 0.17 |
| CO-4499 | 1.202 | 0.34 | CO-4044 | 0.974 | 0.22 | CO-7009 | 1.289 | 0.11 | CO-7110 | 1.035 | 0.06 |
| CO-4500 | 1.109 | 0.2 | CO-4042 | 0.605 | 0.1 | CO-7010 | 1.293 | 0.19 | CO-7111 | 1.027 | 0.12 |
| CO-4501 | 1.023 | 0.04 | CO-4057 | 1.245 | 0.09 | CO-7011 | 1.284 | 0.09 | CO-7112 | 0.98 | 0.09 |
| CO-4502 | 1.142 | 0.14 | CO-4054 | 1.269 | 0.32 | CO-7012 | 0.985 | 0.11 | CO-7113 | 1.179 | 0.22 |
| CO-4503 | 1.202 | 0.14 | CO-4058 | 1.134 | 0.26 | CO-7013 | 0.997 | 0.11 | CO-7114 | 1.47 | 0.09 |
| CO-4504 | 1.232 | 0.32 | CO-4060 | 1.117 | 0.15 | CO-7014 | 1.293 | 0.08 | CO-7147 | 1.272 | 0.14 |
| CO-4505 | 1.011 | 0.07 | CO-4052 | 1.054 | 0.07 | CO-7015 | 1.257 | 0.29 | CO-7148 | 1.527 | 0.1 |
| CO-4506 | 1.169 | 0.18 | CO-4056 | 1.115 | 0.17 | CO-7016 | 1.278 | 0.12 | CO-7149 | 1.719 | 0.28 |
| CO-4507 | 0.992 | 0.13 | CO-4055 | 1.287 | 0.37 | CO-7017 | 1.182 | 0.23 | CO-7150 | 1.505 | 0.17 |
| CO-4508 | 1.185 | 0.14 | CO-4059 | 1.186 | 0.13 | CO-7018 | 1.249 | 0.42 | CO-7151 | 1.498 | 0.18 |
| CO-4509 | 1.233 | 0.09 | CO-3974 | 1.2 | 0.33 | CO-7019 | 1.284 | 0.31 | CO-7152 | 1.28 | 0.11 |
| CO-4510 | 1.084 | 0.24 | CO-3986 | 1.27 | 0.23 | CO-7020 | 1.133 | 0.1 | CO-7153 | 1.409 | 0.09 |
| CO-4511 | 1.182 | 0.08 | CO-3996 | 2.097 | 0.76 | CO-7022 | 1.224 | 0.13 | CO-7154 | 1.274 | 0.13 |
| CO-4512 | 1.087 | 0.1 | CO-4018 | 1.124 | 0.22 | CO-7023 | 1.338 | 0.11 | CO-7155 | 1.253 | 0.11 |
| CO-4513 | 1.122 | 0.41 | CO-4020 | 1.201 | 0.19 | CO-7024 | 1.415 | 0.57 | CO-7157 | 1.195 | 0.03 |
| CO-4514 | 1.175 | 0.16 | CO-4062 | 1.108 | 0.08 | CO-7025 | 1.259 | 0.19 | CO-7158 | 1.229 | 0.06 |
| CO-4515 | 0.978 | 0.24 | CO-4051 | 0.908 | 0.1 | CO-7026 | 1.158 | 0.11 | CO-7159 | 1.326 | 0.07 |
| CO-4516 | 1.033 | 0.1 | CO-4053 | 1.218 | 0.08 | CO-7027 | 1.304 | 0.29 | CO-7160 | 1.312 | 0.07 |
| CO-4517 | 0.914 | 0.07 | CO-4040 | 0.921 | 0.39 | CO-7028 | 1.49 | 0.28 | CO-7161 | 1.324 | 0.08 |
| CO-4518 | 0.981 | 0.07 | CO-4007 | 1.276 | 0.28 | CO-7030 | 1.372 | 0.3 | CO-7163 | 1.308 | 0.11 |
| CO-4519 | 1.061 | 0.18 | CO-4031 | 1.427 | 0.2 | CO-7031 | 1.212 | 0.05 | CO-7164 | 1.178 | 0.12 |
| CO-4520 | 0.945 | 0.13 | CO-4025 | 0.832 | 0.24 | CO-7032 | 1.093 | 0.08 | CO-7165 | 1.329 | 0.09 |
| CO-4521 | 1.092 | 0.16 | CO-4045 | 0.815 | 0.09 | CO-7033 | 1.316 | 0.1 | CO-7166 | 1.517 | 0.22 |
| CO-4522 | 1.027 | 0.11 | CO-3988 | 1.827 | 0.63 | CO-7034 | 1.097 | 0.27 | CO-7167 | 1.403 | 0.26 |
| CO-4523 | 1.037 | 0.21 | CO-4000 | 1.533 | 0.57 | CO-7035 | 1.047 | 0.09 | CO-7168 | 1.301 | 0.17 |
| CO-4524 | 1.243 | 0.11 | CO-4032 | 1.311 | 0.3 | CO-7036 | 1.129 | 0.17 | CO-7169 | 1.289 | 0.19 |
| CO-4525 | 0.995 | 0.12 | CO-4029 | 0.908 | 0.24 | CO-7037 | 1.136 | 0.28 | CO-7170 | 1.477 | 0.22 |
| CO-4526 | 1.048 | 0.09 | CO-4061 | 1.233 | 0.25 | CO-7038 | 1.189 | 0.25 | CO-7171 | 1.46 | 0.35 |
| CO-4527 | 0.97 | 0.09 | CO-4010 | 0.898 | 0.23 | CO-7040 | 1.267 | 0.29 | CO-7177 | 1.455 | 0.11 |
| CO-4528 | 0.883 | 0.14 | CO-4063 | 1.457 | 0.29 | CO-7041 | 1.15 | 0.18 | CO-7178 | 1.737 | 0.32 |
| CO-4529 | 1.05 | 0.1 | CO-6669 | 1.291 | 0.15 | CO-7042 | 1.132 | 0.09 | CO-7179 | 2.344 | 0.38 |
| CO-3984 | 1.111 | 0.13 | CO-6670 | 1.157 | 0.13 | CO-7043 | 1.1 | 0.13 | CO-7180 | 2.04 | 0.56 |
| CO-3989 | 1.19 | 0.36 | CO-6671 | 1.163 | 0.02 | CO-7044 | 0.943 | 0.06 | CO-7181 | 1.603 | 0.11 |
| CO-3994 | 1.988 | 0.53 | CO-6672 | 1.391 | 0.11 | CO-7045 | 1.112 | 0.23 | CO-7182 | 1.572 | 0.25 |
| CO-3993 | 1 | 0.29 | CO-6673 | 1.313 | 0.11 | CO-7046 | 1.243 | 0.19 | CO-7183 | 1.295 | 0.07 |
| CO-3995 | 1.683 | 0.15 | CO-6674 | 1.175 | 0.08 | CO-7047 | 1.175 | 0.13 | CO-7184 | 1.582 | 0.3 |
| CO-3991 | 1.614 | 0.34 | CO-6675 | 1.346 | 0.16 | CO-7048 | 1.175 | 0.17 | CO-7185 | 1.327 | 0.14 |
| CO-3992 | 1.388 | 0.28 | CO-6676 | 1.204 | 0.13 | CO-7050 | 1.026 | 0.16 | CO-7186 | 1.85 | 0.25 |
| CO-3999 | 1.076 | 0.09 | CO-6966 | 1.177 | 0.08 | CO-7051 | 1.201 | 0.21 | CO-7187 | 1.544 | 0.29 |
| CO-4006 | 1.044 | 0.13 | CO-6967 | 1.405 | 0.4 | CO-7052 | 1.046 | 0.09 | CO-7188 | 1.697 | 0.2 |
| CO-4003 | 1.181 | 0.3 | CO-6968 | 1.373 | 0.16 | CO-7053 | 1.145 | 0.25 | CO-7189 | 1.487 | 0.07 |
| CO-3998 | 1.319 | 0.11 | CO-6969 | 1.266 | 0.18 | CO-7054 | 1.323 | 0.18 | CO-7190 | 1.641 | 0.24 |
| CO-4005 | 0.913 | 0.18 | CO-6971 | 1.091 | 0.17 | CO-7055 | 1.498 | 0.28 | CO-7191 | 1.676 | 0.38 |
| CO-3997 | 1.501 | 0.38 | CO-6972 | 1.207 | 0.14 | CO-7056 | 1.491 | 0.53 | CO-7192 | 1.591 | 0.37 |
| CO-4001 | 2.08 | 0.31 | CO-6973 | 1.325 | 0.24 | CO-7057 | 1.145 | 0.15 | CO-7193 | 1.555 | 0.2 |
| CO-4002 | 1.831 | 0.64 | CO-6974 | 1.394 | 0.14 | CO-7058 | 1.052 | 0.08 | CO-7195 | 1.443 | 0.12 |
| CO-4004 | 1.103 | 0.11 | CO-6975 | 1.34 | 0.14 | CO-7059 | 1.023 | 0.09 | CO-7196 | 1.33 | 0.03 |
| CO-4013 | 1.166 | 0.11 | CO-6976 | 1.373 | 0.16 | CO-7060 | 1.17 | 0.24 | CO-7197 | 1.364 | 0.24 |
| CO-4017 | 1.228 | 0.11 | CO-6977 | 1.15 | 0.14 | CO-7061 | 1.261 | 0.24 | CO-7198 | 1.075 | 0.1 |
| CO-4008 | 1.1 | 0.14 | CO-6978 | 1.456 | 0.19 | CO-7062 | 1.218 | 0.18 | CO-7199 | 1.166 | 0.1 |
| CO-4014 | 1.003 | 0.12 | CO-6979 | 1.201 | 0.18 | CO-7063 | 1.291 | 0.22 | CO-7200 | 1.277 | 0.06 |
| CO-4016 | 1.294 | 0.25 | CO-6985 | 1.414 | 0.15 | CO-7064 | 1.017 | 0.1 | CO-7201 | 1.253 | 0.17 |
| CO-4015 | 1.043 | 0.23 | CO-6986 | 0.998 | 0.04 | CO-7065 | 1.262 | 0.13 | CO-7205 | 1.278 | 0.21 |
| CO-4011 | 1.184 | 0.14 | CO-6987 | 1.026 | 0.23 | CO-7066 | 1.2 | 0.11 | CO-7206 | 1.164 | 0.15 |
| CO-4009 | 1.011 | 0.09 | CO-6988 | 1.093 | 0.07 | CO-7067 | 1.081 | 0.13 | CO-7207 | 1.221 | 0.08 |
| CO-4012 | 1.15 | 0.15 | CO-6989 | 1.469 | 0.31 | CO-7068 | 1.077 | 0.23 | CO-7208 | 1.339 | 0.06 |
| CO-4024 | 0.997 | 0.26 | CO-6981 | 1.164 | 0.04 | CO-7069 | 1.011 | 0.11 | CO-7209 | 1.262 | 0.2 |
| CO-4023 | 1.092 | 0.47 | CO-6982 | 0.976 | 0.11 | CO-7070 | 1.044 | 0.14 | CO-7210 | 1.362 | 0.26 |
| CO-4028 | 1.418 | 0.44 | CO-6983 | 1.065 | 0.14 | CO-7088 | 1.177 | 0.21 | | | |
| CO-4021 | 1.301 | 0.33 | CO-6984 | 1.127 | 0.08 | CO-7089 | 1.249 | 0.16 | | | |

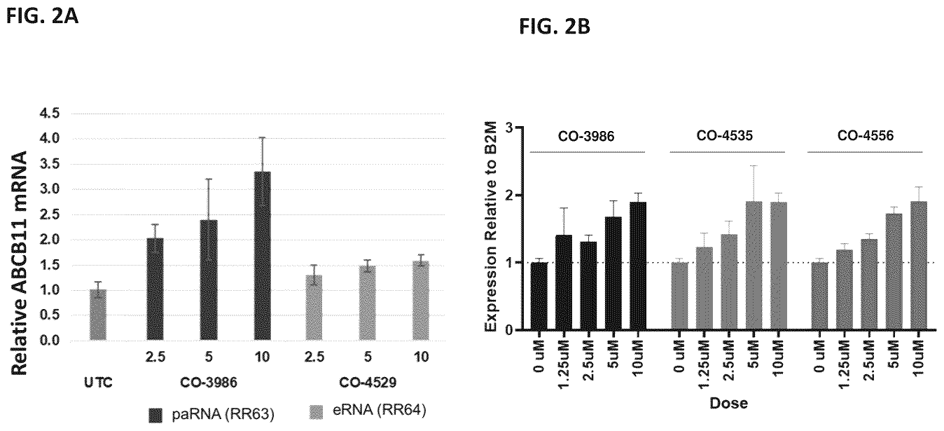

ASOs CO-3986 and CO-4529 were selected for further characterization in dose titration studies in PHHs and were tested at 2.5 M, 5 μM, and 10 μM. Both ASO CO-3986 (targeting paRNA RR63) and CO-4529 (targeting eRNA RR64) induced a dose-dependent increase in ABCB11 mRNA (FIG. 2A). In addition, ASOs CO-4535 (targeting eRNA RR64) and CO-4556 (targeting eRNA RR74) also induced a dose-dependent increase in ABCB11 mRNA in PHHs (FIG. 2B). Each of ASOs CO-3986, CO-4529, CO-4535, and CO-4556 targets a different ABCB11 regRNA, suggesting that all four regRNAs have a role in regulating ABCB11 gene expression.

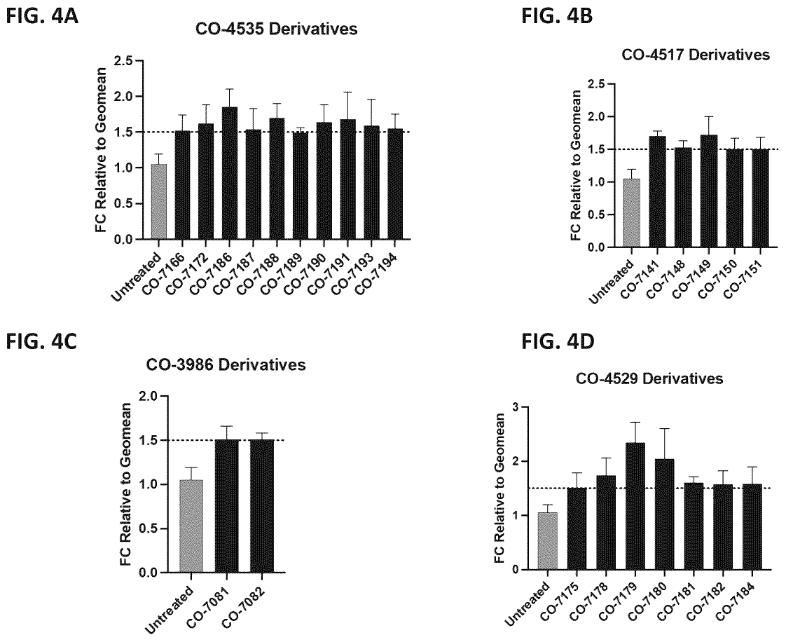

Additional chemical modifications were made to ASOs CO-4535 (i.e., CO-7166, CO-7172, CO-7186, CO-7187, CO-7188, CO-7189, CO-7190, CO-7191, CO-7193, and CO-7194), CO-4529 (i.e., CO-7175, CO-7178, CO-7179, CO-7180, CO-7181, CO-7182, and CO-7184), CO-4517 (i.e., CO-7141, CO-7148, CO-7149, CO7150, and CO-7151), and CO-3986 (i.e., CO-7081 and CO-7082). These ASOs were tested for their ability to induce increased expression of human ABCB11 in PHHs as described above. As shown in FIG. 4, treatment of PHHs with these ASOs resulted in upregulated human ABCB11 expression greater than or equal to 1.5-fold as compared to untreated PHHs (FIG. 4). Visual representations of the chemical modifications are provided in FIGS. 11 and 13.

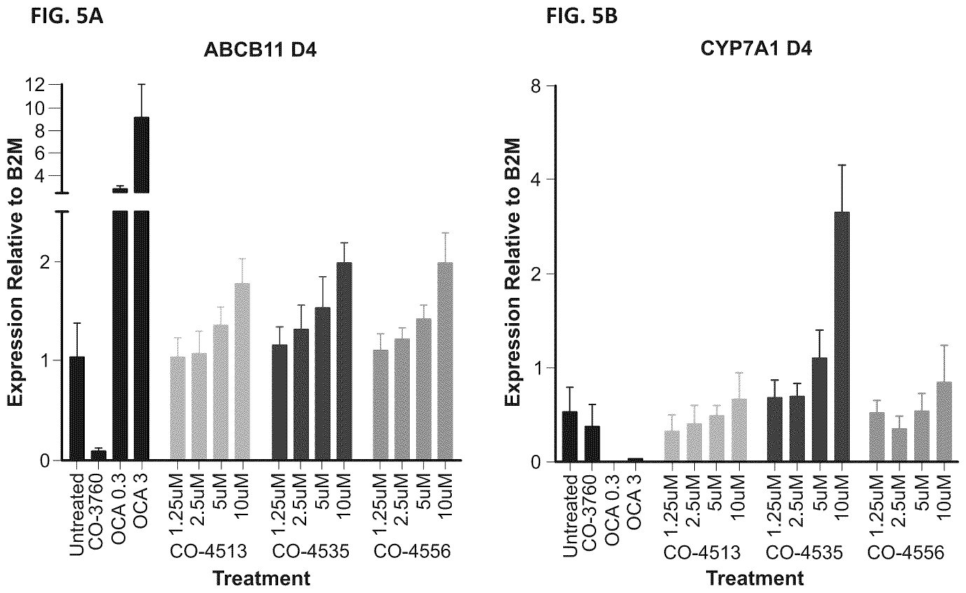

Moreover, a dose-responsive increase in ABCB11 mRNA expression was also observed with ASOs targeting a downstream regRNA from enhancer RR62_v2 (CO-4513), as well as upstream regRNAs from enhancers RR64_v2 (CO-4535) and RR74_v1 (CO-4556) (FIG. 5). Obeticholic acid (OCA), the second-line treatment for PBC, was used as a positive control for ABCB11 upregulation at doses of 0.3 μM and 3 μM. ASO CO-3760, a gapmer targeting ABCB11 mRNA to knock down ABCB11, was used as an uptake control at a concentration of 5 μM ABCB11 and CYP7A1 mRNA expression on Day 4 after ASO treatment is shown.

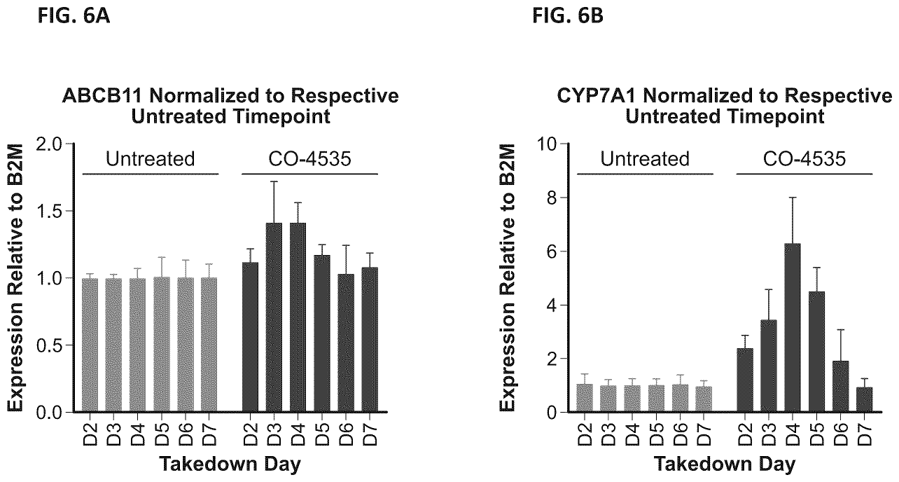

To further assess the ability of ASO CO-4535 to upregulate the expression of ABCB11 and CYP7A1 mRNA, a time course assay was performed over 7 days in PHHs contacted with ASO CO-4535. Cells were treated with CO-4535 for two days, and were collected daily from Day 2 to Day 7. As shown in FIG. 6, on Day 4, PHHs contacted with ASO CO-4535 showed strong ABCB11 and CYP7A1 mRNA upregulation, indicating effective bile acid efflux (FIG. 6). Increasing ABCB11 expression promotes clearance of intracellular bile acids (BA), which reduces the expression of the gene encoding Small Heterodimer Partner (SHP), which in turn increases CYP7A1 expression.

ASOs based on CO-3986, CO-7179, CP-7180, CO-7184, and CO-4513, and containing PO-modifications as shown in Table 13 below were synthesized and screened in PHHs at 5 μM to determine their efficacy in increasing human ABCB11 mRNA expression. Moreover, ASOs having efficacy in this screen were tested for dose-dependent efficacy at 1.25 μM, 2.5 μM, 5 μM, and 10 μM in PHHs. Changes to ABCB11 mRNA levels (fold change (FC) and standard deviation (SD)) following ASO treatment (normalized to untreated hepatocytes) is provided in Table 13 below.

TABLE 13

| Name | FC mRNA (5 μM ASO vs. untreated) | SD |
|---|---|---|
| CO-11198 | 0.97 | 0.19 |
| CO-11199 | 0.97 | 0.22 |
| CO-11200 | 1.34 | 0.38 |
| CO-11201 | 0.99 | 0.13 |
| CO-11202 | 1.50 | 0.62 |
| CO-11203 | 1.38 | 0.36 |
| CO-11204 | 1.15 | 0.32 |
| CO-11205 | 1.11 | 0.45 |
| CO-11206 | 1.02 | 0.40 |
| CO-11207 | 0.91 | 0.23 |
| CO-11208 | 1.04 | 0.26 |
| CO-11209 | 1.11 | 0.21 |
| CO-11210 | 2.79 | 0.97 |
| CO-11211 | 2.80 | 0.82 |
| CO-11212 | 2.44 | 0.61 |
| CO-11213 | 1.95 | 0.53 |
| CO-11214 | 2.90 | 1.01 |
| CO-11215 | 2.68 | 0.35 |
| CO-11216 | 2.58 | 0.55 |
| CO-11217 | 2.10 | 0.39 |
| CO-11218 | 2.86 | 0.71 |
| CO-11219 | 2.45 | 0.54 |
| CO-11220 | 2.22 | 0.49 |
| CO-11221 | 2.00 | 0.68 |
| CO-11222 | 1.79 | 0.51 |
| CO-11223 | 1.80 | 0.47 |
| CO-11224 | 1.88 | 0.50 |
| CO-11225 | 1.66 | 0.30 |
| CO-11226 | 2.31 | 0.60 |
| CO-11227 | 2.11 | 0.64 |
| CO-11228 | 1.99 | 0.77 |
| CO-11229 | 2.83 | 1.05 |
| CO-11230 | 1.63 | 0.49 |
| CO-11231 | 1.85 | 0.15 |
| CO-11232 | 1.72 | 0.32 |
| CO-11233 | 1.60 | 0.22 |
| CO-11234 | 1.55 | 0.45 |
| CO-11235 | 1.47 | 0.49 |
| CO-11236 | 1.60 | 0.44 |
| CO-11237 | 1.56 | 0.36 |
| CO-11238 | 1.26 | 0.25 |
| CO-11239 | 1.53 | 0.15 |
| CO-11240 | 1.31 | 0.21 |
| CO-11241 | 1.71 | 0.39 |
| CO-11242 | 1.49 | 0.53 |
| CO-11243 | 1.71 | 0.40 |
| CO-11244 | 1.29 | 0.56 |
| CO-11245 | 1.59 | 0.46 |
| CO-12745 | 0.92 | 0.09 |
| CO-12746 | 1.09 | 0.09 |
| CO-12747 | 0.97 | 0.11 |
| CO-12748 | 0.91 | 0.10 |
| CO-12749 | 0.95 | 0.04 |
| CO-12750 | 1.39 | 0.14 |
| CO-12751 | 0.93 | 0.08 |
| CO-12752 | 0.99 | 0.08 |
| CO-12753 | 1.03 | 0.14 |
| CO-12754 | 1.04 | 0.11 |
| CO-12755 | 0.99 | 0.18 |
| CO-12756 | 1.33 | 0.01 |

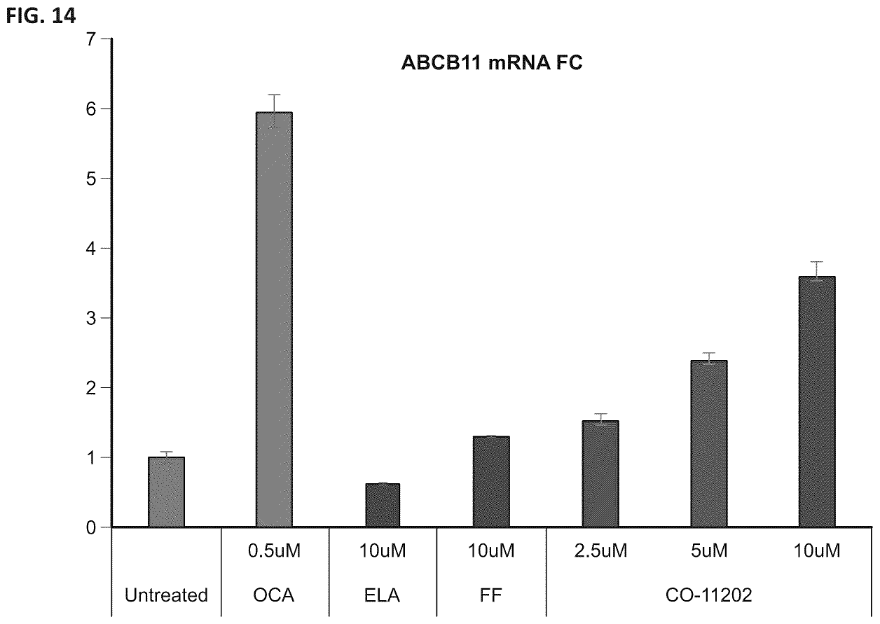

As shown in Table 13, the screen identified several ASOs that upregulate ABCB11 gene expression by at least 1.5-fold. One of the ASOs identified in the screen above was CO-11202. A study to compare the effect of CO-11202 on ABCB11 mRNA expression with obeticholic acid (OCA), an FXR agonist, and the PPARa agonists elafibranor and fenofibrate was performed using PHHs. As shown in FIG. 14, CO-11202 induced a dose-dependent increase in ABCB11 mRNA expression following treatment, with about a 2-fold change observed at the 5 μM dose and about a 3.5-fold change observed at 10 μM dose.

ASOs targeting ABCB11 regRNA enhance bile acid efflux in vitro. Levels of cholesterol 7 alpha-hydroxylase (CYP7A1) mRNA (or the enzymatic activity of its protein) can be used to track changes in bile acid synthesis in the liver since the CYP7A1 protein is the first and rate-limiting step in the conversion of cholesterol to bile acids (see, e.g., Chiang and Ferrel (2020) Liver Res. 4 (2): 47-63). As bile acid levels in hepatocytes decline, Cyp7a1 is transcriptionally upregulated to restore bile acid synthesis. Conversely, when bile acid levels reach high levels, this gene is actively down regulated. These changes are mediated by farnesoid X receptor (FXR), a sensor of bile acid levels, and the master regulator of intracellular bile acid concentrations. CYP7A1 mRNA can therefore be used as a surrogate for monitoring changes in hepatic bile acids in response to ASO treatment. Accordingly, an ASO that successfully increases ABCB11 expression would be expected to promote increased bile acid efflux, thus reducing bile acid levels in the cell. In a wild-type (non-disease) setting, this reduction promotes a compensatory increase in CYP7A1 to restore normal levels.

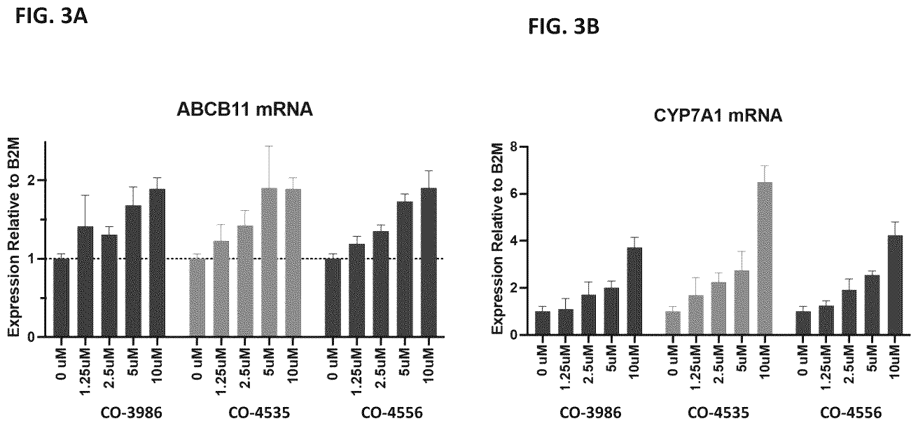

As shown in FIG. 3, ABCB11-upregulating ASOs CO-3986, CO-4535 and CO-4556 also induce increased expression of CYP7A1 mRNA in a dose-dependent manner, indicating that these ASOs are effectively enhancing bile acid efflux. Moreover, ASOs that increased ABCB11 and CYP7A1 mRNA were effective in PHHs from multiple human donors (data not shown).

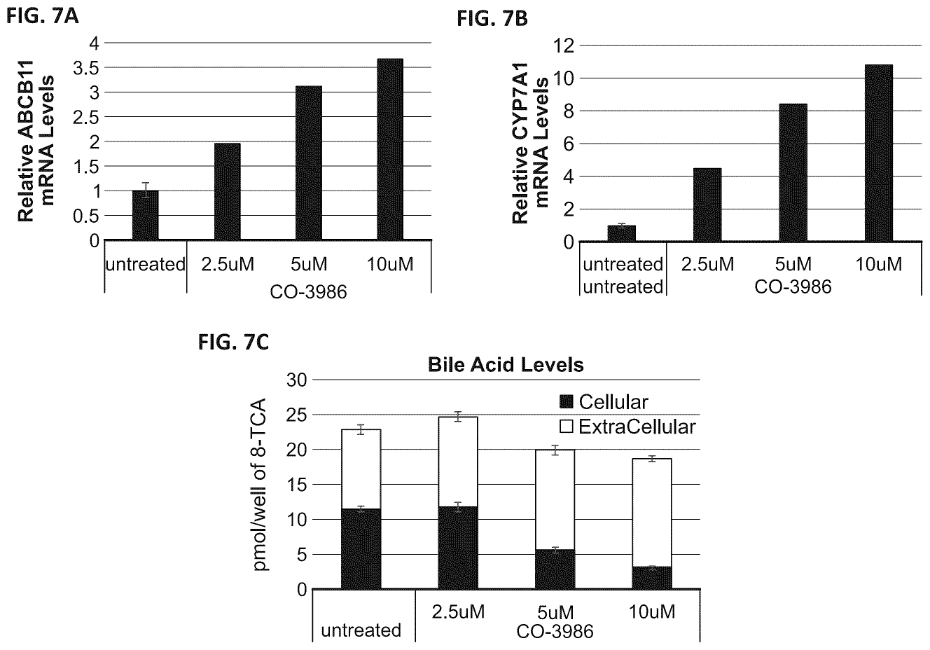

The ability of ASO CO-3986 to induce increased bile acid efflux was assessed in PHHs using the in vitro biliary clearance assay (B-CLEAR), as described above. As shown in FIG. 7, treatment with ASO CO-3986 increased ABCB11 expression (measured in duplicate wells by qRT-PCR) in a dose-responsive manner and the efflux of bile acids from the intracellular compartment to the bile cannuliculi of PHHs. CYP7A1 was also upregulated as a compensatory response to decreased intracellular bile acid levels. Moreover, treatment of PHHs with 5 μM and 10 μM of ASO CO-3986 reduced intracellular and extracellular bile acid levels in hepatocytes.

### Example 2: In Vivo Characterization of Human and Mouse ASOs

### Materials and Methods

### Aso Treatment of Mouse Primary Hepatocytes (MPH)

MPHs were isolated from C57Bl/6 mice and seeded at 2×10⁴ cells/well in a collagen-coated 96-well plate. Cells were treated with ASOs at a concentration of 5 μM in Cellartis® Power™ Primary HEP Media (Takara Bio) 24 hours after seeding. Cells were harvested 48 hours post ASO treatment by washing once with PBS, followed by collection in 100 μL of Buffer RLT lysis buffer (Qiagen).

### Liver Humanized Mice

In a first in vivo study, female liver-humanized FRG® (Fah^{-/-}; Rag2^{-/-}; Il2rg^{-/-}) knockout mice (Yecuris™) were used to assess the efficacy of the human ABCB11 regRNA-targeting ASOs CO-3986 and CO-4535 in the context of cholestasis. The mice were grouped evenly based on albumin and body weight.

A. First Study. Two groups of five mice were dosed with GalNAc3—CO-7378 (CO-3986 conjugated to GalNAc3) or GalNAc3—CO-7379 (GalNAc3 conjugated to GalNAc3) respectively, control via subcutaneous (SC) injection on Days 0 and 7. One group of five mice was dosed with PBS control via SC injection on Days 0 and 7. A fourth group of five mice was treated with ursodeoxycholic acid (UDCA) via intraperitoneal (IP) injection on Days 14-21. After fasting overnight (6 pm-9 am) the animals had a blood draw of 100 μL processed to serum on Days 0, 7, and 14 (all pre-dose). On Day 21, the animals were euthanized for terminal harvest after fasting overnight and >350 μL of whole blood was processed to serum. 100 μL of whole blood was collected in the same manner as the previous bleeds, prior to euthanasia and final exsanguination. Harvested livers were perfused with cold PBS with the left lobe and remnant liver frozen separately after bile salt collection. A piece of the ileum tissue was removed and flushed with PBS and snap-frozen. The serum (two tubes for each animal, 15 μL and 25 μL, for each timepoint) was analyzed using LC-MS (Creative Proteomics) to assess bile acids and 7αC4 levels. The remaining samples collected were analyzed for total serum bile acid levels using a commercial kit.

B. Second Study. In a second in vivo study was conducted to assess the ability of CO-11202 (CO-12367 conjugated with GalNAc3). Mice were dosed with 10 mg/CO-12367 on Days 0 and 7 via SC injection. One group of five mice were dosed with PBS control via SC injection on Days 0 and 7. One group of five mice received 10 mg/kg of obeticholic acid (OCA) via oral gavage daily for 21 days. Fasting blood was collected from the mice on Days 0, 7, 14 and 21 (collection performed prior to any SC injections) and processed to serum to analyze bile acid levels. On Day 21, mice were euthanized and livers and ileum tissue harvested and processed as described above.

Frozen liver pieces were pulverized on dry ice and RNA extracted using TRIzol™ reagent. cDNA was generated and analyzed using qPCR to detect ABCB11 mRNA levels and other genes of interest as described above. Serum 7αC4 levels were detected using LC-MS (Creative Proteomics). Total serum bile acids were measured using the Total Bile Acid Fluorometric Assay Kit (Cell BioLabs). Liver bile acids were measured using the Total Bile Acid Fluorometric Assay Kit.

### Anit Mouse Study

A study to assess the ability of mouse-specific ASOs targeting murine ABCB11 regRNA RR54 v2, CO-4758 and CO-4765, both conjugated to GalNAc, to upregulate murine Abcb11 expression in an induced-cholestasis model was performed. Male C57Bl/6 mice were divided into 7 groups of 6 animals and dosed SC with ASO on Day 0 and Day 5, followed by sacrifice on Day 10. One group of mice received UDCA (ursodeoxycholic acid, the first-line treatment for PBC) via IP injection at a dose of 30 mg/kg on Days 0-10 as a positive control. Four groups of mice were treated with alpha-naphthylisothiocyanate (ANIT) to induce chemically induce cholestasis. The ANIT-treated mice were dosed on Day 8 with 75 mg/kg of ANIT by oral gavage, and three groups of mice were mock-treated with olive oil (the vehicle used to dissolve ANIT, used as a control for the non-ANIT treated groups). Fasting serum was collected prior to the start of the study, as well as on Days 5 and 10. Serum was used to assess total bile acid and liver enzyme levels at Idexx Bioanalytics (Columbia, MO) using LC-MS. Liver bile acids were measured using the Total Bile Acid Fluorometric Assay Kit. Total RNA was also extracted from liver to measure Abcb11 and Cyp7a1 mRNA, as described above.

### Results

Asos Targeting Mouse Abcb11 regRNA Induce Increased Abcb11 Gene Expression

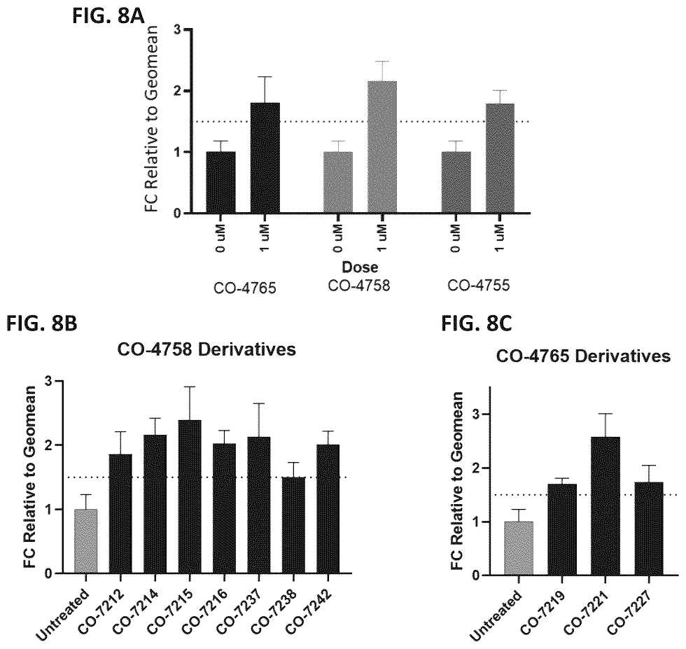

A mouse Abcb11 regRNA was identified. ASOs targeting this regRNA (i.e., CO-4755, CO-4765 and CO-4758) were synthesized and characterized as previously described for the ASOs targeting the human ABCB11 regRNAs using MPHs. Table 14 and FIG. 8 provide mRNA fold change (FC) and standard deviation (SD) induced by the ASOs when tested at 1 μM, 3 μM, or 9 μM, as indicated.

TABLE 14
Mouse regRNA ASOs

| | FC | SD |
|---|---|---|
| sNTC3 0 μM | 1.012 | 0.173 |
| sNTC3 3M | 1.469 | 0.113 |
| CO-4755 0 μM | 1.012 | 0.173 |
| CO-4755 1 μM | 1.796 | 0.212 |
| CO-4755 3 μM | 1.895 | 0.447 |
| CO-4755 9 μM | 1.775 | 0.409 |
| CO-4765 0 μM | 1.012 | 0.173 |
| CO-4765 1 μM | 1.812 | 0.418 |
| CO-4765 3 μM | 1.697 | 0.199 |
| CO-4765 9 μM | 1.755 | 0.627 |
| CO-4758 0 μM | 1.012 | 0.173 |
| CO-4758 1 μM | 2.164 | 0.32 |
| CO-4758 3 μM | 1.643 | 0.36 |
| CO-4758 9 μM | 1.517 | 0.371 |

Overall, these studies demonstrate that the mouse Abcb11 gene expression is regulated by a regRNA which can be targeted with an ASO to increase Abcb11 mRNA levels.

Additional chemical modifications were made to CO-4758 (i.e., CO-7212, CO-7214, CO-7215, CO-7216, CO-7237, CO-7238, and CO-7242) and CO-4765 (i.e., CO-7219, CO-7221, and CO-7227). These ASOs were tested in MPHs to assess their efficacy in upregulating the expression of the murine Abcb11 gene. Each of the tested ASOs upregulated mouse ABCB11 mRNA expression greater than or equal to 1.5-fold as compared to untreated hepatocytes (FIG. 8). Visual representations of the chemical modifications are provided in FIG. 12.

### In Vivo Humanized Mice Assay

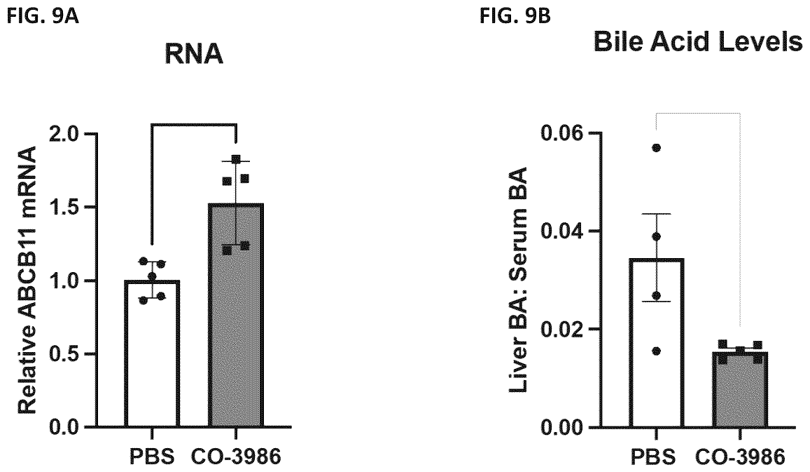

In a first in vivo study, the administration of ASO CO-3986 in the liver-humanized FRGR knockout mouse model resulted in increased human ABCB11 expression and liver bile acid efflux (FIG. 9). ABCB11 mRNA was increased which promoted efflux of hepatic bile acids. Moreover, lower levels of intracellular bile acids were observed in ASO-treated mice, which indicates an increase in bile acid efflux. Thus, ASO CO-3986 successfully upregulated ABCB11 RNA which lead to an increase in transporter protein expression and an increased efflux of bile acid.

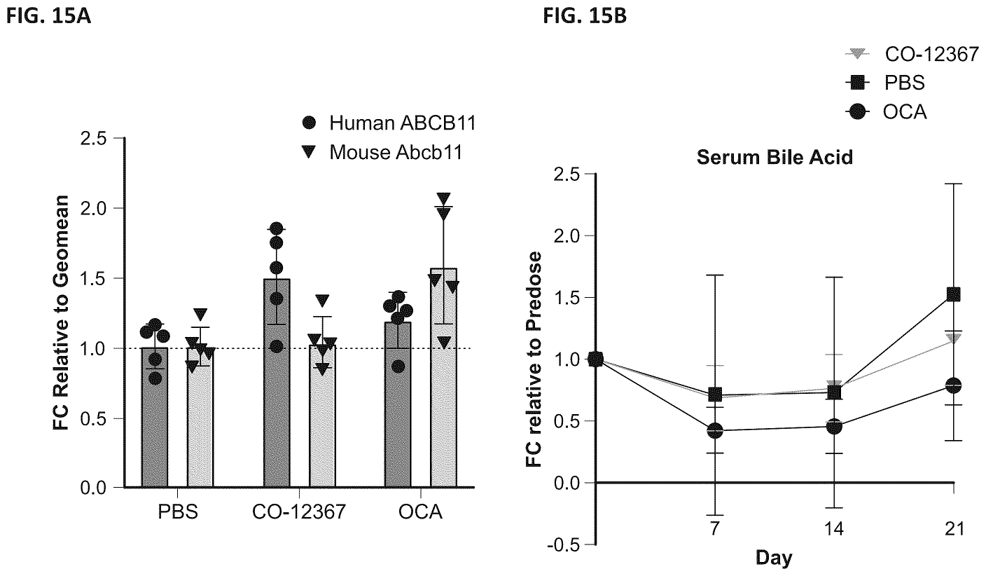

In a second in vivo administration of CO-12367 in the liver humanized FRGR knockout mouse model resulted in increased human ABCB11 expression without affecting mouse Abcb11 expression (FIG. 15A). A decrease in serum bile acid levels was also observed (FIG. 15B), trending in the same direction as the positive control, OCA. Thus, ASO CO-12367 upregulated ABCB11 mRNA and had an impact on the bile acid metabolism pathway, indicated by a decrease in serum bile acids.

### ANIT Mouse Study

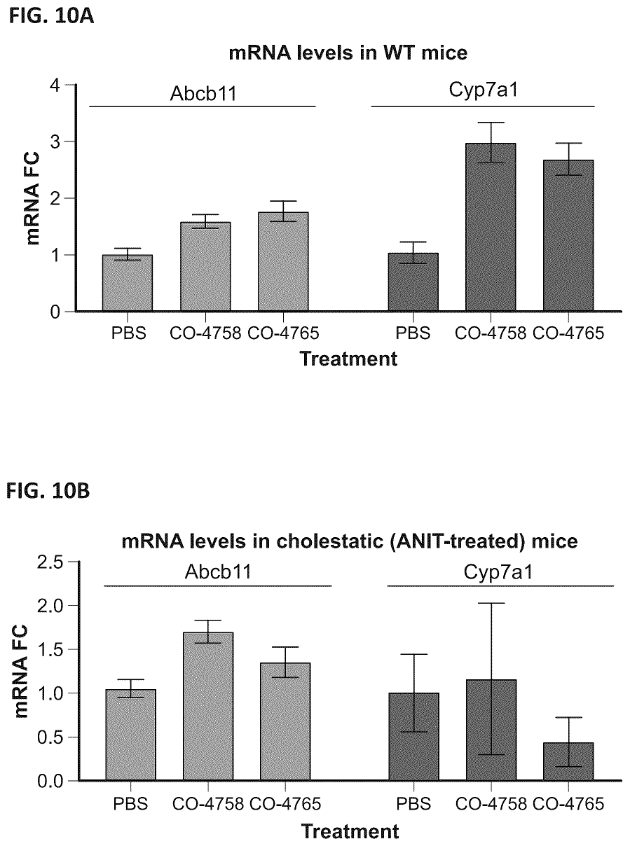

The ASOs targeting mouse ABCB11 regRNA, CO-4758 and CO-4765, were also tested for their ability to upregulate Abcb11 expression in an induced-cholestasis model. As shown in FIG. 10, CO-4758 increased both Abcb11 and Cyp7a1 gene expression in vivo in an induced cholestasis model (ANIT). Abcb11 mRNA levels were upregulated by ASO treatment in both conditions. Compensatory Cyp7a1 upregulation occurred only in non-cholestatic animals.

This data demonstrates a therapeutically relevant increase in target gene expression in Abcb11 regRNA targeting-ASO-treated mice versus controls in the well-established ANIT-induced mouse model. Furthermore, human ABCB11 regRNA-targeting ASOs likewise increase ABCB11 gene expression and decrease hepatic bile acids in a humanized liver mouse model.

Overall, ASOs targeting mouse and human regRNAs increased ABCB11 expression in primary human hepatocytes in vitro, in humanized liver cells in vivo, and mouse liver cells in vivo. The ASO can also increase bile acid export both in vitro and in vivo. Therefore, the ABCB11 regRNA-targeting ASOs can be used to augment expression of a healthy ABCB11 gene to treat diseases associated with excess bile acid such as PBC and cholestasis. Upregulation of ABCB11 enhances bile acid efflux from primary human hepatocytes in vitro, as well as in human hepatocytes cells in a humanized mouse liver model. Likewise, a mouse Abcb11 regRNA-specific ASOs upregulated bile acid regulator Abcb11 expression in a mouse model of PBC and can be used as surrogate for in vivo development studies.

### INCORPORATION BY REFERENCE

Unless stated to the contrary, the entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

What is claimed is:

1. An antisense oligonucleotide (ASO) complementary to at least 8 contiguous nucleotides of a regulatory RNA of human ABCB11, wherein the regulatory RNA has a nucleotide sequence selected from the group consisting of SEQ ID NOs: 1-5 and 7-10.

2. The ASO of claim 1, wherein the ASO is complementary to a sequence in the regRNA that is no more than 200 nucleotides from the 3′ end of the regRNA.

3. The ASO of claim 1, wherein the ASO is complementary to a sequence in the regRNA that is no more than 200 nucleotides from the 5′ end of the regRNA.

4. The ASO of claims 1-3, wherein the regRNA is not a polyadenylated RNA.

5. The ASO of any one of claims 1-4, wherein the regulatory RNA has a nucleotide sequence of SEQ ID NO: 1, and the ASO comprises a nucleotide sequence selected from the group consisting of SEQ ID NOs: 104-138, 217-272, 516-528, 645-1517, 5610-5644, 5723-5778, and 6018-6021.

6. The ASO of any one of claims 1-4, wherein the regulatory RNA has a nucleotide sequence of SEQ ID NO: 2, and the ASO comprises a nucleotide sequence selected from the group consisting of SEQ ID NOs: 14-103, 273-381, 390-407, 463, 467-478, 515, 644, 1518-2057, 5520-5609, 5779-5887, 5896-5913, 5969, 5971-5980, and 6017.

7. The ASO of any one of claims 1-4, wherein the regulatory RNA has a nucleotide sequence of SEQ ID NO: 3, and the ASO comprises a nucleotide sequence selected from the group consisting of SEQ ID NOs: 14-103, 139-148, 273-407, 463, 467-478, 515, 530-623, 625-644, 1518-2308, 5520-5609, 5645-5654, 5779-5913, 5969, 5971-5980, and 6017.

8. The ASO of any one of claims 1-4, wherein the regulatory RNA has a nucleotide sequence of SEQ ID NO: 4, and the ASO comprises a nucleotide sequence selected from the group consisting of SEQ ID NOs: 149-168, 408-462, 464, 479-514, 2309-3956, 5655-5674, 5914-5968, 5970, and 5981-6016.

9. The ASO of any one of claims 1-4, wherein the regulatory RNA has a nucleotide sequence of SEQ ID NO: 5, and the ASO comprises a nucleotide sequence selected from the group consisting of SEQ ID NOs: 169-216, 4062-5228, and 5675-5722.

10. The ASO of any one of claims 1-4, wherein the regulatory RNA has a nucleotide sequence of SEQ ID NO: 7, and the ASO comprises a nucleotide sequence selected from the group consisting of SEQ ID NOs: 645-954.

11. The ASO of any one of claims 1-4, wherein the regulatory RNA has a nucleotide sequence of SEQ ID NO: 8, and the ASO comprises a nucleotide sequence selected from the group consisting of SEQ ID NOs: 2309-2806.

12. The ASO of any one of claims 1-4, wherein the regulatory RNA has a nucleotide sequence of SEQ ID NO: 9, and the ASO comprises a nucleotide sequence selected from the group consisting of SEQ ID NOs: 3957-4061.

13. The ASO of any one of claims 1-4, wherein the regulatory RNA has a nucleotide sequence of SEQ ID NO: 10, and the ASO comprises a nucleotide sequence selected from the group consisting of SEQ ID NOs: 5229-5519.

14. The ASO of any one of claims 1-13, wherein the ASO is no more than 50, 40, 30, 25, 20, or 16 nucleotides in length.

15. The ASO of any one of claims 1-14, wherein the ASO comprises a RNA polynucleotide comprising one or more chemical modifications.

16. The ASO of claim 15, wherein at least 3, 4, or 5 nucleotides at the 5′ end and at least 3, 4, or 5 nucleotides at the 3′ end of the ASO comprise ribonucleotides with one or more chemical modifications.

17. The ASO of claim 15 or 16, wherein the one or more chemical modifications comprise a nucleotide sugar modification comprising one or more of 2′-O—C1-4alkyl such as 2′-O-methyl (2′-OMe), 2′-deoxy (2′-H), 2′-O—C1-3alkyl-O—C1-3alkyl such as 2′-methoxyethyl (“2′-MOE”), 2′-fluoro (“2′-F”), 2′-amino (“2′—NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (CET), 2′-fluoro-arabino (FANA), or thiomorpholino.

18. The ASO of any one of claims 15-17, wherein the one or more chemical modifications comprise an internucleotide linkage modification comprising one or more of phosphorothioate (“PS” or (P(S))), phosphoramidate (P(NR1R2) such as dimethylaminophosphoramidate (P(N(CH3)2)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2COO—)), alkylphosphonate (P(C1-3alkyl) such as methylphosphonate —P(CH3), boranophosphonate (P(BH3)), or phosphorodithioate (P(S)2).

19. The ASO of any one of claims 15-18, wherein the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

20. The ASO of any one of claims 15-19, wherein the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

21. The ASO of any one of claims 15-20, wherein the ASO further comprises a GalNAc moiety, optionally a GalNAc3 moiety.

22. The ASO of any one of claims 15-21, wherein the ASO does not comprise 10 or more contiguous nucleotides of unmodified DNA.

23. The ASO of claim 22, wherein the ASO does not comprise a deoxyribonucleotide.

24. The ASO of any one of claims 15-23, wherein the ASO does not comprise an unmodified ribonucleotide.

25. The ASO of any one of claims 15-24, wherein the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

26. The ASO of any one of claims 15-24, wherein the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

27. The ASO of any one of claims 15-24, wherein each ribonucleotide of the ASO is modified by 2′-O-methoxyethyl.

28. The ASO of any one of claims 15-24, wherein each nucleotide of the ASO is a ribonucleotide modified by 2′-O-methoxyethyl.

29. The ASO of any one of claims 15-28, wherein the ASO comprises 10 or more contiguous nucleotides of unmodified DNA flanked by at least 3 nucleotides of modified ribonucleotides at each of the 5′ end and the 3′ end.

30. The ASO of any one of claims 15-29, wherein each cytidine in the ASO is modified by 5-methyl.

31. The ASO of any one of claims 15-30, wherein the ASO comprises 2 or more contiguous nucleotides of unmodified DNA flanked by at least 3 nucleotides of modified ribonucleotides at each of the 5′ end and the 3′ end.

32. The ASO of any one of claim 1-5 or 8-30, wherein the regRNA is an eRNA.

33. The ASO of any one of claim 1-4, 6, 7, or 14-30, wherein the regRNA is an paRNA.

34. A pharmaceutical composition comprising the ASO of any one of claims 1-33 and a pharmaceutically acceptable carrier.

35. A method of increasing transcription of ABCB11 in a human cell, the method comprising contacting the cell with the ASO of any one of claims 1-33 or the pharmaceutical composition of claim 34.

36. The method of claim 35, wherein the cell is a hepatocyte.

37. The method of claim 35 or 36, wherein the ASO increases the amount of the regulatory RNA in the cell.

38. The method of any one of claims 35-37, wherein the ASO increases the stability of the regulatory RNA in the cell.

39. The method of any one of claims 35-38, wherein the method results in increased ABCB11 mRNA in the cell.

40. The method of any one of claims 35-39, wherein the method results in increased ABCB11 protein in the cell.

41. A method of treating or preventing cholestasis in a subject, the method comprising administering to a subject in need thereof an effective amount of the ASO of any one of claims 1-33 or the pharmaceutical composition of claim 34.

42. The method of claim 41, wherein the subject has or is at risk of developing a cholestatic liver disease.

43. The method of claim 41, wherein the cholestatic liver disease is progressive familial intrahepatic cholestasis (PFIC), Alagille Syndrome (ALGS), primary sclerosing cholangitis (PSC), neonatal sclerosing cholangitis, pediatric intrahepatic cholestasis (e.g., pediatric primary intrahepatic cholestasis or pediatric secondary intrahepatic cholestasis), benign recurrent intrahepatic cholestasis (BRIC), total parenteral nutrition associated cholestasis, paraneoplastic cholestasis, Stauffer syndrome, drug-associated cholestasis, infection-associated cholestasis, biliary atresia, intrahepatic cholestasis of pregnancy, primary biliary cholangitis (PBC), Dubin-Johnson syndrome, post-Kasai biliary atresia, post-liver transplantation biliary atresia, post-liver transplantation cholestasis, post-liver transplantation associated liver disease, intestinal failure associated liver disease, bile acid mediated liver injury, MRP3 deficiency syndrome, or gallstone disease.

44. The method of any one of claims 41-43, wherein the subject has or is at risk of developing PFIC, and wherein the PFIC is PFIC type 3.

45. The method of any one of claims 41-43, wherein the subject has or is at risk of developing PBC.

46. The method of any one of claims 43-45, wherein the method results in a reduction in a symptom or a change in a disease-relevant laboratory measure of a cholestatic liver disease in the subject.

47. The method of claim 46, wherein the reduction in a symptom or change in the disease-relevant laboratory measure of cholestatic liver disease comprises a reduction in serum bile acid concentration, an increase in serum concentration of 7α-hydroxy-4-cholesten-3-one (7αC4), an increase in the ratio of serum 7αC4: serum bile acid, an increase in fecal bile acid excretion, a reduction in pruritis, a reduction in serum alanine aminotransferase (ALT) concentration, or a combination thereof.

48. The method of claim 47, wherein administration of the ASO increases ABCB11 gene expression relative a pre-administration baseline level.
